(19) United States
(12) Patent Application Publication
(10) Pub. No.: US 2013/0326731 A1
(43) Pub. Date: Dec. 5, 2013

(54) METHODS AND COMPOSITIONS FOR WEED CONTROL

(71) Applicants: Daniel Ader, St. Louis, MO (US); John J. Finnessy, Des Peres, MO (US); Zhaoling Li, St. Charles, MO (US); Christina Marie Taylor, Chesterfield, MO (US); Jennifer Chou Taylor, Ballwin, MO (US); Ronak Hasmukh Shah, Chesterfield, MO (US); Nengbing Tao, O'Fallon, MO (US)

(72) Inventors: Daniel Ader, St. Louis, MO (US); John J. Finnessy, Des Peres, MO (US); Zhaoling Li, St. Charles, MO (US); Christina Marie Taylor, Chesterfield, MO (US); Jennifer Chou Taylor, Ballwin, MO (US); Ronak Hasmukh Shah, Chesterfield, MO (US); Nengbing Tao, O'Fallon, MO (US)

(21) Appl. No.: 13/612,995

(22) Filed: Sep. 13, 2012

Related U.S. Application Data
(60) Provisional application No. 61/534,097, filed on Sep. 13, 2011.

(51) Int. Cl. C12N 15/113; A01N 57/16; A01N 33/18; A01N 41/06

(52) U.S. Cl. CPC C12N 15/1137; A01N 41/06; A01N 57/16; A01N 33/18
USPC 800/286; 504/333; 504/118; 504/196; 504/347; 800/278; 800/285; 536/23.6; 435/91.1

(57) ABSTRACT

Novel compositions for use to enhance weed control. Specifically, methods and compositions that modulate dihydropteroate synthase in weed species. The present invention also provides for combinations of compositions and methods that enhance weed control.

This application claims benefit under 35 USC §119(e) of U.S. provisional application Ser. No. 61/534,097 filed Sep. 13, 2011, herein incorporated by reference in it's entirety. The sequence listing that is contained in the file named “40_{—}21(58641)B seq listing.txt”, which is 513,100 bytes (measured in operating system MS-Windows) and was created on 4 Sep. 2012, is filed herewith and incorporated herein by reference.

Table 1 is provided herewith as a part of this U.S. patent application via the USPTO's EFS system in file named “40_{—}21(58641)Btable 1.doxc” which is 39,989 bytes in size (measured in MS-Windows®). Table 1 (file “40_{—}21(58641)Btable1.doxc” comprises 54 sequences and is herein incorporated by reference in its entirety.
Table 2 is provided herewith as a part of this U.S. patent application via the USPTO's EFS system in file named “40_{—}21(58641)Btable2.doxc” which is 107,841 bytes in size (measured in MS-Windows®). Table 2 (file “40_{—}21(58641)Btable2.doxc” comprises 848 sequences and is herein incorporated by reference in its entirety.
Table 3 is provided herewith as a part of this U.S. patent application via the USPTO's EFS system in file named “40_{—}21(58641)Btable3.doxc” which is 21,718 bytes in size (measured in MS-Windows®). Table 3 (file “40_{—}21(58641)Btable3.doxc” comprises 269 sequences and is herein incorporated by reference in its entirety.

### FIELD

The methods and compositions generally relate to the field of weed management. More specifically, relate to 7,8-dihydropteroate synthase inhibitors (DHPS) genes in plants and compositions containing polynucleotide molecules for modulating and/or regulating their expression. Further provided are methods and compositions useful for weed control.

### BACKGROUND

Weeds are plants that compete with cultivated plants in an agronomic environment and cost farmers billions of dollars annually in crop losses and the expense of efforts to keep weeds under control. Weeds also serve as hosts for crop diseases and insect pests. The losses caused by weeds in agricultural production environments include decreases in crop yield, reduced crop quality, increased irrigation costs, increased harvesting costs, reduced land value, injury to livestock, and crop damage from insects and diseases harbored by the weeds. The principal means by which weeds cause these effects are: 1) competing with crop plants for water, nutrients, sunlight and other essentials for growth and development, 2) production of toxic or irritant chemicals that cause human or animal health problem, 3) production of immense quantities of seed or vegetative reproductive parts or both that contaminate agricultural products and perpetuate the species in agricultural lands, and 4) production on agricultural and nonagricultural lands of vast amounts of vegetation that must be disposed of. Herbicide tolerant weeds are a problem with nearly all herbicides in use, there is a need to effectively manage these weeds. There are over 365 weed biotypes currently identified as being herbicide resistant to one or more herbicides by the Herbicide Resistance Action Committee (HRAC), the North American Herbicide Resistance Action Committee (NAHRAC), and the Weed Science Society of America (WSSA).

The 7,8-dihydropteroate synthase inhibitors (DHPS) is an enzyme involved in folic acid synthesis which is needed for purine nucleotide biosynthesis. This enzyme is the target of herbicides that include the carbamate chemical family.

### BRIEF DESCRIPTION OF THE FIGURES

The following drawings form part of the present specification and are included to further demonstrate certain methods, compositions or results. They may be better understood by reference to one or more of these drawings in combination with the detailed description of specific embodiments presented herein. The invention can be more fully understood from the following description of the figures:

FIG. 1. Treatment with oligonucleotide pools followed by Prowl (pendimethalin)

FIG. 2. Treatment of Palmer Amaranth with 3 oligonucleotide pools followed by Prowl herbicide at 12 lb/ac rate

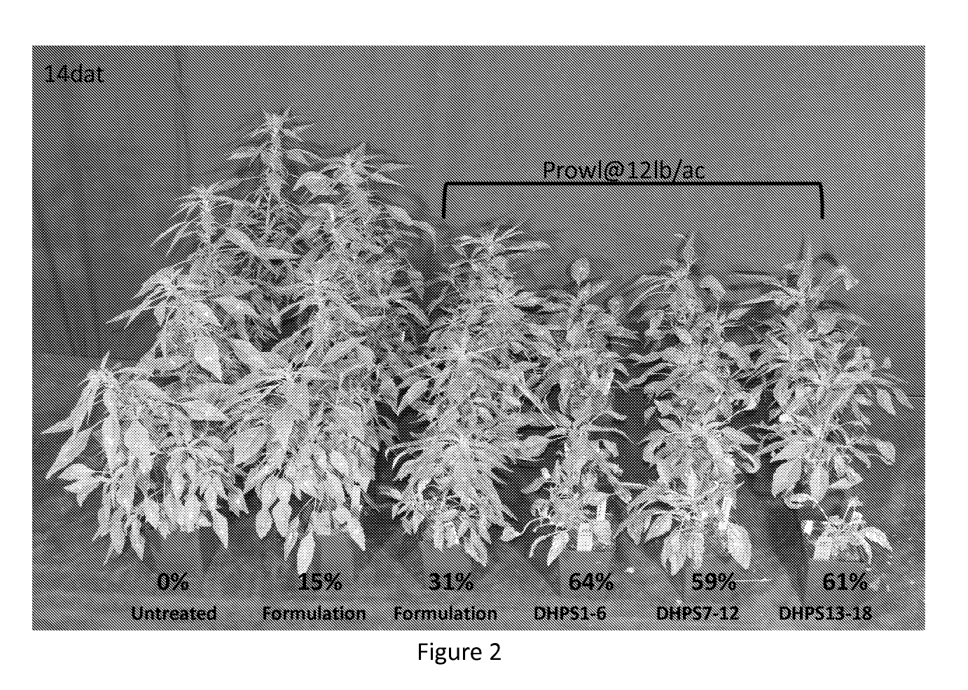

### SUMMARY

In one aspect, the invention provides a method of plant control comprising an external application to a plant or plant part a composition comprising a polynucleotide and a transfer agent, wherein the polynucleotide is essentially identical or essentially complementary to a DHPS (dihydropteroate synthase, DHP or DHPS) gene sequence or fragment thereof, or to the RNA transcript of said DHPS gene sequence or fragment thereof, wherein said DHPS gene sequence is selected from the group consisting of SEQ ID NO:1-54 or a polynucleotide fragment thereof. As a result of such application, the plant growth or development or reproductive ability is reduced or the plant is made more sensitive to a DHPS inhibitor herbicide relative to a plant not treated with said composition. In this manner, plants that have become resistant to the application of DHPS inhibitor containing herbicides are made more susceptible to the herbicidal effects the herbicides, thus potentiating the effect of the herbicides. The polynucleotide fragment is at least 18 contiguous nucleotides, at least 19 contiguous nucleotides, at least 20 contiguous nucleotides or at least 21 contiguous nucleotides in length and at least 85 percent identical to a DHPS gene sequence selected from the group consisting of SEQ ID NO:1-54 and the transfer agent comprises an organosilicone composition or compound. The polynucleotide fragment can be sense or anti-sense ssDNA or ssRNA, dsRNA, or dsDNA, or dsDNA/RNA hybrids. The composition can include various components that include more than one polynucleotide fragments, a DHPS inhibitor herbicide and/or other herbicides that enhance the plant control activity of the composition.

In another aspect, polynucleotide molecules and methods for modulating DHPS gene expression in a plant species are provided. The method reduces, represses or otherwise delays expression of a DHPS gene in a plant comprising an external application to such plant of a composition comprising a polynucleotide and a transfer agent, wherein the polynucleotide is essentially identical or essentially complementary to a DHPS gene sequence or fragment thereof, or to the RNA transcript of the DHPS gene sequence or fragment thereof, wherein the DHPS gene sequence is selected from the group consisting of SEQ ID NO:1-54 or a polynucleotide fragment thereof. The polynucleotide fragment is at least 18 contiguous nucleotides, at least 19 contiguous nucleotides, at least 20 contiguous nucleotides or at least 21 contiguous nucleotides in length and at least 85 percent identical to a DHPS gene sequence selected from the group consisting of SEQ ID NO:1-54 and the transfer agent is an organosilicone compound. The polynucleotide fragment can be sense or anti-sense ssDNA or ssRNA, dsRNA, or dsDNA, or dsDNA/RNA hybrids. Polynucleotide molecules comprising SEQ ID NOs 55-1175 are fragments of the DHPS gene.

In a further aspect, the polynucleotide molecule composition may be combined with other herbicidal compounds to provide additional control of unwanted plants in a field of cultivated plants.

In a further aspect, the polynucleotide molecule composition may be combined with any one or more additional agricultural chemicals, such as, insecticides, fungicides, nematocides, bactericides, acaricides, growth regulators, chemosterilants, semiochemicals, repellents, attractants, pheromones, feeding stimulants, biopesticides, microbial pesticides or other biologically active compounds to form a multi-component pesticide giving an even broader spectrum of agricultural protection.

### DETAILED DESCRIPTION

Provided are methods and compositions containing a polynucleotide that provide for regulation, repression or delay of 7,8-dihydropteroate synthase (DHPS) gene expression and enhanced control of weedy plant species and importantly DHPS inhibitor resistant weed biotypes. Aspects of the method can be applied to manage various weedy plants in agronomic and other cultivated environments.

The following definitions and methods are provided to better define the present invention and to guide those of ordinary skill in the art. Unless otherwise noted, terms are to be understood according to conventional usage by those of ordinary skill in the relevant art. Where a term is provided in the singular, the inventors also contemplate aspects described by the plural of that term.

By “non-transcribable” polynucleotides is meant that the polynucleotides do not comprise a complete polymerase II transcription unit.

As used herein “solution” refers to homogeneous mixtures and non-homogeneous mixtures such as suspensions, colloids, micelles, and emulsions.

Weedy plants are plants that compete with cultivated plants, those of particular importance include, but are not limited to important invasive and noxious weeds and herbicide resistant biotypes in crop production, such as, Amaranthus species—A. albus, A. blitoides, A. hybridus, A. palmeri, A. powellii, A. retroflexus, A. spinosus, A. tuberculatus, and A. viridis; Ambrosia species—A. trifida, A. artemisifolia; Lolium species—L. multiflorum, L. rigidium, L perenne; Digitaria species—D. insularis; Euphorbia species—E. heterophylla; Kochia species—K. scoparia; Sorghum species—S. halepense; Conyza species—C. bonariensis, C. canadensis, C. sumatrensis; Chloris species—C. truncate; Echinochola species—E. colona, E. crus-galli; Eleusine species—E. indica; Poa species—P. annua; Plantago species—P. lanceolata; Avena species—A. fatua; Chenopodium species—C. album; Setaria species—S. viridis, Abutilon theophrasti, Ipomoea species, Sesbania, species, Cassia species, Sida species, Brachiaria, species and Solanum species.

Additional weedy plant species found in cultivated areas include Alopecurus myosuroides, Avena sterilis, Avena sterilis ludoviciana, Brachiaria plantaginea, Bromus diandrus, Bromus rigidus, Cynosurus echinatus, Digitaria ciliaris, Digitaria ischaemum, Digitaria sanguinalis, Echinochloa oryzicola, Echinochloa phyllopogon, Eriochloa punctata, Hordeum glaucum, Hordeum leporinum, Ischaemum rugosum, Leptochloa chinensis, Lolium persicum, Phalaris minor, Phalaris paradoxa, Rottboellia exalta, Setaria faberi, Setaria viridis var, robusta-alba schreiber, Setaria viridis var, robusta-purpurea, Snowdenia polystachea, Sorghum sudanese, Alisma plantago-aquatica, Amaranthus lividus, Amaranthus quitensis, Ammania auriculata, Ammania coccinea, Anthemis cotula, Apera spica-venti, Bacopa rotundifolia, Bidens pilosa, Bidens subalternans, Brassica tournefortii, Bromus tectorum, Camelina microcarpa, Chrysanthemum coronarium, Cuscuta campestris, Cyperus difformis, Damasonium minus, Descurainia sophia, Diplotaxis tenuifolia, Echium plantagineum, Elatine triandra var, pedicellate, Euphorbia heterophylla, Fallopia convolvulus, Fimbristylis miliacea, Galeopsis tetrahit, Galium spurium, Helianthus annuus, Iva xanthifolia, Ixophorus unisetus, Ipomoea indica, Ipomoea purpurea, Ipomoea sepiaria, Ipomoea aquatic, Ipomoea triloba, Lactuca serriola, Limnocharis flava, Limnophila erecta, Limnophila sessiliflora, Lindernia dubia, Lindernia dubia var, major, Lindernia micrantha, Lindernia procumbens, Mesembryanthemum crystallinum, Monochoria korsakowii, Monochoria vaginalis, Neslia paniculata, Papaver rhoeas, Parthenium hysterophorus, Pentzia suffruticosa, Phalaris minor, Raphanus raphanistrum, Raphanus sativus, Rapistrum rugosum, Rotala indica var, uliginosa, Sagittaria guyanensis, Sagittaria montevidensis, Sagittaria pygmaea, Salsola iberica, Scirpus juncoides var, ohwianus, Scirpus mucronatus, Setaria lutescens, Sida spinosa, Sinapis arvensis, Sisymbrium orientale, Sisymbrium thellungii, Solanum ptycanthum, Sonchus aspen, Sonchus oleraceus, Sorghum bicolor, Stellaria media, Thlaspi arvense, Xanthium strumarium, Arctotheca calendula, Conyza sumatrensis, Crassocephalum crepidiodes, Cuphea carthagenenis, Epilobium adenocaulon, Erigeron philadelphicus, Landoltia punctata, Lepidium virginicum, Monochoria korsakowii, Solanum americanum, Solanum nigrum, Vulpia bromoides, Youngia japonica, Hydrilla verticillate, Carduus nutans, Carduus pycnocephalus, Centaurea solstitialis, Cirsium arvense, Commelina diffusa, Convolvulus arvensis, Daucus carota, Digitaria ischaemum, Echinochloa crus-pavonis, Fimbristylis miliacea, Galeopsis tetrahit, Galium spurium, Limnophila erecta, Matricaria perforate, Papaver rhoeas, Ranunculus acris, Soliva sessilis, Sphenoclea zeylanica, Stellaria media, Nassella trichotoma, Stipa neesiana, Agrostis stolonifera, Polygonum aviculare, Alopecurus japonicus, Beckmannia syzigachne, Bromus tectorum, Chloris inflate, Echinochloa erecta, Portulaca oleracea, and Senecio vulgaris. It is believed that all plants contain an DHPS gene in their genome, which can be isolated and polynucleotides made according to the methods that are useful for regulating, suppressing or delaying the expression of the target DHPS gene in the plants and the growth or development of the treated plants.

Some cultivated plants may also be weedy plants when they occur in unwanted environments. For example, corn plants growing in a soybean field. Transgenic crops with one or more herbicide tolerances will need specialized methods of management to control weeds and volunteer crop plants.

A “trigger” or “trigger polynucleotide” is a polynucleotide molecule that is homologous or complementary to a target gene polynucleotide. The trigger polynucleotide molecules modulate expression of the target gene when topically applied to a plant surface with a transfer agent, whereby a plant treated with said composition has its growth or development or reproductive ability regulated, suppressed or delayed or said plant is more sensitive to a DHPS inhibitor herbicide or mitosis inhibitor herbicide as a result of said polynucleotide containing composition relative to a plant not treated with a composition containing the trigger molecule. Trigger polynucleotides disclosed herein are generally described in relation to the target gene sequence and maybe used in the sense (homologous) or antisense (complementary) orientation as single stranded molecules or comprise both strands as double stranded molecules or nucleotide variants and modified nucleotides thereof depending on the various regions of a gene being targeted.

It is contemplated that the compositions will contain one or more polynucleotides and one or more herbicides that include but not limited to DHPS gene trigger polynucleotides and a DHPS inhibitor herbicide and anyone or more additional herbicide target gene trigger polynucleotides and the related herbicides and anyone or more additional essential gene trigger polynucleotides. Essential genes are genes in a plant that provide key enzymes or other proteins, for example, a biosynthetic enzyme, metabolizing enzyme, receptor, signal transduction protein, structural gene product, transcription factor, or transport protein; or regulating RNAs, such as, microRNAs, that are essential to the growth or survival of the organism or cell or involved in the normal growth and development of the plant (Meinke, et al., Trends Plant Sci. 2008 September; 13(9):483-91). The suppression of an essential gene enhances the effect of a herbicide that affects the function of a gene product different than the suppressed essential gene. The compositions can include various trigger polynucleotides that modulate the expression of an essential gene other than DHPS.

Herbicides, for which transgenes for plant tolerance have been demonstrated, include but are not limited to: auxin-like herbicides, glyphosate, glufosinate, sulfonylureas, imidazolinones, bromoxynil, delapon, dicamba, cyclohezanedione, protoporphyrionogen oxidase inhibitors, 4-hydroxyphenyl-pyruvate-dioxygenase inhibitors herbicides. For example, transgenes and their polynucleotide molecules that encode proteins involved in herbicide tolerance are known in the art, and include, but are not limited to an 5-enolpyruvylshikimate-3-phosphate synthase (EPSPS), for example, as more fully described in U.S. Pat. Nos. 7,807,791 (SEQ ID NO:5); 6,248,876 B1; 5,627,061; 5,804,425; 5,633,435; 5,145,783; 4,971,908; 5,392,910; 5,188,642; 4,940,835; 5,866,775; 6,225,114 B1; 6,130,366; 5,390,667; 4,535,060; 4,769,061; 5,633,448; 5,510,471; U.S. Pat. No. RE36,449; U.S. Pat. Nos. RE37,287 E; and 5,491,288; tolerance to sulfonylurea and/or imidazolinone, for example, as described more fully in U.S. Pat. Nos. 5,605,011; 5,013,659; 5,141,870; 5,767,361; 5,739,180; 5,304,732; 4,761,373; 5,339,107; 5,928,937; and 5,378,824; and international publication WO 96/33270; tolerance to hydroxyphenylpyruvatedioxygenases inhibitiong herbicides in plants are described in U.S. Pat. Nos. 6,245,968 B1; 6,268,549, and 6,069,115; US Pat. Pub. 20110191897 and U.S. Pat. No. 7,392,379 SEQ ID NO:3; U.S. Pat. No. 7,935,869; U.S. Pat. No. 7,304,209, SEQ ID NO:1, 3, 5 and 15; aryloxyalkanoate dioxygenase polynucleotides, which confer tolerance to 2,4-D and other phenoxy auxin herbicides as well as to aryloxyphenoxypropionate herbicides as described, for example, in WO2005/107437; U.S. Pat. No. 7,838,733 SEQ ID NO:5;) and dicamba-tolerance polynucleotides as described, for example, in Herman et al. (2005) J. Biol. Chem. 280: 24759-24767. Other examples of herbicide-tolerance traits include those conferred by polynucleotides encoding an exogenous phosphinothricin acetyltransferase, as described in U.S. Pat. Nos. 5,969,213; 5,489,520; 5,550,398; 5,874,265; 5,919,675; 5,561,236; 5,648,477; 5,639,024; 6,177,616; and 5,879,903. Plants containing an exogenous phosphinothricin acetyltransferase can exhibit improved tolerance to glufosinate herbicides, which inhibit the enzyme glutamine synthase. Additionally, herbicide-tolerance polynucleotides include those conferred by polynucleotides conferring altered protoporphyrinogen oxidase (protox) activity, as described in U.S. Pat. Nos. 6,288,306 B1; 6,282,837 B1; and 5,767,373; and WO 01/12825. Plants containing such polynucleotides can exhibit improved tolerance to any of a variety of herbicides which target the protox enzyme (also referred to as protox inhibitors). Polynucleotides encoding a glyphosate oxidoreductase and a glyphosate-N-acetyl transferase (GOX described in U.S. Pat. No. 5,393,175 and GAT described in U.S. Patent publication 20030083480, dicamba monooxygenase U.S. Patent publication 20030135879, all of which are incorporated herein by reference); a polynucleotide molecule encoding bromoxynil nitrilase (Bxn described in U.S. Pat. No. 4,810,648 for Bromoxynil tolerance, which is incorporated herein by reference); a polynucleotide molecule encoding phytoene desaturase (crtI) described in Misawa et al, (1993) Plant J. 4:833-840 and Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:398-2193 for tolerance to sulfonylurea herbicides; and the bar gene described in DeBlock, et al. (1987) EMBO J. 6:2513-2519 for glufosinate and bialaphos tolerance. The transgenic coding regions and regulatory elements of the herbicide tolerance genes are targets in which polynucleotide triggers and herbicides can be included in the compositions.

DHPS inhibitor herbicides include but are not limited to carbamates and asulam. Mitosis inhibitor herbicides include but are not limited to dinitroaniline herbicides for example benfluralin, butralin, dinitramine, ethalfluralin, oryzalin, pendimethalin, and trifluralin. Additional mitosis inhibitor herbicides also include but are not limited to Phosphoroamidates, Pyridines, Benzamides, and Benzenedicarboxylic acids.

Numerous herbicides with similar or different modes of action (herein referred to as co-herbicides) are available that can be added to the compositions, for example, members of the herbicide families that include but are not limited to amide herbicides, aromatic acid herbicides, arsenical herbicides, benzothiazole herbicides, benzoylcyclohexanedione herbicides, benzofuranyl alkylsulfonate herbicides, carbamate herbicides, cyclohexene oxime herbicides, cyclopropylisoxazole herbicides, dicarboximide herbicides, dinitroaniline herbicides, dinitrophenol herbicides, diphenyl ether herbicides, dithiocarbamate herbicides, halogenated aliphatic herbicides, imidazolinone herbicides, inorganic herbicides, nitrile herbicides, organophosphorus herbicides, oxadiazolone herbicides, oxazole herbicides, phenoxy herbicides, phenylenediamine herbicides, pyrazole herbicides, pyridazine herbicides, pyridazinone herbicides, pyridine herbicides, pyrimidinediamine herbicides, pyrimidinyloxybenzylamine herbicides, quaternary ammonium herbicides, thiocarbamate herbicides, thiocarbonate herbicides, thiourea herbicides, triazine herbicides, triazinone herbicides, triazole herbicides, triazolone herbicides, triazolopyrimidine herbicides, uracil herbicides, and urea herbicides. In particular, the rates of use of the added herbicides can be reduced in compositions comprising the polynucleotides. Use rate reductions of the additional added herbicides can be 10-25 percent, 26-50 percent, 51-75 percent or more can be achieved that enhance the activity of the polynucleotides and herbicide composition and is contemplated. Representative co-herbicides of the families include but are not limited to acetochlor, acifluorfen, acifluorfen-sodium, aclonifen, acrolein, alachlor, alloxydim, allyl alcohol, ametryn, amicarbazone, amidosulfuron, aminopyralid, amitrole, ammonium sulfamate, anilofos, asulam, atraton, atrazine, azimsulfuron, BCPC, beflubutamid, benazolin, benfluralin, benfuresate, bensulfuron, bensulfuron-methyl, bensulide, bentazone, benzfendizone, benzobicyclon, benzofenap, bifenox, bilanafos, bispyribac, bispyribac-sodium, borax, bromacil, bromobutide, bromoxynil, butachlor, butafenacil, butamifos, butralin, butroxydim, butylate, cacodylic acid, calcium chlorate, cafenstrole, carbetamide, carfentrazone, carfentrazone-ethyl, CDEA, CEPC, chlorflurenol, chlorflurenol-methyl, chloridazon, chlorimuron, chlorimuron-ethyl, chloroacetic acid, chlorotoluron, chlorpropham, chlorsulfuron, chlorthal, chlorthal-dimethyl, cinidon-ethyl, cinmethylin, cinosulfuron, cisanilide, clethodim, clodinafop, clodinafop-propargyl, clomazone, clomeprop, clopyralid, cloransulam, cloransulam-methyl, CMA, 4-CPB, CPMF, 4-CPP, CPPC, cresol, cumyluron, cyanamide, cyanazine, cycloate, cyclosulfamuron, cycloxydim, cyhalofop, cyhalofop-butyl, 2,4-D, 3,4-DA, daimuron, dalapon, dazomet, 2,4-DB, 3,4-DB, 2,4-DEB, desmedipham, dicamba, dichlobenil, ortho-dichlorobenzene, para-dichlorobenzene, dichlorprop, dichlorprop-P, diclofop, diclofop-methyl, diclosulam, difenzoquat, difenzoquat metilsulfate, diflufenican, diflufenzopyr, dimefuron, dimepiperate, dimethachlor, dimethametryn, dimethenamid, dimethenamid-P, dimethipin, dimethylarsinic acid, dinitramine, dinoterb, diphenamid, diquat, diquat dibromide, dithiopyr, diuron, DNOC, 3,4-DP, DSMA, EBEP, endothal, EPTC, esprocarb, ethalfluralin, ethametsulfuron, ethametsulfuron-methyl, ethofumesate, ethoxyfen, ethoxysulfuron, etobenzanid, fenoxaprop-P, fenoxaprop-P-ethyl, fentrazamide, ferrous sulfate, flamprop-M, flazasulfuron, florasulam, fluazifop, fluazifop-butyl, fluazifop-P, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, fluoroglycofen, fluoroglycofen-ethyl, flupropanate, flupyrsulfuron, flupyrsulfuron-methyl-sodium, flurenol, fluridone, fluorochloridone, fluoroxypyr, flurtamone, fluthiacet, fluthiacet-methyl, fomesafen, foramsulfuron, fosamine, glufosinate, glufosinate-ammonium, glyphosate, halosulfuron, halosulfuron-methyl, haloxyfop, haloxyfop-P, HC-252, hexazinone, imazamethabenz, imazamethabenz-methyl, imazamox, imazapic, imazapyr, imazaquin, imazethapyr, imazosulfuron, indanofan, iodomethane, iodosulfuron, iodosulfuron-methyl-sodium, ioxynil, isoproturon, isouron, isoxaben, isoxachlortole, isoxaflutole, karbutilate, lactofen, lenacil, linuron, MAA, MAMA, MCPA, MCPA-thioethyl, MCPB, mecoprop, mecoprop-P, mefenacet, mefluidide, mesosulfuron, mesosulfuron-methyl, mesotrione, metam, metamifop, metamitron, metazachlor, methabenzthiazuron, methylarsonic acid, methyldymron, methyl isothiocyanate, metobenzuron, metolachlor, S-metolachlor, metosulam, metoxuron, metribuzin, metsulfuron, metsulfuron-methyl, MK-66, molinate, monolinuron, MSMA, naproanilide, napropamide, naptalam, neburon, nicosulfuron, nonanoic acid, norflurazon, oleic acid (fatty acids), orbencarb, orthosulfamuron, oryzalin, oxadiargyl, oxadiazon, oxasulfuron, oxaziclomefone, oxyfluorfen, paraquat, paraquat dichloride, pebulate, pendimethalin, penoxsulam, pentachlorophenol, pentanochlor, pentoxazone, pethoxamid, petrolium oils, phenmedipham, phenmedipham-ethyl, picloram, picolinafen, pinoxaden, piperophos, potassium arsenite, potassium azide, pretilachlor, primisulfuron, primisulfuron-methyl, prodiamine, profluazol, profoxydim, prometon, prometryn, propachlor, propanil, propaquizafop, propazine, propham, propisochlor, propoxycarbazone, propoxycarbazone-sodium, propyzamide, prosulfocarb, prosulfuron, pyraclonil, pyraflufen, pyraflufen-ethyl, pyrazolynate, pyrazosulfuron, pyrazosulfuron-ethyl, pyrazoxyfen, pyribenzoxim, pyributicarb, pyridafol, pyridate, pyriftalid, pyriminobac, pyriminobac-methyl, pyrimisulfan, pyrithiobac, pyrithiobac-sodium, quinclorac, quinmerac, quinoclamine, quizalofop, quizalofop-P, rimsulfuron, sethoxydim, siduron, simazine, simetryn, SMA, sodium arsenite, sodium azide, sodium chlorate, sulcotrione, sulfentrazone, sulfometuron, sulfometuron-methyl, sulfosate, sulfosulfuron, sulfuric acid, tar oils, 2,3,6-TBA, TCA, TCA-sodium, tebuthiuron, tepraloxydim, terbacil, terbumeton, terbuthylazine, terbutryn, thenylchlor, thiazopyr, thifensulfuron, thifensulfuron-methyl, thiobencarb, tiocarbazil, topramezone, tralkoxydim, tri-allate, triasulfuron, triaziflam, tribenuron, tribenuron-methyl, tricamba, triclopyr, trietazine, trifloxysulfuron, trifloxysulfuron-sodium, trifluralin, triflusulfuron, triflusulfuron-methyl, trihydroxytriazine, tritosulfuron, [3-[2-chloro-4-fluoro-5-(-methyl-6-trifluoromethyl-2,4-dioxo-,2,3,4-t-etrahydropyrimidin-3-yl)phenoxy]-2-pyridyloxy]acetic acid ethyl ester (CAS RN 353292-3-6), 4-[(4,5-dihydro-3-methoxy-4-methyl-5-oxo)-H-,2,4-triazol-1-ylcarbonyl-sulfamoyl]-5-methylthiophene-3-carboxylic acid (BAY636), BAY747 (CAS RN 33504-84-2), topramezone (CAS RN 2063-68-8), 4-hydroxy-3-[[2-[(2-methoxyethoxy)methyl]-6-(trifluoro-methyl)-3-pyridinyl]carbonyl]-bicyclo[3.2.]oct-3-en-2-one (CAS RN 35200-68-5), and 4-hydroxy-3-[[2-(3-methoxypropyl)-6-(difluoromethyl)-3-pyridinyl]carbon-yl]-bicyclo[3.2.]oct-3-en-2-one. Additionally, including herbicidal compounds of unspecified modes of action as described in CN101279950A, CN101279951A, DE10000600A1, DE10116399A1, DE102004054666A1, DE102005014638A1, DE102005014906A1, DE102007012168A1, DE102010042866A1, DE10204951A1, DE10234875A1, DE10234876A1, DE10256353A1, DE10256354A1, DE10256367A1, EP1157991A2, EP1238586A1, EP2147919A1, EP2160098A2, JP03968012B2, JP2001253874A, JP2002080454A, JP2002138075A, JP2002145707A, JP2002220389A, JP2003064059A, JP2003096059A, JP2004051628A, JP2004107228A, JP2005008583A, JP2005239675A, JP2005314407A, JP2006232824A, JP2006282552A, JP2007153847A, JP2007161701A, JP2007182404A, JP2008074840A, JP2008074841A, JP2008133207A, JP2008133218A, JP2008169121A, JP2009067739A, JP2009114128A, JP2009126792A, JP2009137851A, US20060111241A1, US20090036311A1, US20090054240A1, US20090215628A1, US20100099561A1, US20100152443A1, US20110105329A1, US20110201501A1, WO2001055066A2, WO2001056975A1, WO2001056979A1, WO2001090071A2, WO2001090080A1, WO2002002540A1, WO2002028182A1, WO2002040473A1, WO2002044173A2, WO2003000679A2, WO2003006422A1, WO2003013247A1, WO2003016308A1, WO2003020704A1, WO2003022051A1, WO2003022831A1, WO2003022843A1, WO2003029243A2, WO2003037085A1, WO2003037878A1, WO2003045878A2, WO2003050087A2, WO2003051823A1, WO2003051824A1, WO2003051846A2, WO2003076409A1, WO2003087067A1, WO2003090539A1, WO2003091217A1, WO2003093269A2, WO2003104206A2, WO2004002947A1, WO2004002981A2, WO2004011429A1, WO2004029060A1, WO2004035545A2, WO2004035563A1, WO2004035564A1, WO2004037787A1, WO2004067518A1, WO2004067527A1, WO2004077950A1, WO2005000824A1, WO2005007627A1, WO2005040152A1, WO2005047233A1, WO2005047281A1, WO2005061443A2, WO2005061464A1, WO2005068434A1, WO2005070889A1, WO2005089551A1, WO2005095335A1, WO2006006569A1, WO2006024820A1, WO2006029828A1, WO2006029829A1, WO2006037945A1, WO2006050803A1, WO2006090792A1, WO2006123088A2, WO2006125687A1, WO2006125688A1, WO2007003294A1, WO2007026834A1, WO2007071900A1, WO2007077201A1, WO2007077247A1, WO2007096576A1, WO2007119434A1, WO2007134984A1, WO2008009908A1, WO2008029084A1, WO2008059948A1, WO2008071918A1, WO2008074991A1, WO2008084073A1, WO2008100426A2, WO2008102908A1, WO2008152072A2, WO2008152073A2, WO2009000757A1, WO2009005297A2, WO2009035150A2, WO2009063180A1, WO2009068170A2, WO2009068171A2, WO2009086041A1, WO2009090401A2, WO2009090402A2, WO2009115788A1, WO2009116558A1, WO2009152995A1, WO2009158258A1, WO2010012649A1, WO2010012649A1, WO2010026989A1, WO2010034153A1, WO2010049270A1, WO2010049369A1, WO2010049405A1, WO2010049414A1, WO2010063422A1, WO2010069802A1, WO2010078906A2, WO2010078912A1, WO2010104217A1, WO2010108611A1, WO2010112826A3, WO2010116122A3, WO2010119906A1, WO2010130970A1, WO2011003776A2, WO2011035874A1, WO2011065451A1, all of which are incorporated herein by reference.

An agronomic field in need of plant control is treated by application of the composition directly to the surface of the growing plants, such as by a spray. For example, the method is applied to control weeds in a field of crop plants by spraying the field with the composition. The composition can be provided as a tank mix, a sequential treatment of components (generally the polynucleotide containing composition followed by the herbicide), or a simultaneous treatment or mixing of one or more of the components of the composition from separate containers. Treatment of the field can occur as often as needed to provide weed control and the components of the composition can be adjusted to target specific weed species or weed families through utilization of specific polynucleotides or polynucleotide compositions capable of selectively targeting the specific species or plant family to be controlled. The composition can be applied at effective use rates according to the time of application to the field, for example, preplant, at planting, post planting, postharvest. DHPS inhibitor herbicides can be applied to a field at rates of 500 to 3000 g ai/ha (active ingredient per hectare) or more. The polynucleotides of the composition can be applied at rates of 1 to 30 grams per acre depending on the number of trigger molecules needed for the scope of weeds in the field.

Crop plants in which weed control is needed include but are not limited to, i) corn, soybean, cotton, canola, sugar beet, alfalfa, sugarcane, rice, and wheat; ii) vegetable plants including, but not limited to, tomato, sweet pepper, hot pepper, melon, watermelon, cucumber, eggplant, cauliflower, broccoli, lettuce, spinach, onion, peas, carrots, sweet corn, Chinese cabbage, leek, fennel, pumpkin, squash or gourd, radish, Brussels sprouts, tomatillo, garden beans, dry beans, or okra; iii) culinary plants including, but not limited to, basil, parsley, coffee, or tea; or, iv) fruit plants including but not limited to apple, pear, cherry, peach, plum, apricot, banana, plantain, table grape, wine grape, citrus, avocado, mango, or berry; v) a tree grown for ornamental or commercial use, including, but not limited to, a fruit or nut tree; or, vi) an ornamental plant (e.g., an ornamental flowering plant or shrub or turf grass). The methods and compositions provided herein can also be applied to plants produced by a cutting, cloning, or grafting process (i.e., a plant not grown from a seed) include fruit trees and plants that include, but are not limited to, citrus, apples, avocados, tomatoes, eggplant, cucumber, melons, watermelons, and grapes as well as various ornamental plants.

### Pesticidal Mixtures

The polynucleotide compositions may also be used as mixtures with various agricultural chemicals and/or insecticides, miticides and fungicides, pesticidal and biopesticidal agents. Examples include but are not limited to azinphos-methyl, acephate, isoxathion, isofenphos, ethion, etrimfos, oxydemeton-methyl, oxydeprofos, quinalphos, chlorpyrifos, chlorpyrifos-methyl, chlorfenvinphos, cyanophos, dioxabenzofos, dichlorvos, disulfoton, dimethylvinphos, dimethoate, sulprofos, diazinon, thiometon, tetrachlorvinphos, temephos, tebupirimfos, terbufos, naled, vamidothion, pyraclofos, pyridafenthion, pirimiphos-methyl, fenitrothion, fenthion, phenthoate, flupyrazophos, prothiofos, propaphos, profenofos, phoxime, phosalone, phosmet, formothion, phorate, malathion, mecarbam, mesulfenfos, methamidophos, methidathion, parathion, methyl parathion, monocrotophos, trichlorphon, EPN, isazophos, isamidofos, cadusafos, diamidaphos, dichlofenthion, thionazin, fenamiphos, fosthiazate, fosthietan, phosphocarb, DSP, ethoprophos, alanycarb, aldicarb, isoprocarb, ethiofencarb, carbaryl, carbosulfan, xylylcarb, thiodicarb, pirimicarb, fenobucarb, furathiocarb, propoxur, bendiocarb, benfuracarb, methomyl, metolcarb, XMC, carbofuran, aldoxycarb, oxamyl, acrinathrin, allethrin, esfenvalerate, empenthrin, cycloprothrin, cyhalothrin, gamma-cyhalothrin, lambda-cyhalothrin, cyfluthrin, beta-cyfluthrin, cypermethrin, alpha-cypermethrin, zeta-cypermethrin, silafluofen, tetramethrin, tefluthrin, deltamethrin, tralomethrin, bifenthrin, phenothrin, fenvalerate, fenpropathrin, furamethrin, prallethrin, flucythrinate, fluvalinate, flubrocythrinate, permethrin, resmethrin, ethofenprox, cartap, thiocyclam, bensultap, acetamiprid, imidacloprid, clothianidin, dinotefuran, thiacloprid, thiamethoxam, nitenpyram, chlorfluazuron, diflubenzuron, teflubenzuron, triflumuron, novaluron, noviflumuron, bistrifluoron, fluazuron, flucycloxuron, flufenoxuron, hexaflumuron, lufenuron, chromafenozide, tebufenozide, halofenozide, methoxyfenozide, diofenolan, cyromazine, pyriproxyfen, buprofezin, methoprene, hydroprene, kinoprene, triazamate, endosulfan, chlorfenson, chlorobenzilate, dicofol, bromopropylate, acetoprole, fipronil, ethiprole, pyrethrin, rotenone, nicotine sulphate, BT (Bacillus Thuringiensis) agent, spinosad, abamectin, acequinocyl, amidoflumet, amitraz, etoxazole, chinomethionat, clofentezine, fenbutatin oxide, dienochlor, cyhexatin, spirodiclofen, spiromesifen, tetradifon, tebufenpyrad, binapacryl, bifenazate, pyridaben, pyrimidifen, fenazaquin, fenothiocarb, fenpyroximate, fluacrypyrim, fluazinam, flufenzin, hexythiazox, propargite, benzomate, polynactin complex, milbemectin, lufenuron, mecarbam, methiocarb, mevinphos, halfenprox, azadirachtin, diafenthiuron, indoxacarb, emamectin benzoate, potassium oleate, sodium oleate, chlorfenapyr, tolfenpyrad, pymetrozine, fenoxycarb, hydramethylnon, hydroxy propyl starch, pyridalyl, flufenerim, flubendiamide, flonicamid, metaflumizole, lepimectin, TPIC, albendazole, oxibendazole, oxfendazole, trichlamide, fensulfothion, fenbendazole, levamisole hydrochloride, morantel tartrate, dazomet, metam-sodium, triadimefon, hexaconazole, propiconazole, ipconazole, prochloraz, triflumizole, tebuconazole, epoxiconazole, difenoconazole, flusilazole, triadimenol, cyproconazole, metconazole, fluquinconazole, bitertanol, tetraconazole, triticonazole, flutriafol, penconazole, diniconazole, fenbuconazole, bromuconazole, imibenconazole, simeconazole, myclobutanil, hymexazole, imazalil, furametpyr, thifluzamide, etridiazole, oxpoconazole, oxpoconazole fumarate, pefurazoate, prothioconazole, pyrifenox, fenarimol, nuarimol, bupirimate, mepanipyrim, cyprodinil, pyrimethanil, metalaxyl, mefenoxam, oxadixyl, benalaxyl, thiophanate, thiophanate-methyl, benomyl, carbendazim, fuberidazole, thiabendazole, manzeb, propineb, zineb, metiram, maneb, ziram, thiuram, chlorothalonil, ethaboxam, oxycarboxin, carboxin, flutolanil, silthiofam, mepronil, dimethomorph, fenpropidin, fenpropimorph, spiroxamine, tridemorph, dodemorph, flumorph, azoxystrobin, kresoxim-methyl, metominostrobin, orysastrobin, fluoxastrobin, trifloxystrobin, dimoxystrobin, pyraclostrobin, picoxystrobin, iprodione, procymidone, vinclozolin, chlozolinate, flusulfamide, dazomet, methyl isothiocyanate, chloropicrin, methasulfocarb, hydroxyisoxazole, potassium hydroxyisoxazole, echlomezol, D-D, carbam, basic copper chloride, basic copper sulfate, copper nonylphenolsulfonate, oxine copper, DBEDC, anhydrous copper sulfate, copper sulfate pentahydrate, cupric hydroxide, inorganic sulfur, wettable sulfur, lime sulfur, zinc sulfate, fentin, sodium hydrogen carbonate, potassium hydrogen carbonate, sodium hypochlorite, silver, edifenphos, tolclofos-methyl, fosetyl, iprobenfos, dinocap, pyrazophos, carpropamid, fthalide, tricyclazole, pyroquilon, diclocymet, fenoxanil, kasugamycin, validamycin, polyoxins, blasticiden S, oxytetracycline, mildiomycin, streptomycin, rape seed oil, machine oil, benthiavalicarbisopropyl, iprovalicarb, propamocarb, diethofencarb, fluoroimide, fludioxanil, fenpiclonil, quinoxyfen, oxolinic acid, chlorothalonil, captan, folpet, probenazole, acibenzolar-S-methyl, tiadinil, cyflufenamid, fenhexamid, diflumetorim, metrafenone, picobenzamide, proquinazid, famoxadone, cyazofamid, fenamidone, zoxamide, boscalid, cymoxanil, dithianon, fluazinam, dichlofluanide, triforine, isoprothiolane, ferimzone, diclomezine, tecloftalam, pencycuron, chinomethionat, iminoctadine acetate, iminoctadine albesilate, ambam, polycarbamate, thiadiazine, chloroneb, nickel dimethyldithiocarbamate, guazatine, dodecylguanidine-acetate, quintozene, tolylfluanid, anilazine, nitrothalisopropyl, fenitropan, dimethirimol, benthiazole, harpin protein, flumetover, mandipropamide and penthiopyrad.

### Polynucleotides

As used herein, the term “DNA”, “DNA molecule”, “DNA polynucleotide molecule” refers to a single-stranded DNA (ssDNA) or double-stranded DNA (dsDNA) molecule of genomic or synthetic origin, such as, a polymer of deoxyribonucleotide bases or a DNA polynucleotide molecule. As used herein, the term “DNA sequence”, “DNA nucleotide sequence” or “DNA polynucleotide sequence” refers to the nucleotide sequence of a DNA molecule. As used herein, the term “RNA”, “RNA molecule”, “RNA polynucleotide molecule” refers to a single-stranded RNA (ssRNA) or double-stranded RNA (dsRNA) molecule of genomic or synthetic origin, such as, a polymer of ribonucleotide bases that comprise single or double stranded regions. Unless otherwise stated, nucleotide sequences in the text of this specification are given, when read from left to right, in the 5′ to 3′ direction. The nomenclature used herein is that required by Title 37 of the United States Code of Federal Regulations §1.822 and set forth in the tables in WIPO Standard ST.25 (1998), Appendix 2, Tables 1 and 3.

As used herein, “polynucleotide” refers to a DNA or RNA molecule containing multiple nucleotides and generally refers both to “oligonucleotides” (a polynucleotide molecule of typically 50 or fewer nucleotides in length) and polynucleotides of 51 or more nucleotides. Embodiments include compositions including oligonucleotides having a length of 18-25 nucleotides (18-mers, 19-mers, 20-mers, 21-mers, 22-mers, 23-mers, 24-mers, or 25-mers), for example, oligonucleotides of Table 3 (SEQ ID NO:907-1175) pr fragments thereof or medium-length polynucleotides having a length of 26 or more nucleotides (polynucleotides of 26, 27, 28, 29, 30, 39, 32, 33, 34, 35, 36, 37, 38, 39, 40, 41, 42, 43, 44, 45, 39, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), for example, polynucleotides of Table 2 (SEQ ID NO: 55-906) or fragments thereof or long polynucleotides having a length greater than about 300 nucleotides (for example, polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length, for example up to the entire length of a target gene including coding or non-coding or both coding and non-coding portions of the target gene), for example, polynucleotides of Table 1 (SEQ ID NO:1-54), wherein the selected polynucleotides or fragments thereof homologous or complementary to SEQ ID NO:1-54 suppresses, represses or otherwise delay the expression of the target DHPS gene. A target gene comprises any polynucleotide molecule in a plant cell or fragment thereof for which the modulation of the expression of the target gene is provided by the methods and compositions. Where a polynucleotide is double-stranded, its length can be similarly described in terms of base pairs. Oligonucleotides and polynucleotides can be made that are essentially identical or essentially complementary to adjacent genetic elements of a gene, for example, spanning the junction region of an intron and exon, the junction region of a promoter and a transcribed region, the junction region of a 5′ leader and a coding sequence, the junction of a 3′ untranslated region and a coding sequence.

Polynucleotide compositions used in the various embodiments include compositions including oligonucleotides or polynucleotides or a mixture of both, including RNA or DNA or RNA/DNA hybrids or chemically modified oligonucleotides or polynucleotides or a mixture thereof. In some embodiments, the polynucleotide may be a combination of ribonucleotides and deoxyribonucleotides, for example, synthetic polynucleotides consisting mainly of ribonucleotides but with one or more terminal deoxyribonucleotides or synthetic polynucleotides consisting mainly of deoxyribonucleotides but with one or more terminal dideoxyribonucleotides. In some embodiments, the polynucleotide includes non-canonical nucleotides such as inosine, thiouridine, or pseudouridine. In some embodiments, the polynucleotide includes chemically modified nucleotides. Examples of chemically modified oligonucleotides or polynucleotides are well known in the art; see, for example, US Patent Publication 20110171287, US Patent Publication 20110171176, and US Patent Publication 20110152353, US Patent Publication, 20110152339, US Patent Publication 20110160082, herein incorporated by reference. For example, including but not limited to the naturally occurring phosphodiester backbone of an oligonucleotide or polynucleotide can be partially or completely modified with phosphorothioate, phosphorodithioate, or methylphosphonate internucleotide linkage modifications, modified nucleoside bases or modified sugars can be used in oligonucleotide or polynucleotide synthesis, and oligonucleotides or polynucleotides can be labeled with a fluorescent moiety (for example, fluorescein or rhodamine) or other label (for example, biotin).

The polynucleotides can be single- or double-stranded RNA or single- or double-stranded DNA or double-stranded DNA/RNA hybrids or modified analogues thereof, and can be of oligonucleotide lengths or longer. In more specific embodiments, the polynucleotides that provide single-stranded RNA in the plant cell are selected from the group consisting of (a) a single-stranded RNA molecule (ssRNA), (b) a single-stranded RNA molecule that self-hybridizes to form a double-stranded RNA molecule, (c) a double-stranded RNA molecule (dsRNA), (d) a single-stranded DNA molecule (ssDNA), (e) a single-stranded DNA molecule that self-hybridizes to form a double-stranded DNA molecule, and (f) a single-stranded DNA molecule including a modified Pol III gene that is transcribed to an RNA molecule, (g) a double-stranded DNA molecule (dsDNA), (h) a double-stranded DNA molecule including a modified Pol III gene that is transcribed to an RNA molecule, (i) a double-stranded, hybridized RNA/DNA molecule, or combinations thereof. In some embodiments these polynucleotides include chemically modified nucleotides or non-canonical nucleotides. In some embodiments, the oligonucleotides may be blunt-ended or may comprise a 3′ overhang of from 1-5 nucleotides of at least one or both of the strands. Other configurations of the oligonucleotide are known in the field and are contemplated herein. In embodiments of the method the polynucleotides include double-stranded DNA formed by intramolecular hybridization, double-stranded DNA formed by intermolecular hybridization, double-stranded RNA formed by intramolecular hybridization, or double-stranded RNA formed by intermolecular hybridization. In one embodiment the polynucleotides include single-stranded DNA or single-stranded RNA that self-hybridizes to form a hairpin structure having an at least partially double-stranded structure including at least one segment that will hybridize to RNA transcribed from the gene targeted for suppression. Not intending to be bound by any mechanism, it is believed that such polynucleotides are or will produce single-stranded RNA with at least one segment that will hybridize to RNA transcribed from the gene targeted for suppression. In certain other embodiments the polynucleotides further includes a promoter, generally a promoter functional in a plant, for example, a pol II promoter, a pol III promoter, a pol IV promoter, or a pol V promoter.

The term “gene” refers to components that comprise chromosomal DNA, plasmid DNA, cDNA, intron and exon DNA, artificial DNA polynucleotide, or other DNA that encodes a peptide, polypeptide, protein, or RNA transcript molecule, and the genetic elements flanking the coding sequence that are involved in the regulation of expression, such as, promoter regions, 5′ leader regions, 3′ untranslated region that may exist as native genes or transgenes in a plant genome. The gene or a fragment thereof is isolated and subjected to polynucleotide sequencing methods that determines the order of the nucleotides that comprise the gene. Any of the components of the gene are potential targets for a trigger oligonucleotide and polynucleotides.

The trigger polynucleotide molecules are designed to modulate expression by inducing regulation or suppression of an endogenous DHPS gene in a plant and are designed to have a nucleotide sequence essentially identical or essentially complementary to the nucleotide sequence of an endogenous DHPS gene of a plant or to the sequence of RNA transcribed from an endogenous DHPS gene of a plant, including a transgene in a plant that provides for a herbicide resistant DHPS enzyme, which can be coding sequence or non-coding sequence. Effective molecules that modulate expression are referred to as “a trigger molecule, or trigger polynucleotides”. By “essentially identical” or “essentially complementary” is meant that the trigger polynucleotides (or at least one strand of a double-stranded polynucleotide or portion thereof, or a portion of a single strand polynucleotide) are designed to hybridize to the endogenous gene noncoding sequence or to RNA transcribed (known as messenger RNA or an RNA transcript) from the endogenous gene to effect regulation or suppression of expression of the endogenous gene. Trigger molecules are identified by “tiling” the gene targets with partially overlapping probes or non-overlapping probes of antisense or sense polynucleotides that are essentially identical or essentially complementary to the nucleotide sequence of an endogenous gene. Multiple target sequences can be aligned and sequence regions with homology in common are identified as potential trigger molecules for the multiple targets. Multiple trigger molecules of various lengths, for example 18-25 nucleotides, 26-50 nucleotides, 51-100 nucleotides, 101-200 nucleotides, 201-300 nucleotides or more can be pooled into a few treatments in order to investigate polynucleotide molecules that cover a portion of a gene sequence (for example, a portion of a coding versus a portion of a noncoding region, or a 5′ versus a 3′ portion of a gene) or an entire gene sequence including coding and noncoding regions of a target gene. Polynucleotide molecules of the pooled trigger molecules can be divided into smaller pools or single molecules inorder to identify trigger molecules that provide the desired effect.

The target gene RNA and DNA polynucleotide molecules (Table 1, SEQ ID NO:1-54) are sequenced by any number of available methods and equipment. Some of the sequencing technologies are available commercially, such as the sequencing-by-hybridization platform from Affymetrix Inc. (Sunnyvale, Calif.) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif.), as described below. In addition to the single molecule sequencing performed using sequencing-by-synthesis of Helicos Biosciences, other single molecule sequencing technologies are encompassed by the method and include the SMRT™ technology of Pacific Biosciences, the Ion Torrent™. technology, and nanopore sequencing being developed for example, by Oxford Nanopore Technologies. A DHPS target gene comprising DNA or RNA can be isolated using primers or probes essentially complementary or essentially homologous to SEQ ID NO:1-54 or a fragment thereof A polymerase chain reaction (PCR) gene fragment can be produced using primers essentially complementary or essentially homologous to SEQ ID NO:1-54 or a fragment thereof that is useful to isolate a DHPS gene from a plant genome.

Embodiments of functional single-stranded polynucleotides functional have sequence complementarity that need not be 100 percent, but is at least sufficient to permit hybridization to RNA transcribed from the target gene or DNA of the target gene to form a duplex to permit a gene silencing mechanism. Thus, in embodiments, a polynucleotide fragment is designed to be essentially identical to, or essentially complementary to, a sequence of 18 or more contiguous nucleotides in either the target DHPS gene sequence or messenger RNA transcribed from the target gene. By “essentially identical” is meant having 100 percent sequence identity or at least about 83, 84, 85, 86, 87, 88, 89, 90, 91, 92, 93, 94, 95, 96, 97, 98, or 99 percent sequence identity when compared to the sequence of 18 or more contiguous nucleotides in either the target gene or RNA transcribed from the target gene; by “essentially complementary” is meant having 100 percent sequence complementarity or at least about 83, 84, 85, 86, 87, 88, 89, 90, 91, 92, 93, 94, 95, 96, 97, 98, or 99 percent sequence complementarity when compared to the sequence of 18 or more contiguous nucleotides in either the target gene or RNA transcribed from the target gene. In some embodiments, polynucleotide molecules are designed to have 100 percent sequence identity with or complementarity to one allele or one family member of a given target gene (coding or non-coding sequence of a gene); in other embodiments the polynucleotide molecules are designed to have 100 percent sequence identity with or complementarity to multiple alleles or family members of a given target gene. The trigger polynucleotide sequences in the sequence listing SEQ ID NO: 1-1222 or table 1, 2 or 3 maybe complementary or homologous to a portion of the DHPS target gene sequence.

In certain embodiments, the polynucleotides used in the compositions that are essentially identical or essentially complementary to the target gene or transcript will comprise the predominant nucleic acid in the composition. Thus in certain embodiments, the polynucleotides that are essentially identical or essentially complementary to the target gene or transcript will comprise at least about 50%, 75%, 95%, 98% or 100% of the nucleic acids provided in the composition by either mass or molar concentration. However, in certain embodiments, the polynucleotides that are essentially identical or essentially complementary to the target gene or transcript can comprise at least about 1% to about 50%, about 10% to about 50%, about 20% to about 50%, or about 30% to about 50% of the nucleic acids provided in the composition by either mass or molar concentration. Also provided are compositions where the polynucleotides that are essentially identical or essentially complementary to the target gene or transcript can comprise at least about 1% to 100%, about 10% to 100%, about 20% to about 100%, about 30% to about 50%, or about 50% to a 100% of the nucleic acids provided in the composition by either mass or molar concentration.

“Identity” refers to the degree of similarity between two polynucleic acid or protein sequences. An alignment of the two sequences is performed by a suitable computer program. A widely used and accepted computer program for performing sequence alignments is CLUSTALW v1.6 (Thompson, et al. Nucl. Acids Res., 22: 3973-3980, 1994). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there are 100 matched amino acids between a 200 and a 400 amino acid protein, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

Trigger molecules for specific gene family members can be identified from coding and/or non-coding sequences of gene families of a plant or multiple plants, by aligning and selecting 200-300 polynucleotide fragments from the least homologous regions amongst the aligned sequences and evaluated using topically applied polynucleotides (as sense or anti-sense ssDNA or ssRNA, dsRNA, or dsDNA) to determine their relative effectiveness in inducing the herbicidal phenotype. The effective segments are further subdivided into 50-60 polynucleotide fragments, prioritized by least homology, and reevaluated using topically applied polynucleotides. The effective 50-60 polynucleotide fragments are subdivided into 19-30 polynucleotide fragments, prioritized by least homology, and again evaluated for induction of the yield/quality phenotype. Once relative effectiveness is determined, the fragments are utilized singly, or again evaluated in combination with one or more other fragments to determine the trigger composition or mixture of trigger polynucleotides for providing the yield/quality phenotype.

Trigger molecules for broad activity can be identified from coding and/or non-coding sequences of gene families of a plant or multiple plants, by aligning and selecting 200-300 polynucleotide fragments from the most homologous regions amongst the aligned sequences and evaluated using topically applied polynucleotides (as sense or anti-sense ssDNA or ssRNA, dsRNA, or dsDNA) to determine their relative effectiveness in inducing the yield/quality phenotype. The effective segments are subdivided into 50-60 polynucleotide fragments, prioritized by most homology, and reevaluated using topically applied polynucleotides. The effective 50-60 polynucleotide fragments are subdivided into 19-30 polynucleotide fragments, prioritized by most homology, and again evaluated for induction of the yield/quality phenotype. Once relative effectiveness is determined, the fragments may be utilized singly, or in combination with one or more other fragments to determine the trigger composition or mixture of trigger polynucleotides for providing the yield/quality phenotype.

Methods of making polynucleotides are well known in the art. Chemical synthesis, in vivo synthesis and in vitro synthesis methods and compositions are known in the art and include various viral elements, microbial cells, modified polymerases, and modified nucleotides. Commercial preparation of oligonucleotides often provides two deoxyribonucleotides on the 3′ end of the sense strand. Long polynucleotide molecules can be synthesized from commercially available kits, for example, kits from Applied Biosystems/Ambion (Austin, Tex.) have DNA ligated on the 5′ end in a microbial expression cassette that includes a bacterial T7 polymerase promoter that makes RNA strands that can be assembled into a dsRNA and kits provided by various manufacturers that include T7 RiboMax Express (Promega, Madison, Wis.), AmpliScribe T7-Flash (Epicentre, Madison, Wis.), and TranscriptAid T7 High Yield (Fermentas, Glen Burnie, Md.). dsRNA molecules can be produced from microbial expression cassettes in bacterial cells (Ongvarrasopone et al. ScienceAsia 33:35-39; Yin, Appl. Microbiol. Biotechno184:323-333, 2009; Liu et al., BMC Biotechnology 10:85, 2010) that have regulated or deficient RNase III enzyme activity or the use of various viral vectors to produce sufficient quantities of dsRNA. DHPS gene fragments are inserted into the microbial expression cassettes in a position in which the fragments are express to produce ssRNA or dsRNA useful in the methods described herein to regulate expression on a target DHPS gene. Long polynucleotide molecules can also be assembled from multiple RNA or DNA fragments. In some embodiments design parameters such as Reynolds score (Reynolds et al. Nature Biotechnology 22, 326-330 (2004), Tuschl rules (Pei and Tuschl, Nature Methods 3(9): 670-676, 2006), i-score (Nucleic Acids Res 35: e123, 2007), i-Score Designer tool and associated algorithms (Nucleic Acids Res 32: 936-948, 2004. Biochem Biophys Res Commun 316: 1050-1058, 2004, Nucleic Acids Res 32: 893-901, 2004, Cell Cycle 3: 790-5, 2004, Nat Biotechnol 23: 995-1001, 2005, Nucleic Acids Res 35: e27, 2007, BMC Bioinformatics 7: 520, 2006, Nucleic Acids Res 35: e123, 2007, Nat Biotechnol 22: 326-330, 2004) are known in the art and may be used in selecting polynucleotide sequences effective in gene silencing. In some embodiments random design or empirical selection of polynucleotide sequences is used in selecting polynucleotide sequences effective in gene silencing. In some embodiments the sequence of a polynucleotide is screened against the genomic DNA of the intended plant to minimize unintentional silencing of other genes.

The polynucleotide compositions are useful in compositions, such as solutions of polynucleotide molecules, at low concentrations, alone or in combination with other components either in the same solution or in separately applied solutions that provide a permeability-enhancing agent. While there is no upper limit on the concentrations and dosages of polynucleotide molecules that can useful in the methods, lower effective concentrations and dosages will generally be sought for efficiency. The concentrations can be adjusted in consideration of the volume of spray or treatment applied to plant leaves or other plant part surfaces, such as flower petals, stems, tubers, fruit, anthers, pollen, or seed. In one embodiment, a useful treatment for herbaceous plants using 25-mer oligonucleotide molecules is about 1 nanomole (nmol) of oligonucleotide molecules per plant, for example, from about 0.05 to 1 nmol per plant. Other embodiments for herbaceous plants include useful ranges of about 0.05 to about 100 nmol, or about 0.1 to about 20 nmol, or about 1 nmol to about 10 nmol of polynucleotides per plant. Very large plants, trees, or vines may require correspondingly larger amounts of polynucleotides. When using long dsRNA molecules that can be processed into multiple oligonucleotides, lower concentrations can be used. To illustrate embodiments, the factor IX, when applied to oligonucleotide molecules is arbitrarily used to denote a treatment of 0.8 nmol of polynucleotide molecule per plant; 10×, 8 nmol of polynucleotide molecule per plant; and 100×, 80 nmol of polynucleotide molecule per plant.

The trigger polynucleotide and oligonucleotide molecule compositions are useful in compositions, such as liquids that comprise these polynucleotide molecules, alone or in combination with other components, for example one or more herbicide molecules, either in the same liquid or in separately applied liquids that also provide a transfer agent. As used herein, a transfer agent is an agent that, when combined with a polynucleotide in a composition that is topically applied to a target plant surface, enables the polynucleotide to enter a plant cell. In certain embodiments, a transfer agent is an agent that conditions the surface of plant tissue, e.g., leaves, stems, roots, flowers, or fruits, to permeation by the polynucleotide molecules into plant cells. The transfer of polynucleotides into plant cells can be facilitated by the prior or contemporaneous application of a polynucleotide-transferring agent to the plant tissue. In some embodiments the transferring agent is applied subsequent to the application of the polynucleotide composition. The polynucleotide transfer agent enables a pathway for polynucleotides through cuticle wax barriers, stomata and/or cell wall or membrane barriers into plant cells. Suitable transfer agents to facilitate transfer of the polynucleotide into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof. Chemical agents for conditioning or transfer include (a) surfactants, (b) an organic solvent or an aqueous solution or aqueous mixtures of organic solvents, (c) oxidizing agents, (d) acids, (e) bases, (f) oils, (g) enzymes, or combinations thereof. Embodiments of the method can optionally include an incubation step, a neutralization step (e.g., to neutralize an acid, base, or oxidizing agent, or to inactivate an enzyme), a rinsing step, or combinations thereof. Embodiments of agents or treatments for conditioning of a plant to permeation by polynucleotides include emulsions, reverse emulsions, liposomes, and other micellar-like compositions. Embodiments of agents or treatments for conditioning of a plant to permeation by polynucleotides include counter-ions or other molecules that are known to associate with nucleic acid molecules, e.g., inorganic ammonium ions, alkyl ammonium ions, lithium ions, polyamines such as spermine, spermidine, or putrescine, and other cations. Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e.g., plant-sourced oils, crop oils (such as those listed in the 9^{th} Compendium of Herbicide Adjuvants, publicly available on the worldwide web (internet) at herbicide.adjuvants.com can be used, e.g., paraffinic oils, polyol fatty acid esters, or oils with short-chain molecules modified with amides or polyamines such as polyethyleneimine or N-pyrrolidine. Transfer agents include, but are not limited to, organosilicone preparations.

Ligands can be tethered to a polynucleotide, for example a dsRNA, ssRNA, dsDNA or ssDNA. Ligands in general can include modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids (e.g., cholesterol, a bile acid, or a fatty acid (e.g., lithocholic-oleyl, lauroyl, docosnyl, stearoyl, palmitoyl, myristoyl oleoyl, linoleoyl), steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. The ligand may also be a recombinant or synthetic molecule, such as a synthetic polymer, e.g., polyethylene glycol (PEG), PEG-40K, PEG-20K and PEG-5K. Other examples of ligands include lipophilic molecules, e.g, cholesterol, cholic acid, adamantane acetic acid, 1-pyrene butyric acid, dihydrotestosterone, glycerol (e.g., esters and ethers thereof, e.g., C.sub.10, C.sub.11, C.sub.12, C.sub.13, C.sub.14, C.sub.15, C.sub.16, C.sub.17, C.sub.18, C.sub.19, or C.sub.20 alkyl; e.g., lauroyl, docosnyl, stearoyl, oleoyl, linoleoyl 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dodecanoyl, lithocholyl, 5.beta.-cholanyl, N,N-distearyl-lithocholamide, 1,2-di-O-stearoylglyceride, dimethoxytrityl, or phenoxazine) and PEG (e.g., PEG-5K, PEG-20K, PEG-40K). Preferred lipophilic moieties include lipid, cholesterols, oleyl, retinyl, or cholesteryl residues.

Conjugating a ligand to a dsRNA can enhance its cellular absorption, lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-radiated endocytosis. dsRNA compounds bearing folic acid would be efficiently transported into the cell via the folate-receptor-mediated endocytosis. Other ligands that have been conjugated to oligonucleotides include polyethylene glycols, carbohydrate clusters, cross-linking agents, porphyrin conjugates, delivery peptides and lipids such as cholesterol. In certain instances, conjugation of a cationic ligand to oligonucleotides results in improved resistance to nucleases. Representative examples of cationic ligands are propylammonium and dimethylpropylammonium. Interestingly, antisense oligonucleotides were reported to retain their high binding affinity to mRNA when the cationic ligand was dispersed, throughout the oligonucleotide. See M. Manoharan Antisense & Nucleic Acid Drug Development 2002, 12, 103 and references therein.

A biologic delivery can be accomplished by a variety of methods including, without limitation, (1) loading liposomes with a dsRNA acid molecule provided herein and (2) complexing a dsRNA molecule with lipids or liposomes to form nucleic acid-lipid or nucleic acid-liposome complexes. The liposome can be composed of cationic and neutral lipids commonly used to transfect cells in vitro. Cationic lipids can complex (e.g., charge-associate) with negatively charged, nucleic acids to form liposomes. Examples of cationic liposomes include, without limitation, lipofectin, lipofectamine, lipofectace, and DOTAP. Procedures for forming liposomes are well known in the art. Liposome compositions can be formed, for example, from phosphatidylcholine, dimyristoyl phosphatidylcholine, dipalmitoyl phosphatidylcholine, dimyristoyl phosphatidyl glycerol, dioleoyl phosphatidylethanolamine or liposomes comprising dihydrosphingomyelin (DHSM) Numerous lipophilic agents are commercially available, including Lipofectin® (Invitrogen/Life Technologies, Carlsbad, Calif.) and Effectene™ (Qiagen, Valencia, Calif.), In addition, systemic delivery methods can be optimized using commercially available cationic lipids such as DDAB or DOTAP, each of which can be mixed with a neutral lipid such as DOPE or cholesterol. In some eases, liposomes such as those described by Templeton et al. Nature Biotechnology, 15:647-652 (1997) can be used. In other embodiments, polycations such as polyethyleneimine can be used to achieve delivery in vivo and ex vivo (Boletta et al., J. Am Soc. Nephrol. 7:1728, 1996). Additional information regarding the use of liposomes to deliver nucleic acids can be found in U.S. Pat. No. 6,271,359, PCT Publication WO 96/40964 and Morrissey, D. et al., 2005. Nature Biotechnol. 23(8):1002-7.

In certain embodiments, an organosilicone preparation that is commercially available as Silwet® L-77 surfactant having CAS Number 27306-78-1 and EPA Number: CAL.REG.NO. 5905-50073-AA, and currently available from Momentive Performance Materials, Albany, New York can be used to prepare a polynucleotide composition. In certain embodiments where a Silwet L-77 organosilicone preparation is used as a pre-spray treatment of plant leaves or other plant surfaces, freshly made concentrations in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a leaf or other plant surface for transfer of polynucleotide molecules into plant cells from a topical application on the surface. In certain embodiments of the methods and compositions provided herein, a composition that comprises a polynucleotide molecule and an organosilicone preparation comprising Silwet L-77 in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

In certain embodiments, any of the commercially available organosilicone preparations provided such as the following Breakthru S 321, Breakthru S 200 Cat#67674-67-3, Breakthru OE 441 Cat#68937-55-3, Breakthru S 278 Cat #27306-78-1, Breakthru S 243, Breakthru S 233 Cat#134180-76-0, available from manufacturer Evonik Goldschmidt (Germany), Silwet® HS 429, Silwet® HS 312, Silwet® HS 508, Silwet® HS 604 (Momentive Performance Materials, Albany, N.Y.) can be used as transfer agents in a polynucleotide composition. In certain embodiments where an organosilicone preparation is used as a pre-spray treatment of plant leaves or other surfaces, freshly made concentrations in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a leaf or other plant surface for transfer of polynucleotide molecules into plant cells from a topical application on the surface. In certain embodiments of the methods and compositions provided herein, a composition that comprises a polynucleotide molecule and an organosilicone preparation in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

Organosilicone preparations used in the methods and compositions provided herein can comprise one or more effective organosilicone compounds. As used herein, the phrase “effective organosilicone compound” is used to describe any organosilicone compound that is found in an organosilicone preparation that enables a polynucleotide to enter a plant cell. In certain embodiments, an effective organosilicone compound can enable a polynucleotide to enter a plant cell in a manner permitting a polynucleotide mediated suppression of a target gene expression in the plant cell. In general, effective organosilicone compounds include, but are not limited to, compounds that can comprise: i) a trisiloxane head group that is covalently linked to, ii) an alkyl linker including, but not limited to, an n-propyl linker, that is covalently linked to, iii) a poly glycol chain, that is covalently linked to, iv) a terminal group. Trisiloxane head groups of such effective organosilicone compounds include, but are not limited to, heptamethyltrisiloxane. Alkyl linkers can include, but are not limited to, an n-propyl linker Poly glycol chains include, but are not limited to, polyethylene glycol or polypropylene glycol. Poly glycol chains can comprise a mixture that provides an average chain length “n” of about “7.5”. In certain embodiments, the average chain length “n” can vary from about 5 to about 14. Terminal groups can include, but are not limited to, alkyl groups such as a methyl group. Effective organosilicone compounds are believed to include, but are not limited to, trisiloxane ethoxylate surfactants or polyalkylene oxide modified heptamethyl trisiloxane.

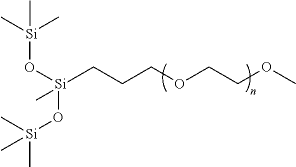

(Compound I: polyalkyleneoxide heptamethyltrisiloxane, average n=7.5).

In certain embodiments, an organosilicone preparation that comprises an organosilicone compound comprising a trisiloxane head group is used in the methods and compositions provided herein. In certain embodiments, an organosilicone preparation that comprises an organosilicone compound comprising a heptamethyltrisiloxane head group is used in the methods and compositions provided herein. In certain embodiments, an organosilicone composition that comprises Compound I is used in the methods and compositions provided herein. In certain embodiments, an organosilicone composition that comprises Compound I is used in the methods and compositions provided herein. In certain embodiments of the methods and compositions provided herein, a composition that comprises a polynucleotide molecule and one or more effective organosilicone compound in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

Compositions include but are not limited components that are one or more polynucleotides essentially identical to, or essentially complementary to a DHPS gene sequence (promoter, intron, exon, 5′ untranslated region, 3′ untranslated region), a transfer agent that provides for the polynucleotide to enter a plant cell, a herbicide that complements the action of the polynucleotide, one or more additional herbicides that further enhance the herbicide activity of the composition or provide an additional mode of action different from the complementing herbicide, various salts and stabilizing agents that enhance the utility of the composition as an admixture of the components of the composition.

The methods include one or more applications of a polynucleotide composition and one or more applications of a permeability-enhancing agent for conditioning of a plant to permeation by polynucleotides. When the agent for conditioning to permeation is an organosilicone composition or compound contained therein, embodiments of the polynucleotide molecules are double-stranded RNA oligonucleotides, single-stranded RNA oligonucleotides, double-stranded RNA polynucleotides, single-stranded RNA polynucleotides, double-stranded DNA oligonucleotides, single-stranded DNA oligonucleotides, double-stranded DNA polynucleotides, single-stranded DNA polynucleotides, chemically modified RNA or DNA oligonucleotides or polynucleotides or mixtures thereof.

Compositions and methods are useful for modulating the expression of an endogenous DHPS gene or transgenic DHPS gene (for example U.S. Pat. No. 6,121,513) in a plant cell. In various embodiments, a DHPS gene includes coding (protein-coding or translatable) sequence, non-coding (non-translatable) sequence, or both coding and non-coding sequence. Compositions can include polynucleotides and oligonucleotides designed to target multiple genes, or multiple segments of one or more genes. The target gene can include multiple consecutive segments of a target gene, multiple non-consecutive segments of a target gene, multiple alleles of a target gene, or multiple target genes from one or more species.

An aspect provides a method for modulating expression of a DHPS gene in a plant including (a) conditioning of a plant to permeation by polynucleotides and (b) treatment of the plant with the polynucleotide molecules, wherein the polynucleotide molecules include at least one segment of 18 or more contiguous nucleotides cloned from or otherwise identified from the target DHPS gene in either anti-sense or sense orientation, whereby the polynucleotide molecules permeate the interior of the plant and induce modulation of the target gene. The conditioning and polynucleotide application can be performed separately or in a single step. When the conditioning and polynucleotide application are performed in separate steps, the conditioning can precede or can follow the polynucleotide application within minutes, hours, or days. In some embodiments more than one conditioning step or more than one polynucleotide molecule application can be performed on the same plant. In embodiments of the method, the segment can be cloned or identified from (a) coding (protein-encoding), (b) non-coding (promoter and other gene related molecules), or (c) both coding and non-coding parts of the target gene. Non-coding parts include DNA, such as promoter regions or the RNA transcribed by the DNA that provide RNA regulatory molecules, including but not limited to: introns, 5′ or 3′ untranslated regions, and microRNAs (miRNA), trans-acting siRNAs, natural anti-sense siRNAs, and other small RNAs with regulatory function or RNAs having structural or enzymatic function including but not limited to: ribozymes, ribosomal RNAs, t-RNAs, aptamers, and riboswitches.

All publications, patents and patent applications are herein incorporated by reference to the same extent as if each individual publication or patent application was specifically and individually indicated to be incorporated by reference.

The following examples are included to demonstrate examples of certain preferred embodiments. It should be appreciated by those of skill in the art that the techniques disclosed in the examples that follow represent approaches the inventors have found function well in the practice, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope.

### EXAMPLES

### Example 1

### Polynucleotides Related to the DHPS Gene Sequences

The target DHPS polynucleotide molecule naturally occurs in the genome of plants that include but are not limited to Amaranthus palmeri, Amaranthus rudis, Amaranthus hybridus, Amaranthus lividus, Amaranthus viridis, Ambrosia trifida, Conyza candensis, Digitaria sanguinalis, Euphorbia heterophylla, Kochia scoparia, Lolium multiflorum, and include molecules related to the expression of a polypeptide identified as a DHPS, that include genomic DNA (gDNA) and coding cDNAs comprising coding and noncoding regions of a DHPS gene and fragments thereof as shown in Table 1.

Polynucleotide molecules were extracted from these plant species by methods standard in the field, for example, total RNA was extracted using Trizol Reagent (Invitrogen Corp, Carlsbad, Calif. Cat. No. 15596-018), following the manufacturer's protocol or modifications thereof by those skilled in the art of polynucleotide extraction that may enhance recover or purity of the extracted RNA. Briefly, start with 1 gram of ground plant tissue for extraction. Prealiquot 10 milliliters (mL) Trizol reagent to 15 mL conical tubes, add ground powder to tubes and shake to homogenize. Incubate the homogenized samples for 5 minutes (min) at room temperature (RT) and then add 3 mL of chloroform. Shakes tubes vigorously by hand for 15-30 seconds (sec) and incubate at RT for 3 min. Centrifuge the tubes at 7,000 revolutions per minute (rpm) for 10 min at 4 degrees C. Transfer the aqueous phase to a new 1.5 mL tube and add 1 volume of cold isopropanol. Incubate the samples for 20-30 min at RT and centrifuge at 10,000 rpm for 10 min at 4 degrees C. Wash pellet with Sigma-grade 80 percent ethanol. Remove the supernatant and briefly air-dry the pellet. Dissolve the RNA pellet in approximately 200 microliters of DEPC treated water. Heat briefly at 65 C to dissolve pellet and vortex or pipet to resuspend RNA pellet and then adjust RNA concentration to 1-2 microgram/microliter.

DNA was extracted using EZNA SP Plant DNA Mini kit (Omega Biotek, Norcross Ga., Cat#D5511) and Lysing Matrix E tubes (Q-Biogen, Cat#6914), following the manufacturer's protocol or modifications thereof by those skilled in the art of polynucleotide extraction that may enhance recover or purity of the extracted DNA. Briefly, aliquot ground tissue to a Lysing Matrix E tube on dry ice, add 800 μl Buffer SP1 to each sample, homogenize in a bead beater for 35-45 sec, incubate on ice for 45-60 sec, centrifuge at ≧14000 rpm for 1 min at RT, add 10 microliter RNase A to the lysate, incubate at 65° C. for 10 min, centrifuge for 1 min at RT, add 280 μl Buffer SP2 and vortex to mix, incubate the samples on ice for 5 min, centrifuge at ≧10,000 g for 10 min at RT, transfer the supernatant to a homogenizer column in a 2 ml collection tube, centrifuge at 10,000 g for 2 min at RT, transfer the cleared lysate into a 1.5 ml microfuge tube, add 1.5 volumes Buffer SP3 to the cleared lysate, vortex immediately to obtain a homogeneous mixture, transfer up to 650 μl supernatant to the Hi-Bind column, centrifuge at 10,000 g for 1 min, repeat, apply 100 μl 65° C. Elution Buffer to the column, centrifuge at 10,000 g for 5 min at RT.

Next-generation DNA sequencers, such as the 454-FLX (Roche, Branford, Conn.), the SOLiD (Applied Biosystems,), and the Genome Analyzer (HiSeq2000, Illumina, San Diego, Calif.) were used to provide polynucleotide sequence from the DNA and RNA extracted from the plant tissues. Raw sequence data was assembled into contigs. The contig sequence was used to identify trigger molecules that can be applied to a plant to enable regulation of the gene expression.

The target DNA sequence isolated from genomic (gDNA) and coding DNA (cDNA) from the various weedy plant species for the DHPS gene and the assembled contigs as set forth in SEQ ID NOs 1-54 and Table 1 (See supplemental attachment 40_{—}21(58641)Btable1.doxc)

### Example 2

### Polynucleotides Related to the Trigger Molecules

The gene sequences and fragments of Table 1 were divided into 200 polynucleotide (200-mer) lengths with 25 polynucleotide overlapping regions and are shown in Table 2, SEQ ID NO:55-906. These polynucleotides are tested to select the most efficacious trigger regions across the length of any target sequence. The trigger polynucleotides are constructed as sense or anti-sense ssDNA or ssRNA, dsRNA, or dsDNA, or dsDNA/RNA hybrids and combined with an organosilicone based transfer agent to provide a polynucleotide preparation. The polynucleotides are combined into sets of two to three polynucleotides per set, using 4-8 nmol of each polynucleotide. Each polynucleotide set is prepared with the transfer agent and applied to a plant or a field of plants in combination with a DHPS inhibitor containing herbicide, or followed by a DHPS inhibitor treatment one to three days after the polynucleotide application, to determine the effect on the plant's susceptibility to an DHPS inhibitor. The effect is measured as stunting the growth and/or killing of the plant and is measured 8-14 days after treatment with the polynucleotide set and DHPS inhibitor. The most efficacious sets are identified and the individual polynucleotides are tested in the same methods as the sets are and the most efficacious single 200-mer identified. The 200-mer sequence is divided into smaller sequences of 50-70-mer regions with 10-15 polynucleotide overlapping regions and the polynucleotides tested individually. The most efficacious 50-70-mer is further divided into smaller sequences of 25-mer regions with a 12 to 13 polynucleotide overlapping region and tested for efficacy in combination with DHPS inhibitor treatment. By this method it is possible to identify an oligonucleotide or several oligonucleotides that are the most efficacious trigger molecule to effect plant sensitivity to a DHPS inhibitor or modulation of an DHPS gene expression. The modulation of DHPS gene expression is determined by the detection of DHPS siRNA moleclules specific to a DHPS gene or by an observation of a reduction in the amount of DHPS RNA transcript produced relative to an untreated plant or by merely observing the anticipated phenotype of the application of the trigger with the DHPS inhibitor containing herbicide. Detection of siRNA can be accomplished, for example, using kits such as mirVana (Ambion, Austin Tex.) and mirPremier (Sigma-Aldrich, St Louis, Mo.).

The target DNA sequence isolated from genomic (gDNA) and coding DNA (cDNA) from the various weedy plant species for the DHPS gene and the assembled contigs as set forth in SEQ ID NOs 1-54 were divided into polynucleotide fragments as shown in Table 2 (See supplemental attachment 40_{—}21(58641)Btable2.doxc) and as set forth in SEQ ID NOs 55-906.

The gene sequences and fragments of Table 1 were compared and 21-mers of contiguous polynucleotides were identified that had homology across the various DHPS gene sequences. The purpose is to identify trigger molecules that are useful as herbicidal molecules or in combination with a DHPS inhibitor herbicide across a broad range of weed species. The sequences shown in Table 3 represent the 21-mers that were present in the DHPS gene of at least eight of the weed species of Table 1. It is contemplated that additional 21-mers can be selected from the sequences of Table 1 that are specific for a single weed species or a few weeds species within a genus or trigger molecules that are at least 18 contiguous nucleotides, at least 19 contiguous nucleotides, at least 20 contiguous nucleotides or at least 21 contiguous nucleotides in length and at least 85 percent identical to an DHPS gene sequence selected from the group consisting of SEQ ID NO:1-54 or fragment thereof.

By this method it is possible to identify an oligonucleotide or several oligonucleotides that are the most efficacious trigger molecule to effect plant sensitivity to DHPS inhibitor or modulation of DHPS gene expression. The modulation of DHPS gene expression is determined by the detection of DHPS siRNA molecules specific to DHPS gene or by an observation of a reduction in the amount of DHPS RNA transcript produced relative to an untreated plant. Detection of siRNA can be accomplished, for example, using kits such as mirVana (Ambion, Austin Tex.) and mirPremier (Sigma-Aldrich, St Louis, Mo.).

The target DNA sequence isolated from genomic (gDNA) and coding DNA (cDNA) from the various weedy plant species for the DHPS gene and the assembled contigs as set forth in SEQ ID NOs 1-54 were divided into fragments as shown in Table 3 (See supplemental attachment 40_{—}21(58641)Btable3.doxc) and as set forth in SEQ ID NOs 907-1175.

### Example 3

### Methods Related to Treating Plants or Plant Parts with a Topical Mixture of the Trigger Molecules

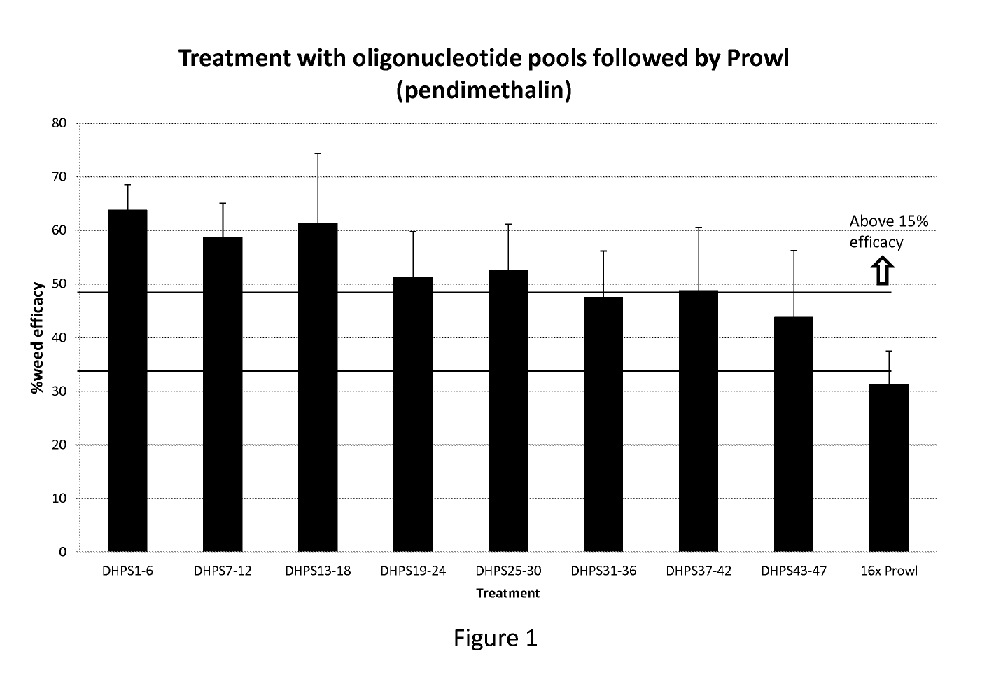

Glyphosate-sensitive Palmer amaranth (A. palmeri R-22) plants were grown in the greenhouse (30/20 C day/night T; 14 hour photoperiod) in 4 inch square pots containing Sun Gro® Redi-Earth and 3.5 kg/cubic meter Osmocote® 14-14-14 fertilizer. Palmer amaranth plants at 5 to 10 cm in height were pre-treated with a mixture of short (25mer) dsDNA trigger oligonucleotides targeting DHPS coding or noncoding regions using 4 nmol of each oligonucleotides and pooling 5-6 oligonucleotides in each treatment, formulated in 20 millimolar sodium phosphate buffer (pH 6.8) containing 2% ammonium sulfate and 1% Silwet L-77. Plants were treated manually by pipetting 10 μL of polynucleotide solution on four fully expanded mature leaves, for a total of 40 microliters of solution per plant. There were eight treatment pools, DHPS1-6, DHPS7-12, DHPS13-18, DHPS19-24, DHPS25-30, DHPS31-36, DHPS37-42, and DHPS43-47 (Table 4). Twenty-four hours later, the plants were treated with pendimethalin (Prowl®, BASF, this herbicide functions as a mitosis inhibitor similar to asulam a known DHPS inhibitor, other dinitroaniline herbicides that function to inhibit mitosis include but are not limited to Sonalan® (ethalfluralin), Squadron1®, Steel1®, Treflan®/Trilin®/Tri-4) at a rate of 12 lb/ac. Four replications of each treatment was conducted. Plant height was determined just before polynucleotide treatment and at intervals upto fourteen days after herbicide treatments to determine effect of the oligonucleotide and herbicide treatments. The results were expressed as percent reduction in height relative to the untreated control (no formulation and no trigger molecules), another treatment was the formulation control which is herbicide plus buffer plus ammonium plus Silwet. FIG. 1 illustrates the results of this test. Three of the pooled oligonucleotides demonstrated an enhancement of the herbicide activity, these are DHPS1-6, DHPS7-12, and DHPS13-18. Further testing of single oligonucleotides from DHPS1-6 and DHPS7-12 demonstrated that DHPS1 (SEQ ID NO:1176) and DHPS11 (SEQ ID NO:1186) had the highest activity among the oligonucleotides in those respective pools with these 2 oligonucleotides providing greater than 15 percent increase in herbicide injury.

TABLE 4
DHPS dsDNA oligonucleotides.

| | SEQ | | SEQ | |
|---|---|---|---|---|
| trigger | ID | | ID | |
| name | NO: | Sense sequence | NO: | Antisense sequence |
| DHPS1 | 1176 | TTTTATTCTAAAGTTGCttcGGAGG | 1223 | CCTCCGAAGCAACTTTAGAATAAAA |
| DHPS2 | 1177 | CCGTCAAGAAGGGGGCACACATTGT | 1224 | ACAATGTGTGCCCCCTTCTTGACGG |
| DHPS3 | 1178 | GAATGATGTCTCtaGTGGgAAACTC | 1225 | GAGTTTCCCACTAGAGACATCATTC |
| DHPS4 | 1179 | GATTCCGAGATGTTTAATGTTGTTG | 1226 | CAACAACATTAAACATCTCGGAATC |
| DHPS5 | 1180 | CGGACCTTAAAGTTCCTTATATAGC | 1227 | GCTATATAAGGAACTTTAAGGTCCG |
| DHPS6 | 1181 | AATGCACATGCGAGGAGATCCGACT | 1228 | AGTCGGATCTCCTCGCATGTGCATT |
| DHPS7 | 1182 | TCAATGCAAAACTCTGAGAACTTGA | 1229 | TCAAGTTCTCAGAGTTTTGCATTGA |
| DHPS8 | 1183 | CCTACAATGATGTTTGTAAGCAAGT | 1230 | ACTTGCTTACAAACATCATTGTAGG |
| DHPS9 | 1184 | GGCTTCGGAGTTGAGTTCTAGGGTc | 1231 | GACCCTAGAACTCAACTCCGAAGCC |
| DHPS10 | 1185 | ATAGATGCAGAATTATCGGGAATTC | 1232 | GAATTCCCGATAATTCTGCATCTAT |
| DHPS11 | 1186 | CTGCTTGGAGGATAGTTATTGATCC | 1233 | GGATCAATAACTATCCTCCAAGCAG |
| DHPS12 | 1187 | CGGCATCGGATTTTCTAAGAATACG | 1234 | CGTATTCTTAGAAAATCCGATGCCG |
| DHPS13 | 1188 | AATCAAAATTTGGAAATTCTTAGTG | 1235 | CACTAAGAATTTCCAAATTTTGATT |
| DHPS14 | 1189 | GTTTACAAAAGATACGGGAAGAGAT | 1236 | ATCTCTTCCCGTATCTTTTGTAAAC |
| DHPS15 | 1190 | AGCTAAGAAGAGTTTGGCGGTGGCT | 1237 | AGCCACCGCCAAACTCTTCTTAGCT |
| DHPS16 | 1191 | CATTGCCCCTTGCTAATTGGACCTT | 1238 | AAGGTCCAATTAGCAAGGGGCAATG |
| DHPS17 | 1192 | CAAGAAAGAGGTTTCTGGGCGAGAT | 1239 | ATCTCGCCCAGAAACCTCTTTCTTG |
| DHPS18 | 1193 | TTGTGAGCGCCCTGTAGCAGCTGAC | 1240 | GTCAGCTGCTACAGGGCGCTCACAA |
| DHPS19 | 1194 | AGGGATCCTGCTACCATTGCTTCTA | 1241 | TAGAAGCAATGGTAGCAGGATCCCT |
| DHPS20 | 1195 | TAACTGCTGGAGTTTTAGGTGGTGC | 1242 | GCACCACCTAAAACTCCAGCAGTTA |
| DHPS21 | 1196 | AAACATTGTAAGAGTACATAATGTt | 1243 | AACATTATGTACTCTTACAATGTTT |
| DHPS22 | 1197 | AGGGATAACCTTGATGCTGTCAAGT | 1244 | ACTTGACAGCATCAAGGTTATCCCT |
| DHPS23 | 1198 | TATGTGATGCCATACTCGGAAAAAC | 1245 | GTTTTTCCGAGTATGGCATCACATA |
| DHPS24 | 1199 | TGATTAACTGCTTGTTTGTACCACC | 1246 | GGTGGTACAAACAAGCAGTTAATCA |
| DHPS25 | 1200 | TTGTGAATGATGTCTAGTGGAAGGT | 1247 | ACCTTCCACTAGACATCATTCACAA |
| DHPS26 | 1201 | TCGATTGGGATTATGATGAAGTCCG | 1248 | CGGACTTCATCATAATCCCAATCGA |
| DHPS27 | 1202 | TTAGTTTGCTCGAAAGtaGAGCTTT | 1249 | AAAGCTCTACTTTCGAGCAAACTAA |
| DHPS28 | 1203 | AGCCCGTAAGGctATAGTTCTTACA | 1250 | TGTAAGAACTATAGCCTTACGGGCT |
| DHPS29 | 1204 | GATGTAGAATCATTGGCTATTTGCC | 1251 | GGCAAATAGCCAATGATTCTACATC |
| DHPS30 | 1205 | TATTTCTGCTTAGAAGATCATAGCA | 1252 | TGCTATGATCTTCTAAGCAGAAATA |
| DHPS31 | 1206 | TTGATCCAGACTTTGGATTCCATGT | 1253 | ACATGGAATCCAAAGTCTGGATCAA |
| DHPS32 | 1207 | AGAATTATGTTGCGACTCATGGTCG | 1254 | CGACCATGAGTCGCAACATAATTCT |
| DHPS33 | 1208 | AATTGGCCCTTTGAGGAATTGTTGA | 1255 | TCAACAATTCCTCAAAGGGCCAATT |
| DHPS34 | 1209 | AGTTCCTTGAGTCTATGGTTCAATC | 1256 | GATTGAACCATAGACTCAAGGAACT |
| DHPS35 | 1210 | GGCTCGGTTCGATGAAGGTTGGACT | 1257 | AGTCCAACCTTCATCGAACCGAGCC |
| DHPS36 | 1211 | GTGTCGAGACAAGATGGGTCAAAGA | 1258 | TCTTTGACCCATCTTGTCTCGACAC |
| DHPS37 | 1212 | GACCAATGGACAAGAAGTCGACCTT | 1259 | AAGGTCGACTTCTTGTCCATTGGTC |
| DHPS38 | 1213 | GGGCAAGCTTGATCgaGTTACTTGG | 1260 | CCAAGTAACTCGATCAAGCTTGCCC |
| DHPS39 | 1214 | ACAaGCAGATAACAGCAGGCTCTGG | 1261 | CCAGAGCCTGCTGTTATCTGCTTGT |
| DHPS40 | 1215 | CGACcATTAGACATACATATCATTt | 1262 | AAATGATATGTATGTCTAATGGTCG |
| DHPS41 | 1216 | GTTTTTtGAAGTCAGATCTGCAATC | 1263 | GATTGCAGATCTGACTTCAAAAAAC |
| DHPS42 | 1217 | AATCAGTTGCAATGGACAAaCCATA | 1264 | TATGGTTTGTCCATTGCAACTGATT |
| DHPS43 | 1218 | TACGGTTaTTagTTGTTCCTGTCAC | 1265 | GTGACAGGAACAACTAATAACCGTA |
| DHPS44 | 1219 | TgTTTGAGTTGAATTAGATCATGCA | 1266 | TGCATGATCTAATTCAACTCAAACA |
| DHPS45 | 1220 | AACATTGTAAGAATCTTGATCATGT | 1267 | ACATGATCAAGATTCTTACAATGTT |
| DHPS46 | 1221 | GAAGCTATGTGATACTGTACTTCTC | 1268 | GAGAAGTACAGTATCACATAGCTTC |
| DHPS47 | 1222 | AAAATGAAATCTTTGATATGATGTT | 1269 | AACATCATATCAAAGATTTCATTTT |

### Example 4

### A Method to Control Weeds in a Field

A method to control weeds in a field comprises the use of trigger polynucleotides that can modulate the expression of a DHPS gene in one or more target weed plant species. In Table 3, an analysis of DHPS gene sequences from multiple plant species provided a collection of 21-mer polynucleotides that were common to at least 4 of the species and can be used in compositions to affect the growth or develop or sensitivity to DHPS inhibitor herbicide to control multiple weed species in a field. Other oligonucleotide segments can be selected from the disclosed gene sequences that are more specific to a particular weed species, for example, an oligonucleotide that has a sequence that is homologous or complementary to a DHPS gene of three weeds species, or of two weed species, or of only one weed species. A composition containing 1 or 2 or 3 or 4 or more of the polynucleotides of Table 3 would enable broad activity of the composition against the multiple weed species that occur in a field environment.

The method includes creating a composition that comprises components that include at least one polynucleotide of Table 3 (SEQ ID NO:907-1175) or any other effective gene expression modulating polynucleotide essentially identical or essentially complementary to SEQ ID NO:1-54 or fragment thereof, a transfer agent that mobilizes the polynucleotide into a plant cell and a DHPS inhibiting herbicide and optionally a polynucleotide that modulates the expression of an essential gene and optionally a co-herbicide that has a different mode of action relative to a DHPS inhibitor. The polynucleotide of the composition includes a dsRNA, ssDNA or dsDNA or a combination thereof. A composition containing a polynucleotide can have a use rate of about 1 to 30 grams or more per acre depending on the size of the polynucleotide and the number of polynucleotides in the composition. The composition may include one or more additional co-herbicides as needed to provide effective multi-species weed control. Crop safety can be enhanced by reducing the amount of effective herbicide needed to control weeds in the field. A field of crop plants or a turf grass environment in need of weedy plant control is treated by spray application of the composition. The composition can be provided as a tank mix, a sequential treatment of components (generally the polynucleotide followed by the herbicide), a simultaneous treatment or mixing of one or more of the components of the composition from separate containers. Treatment of the field can occur as often as needed to provide weed control and the components of the composition can be adjusted to target specific weed species or weed families.

### Example 5

### Herbicidal Compositions Comprising Pesticidal Agents

A method of controlling weeds and plant pest and pathogens in a field of DHPS inhibitor tolerant crop plants is provided, wherein the method comprises applying a composition comprising a DHPS trigger oligonucleotide, a DHPS inhibitor composition and an admixture of a pest control agent. For example, the admixture comprises insecticides, fungicides, nematocides, bactericides, acaricides, growth regulators, chemosterilants, semiochemicals, repellents, attractants, pheromones, feeding stimulants or other biologically active compounds or biological agents, such as, microorganisms.

For example, the admixture comprises a fungicide compound for use on a DHPS inhibitor tolerant crop plant to prevent or control plant disease caused by a plant fungal pathogen, The fungicide compound of the admixture may be a systemic or contact fungicide or mixtures of each. More particularly the fungicide compound includes, but is not limited to members of the chemical groups strobilurins, triazoles, chloronitriles, carboxamides and mixtures thereof. The composition may additional have an admixture comprises an insecticidal compound or agent.

The DHPS trigger oligonucleotides and DHPS inhibitor or mitosis inhibitor herbicide (for example, asulam, or other dinitroaniline herbicides) tank mixes with fungicides, insecticides or both are tested for use in soybean and corn for control of foliar diseases and pests. Testing is conducted to develop a method for use of mixtures of the trigger oligonucleotides and asulam formulation and various commercially available fungicides for weed control and pest control. The field plots are planted with soybeans or corn. All plots receive a post plant application of the DHPS trigger+asulam about 3 weeks after planting. The mixtures of trigger+asulam or trigger+asulam+fungicide+insecticides are used to treat the plots at the R1 stage of soybean development (first flowering) or tassel stage of corn. Data is taken for percent weed control at 7 and 21 days after R1 treatment, soybean safety (% necrosis, chlorosis, growth rate): 5 days after treatment, disease rating, pest ratings and yield (bushels/Acre). These mixtures and treatments are designed to provide simultaneous weed and pest control, such as fungal pest control, for example, leaf rust disease; and insect pest control, for example, aphids, armyworms, loopers, beetles, stinkbugs, and leaf hoppers.

Agricultural chemicals are provided in containers suitable for safe storage, transportation and distribution, stability of the chemical compositions, mixing with solvents and instructions for use. A container of a mixture of a trigger oligonucleotide+herbicide+fungicide compound, or a mixture of a trigger oligonucleotide+herbicide compound and an insecticide compound, or a trigger oligonucleotide+a herbicide compound and a fungicide compound and an insecticide compound (for example, lambda-cyhalothrin, Warrier®). The container may further provide instructions on the effective use of the mixture. Containers of the present invention can be of any material that is suitable for the storage of the chemical mixture. Containers of the present invention can be of any material that is suitable for the shipment of the chemical mixture. The material can be of cardboard, plastic, metal, or a composite of these materials. The container can have a volume of 0.5 liter, 1 liter, 2 liter, 3-5 liter, 5-10 liter, 10-20 liter, 20-50 liter or more depending upon the need. A tank mix of a trigger oligonucleotide+herbicide compound and a fungicide compound is provided, methods of application to the crop to achieve an effective dose of each compound are known to those skilled in the art and can be refined and further developed depending on the crop, weather conditions, and application equipment used.

Insecticides, fungicides, nematocides, bactericides, acaricides, growth regulators, chemosterilants, semiochemicals, repellents, attractants, pheromones, feeding stimulants or other biologically active compounds can be added to the trigger oligonucleotide to form a multi-component pesticide giving an even broader spectrum of agricultural protection. Examples of such agricultural protectants with which compounds of this invention can be formulated are: insecticides such as abamectin, acephate, azinphos-methyl, bifenthrin, buprofezin, carbofuran, chlorfenapyr, chlorpyrifos, chlorpyrifos-methyl, cyfluthrin, beta-cyfluthrin, cyhalothrin, lambda-cyhalothrin, deltamethrin, diafenthiuron, diazinon, diflubenzuron, dimethoate, esfenvalerate, fenoxycarb, fenpropathrin, fenvalerate, fipronil, flucythrinate, tau-fluvalinate, fonophos, imidacloprid, isofenphos, malathion, metaldehyde, methamidophos, methidathion, methomyl, methoprene, methoxychlor, methyl 7-chloro-2,5-dihydro-2-[[N-(methoxycarbonyl)-N-[4-(trifluoromethoxy)phenyl]amino]carbonyl]indeno[1,2-e][1,3,4]oxadiazine-4a(3H)-carboxylate (DPX-JW062), monocrotophos, oxamyl, parathion, parathion-methyl, permethrin, phorate, phosalone, phosmet, phosphamidon, pirimicarb, profenofos, rotenone, sulprofos, tebufenozide, tefluthrin, terbufos, tetrachlorvinphos, thiodicarb, tralomethrin, trichlorfon and triflumuron; most preferably a DHPS inhibitor compound is formulated with a fungicide compound or combinations of fungicides, such as azoxystrobin, benomyl, blasticidin-S, Bordeaux mixture (tribasic copper sulfate), bromuconazole, captafol, captan, carbendazim, chloroneb, chlorothalonil, copper oxychloride, copper salts, cymoxanil, cyproconazole, cyprodinil (CGA 219417), diclomezine, dicloran, difenoconazole, dimethomorph, diniconazole, diniconazole-M, dodine, edifenphos, epoxiconazole (BAS 480F), famoxadone, fenarimol, fenbuconazole, fenpiclonil, fenpropidin, fenpropimorph, fluazinam, fluquinconazole, flusilazole, flutolanil, flutriafol, folpet, fosetyl-aluminum, furalaxyl, hexaconazole, ipconazole, iprobenfos, iprodione, isoprothiolane, kasugamycin, kresoxim-methyl, mancozeb, maneb, mepronil, metalaxyl, metconazole, S-methyl 7-benzothiazolecarbothioate (CGA 245704), myclobutanil, neo-asozin (ferric methanearsonate), oxadixyl, penconazole, pencycuron, probenazole, prochloraz, propiconazole, pyrifenox, pyroquilon, quinoxyfen, spiroxamine (KWG4168), sulfur, tebuconazole, tetraconazole, thiabendazole, thiophanate-methyl, thiram, triadimefon, triadimenol, tricyclazole, trifloxystrobin, triticonazole, validamycin and vinclozolin; combinations of fungicides are common for example, cyproconazole and azoxystrobin, difenoconazole, and metalaxyl-M, fludioxonil and metalaxyl-M, mancozeb and metalaxyl-M, copper hydroxide and metalaxyl-M, cyprodinil and fludioxonil, cyproconazole and propiconazole; commercially available fungicide formulations for control of Asian soybean rust disease include, but are not limited to Quadris® (Syngenta Corp), Bravo® (Syngenta Corp), Echo 720® (Sipcam Agro Inc), Headline® 2.09EC (BASF Corp), Tilt® 3.6EC (Syngenta Corp), PropiMax™ 3.6EC (Dow AgroSciences), Bumper® 41.8EC (MakhteshimAgan), Folicur® 3.6F (Bayer CropScience), Laredo® 25EC (Dow AgroSciences), Laredo™ 25EW (Dow AgroSciences), Stratego® 2.08F (Bayer Corp), Domark™ 125SL (Sipcam Agro USA), and Pristine®38% WDG (BASF Corp) these can be combined with DHPS inhibitor compositions as described in the present invention to provide enhanced protection from fungal disease; nematocides such as aldoxycarb and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents such as Bacillus thuringiensis, Bacillus thuringiensis delta endotoxin, baculovirus, and entomopathogenic bacteria, virus and fungi.

Lengthy table referenced here
US20130326731A1-20131205-T00001
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20130326731A1). An electronic copy of the table will also be available from the USPTO upon request and payment of the fee set forth in 37 CFR 1.19(b)(3).

We claim:

1. A method of plant control comprising: treating a plant with a composition comprising a polynucleotide and a transfer agent, wherein said polynucleotide is essentially identical or essentially complementary to a DHPS gene sequence or fragment thereof, or to an RNA transcript of said DHPS gene sequence or fragment thereof, wherein said DHPS gene sequence is selected from the group consisting of SEQ ID NO:1-54 or a polynucleotide fragment thereof, whereby said plant growth or development or reproductive ability is regulated, suppressed or delayed or said plant is more sensitive to a DHPS inhibitor herbicide or mitosis inhibitor herbicide as a result of said polynucleotide containing composition relative to a plant not treated with said composition.

2. The method as claimed in claim 1, wherein said transfer agent comprises an organosilicone surfactant composition or compound contained therein.

3. The method as claimed in claim 1, wherein said polynucleotide fragment is 18 contiguous, 19 contiguous nucleotides, 20 contiguous nucleotides, or at least 21 contiguous nucleotides in length and at least 85 percent identical to an DHPS gene sequence selected from the group consisting of SEQ ID NO:1-54.

4. The method as claimed in claim 3, wherein said polynucleotide fragment is selected from the group consisting of sense or anti-sense ssDNA or ssRNA, dsRNA, or dsDNA, or dsDNA/RNA hybrids.

5. The method as claimed in claim 1, wherein said plant is selected from the group consisting of Amaranthus palmeri, Amaranthus rudis, Amaranthus hybridus, Amaranthus lividus, Amaranthus viridis, Ambrosia trifida, Conyza candensis, Digitaria sanguinalis, Euphorbia heterophylla, Kochia scoparia, Lolium multiflorum,

6. The method as claimed in claim 1, wherein said composition further comprises said DHPS inhibitor or mitosis inhibitor herbicide and external application to a plant with said composition.

7. The method as claimed in claim 6, wherein said composition further comprises one or more co-herbicides different from said DHPS inhibitor or mitosis inhibitor herbicide.

8. The method as claimed in claim 3, wherein said composition comprises any combination of two or more of said polynucleotide fragments and external application to a plant with said composition.

9. A composition comprising a polynucleotide and a transfer agent, wherein said polynucleotide is essentially identical or essentially complementary to an DHPS gene sequence, or to an RNA transcript of said DHPS gene sequence, wherein said DHPS gene sequence is selected from the group consisting of SEQ ID NO:1-54 or a polynucleotide fragment thereof and whereby a plant treated with said composition has its growth or development or reproductive ability regulated, suppressed or delayed or said plant is more sensitive to a DHPS inhibitor herbicide or mitosis inhibitor herbicide as a result of said polynucleotide containing composition relative to a plant not treated with said composition.

10. The composition of claim 9, wherein said transfer agent is an organosilicone composition.

11. The composition of claim 9, wherein said polynucleotide fragment is 18 contiguous, 19 contiguous nucleotides, 20 contiguous nucleotides or at least 21 contiguous nucleotides in length and at least 85 percent identical to an DHPS gene sequence selected from the group consisting of SEQ ID NO:1-54.

12. The composition of claim 9, wherein said polynucleotide is selected from the group consisting of SEQ ID NO:55-906.

13. The composition of claim 9, wherein said polynucleotide is selected from the group consisting of SEQ ID NO: 907-1222.

14. The composition of claim 9, further comprising a DHPS inhibitor herbicide or mitosis inhibitor herbicide.

15. The composition of claim 14, wherein said DHPS inhibitor molecule is selected from the group consisting of carbamates and asulam.

16. The composition of claim 14, wherein said mitosis inhibitor molecule is selected from the group consisting of dinitroaniline herbicides.

17. The composition of claim 14, further comprising a co-herbicide.

18. A method of reducing expression of an DHPS gene in a plant comprising: external application to a plant of a composition comprising a polynucleotide and a transfer agent, wherein said polynucleotide is essentially identical or essentially complementary to an DHPS gene sequence, or to the RNA transcript of said DHPS gene sequence, wherein said DHPS gene sequence is selected from the group consisting of SEQ ID NO:1-54 or a polynucleotide fragment thereof, whereby said expression of said DHPS gene is reduced relative to a plant in which the composition was not applied.

19. The method as claimed in claim 18, wherein said transfer agent comprises an organosilicone compound.

20. The method as claimed in claim 18, wherein said polynucleotide fragment is 19 contiguous nucleotides, 20 contiguous nucleotides or at least 21 contiguous nucleotides in length and at least 85 percent identical to an DHPS gene sequence selected from the group consisting of SEQ ID NO:1-54.

21. The method as claimed in 18, wherein said polynucleotide molecule is selected from the group consisting of sense or anti-sense ssDNA or ssRNA, dsRNA, or dsDNA, or dsDNA/RNA hybrids.

22. A microbial expression cassette comprising a polynucleotide fragment of 18 contiguous, 19 contiguous nucleotides, 20 contiguous nucleotides or at least 21 contiguous nucleotides in length and at least 85 percent identical to an DHPS gene sequence selected from the group consisting of SEQ ID NO:1-54.

23. A method of making a polynucleotide comprising a) transforming the microbial expression cassette of claim 21 into a microbe; b) growing said microbe; c) harvesting a polynucleotide from said microbe, wherein said polynucleotide is at least 18 contiguous, at least 19 contiguous nucleotides, at least 20 contiguous nucleotides or at least 21 contiguous nucleotides in length and at least 85 percent identical to an DHPS gene sequence selected from the group consisting of SEQ ID NO:1-54.

24. A method of identifying polynucleotides useful in modulating DHPS gene expression when externally treating a plant comprising: a) providing a plurality of polynucleotides that comprise a region essentially identical or essentially complementary to a polynucleotide fragment of 18 contiguous, 19 contiguous nucleotides, 20 contiguous nucleotides or at least 21 contiguous nucleotides in length and at least 85 percent identical to an DHPS gene sequence selected from the group consisting of SEQ ID NO:1-54; b) externally treating said plant with one or more of said polynucleotides and a transfer agent; c) analyzing said plant or extract for modulation of DHPS gene expression, and whereby a plant treated with said composition has its growth or development or reproductive ability regulated, suppressed or delayed or said plant is more sensitive to a DHPS inhibitor herbicide or mitosis inhibitor herbicide as a result of said polynucleotide containing composition relative to a plant not treated with said composition.

25. The method as claimed in 24, wherein said plant is selected from the group consisting of Amaranthus palmeri, Amaranthus rudis, Amaranthus hybridus, Amaranthus lividus, Amaranthus viridis, Ambrosia trifida, Conyza candensis, Digitaria sanguinalis, Euphorbia heterophylla, Kochia scoparia, Lolium multiflorum,

26. The method as claimed in 24, wherein said DHPS gene expression is reduced relative to a plant not treated with said polynucleotide fragment and a transfer agent.

27. The method as claimed in 24, wherein said transfer agent is an organosilicone compound.

28. An agricultural chemical composition comprising an admixture of a polynucleotide and a DHPS inhibitor or mitosis inhibitor herbicide and a co-herbicide, wherein said polynucleotide is essentially identical or essentially complementary to a portion of a DHPS gene sequence, or to a portion of an RNA transcript of said DHPS gene sequence, wherein said DHPS gene sequence is selected from the group consisting of SEQ ID NO:1-54 or a polynucleotide fragment thereof, and whereby a plant treated with said composition has its growth or development or reproductive ability regulated, suppressed or delayed or said plant is more sensitive to a DHPS inhibitor herbicide or mitosis inhibitor herbicide as a result of said polynucleotide containing composition relative to a plant not treated with said composition.

29. The agricultural chemical composition of claim 28, wherein said co-herbicide is selected from the group consisting of amide herbicides, arsenical herbicides, benzothiazole herbicides, benzoylcyclohexanedione herbicides, benzofuranyl alkylsulfonate herbicides, cyclohexene oxime herbicides, cyclopropylisoxazole herbicides, dicarboximide herbicides, dinitroaniline herbicides, dinitrophenol herbicides, diphenyl ether herbicides, dithiocarbamate herbicides, glycine herbicides, halogenated aliphatic herbicides, imidazolinone herbicides, inorganic herbicides, nitrile herbicides, organophosphorus herbicides, oxadiazolone herbicides, oxazole herbicides, phenoxy herbicides, phenylenediamine herbicides, pyrazole herbicides, pyridazine herbicides, pyridazinone herbicides, pyridine herbicides, pyrimidinediamine herbicides, pyrimidinyloxybenzylamine herbicides, quaternary ammonium herbicides, thiocarbamate herbicides, thiocarbonate herbicides, thiourea herbicides, triazine herbicides, triazinone herbicides, triazole herbicides, triazolone herbicides, triazolopyrimidine herbicides, uracil herbicides, and urea herbicides.

30. An agricultural chemical composition comprising an admixture of a polynucleotide and a DHPS inhibitor herbicide and a pesticide, wherein said polynucleotide is essentially identical or essentially complementary to a portion of a DHPS gene sequence, or to a portion of an RNA transcript of said DHPS gene sequence, wherein said DHPS gene sequence is selected from the group consisting of SEQ ID NO:1-54 or a polynucleotide fragment thereof, whereby a field of crop plants in need of weed and pest control are treated with said composition and whereby a plant treated with said composition has its growth or development or reproductive ability regulated, suppressed or delayed or said plant is more sensitive to a DHPS inhibitor herbicide or mitosis inhibitor herbicide as a result of said polynucleotide containing composition relative to a plant not treated with said composition.

31. The agricultural chemical composition of claim 30, wherein said pesticide is selected from the group consisting of insecticides, fungicides, nematocides, bactericides, acaricides, growth regulators, chemosterilants, semiochemicals, repellents, attractants, pheromones, feeding stimulants, and biopesticides.

32. A composition comprising a polynucleotide and a transfer agent, wherein said polynucleotide is essentially identical or essentially complementary to a DHPS gene sequence, or to an RNA transcript of said DHPS gene sequence, wherein said polynucleotide is selected from the group consisting of SEQ ID NO:1176 and 1186 or a complement or polynucleotide fragment thereof, and whereby a plant treated with said composition has its growth or development or reproductive ability regulated, suppressed or delayed or said plant is more sensitive to a DHPS inhibitor herbicide or mitosis inhibitor herbicide as a result of said polynucleotide containing composition relative to a plant not treated with said composition.


SEQUENCE LISTING (1222 sequences)

SEQ ID NO: 1 - DNA, 672 bp, Amaranthus hybridus
agatttgtgagcgccctgtagcagctgatagggatcctgctaccgttgcttcaataactgctggagttttaggtggtgcaaacattgtaagagtacataatgtgagggataaccttgatgctgtcaagttatgtgatgccatactcggaaaaactgattaactgcttatttgtaccaccttgtgaatgacgtctagtggaaagttcgattgggatattgatgaagtccgttagtttgctcgaaagtagagctttagcccgtaaggttatagttcttacagatgtagaatcattggctatttgcctattcctgcttagaagatcatagcattgatccagactttggattccatgtagaattatgttgcgactcatggtcgaattggccctttgaggaattgttgaagttccttgatctatggttcaatcggctcggttcgatgaaggttggactgtgtcgagacaagatgggtcaaagagaccaatggacaagaagtcgaccttgggaaagcttgatcaagttacttggacgagcagatagcagcaggactctggcgagcattagacatacatatcatttgttttggagtcagatctgcaatcaatcagttgcaatggacaaaccatatacggttgttagttgttactgtcactgcttgagttgaattagatc

SEQ ID NO: 2 - DNA, 458 bp, Amaranthus lividus
aagggaataaggtatggtcctaggccaattgacttggacatactattttatgggaaatttagggtgagctctgagagcctcactatcccccatgaaaggatatgggaaagaccatttgtgatggcaccattgattgattgtattgggtctgatgtagaaaatgacactatttgtacatggcattcattatcaaattttttgggtggaatctttgaagcatggggtaaactcggtggaagttccctaatcgggaaggatggaatgaaaagggttttgcccgttggaaatcgcttatgggattggtctcataaaacctctgtaatgggagtcttgaacttgactcctgatagctttagtgatggcggaagttttcaatctgtcgatactgcggttgctaaggttcgtcagatgatctcagatggggcagatataattgacatcggggcacaatcaaccag

SEQ ID NO: 3 - DNA, 5590 bp, Amaranthus palmeri
ggtgtttatttgggcatggccttatgtgtgatgttggtaaattttgtggcttcgtccactaatctgatgtatcggatctgttaaagggagccctctcatcctatgcttatcaatgactttttcatatgcctatcaaacagatcagattggatcgggttcgagttcaggtcatatataaacatgtcagcaaacccttgacccatacccaacccatttagttaattggcttgaaaatcacaaccttaattcgaccagcgacagatcaggttgacctgatttcgacccacttaacccatttataatttttttttcgatttaagttttttaacattgattaaatctaattttttaggctttaattttaattttatgtttttttgtttaatttgtatttactatgaaaaataagaaactattatatgaactgagtttagcttacacttattaatttaactcgaaattaacacatttaattaaatggttatatagggttttttagttttaaaatttcaacctgaacctaatctatttaataaaaagatcagatctgatcaacccatataattaattgggtcaaaatctcaactcaaacccatttatttcgaattaaattcagatcaaattagcaggtcggatcaactagcgctaatttttcctctcaagttttttcatatgatgccccttgttttctttgaataaaattcaccaatttaaaatccctgcccttgatccgttggtattggtatttggtaaagcacatatggtaaattctactccattgttatctgtagttttggttctaaaccccaaacaaatcctactcctagcaccactaatcctcattactttctgtttgctgtctttctctacattcctactaaattaagacctagatgcagtgttattctgttcgaggttgccgaaaacggtacaatagttgcctaatttgcaattcatgtttgttcttgcgttttctacattgtttactctattggaacaaaggttgaactttcagcctttcagggagtattttgatttttctgtcaaattaccttgccagatttatttggctattttgattgtgtgtatttctttgtcccttgtttccatggggctaaactccttttggttaatcttaaacatttcttctaattttgaattgcagtggtatgaagtgttgtttaatatacaatcaatttttctgaactgggttaatgaagagaagcattgtagacatgaggaatgtgttcaagcatctcaaaacaaccaaattaatcccaaatgggatcttcaactgtaatagaggtaaatcacatatattctcatttaatggttattgttttattttaaggatgttattatgttgttatcaatgatagaaataagagaagcttgagcattcaaatgtatctatcactcactggttgtggacatctaaaatatggaccaagagttttcttcatttcttgtaccagaatttgcaaatgtatgattaaagtttaggttggagttgcaataaaatttcattaccccgacgctattaagtggctcacactgaggcggggtttgagggtcggatggtgcaaccttcaggagaggttctaagcattttcaacatgttcaaccgcagagggtcccgtaaaattggaggcttcaatattaaattatgtagtatgtgttaagtgtttaaagatataaaaatgttaatgaatacataatttttaaaattgcacagggctttcaaagaaatttttgattttgctccgcccatggcaacctttggcaaatctggatagaattagagaaatttacttttatgtatgtataatctgatgaatgaatgaatgaacaatgaagttcatttctgagtcaatgacctatatactatcgaggtttcgttaaataggatatataaataaaattagagacatcttttatccctaatgttgtctgttttcttttctgggcgagactgtttttggagaaaaacgttatgttgaatgcttgatttactcagaactgatgcaaacagatggataaaaaatggtgttttcctagtgtttatgtttcataaaaggatttcaaattccaatcgtatgaagtttgaaatactgctttttgtttttgtagcttcgtgttttgctttccttcactcttcaccagggaacgttgttgaagtttgttccaagaagcaagaagtcgtaattgcaatcgggagcaatgttggtgacagattagaaaatttcaaccaagctctgcaacaaatgaagaaattaggcatagacatcacaaggcatggttgtttatatgagacggaacctgcatacgtgactgatcaaccgaagtttcttaactctgctttaagaggctttacaagacttggacctcatgaattattaggggtattgaagaaaattgagaaggatatgggtagaaccaagggaataaggtatggtcctaggccaattgacttggacatactattttatgggaagtttagggtgagctctgagagcctcactatcccccatgaaaggatatgggaaagaccatttgtgatggcaccattgattgattgtattgggtctgatgtagaaaatgacactatttgtacgtggcattcattatcaaatttttcgggtggaatctttgaagcatggggtaaactcggtggaagctccctaatcgggaaggatggaatgaaaagggttttgcccgttggaaatctcttatgggattggtctcataaaacctctgttatgggagtcttgaacttgactcctgatagctttagtgatggcggaagttttcaatctgtcaatactgcggttgctaaggttcgtcagatgatctcagatggggcagatataattgacatcggggcacaatcaaccagacccatggcaactagaatttcggctgaagaagagctagcaagagtagtacctgtattagaagctgtcaaggatttgatcgaggaagaaggaagaatcttgtcagtggatacgttttattctaaagttgctttggaggccgtcaagaagggggcacacattgtgaatgatgtctctagtggtaaactcgattccgagatgtttaatgttgttgcggaccttaaagttccttatatagcaatgcacatgcgaggagatccgacttcaatgcaaaactctgagaacttgacctacaatgatgtttgtaagcaagtggcttcggagttgagttctagggtcatagatgcagaattatcgggaattcctgcttggaggatagttattgatcccggcatcggattttctaagaatacgaatcaaaatttggaaattcttagtggtttacaaaagatacgggaagagatagctaagaagagtttggcggtggctcattgccccttgctaattggaccttcaagaaagaggtttctgggcgagatttgtgagcgccctgtagcagctgacagggatcctgctaccattgcttctataactgctggagttttaggtggtgcaaacattgtaagagtacataatgttagggataaccttgatgctgtcaagttatgtgatgccatactcggaaaaactgattaactgcttgtttgtaccaccttgtgaatgatgtctagtggaaggttcgattgggattatgatgaagtccgttagtttgctcgaaagaagagctttagcccgtaaggttatagttcttacagatgtagaatcattggctatttgcctatttctgcttagaagatcatagcattgatccagactttggattccatgtagaattatgttgcgactcatggtcgaattggccctttgaggaattgttgaagttccttgagtctatggttcaatcggctcggttcgatgaaggttggactgtgtcgagacaagatgggtcaaagagaccaatggacaagaagtcgaccttgggcaagcttgatcaagttacttggacgagcagataacagcaggctctggcgagcattagacatacatatcatttgttttttgaagtcagatctgcaatcaatcagttgcaatggacagaccatatacggttgttagttgttcctgtcactctttgagttgaattagatcatgcaaacactgtaagagtcttgattttgtgaagctatgtgatactgtacttctcaaaatgaaatctttgatatgatgttagcttcaagagttgtttttccccccctcacattctgttctatgttgtattgttgtgtgctttttggaattgtattcctgtaattagaattattcctgcaaaatggtattcctttatatcatcaaaggagtttttgtacttctaatcattgatctttagatgcaaaactgaattaatacaagtgttcttgatgaaggcttattgaagaactcactttttatttatgtctaaacttgttaaagatgttctgcttccgcccttgccacgggatgtcgggattgactaagggtggtaatcgagggattcctatgcttgaattgactcgtttaagattttgcgttgattttaagtcaaatgttttgctaagcataaactattctagctcgacttgaataatgaagttttcgagtcaagtccgagctgttctgtaagtttatgtatcttgaagcttcttagttcttacttcttatccttatttgtttgacacattgaaccgcgttgatgcttgcttgactcaacccgttaaagtcttgaattcatcacgaattcgagtcgagcaagtttaaacccaacattaactaagctctcgaggcttaatgaggcaggggtagctcaagtttgttattcaagcaacttaccatagtattcttttaaaaggcatatagtcagttagttatttgttaatttcagtagaacgaaagaagtacattattaaacttggccattgatgcttgagctagagagcttcggttgtgatcgaaacgaatcctgatttcgctcttgatctttgatgtgaagtgacaaggccttcataaaactcctttattcttcagcagtgcatacaaaaatcatgtgtgcccagctcctagctcacataccgcgtttttcccctaatctagtgtggtcaaataaaaacagcaagatgtgttgatgtcatgtgcaatagtaattatcaggtgaagtaatgcaatatgtagccagtcacctcatatcaaaaaaatgttgggaggaaaacgtattcgttcatcactaaacctgtgtttatacatctaactgatcgatcagggtttcaaaatccaaattaatacaatggcatttgacgaagatatccagaacacatcccccttgtttctgctagataatcaactattctgattctgctgtaatacctcagcttcggccctagccatgggatcgtcctctttttctccttcacgctcctcggccagtttctttctgtgcagttcttcagcagcttctattgaagctttgttctcggcctgaagacgctgcatttcctcttccatttgctgcctctcaaaccatgtatcagcatccgcccaaactgcagacaagtatacaaaagttaaataacgaaagtcttcaaaactgcagatccagtgaacaa

SEQ ID NO: 4 - DNA, 5515 bp, Amaranthus palmeri
cggatctgttaaagggagccctctcatcctatgcttatcaatgactttttcatatgcctatcaaacagatcagattggatcgggttcgagttcaggtcatatataaacatgtcagcaaacccttgacccatacccaacccatttagttaattggcttgaaaatcacaaccttaattcgaccagcgacagatcaggttgacctgatttcgacccacttaacccatttataatttttttttcgatttaagttttttaacattgattaaatctaattttttaggctttaattttaatttatgtttttttgtttaatttgtatttactatgaaaaataagaaactattatatgaactgagtttagcttacacttattgatttaactcgaaattaacacatttaattaaatggttatatagggttttttagttttaaaatttcaacctgaacctaatctatttaataaaaagatcagatctgatcaacccatataattaattgggtcaaaatctcaactcaaacccatttatttcgaattaaattcagatcaaattagcaggtcggatcaactagcgctaatttttcctctcaagttttttcatatgatgccccttgttttctttgaataaaattcaccaatttaaaatccctgcccttgatccgttggtattggtatttggtaaagcacatatggtaaattctactccattgttatctgtagttttggttctaaaccccaaacaaatcctactcctagcaccactaatcctcattactttctgtttgctgtctttctctacattcctactaaattaagacctagatgcagtgttattctgttcgaggttgccgaaaacggtacaatagttgcctaatttgcaattcatgtttgttcttgcgttttctacattgtttactctattggaacaaaggttgaactttcagcctttcagggagtattttgatttttctgtcaaattaccttgccagatttatttggctattttgattgtgtgtatttctttgtcccttgtttccatggggctaaactccttttggttaatcttaaacatttcttctaattttgaattgcagtggtatgaagtgttgtttaatatacaatcaatttttctgaactgggttaatgaagagaagcattgtagacatgaggaatgtgttcaagcatctcaaaacaaccaaattaatcccaaatgggatcttcaactgtaatagaggtaaatcacatatattctcatttaatggttattgttttattttaaggatgttattatgttgttatcaatgatagaaataagagaagcttgagcattcaaatttatctatcactcactggttgtggacatctaaaatatggaccaagagttttcttcattttttgtaccagaagctgcaaatgtatgattaaagtttaggttggagttgcaataaaattttattaccccgacgctattaagtggctcacactgaggcggggtttgagggtcagatggtgcaaccttcagaagaggttctaagcattttcaacatgttcaaccgcatagggtcccgtaaaattggaggcttcaatattaaattatgtagtatgtgttatgtgtttaaagatataaatatgttaatgaatacataatttttaaattgcacagggccttcaaagaaattttcgattttgctctgcccctggcaacctttggcaaatttggatagaattagagaagtttacttttatgtgtgtataatatgatgaatgaatgagtgaacaatgaagttcatttctgagtcaatgacctatatactatcgaggtttcgttaaaaaggatatataaataaaattagagacatcttttatccctaatgttgtctgttttcttttctgggcgagactgtttttggagaaaaacgttatgttgaatgcttgatttactcagaactgatgcaaacagatggataaaaatggtgttttcctattgtttatgtttcgataacaggtttcaaattccactcgtatgaagtttgatatactgctatgtgtttttgtagcttcgtgttttgctttccttcactcatcaccagtgaatgatgttgaagtttgttccaagaagcaagaagtcgtaattgcaatcgggagcaatgttggtgacagattagaaaatttcaaccaagctctgcaacaaatgaagaaattaggcatagacatcacaaggcatgggtgtttatatgagacggaacctgcatacgtgactgatcaaccgaagtttcttaactctgctttaagaggctttacaaaacttgggcctcatgaattattaggggtattaaagaaaattgagaaggatatgggtagaaccaagggaataaggtatggtcctaggccaattgacttggacatactattttatgggaagtttagggtgagctctgagagcctcactatcccccatgaaaggatatgggaaagaccatttgtgatggcaccattgattgattgtattgggtctgatgtagaaaatgacactatttgtacgtggcattcattatcaaatttttcgggtggaatctttgaagcatggggtaaactcggtggaagttccctaatcgggaaggatggaatgaaaagggttttgcccgttggaaatcgcttatgggattggtctcataaaacctctgttatgggagtcttgaacttgactcctgatagctttagtgatggcggaagttttcaatctgtcgatactgcggttgctaaggttcgtcagatgatctcagatggggcagatataattgacatcggggcacaatcaaccagacccatggcaactagaatttcggctgaagaagagctagaaagagtagtacctgtattggaagctgtcaaggatttgatcgaggaagaaggaagaatcttgtcagtggatacgttttattctaaagttgcttcggaggccgtcaagaagggggcacacattgtgaatgatgtctctagtgggaaactcgattccgagatgtttaatgttgttgcggaccttaaagttccttatatagcaatgcacatgcgaggagatccgacttcaatgcaaaactctgagaacttgacctacaatgatgtttgtaagcaagtggctttggagttgagttctagggtcatagatgcagaattatcgggaattcctgcttggaggatagttattgatcccggcatcggattttctaagaatacgaatcaaaatttggaaattcttagtggtttacaaaagatacgggaagagatagctaagaagagtttggcggtggctcattgccccttgctaattggaccttcaagaaagaggtttctgggcgagatttgtgagcgccctgtagcagctgacagggatcctgctaccattgcttctataactgctggagttttaggtggtgcaaacattgtaagagtacataatgtgagggataaccttgatgctgtcaagttatgtgatgccatactcggaaaaactgattaactgcttgtttgtaccaccttgtgaatgatgtctagtggaaggttcgattgggattatgatgaagtccgttagtttgctcgaaagaagagctttagcccgtaaggttatagttcttacagatgtagaatcattggctatttgcctatttctgcttagaagatcatagcattgatccagactttggattccatgtagaattatgttgcgactcatggtcgaattggccctttgaggaattgttgaagttccttgagtctatggttcaatcggctcggttcgatgaaggttggactgtgtcgagacaagatgggtcaaagagaccaatggacaagaagtcgaccttgggcaagcttgatcaagttacttggacaagcagataacagcaggctctggcgagcattagacatatcatttgttttttggagtcagatctgcaatcaatcagttgcaatggacaaaccatatacggttgttagttgttactgtcactgtttgagttgaattagatcatgcaaacactgtaggagtcttgattatgtgaagctatgtgacactgtacttctcaaaatgaaatctttgatatgatgttagtttcaagagttgtaattcccccctcacatttctgttctatgttgtattgttgtgtgctttttggaattgtattcctgcaatagaattattcccgcaaaatggtattcctttatatcatcaaaggagtttttgtacttctaatcattgatctttagatgcaaaactgaattaatacaagtattcttgatgaaggcttattgaagaactcactttttatttatgtctaaacttgttaaagatgttctgcttccgcccttgccacgggatgtcgggattgactaggggtggtaatcgagggattcctatgcttgaattgactcgtttaagattttgcgttgattttaagtcaaacgttttgctaagcataaactattctagctcgacttgaataatgaagttttcgagtcaagtccgagctgttctataagtttacgtatcttgaagcttcttagttcttacttcttatcctttattttttgacacattgaaccgcattgatgcttgcttgactcaactcgttaaagtcttgagttcatcacgaattcgagtcgagcaagtttaaacccaacattaactaagctctcgaggcttaatgaggcaggggtagctcaagtttgttattcaagcaacttaccatagtattcttttaaaaggcatatagtcagttagttatttgttaatttcagtagaacgaaagaagtacattattaaacttggccattgatgcttgagctagagagtttcggttgtgatcgaaacgaatcctgatttcgctcttgatctttgatgtgaagtgacaaggccttcataaaactcctttattcttcagcagtgcatacaaaaatcatgtgtgcccagctcctagctcacataccgcgtttttcccctaatctagtgtggttaaataaaaacagcaagatgtgttgatgtcatgtgcaatagtaattatcaggtgaagtaattcaatatgtagccagtcacctcatatcaaaaaaatgttgggaggaaaacgtattcgttcatcactaaacctgtttttatacatctaactgatcgatcagggtttcaaaatccaaattaatacaatggcatttgacgaagatatccagaacacatcccccttgtttctgctagataatcaactattctgattctgctgtaatacctcagcttcggccctagccatgggatcgtcctctttttctccttcacgctcctcggccagtttctttctgtgcagttcttcagcagcttctattgaagctttgttctcggcctgaagacgctgcatttcctcttccatttgctgcctctcaaaccatgtatcagcatccgcccaaactgcagacaagtatacaaaagttaaataacgaaagtcttcaaaactgcagatccagtgaacaaagtca

SEQ ID NO: 5 - DNA, 1431 bp, Amaranthus palmeri
gctttccttcactcatcaccagtgaatgatgttgaagtttgttccaagaagcaagaagtcgtaattgcaatcgggagcaatgttggtgacagattagaaaatttcaaccaagctctgcaacaaatgaagaaattaggcatagacatcacaaggcatgggtgtttatatgagacggaacctgcatacgtgactgatcaaccgaagtttcttaactctgctttaagaggctttacaaaacttgggcctcatgaattattaggggtattaaagaaaattgagaaggatatgggtagaaccaagggaataaggtatggtcctaggccaattgacttggacatactattttatgggaagtttagggtgagctctgagagcctcactatcccccatgaaaggatatgggaaagaccatttgtgatggcaccattgattgattgtattgggtctgatgtagaaaatgacactatttgtacgtggcattcattatcaaatttttcgggtggaatctttgaagcatggggtaaactcggtggaagttccctaatcgggaaggatggaatgaaaagggttttgcccgttggaaatcgcttatgggattggtctcataaaacctctgttatgggagtcttgaacttgactcctgatagctttagtgatggcggaagttttcaatctgtcgatactgcggttgctaaggttcgtcagatgatctcagatggggcagatataattgacatcggggcacaatcaaccagacccatggcaactagaatttcggctgaagaagagctagaaagagtagtacctgtattggaagctgtcaaggatttgatcgaggaagaaggaagaatcttgtcagtggatacgttttattctaaagttgcttcggaggccgtcaagaagggggcacacattgtgaatgatgtctctagtgggaaactcgattccgagatgtttaatgttgttgcggaccttaaagttccttatatagcaatgcacatgcgaggagatccgacttcaatgcaaaactctgagaacttgacctacaatgatgtttgtaagcaagtggctttggagttgagttctagggtcatagatgcagaattatcgggaattcctgcttggaggatagttattgatcccggcatcggattttctaagaatacgaatcaaaatttggaaattcttagtggtttacaaaagatacgggaagagatagctaagaagagtttggcggtggctcattgccccttgctaattggaccttcaagaaagaggtttctgggcgagatttgtgagcgccctgtagcagctgacagggatcctgctaccattgcttctataactgctggagttttaggtggtgcaaacattgtaagagtacataatgtgagggataaccttgatgctgtcaagttatgtgatgccata

SEQ ID NO: 6 - DNA, 1431 bp, Amaranthus palmeri
gctttccttcactcttcaccagggaacgttgttgaagtttgttccaagaagcaagaagtcgtaattgcaatcgggagcaatgttggtgacagattagaaaatttcaaccaagctctgcaacaaatgaagaaattaggcatagacatcacaaggcatggttgtttatatgagacggaacctgcatacgtgactgatcaaccgaagtttcttaactctgctttaagaggctttacaagacttggacctcatgaattattaggggtattgaagaaaattgagaaggatatgggtagaaccaagggaataaggtatggtcctaggccaattgacttggacatactattttatgggaagtttagggtgagctctgagagcctcactatcccccatgaaaggatatgggaaagaccatttgtgatggcaccattgattgattgtattgggtctgatgtagaaaatgacactatttgtacgtggcattcattatcaaatttttcgggtggaatctttgaagcatggggtaaactcggtggaagctccctaatcgggaaggatggaatgaaaagggttttgcccgttggaaatctcttatgggattggtctcataaaacctctgttatgggagtcttgaacttgactcctgatagctttagtgatggcggaagttttcaatctgtcaatactgcggttgctaaggttcgtcagatgatctcagatggggcagatataattgacatcggggcacaatcaaccagacccatggcaactagaatttcggctgaagaagagctagcaagagtagtacctgtattagaagctgtcaaggatttgatcgaggaagaaggaagaatcttgtcagtggatacgttttattctaaagttgctttggaggccgtcaagaagggggcacacattgtgaatgatgtctctagtggtaaactcgattccgagatgtttaatgttgttgcggaccttaaagttccttatatagcaatgcacatgcgaggagatccgacttcaatgcaaaactctgagaacttgacctacaatgatgtttgtaagcaagtggcttcggagttgagttctagggtcatagatgcagaattatcgggaattcctgcttggaggatagttattgatcccggcatcggattttctaagaatacgaatcaaaatttggaaattcttagtggtttacaaaagatacgggaagagatagctaagaagagtttggcggtggctcattgccccttgctaattggaccttcaagaaagaggtttctgggcgagatttgtgagcgccctgtagcagctgacagggatcctgctaccattgcttctataactgctggagttttaggtggtgcaaacattgtaagagtacataatgttagggataaccttgatgctgtcaagttatgtgatgccata

SEQ ID NO: 7 - DNA, 1163 bp, Amaranthus palmeri
gaagctgtcaaggatttgatcgaggaagaaggaagaatcttgtcagtggatacgttttattctaaagttgcttcggaggccgtcaagaagggggcacacattgtgaatgatgtctctagtgggaaactcgattccgagatgtttaatgttgttgcggaccttaaagttccttatatagcaatgcacatgcgaggagatccgacttcaatgcaaaactctgagaacttgacctacaatgatgtttgtaagcaagtggctttggagttgagttctagggtcatagatgcagaattatcgggaattcctgcttggaggatagttattgatcccggcatcggattttctaagaatacgaatcaaaatttggaaattcttagtggtttacaaaagatacgggaagagatagctaagaagagtttggcggtggctcattgccccttgctaattggaccttcaagaaagaggtttctgggcgagatttgtgagcgccctgtagcagctgacagggatcctgctaccattgcttctataactgctggagttttaggtggtgcaaacattgtaagagtacataatgttagggataaccttgatgctgtcaagttatgtgatgccatactcggaaaaactgattaactgcttgtttgtaccaccttgtgaatgatgtctagtggaaggttcgattgggattatgatgaagtccgttagtttgctcgaaagtagagctttagcccgtaaggctatagttcttacagatgtagaatcattggctatttgcctatttctgcttagaagatcatagcattgatccagactttggattccatgtagaattatgttgcgactcatggtcgaattggccctttgaggaattgttgaagttccttgagtctatggttcaatcggctcggttcgatgaaggttggactgtgtcgagacaagatgggtcaaagagaccaatggacaagaagtcgaccttgggcaagcttgatcaagttacttggacaagcagataacagcaggctctggcgagcattagacatatcatttgttttttggagtcagatctgcaatcaatcagttgcaatggacaaaccatatacggttgttagttgttactgtcactgtttgagttgaattagatcatgcaaacactgtaggag

SEQ ID NO: 8 - DNA, 1431 bp, Amaranthus rudis
gcgtaccttcactcatcaccagtggacgatgttgaactttgttctaagaagcaagaagtcataattgcaatcgggagcaatgttggtgacagattagaaaattttaaccaagctctgcaacaaatgaagaaattaggcatagacatcacaaggcatggttgtttatatgagacagaacccgcatacgtgactgatcaaccgaagtttcttaactctgctttaagaggctttacaagacttgggcctcatgaattattaggggtattgaagaaaattgagaagaatatgggtagaaccaagggaataaggtatggtcctaggccaattgacttggacatactattttatgggaagtttagggtgagctctgagagcctcactattccccatgaaaggatatgggaaagaccatttgtgatggcaccattaattgattgtattgggtctggtgtagaaaatgacactatttgtacatggcattcgttatcaaatttttcgggtggaatctttgaagcatgggataaactcggtggaagttccctaatcgggaaggatggaatgaaaagggttttgcccgttgggaaccgcttatgggattggtctcataaaacctctgtaatgggagtcttgaacttgactcctgatagctttagtgatggaggaagttttcaatctgtcgatactgcggttgctaaggttcgtcagatgatatcagatggggccgacataattgacattggggcacagtcaacaagacccatggcaactaggatttcggctgaagaagagctagaaagagttgtacctgtattggaagctgttaaggatgtgatcgaggaagaaggaagaatcttatcggtggatactttttattcaaaagttgcttctgaggccgtcaagaagggggcacacattgtgaatgatgtctctagtgggaaattcgatcctgagatgttcaatgttgttgcggaccttaaagttccttatatagcaatgcacatgcgaggagatccgacttcaatgcaaaattctgagaacttgacgtacagtgatgtttgtaagcaagtggcttcggagttgagctctagggtcatagatgcagaattatcgggaattcctgcttggagaatagttattgatcccggcattggattttctaagaatacgaagcaaaatttggaaattcttactggtttacaaaagatacggctagagatagctaagaagagtttggcggtggctcattgccccttgctaattggaccttcacgaaagaggtttctgggtgagatttgtaatcgccctgtagcagctgatagggatcctgctaccgtcgcttctatcactgctggagttttaggtggtgcaaacattgtaagagtacataatgtgagggataaccttgatgcagtcaagttatgtgatgccata

SEQ ID NO: 9 - DNA, 1059 bp, Amaranthus rudis
tgtttcgataaaaggatttcaaatcccaatcgtatgaagtttgaaatacaactatgtgtttttgcagctttgttttttgcgtaccttcactcatcaccagtggacgatgttgaactttgttctaagaagcaagaagtcataattgcaatcgggagcaatgttggtgacagattagaaaattttaaccaagctctgcaacaaatgaagaaattaggcatagacatcacaaggcatggttgtttatatgagacagaacccgcatacgtgactgatcaaccgaagtttcttaactctgctttaagaggctttacaagacttgggcctcatgaattattaggggtattgaagaaaattgagaagaatatgggtagaaccaagggaataaggtatggtcctaggccaattgacttggacatactattttatgggaagtttagggtgagctctgagagcctcactattccccatgaaaggatatgggaaagaccatttgtgatggcaccattaattgattgtattgggtctggtgtagaaaatgacactatttgtacatggcattcgttatcaaatttttcgggtggaatctttgaagcatgggataaactcggtggaagttccctaatcgggaaggatggaatgaaaagggttttgcccgttgggaaccgcttatgggattggtctcataaaacctctgtaatgggagtcttgaacttgactcctgatagctttagtgatggaggaagttttcaatctgtcgatactgcggttgctaaggttcgtcagatgatatcagatggggccgacataattgacattggggcacagtcaacaagacccatggcaactaggatttcggctgaagaagagctagaaagagttgtacctgtattggaagctgttaaggatgtgatcgaggaagaaggaagaatcttatcggtggatactttttattcaaaagttgcttctgaggccgtcaagaagggggcacacattgtgaatgatgtctctagtgggaaattcgatcctgagatgttcaatgttgttgcggaccttaaagtt

SEQ ID NO: 10 - DNA, 792 bp, Amaranthus rudis
aatgggagtcttgaacttgactcctgatagctttagtgatggcggaaggtttcaatctgtcgatactgcggttgctaaggttcgtcagatgatctctgacggggcggacataattgacattggggcgcagtcaacaagacccatggcaactaggatttcggctgaagaagagctagaaagagtcgtacctgtattagaagctgtcaaggatttgatcgaggaagaaggaagaatcttgtcagtggatacgttttattctaaagttgcttcggaggccgtcaagaagggggcacacattgtgaatgatgtctctagtgggaaactcgatcccgagatgtttaatgttgttgcggaccttaaagttccttatatagcaatgcacatgcgaggagatccgacttcaatgcaaaattctgagaacttgacgtacaatgatgtgtgtaagcaagtggcttcggagttgagctctagggtcgtagatgcagaattatcgggaattcctgcttggaggatggttattgatcccggcattggattttctaagaatacgaagcaaaatttggaaattcttagtggtttacaaaagatacggcaagagatagctaagaagagtttggcggtggctcattgccccttgctaattggaccttcaagaaagaggtttctgggcgagatttgcaatcgccctgtagcagctgatagggatcctgctaccgttgcttctatcactgctggagttttaggtggtgcaaacattgtaagagtacataatgtgagggataaccttgatgc

SEQ ID NO: 11 - DNA, 764 bp, Amaranthus rudis
tggcaactaggatttcggctgaagaagagctagaaagagtcgtacctgtattagaagctgtcaaggatttgatcgaggaagaaggaagaatcttgtcagtggatacgttttattctaaagttgcttcggaggccgtcaagaagggggcacacattgtgaatgatgtttctagtgggaaactcgatcccgagatgtttaatgttgttgcggaccttaaagttccttatatagcaatgcacatgcgaggagatccgacttcaatgcaaaattctgagaacttgacgtacaatgatgtgtgtaagcaagtggcttcggagttgagctctagggtcgtagatgcagaattatcgggaattcctgcttggaggatggttattgatcccggcattggattttctaagaatacgaagcaaaatttggaaattcttagtggtttacaaaagatacggcaagagatagctaagaagagtttggcggtggctcattgccccttgctaattggaccttcaagaaagaggtttctgggcgagatttgcaatcgccctgtagcagctgatagggatcctgctaccgttgcttctatcactgctggagttttaggtggtgcaaacattgtaagagtacataatgtgagggataaccttgatgcagtcaagttatgtgatgccatactcagaaaaacggattagccgatttttttggattccagagccgatgttccagattttcctttgctttcatgtgccgaagattcttagtttagg

SEQ ID NO: 12 - DNA, 755 bp, Amaranthus rudis
gtaatgggagtcttgaacttgactcctgatagctttagtgatggcggaaggtttcaatctgtcgatactgcggttgctaaggttcgtcagatgatctctgacggggcggacataattgacattggggcgcagtcaacaagacccttggcaactaggatttcggctgaagaagagctagaaagagtcgtacctgtattagaagctgtcaaggatttgatcgaggaagaaggaagaatcttgtcagtggatacgttttattctaaagttgcttcggaggccgtcaagaagggggcacacattgtgaatgatgtttctagtgggaaactcgatcccgagatgtttaatgttgttgcggaccttaaagttccttatatagcaatgcacatgcgaggagatccgacttcaatgcaaaattctgagaacttgacgtacagtgatgtttgtaagcaagtggcttcggagttgagctctagggtcatagatgcagaattatcgggaattcctgcttggagaatagttattgatcccggcattggattttctaagaatacgaagcaaaatttggaaattcttactggtttacaaaagatacggctagagatagctaagaagagtttggcggtggctcattgccccttgctaattggaccttcacgaaagaggtttctgggtgagatttgtaatcgccctgtagcagctgatagggatcctgctaccgtcgcttctatcactgctggagttttaggtggtgcaaa

SEQ ID NO: 13 - DNA, 602 bp, Amaranthus rudis
aggatttcggctgaagaagagctagaaagagttgtacctgtattggaagctgttaaggatgtgatcgaggaagaaggaagaatcttatcggtggatacgttttattctaaagttgcttcggaggccgtcaagaagggggcacacattgtgaatgatgtttctagtgggaaactcgatcccgagatgtttaatgttgttgcggaccttaaagttccttatatagcaatgcacatgcgaggagatccgacttcaatgcaaaattctgagaacttgacgtacaatgatgtgtgtaagcaagtggcttcggagttgagctctagggtcgtagatgcagaattatcgggaattcctgcttggaggatggttattgatcccggcattggattttctaagaatacgaagcaaaatttggaaattcttagtggtttacaaaagatacggcaagagatagctaagaagagtttggcggtggctcattgccccttgctaattggaccttcaagaaagaaggtttctgggcgagatttgcaatcgccctgtagcagctgatagggatcctgctaccgttgcttctatcactgctggagttttaggtggtgcaa

SEQ ID NO: 14 - DNA, 584 bp, Amaranthus rudis
attagaggcatcttttatccctaatgctgtccgttttcttttctgggtgagactattttggaagaaaaaaatttatgttcaatgtttaatttactcagaattgatgcaaacagatggataaaaatggtgttttcttatggtttatgtttcaataaaaggatttcaaattctaatcatatgaagtttgaaatactatgtgtttttgtagcttcttgtattgctttccttcactcatcaccagtgaacgatgttgaagtttgttccaagaagcaagaagtcgtaattgcaatcgggagcaatgttggtgacagattagaaaatttcaacgaagctctgcaacaaatgaagaaattaggcatagacatcacaaggcatggttgtttatacgagacggaacctgcttacgtgactgatcagccgaagtttcttaactctgctttaagaggctttacaagacttgggcctcatgaattattaggggtattgaagagaatcgagaaggatatgggcagaaccaagggaataaggtatggtcctaggccaattgacttggacatactattttatgggaagtttagggtgag

SEQ ID NO: 15 - DNA, 570 bp, Amaranthus rudis
tgatttactcagaattgatgcaaacaaatggataaaaatggtgttttcctatggtttatgtttcgataaaaggatttcaaatcccaatcgtatgaagtttgaaatacaactatgtgtttttgtagcttcttgtattgctttccttcactcatcaccagtgaacgatgttgaagtttgttccaagaagcaagaagtcgtaattgcaatcgggagcaatgttggtgacagattagaaaatttcaacgaagctctgcaacaaatgaagaaattaggcatagacatcacaaggcatggttgtttatacgagacggaacctgcttacgtgactgatcagccgaagtttcttaactctgctttaagaggctttacaagacttgggcctcatgaattattaggggtattgaagagaatcgagaaggatatgggcagaaccaagggaataaggtatggtcctaggccaattgacttggacatactattttatgggaagtttagggtgagctctgagagcctcactattccccatgaaaggatatgggaaagaccatttgtgatggcaccattaattga

SEQ ID NO: 16 - DNA, 411 bp, Amaranthus rudis
ctgagagcctcactattccccatgaaaggatatgggaaagaccatttgtgatggcaccattaattgattgtattgggtctgatgtagaaaatgacactatttgtacatggcattcgttatcaaatttttcgggtggaatatttgaagcatgggataaactcggaggaagttccttaatcgggaaggatggaatgaaaagggttttgcccgttgggaaccgtttatgggattggtctcataaaacctcagtaatgggagtcttgaacttgactcctgatagctttagtgatggcggaaggtttcaatctgtcgatactgcggttgctaaggttcgtcagatgatctctgacggggcggacataattgacattggggcgcagtcaacaagacccatggcaactaggatttcgg

SEQ ID NO: 17 - DNA, 260 bp, Amaranthus rudis
cgctactgctgcagctgtcaccgctgcaattttgaatggtgctaacatagtaagggtacataatgtaggatatagttcagatgctgcaaagttttgtgatgcacctatactcattttacccatctcgctcctaatggaccccagccccttaataacgtcaatgttgtgcccacatttcttggagaacccaatgccaggatcaagaactatcctccacaatggaatcccagatgggtctgcttctcacctttgcatatagc

SEQ ID NO: 18 - DNA, 1504 bp, Amaranthus viridis
tctgtataacctaaagattgaaacttttgaagatatttttgaagaagtactctttggctttttgatccagaaaatcatattggtatgaagtgttgtttaatatacaatcaatttttctgaactgggttattaaagagaagcattgtagacatgaggaatgtgttcaagcatctcaaaacaaccaaattaatcccaaatgggatcttcaactgtaatagagcttcgtgttttgctttccttcactcatcaccggtgaacgatgttgaagtttgttccaagaagcaagaagtcgtaattgcaatcgggagcaatgttggtgacagattagaaaatttcaaccaagctctgcaacaaatgaagaaattaggcatagacatcacaaggcatgggtgtttatatgagacggaacctgcatacgtgactgatcaaccgaagtttcttaactctgctttaagaggctttacaaaacttgggcctcatgaattattaggggttttaaagaaaattgagaaggatatgggtagaaccaagggaataaggtatggtcctaggccaattgacttggacatactattttatgggaaatttagggtgagctctgagagcctcactatcccccatgaaaggatatgggaaagaccatttgtgatggcaccattgattgattgtattgggtctgatgtagaaaatgacactatttgtacatggcattcattatcaaattttttgggtggaatctttgaagcatggggtaaactcggtggaagttccctaatcgggaaggatggaatgaaaagggttttgcccgttggaaatcgcttatgggattggtctcataaaacctctgtaatgggagtcttgaacttgactcctgatagctttagtgatggcggaagttttcaatctgtcgatactgcggttgctaaggttcgtcagatgatctcagatggggcagatataattgacatcggggcacaatcaaccagacccatggcaactaggatttcggctgaagaagagctagaaagagtagtacctgtattggaagctgtcaaggatttgatcgaggaagaaggaagaatcttgtcagtggatacgttttattctaaagttgcttcggaggccgtcaagaagggggcacacattgtgaatgatgtctctagtgggaaactcgattccgagatgtttaatgttgttgcggaccttaaagttccttatatagcaatgcacatgcgaggagatccgacttcaatgcaaaattctgagaacttgacgtacaatgatgtttgtaagcaagtggctacggagttgagctctagggtcatagatgcagaattatcgggaattcctgcttggaggatagttattgatcccggcattggattttctaagaatacgaatcaaaatttggaaattcttcgtggtttacaaaagatacgggaagagatagctaagaagagtttggcagtggctcattgccccttgctaattggaccttcaagaa

SEQ ID NO: 19 - DNA, 655 bp, Amaranthus viridis
gcagctgatagggatcctgctaccgttgcttctataactgctggagttttaggtggtgcaaacattgtaagagtacataatgtgagggataaccttgatgctgtcaagttatgtgatgccatactcggaaaaactgattaactgcttatttgtaccaccttgtgaatgacgtctagtggaaagttcgattgggatatgatgaagtccgttagtttgctcgaaagtagagctttagcccgtaaggttatagttcttacagatgtagaatcattggctatttgcctatttttgcttagaagatcatagcattgatccagactttggattccatctagaattatgttgcgactcatggtcgaattggccctttgaggaattgttgaagttccttgatctatggttcaatcggcttggttcgatgaaggttggactgtgtcgagacaagatgggtcaaagagaccaatggacaagaagtcgaccttgggaaagcttgatcaagttacttggacgagcagatagcagcaggactctggcgagcattagacatacatatcatttgtttttggagtcagatctgcaatcaatcagttgcaatggacaaaccatatacggttgttagttgttactgtcactgcttgagttgaattagatcatg

SEQ ID NO: 20 - DNA, 1413 bp, Ambrosia trifida
tcgttctttcatttctcttcggatgcttcgatacaagtccattctcaagaacaagaagtagtaattgctttaggtagcaatgtgggtgataggcttaataactttaatgaagccttaagtcaaatgaaaaaatcaggcatagaaataacaagacatgcatgtttatacgaaaccgagccagcttacgtgactgatcaacctcttttcctcaattctgccatcagaggcgttacaaagctgggcccacatgagctactatcggccctcaagaaaattgaaaaagaaatgggccgaaccaaaggtattagatacggcccgcgacccattgacttagatatactgttttatggtaaacacagagttaactcggaaattctcactgttccacatgaaagaatcttcgagaggccatttgttatggctccgttagttgacttattggggtcggaaattgacaatgatacggttctatgctggcattctttctcaaaaagaggactttttgaatcttgggaaaaattaggtggcgaatctttgataggcaaagatgggttaagaagagttttaccagtcaataatcggttatgggattggtcaaagaaaacctctgtcatgggaattttgaacttgactcccgatagttttagcgacggaggaaagtttgacactgtggggtccgctatatctcgtgttagggccatgatatctgaaggggcagacataattgatctgggagctcaatcgacacgtccaatggcaaccaagatttcagtccaagaagaactagataggttaatccctgttctcgaaaagattcttgaattacccgaaattgaaggaaaactgctctctatcgacacgttttactcagaagttgctttagaagcgattaagaaaggggctcatataatcaatgatgtatcgggtggaaatctagattctgatatgtttcgggtagttgctaatcttgatgttccatatattgctatgcacatgagaggggacccatccacaatgcaaaacagtgagaatttgaagtatgatgatgtttgtaaagaagttggtgatgagttgtatgagcgtgtaagagctgcagagttatgcggtgtgcctgcatggaggatcattcttgacccagggatcgggttttcaaagaaaaccgaagataatttggatattttgatgggattaaagagaattaggagtgagattgcacggaagagcttaggggtgtcacgtgcacctttgttaatcggtccttcaagaaagagatttttgggggagatttgtggtcgggcttctgctgtcgagagagatccagcgactgttgctgctgttacatgtgcggttttgggtggtgctaatgttgttcgcgttcataacgttggagataatgcggatgctgtaaagctt

SEQ ID NO: 21 - DNA, 1328 bp, Ambrosia trifida
aatgaagccttaagtcaaatgaaaaaatcaggcatagaaataacaagacatgcatgtttatacgaaaccgagccagcttacgtgactgatcaacctcttttcctcaattctgccatcagaggcgttacaaagctgggcccacatgagctactatcggccctcaagaaaattgaaaaagaaatgggccgaaccaaaggtattagatacggcccgcgacccattgacttagatatactgttttatggtaaacacagagttaactcggaaattctcactgttccacatgaaagaatcttcgagaggccatttgttatggctccgttagttgacttattggggtcggaaattgacaatgatacggttctatgctggcattctttctcaaaaagaggactttttgaatcttgggaaaaattaggtggcgaatctttgataggcaaagatgggttaagaagagttttaccagtcaataatcggttatgggattggtcaaagaaaacctctgtcatgggaattttgaacttgactcccgatagttttagcgacggaggaaagtttgacactgtggggtccgctatatctcgtgttagggccatgatatctgaaggggcagacataattgatctgggagctcaatcgacacgtccaatggcaaccaagatttcagtccaagaagaactagataggttaatccctgttctcgaaaagattcttgaattacccgaaattgaaggaaaactgctctctatcgacacgttttactcagaagttgctttagaagcgattaagaaaggggctcatataatcaatgatgtatcgggtggaaatctagattctgatatgtttcgggtagttgctaatcttgatgttccatatattgctatgcacatgagaggggacccatccacaatgcaaaacagtgagaatttgaagtatgatgatgtttgtaaagaagttggtgatgagttgtatgagcgtgtaagagctgcagagttatgcggtgtgcctgcatggaggatcattcttgacccagggatcgggttttcaaagaaaaccgaagataatttggatattttgatgggattaaagagaattaggagtgagattgcacggaagagcttaggggtgtcacgtgcacctttgttaatcggtccttcaagaaagagatttttgggggagatttgtggtcgggcttctgctgtcgagagagatccagcgactgttgctgctgttacatgtgcggttttgggtggtgctaatgttgttcgcgttcataacgttggagataatgcggatgctgtaaagctttgtgattcaatgttgaaccg

SEQ ID NO: 22 - DNA, 486 bp, Ambrosia trifida
gccttaagtcaaatgaagaaatcaggcatagagataacaagacatgcatgtttatacaaaaccgagccagcttatgtaactgatcaacctctttttcttaattctgccatcagaggcgttacaaagctgggcccacatgaactactatcggccctcaagaaaatcgaaaaagaattgggccgaaccaaaggtattagatacggcccgcgacccattgacttagatatactgttttatggtaaacacagaattaactcggaaattctcactgttccacatgaaagaatcttcgagaggccatttgttatggctccgttagttgacttattggggtcggatattgacaatgatacggttctatgctggcattctttctcaaaaagaggactttttgaatcttgggaaaaattaggtggcgaatctttgataggcaaagatgggttaagaagagttttaccggtcaataatcggttatgggattggtca

SEQ ID NO: 23 - DNA, 486 bp, Ambrosia trifida; identical to SEQ ID NO: 22

SEQ ID NO: 24 - DNA, 410 bp, Ambrosia trifida
gacccatccacaatgcaaaacagtgagaatttgaagtataatgatgtttgtaaagaagttggtgatgagttgtatgagcgtgtaagagctgcagagttatgcggtgtgcctgcatggaggatcattcttgacccagggatcgggttttcaaagaaaaccgaagataatttggatattttgatgggattaaagagaattaggagtgagattgcccggaagagcttaggggtgtcacgtgcacctttgttaatcggtccttcaagaaagagatttttgggagagatttgtggtcgggcttctgctggtgagagagatccaacaactgttgctgctgttacatgtgcggttttgggtggtgctaatgttgttcgcgttcataatgttggagatgatgctgatgctgtaaagct

SEQ ID NO: 25 - DNA, 381 bp, Ambrosia trifida
ttcgtgtatctcatgatctctgtataatgttaaacttattatagttcaatataagtcactgtgtgatctaatttcgtatacaattactttatttgtgttgattctgatcctaaatattgctaatagtttgtgcttaactatatatcccgttaaacatcacatttactttatccctagaagatttgggcaagtttttacaaatttatggcgacgttattgtaattttttaaatcattttcgtgcagcatctttttcgttctttcatttctcttcggatgcttcgatacaagtccattctcaagaacaagaagtagtaattgctttaggtagcaatgtgggtgataggcttaataactttaatgaagccttaagtcaaatgaa

SEQ ID NO: 26 - DNA, 304 bp, Ambrosia trifida
gacccatccacaatgcaaaacagtgagaatttgaagtataatgatgtttgtaaagaagttggtgatgagttgtatgagcgtgtaagagctgcagagttatgcggtgtgcctgcatggaggatcattcttgacccagggatcgggttttcaaagaaaaccgaagataatttggatattttgatgggattaaagagaattaggagtgagattgcccggaagagcttaggggtgtcacgtgcacctttgttaatcggtccttcaagaaagagatttttgggagagatttgtggtcgggcttctgctg

SEQ ID NO: 27 - DNA, 255 bp, Ambrosia trifida
gatttgtggtcgggcttctgctggtgagagagatccaacaactgttgctgctgttacatgtgcggttttgggtggtgctaatgttgttcgcgttcataatgttggagatgatgctgatgctgtaaagctttgtgattcaatgttgaaccgggttggaagaacgtaacagctttttgaaatttgattgtgggttttggtccgggtggtggtgctcgggtgtggtttgggttttggttcgagtgggtggttgctggt

SEQ ID NO: 28 - DNA, 201 bp, Ambrosia trifida
acctcatcacctccacaaatcaccaccacccatctccaccctccaccaccacccacttcattctccccacccaccgtatctcatcaccatcatacctggtggattgcagtttgactctgtggggtccgctatatctcgtgttaggaccatgatatctgaaggggcggacataattgatctgggagctcaatcgacacgtcc

SEQ ID NO: 29 - DNA, 101 bp, Ambrosia trifida
ggattgcagtttgactctgtggggtccgctatatctcgtgttaggaccatgatatctgaaggggcggacataattgatctgggagctcaatcgacacgtcc

SEQ ID NO: 30 - DNA, 5446 bp, Conyza canadensis
tttcttcttctagttggagctcttttacgtggtgtcttcttttccgagtcatcttcttccccatcagatggattttctgcagccttttttctacgaactgctgtaatagcaaatgtactacgcaatctaacatctgggaattgtacaaagtttattgaagatagtgaaggcagattcgactgccacctacacatataagaaaccattaacttctatacttaactcacaatataaaacatcatttgcatgttcgtcaaaaacaataccatttcttatctttgctcaatggatatgaaatctgccaatacaagattattcatttgacctcaatctagcatctgatgtctattcaaggtatcgtttataatgaagattattgtacttcaaaatactttcttgtgtagtggtatcaaattttgagttaaagttactccatttgacctcaaaagccaaataatgagcaacaaacaacacaatttcacctcccttataccctgcgactatattttatcttcaaatcaatccatttccatgtttatcctctccatacagctacataataagttttagggaaaaaacaaaactaaactaaactaagtttacagaattcccatttcttatcctaaaatttacaagcaattcaacatatattctttatttacaactataaatagcatatgggtatgttggtatatagatatagatatagatatacatatgtagatgtgtattatatgtgtgtaaatggggagaaaaaaggaaacatatattacctgtgtaaagaacaaaattgagtaaaagaaggcaaagaagccatttctaaccactaaaatcttgggctttcaagaatcccacttcaaagctaccatttcacataccaaacccccctttctatgggtttaaagatgagggctttacatttatctcttttacaatccaaattttccaaaaccgaaataatgtacgacattcggtgcacaaggtacaaacagaaaaaactacttttgtattttagatctcaagaaaggagtggcctttttggtaataacatactttattcaattgccacttaccttcaagaaattaacaaaattacccacaattaaaaaagggtttcccagtttcactcacacttattcaagattgatgcaaaataaacccataaataagaaggttaaaatagtgttaaagccttaaaggtgcttgctttttgtgttttttgtgatgcctataaaaagattcttattgttttcaaatttttttatgtcaagttttcgtttttaacagtttatatagttatataatttgcagaaagccataaagctctgaacttgaatgttttatagcagctctttgcagtaatatcttgtagaggtaaacctgttaacccatatttattaatttgtacataaaattgttgaattgaatagagctaccactgggacttgtgtagtcaaactgtagagcatccctgtaatgctggaagctttatgacctaaaaggatttgtagacttgttatctgtggattggtataaagattttcaagttgttattttgaaactttgaatagtatattatgctcattactgaatgatacaatagggagtcgcgaatgttagaattgataagaattaggatgaagtttgtgtttgaacgttccttctgtttataatcttcatgtcacttccatagtaccataaccctagggcaacacgagcctttagattatgaatgggcacacccaattgtttagaccttgtataaatgaagtgaatatcattgttataagcattctggtggtggttttgcagatttacctttctctatctaatttgaaggtttgttgattaaggtgatacaaatgagtgttttcaagcagctaatagcttcaagattagtacgcactcatgtctgtggatcttatagaggtacacttcctcgaacattcatatagcgatgttattgttaagttttgaatctgtagcaacattatatataattttttttgcattgttttcctgcagcatccttttcactctttcattcgtcctcggatacttcaatacaagtgcattctccagaacaagaagtagtaattgctttaggtagcaatgtgggtaatagacttaataactttaatgaagccttatcccagatgaagaaatcggggatagaaataacaaggcatgcttgtctatacgaaactgaaccagcttatgtgacggaccagcctctttttctcaactctgccgttagagccactacaaagcttggccctcatgagctactgtccattctcaagaaaattgaaaaggaaatgggtcgaaccaaagggcttaggtacggcccacgacccattgacctagatatattgttctatggtaaatgcaaagttaactctgatattctaactgttcctcatgaaagaatctttgagaggccattcgttatggctccattagttgacttgttaggatcagatgtagacaatgatacggttctatgctggcattctttttcaaaaaatgggctttttggatcttgggaaacattaggtggtgaatcttccataggaaaagatggtttaagaagggttttacctgtcaatgatcgtttatgggattggtcaaagaaaacttccgtcatgggtattttgaatataactcctgatagttttagtgatggagggaagtttgattccatggggtccgctttatctcgtgttcagaccatgatatctgaaggggttgacataattgatctaggagctcaatccacacgcccaatggcgaccaagatctcagtcgaagaggaactagataggctaatacccgttcttgaaaagattcttgaattacctgaaattgaaggaaagttgttgtctgtggacacattttactcggaagttgcttcagaggcaatcaagaaaggggctcatatggtcaatgatgtatcgggtggaatgttagattctgatatgcttcatgttgtggctgatctaaatgttccatatatcactatgcacatgagaggggacccatccacaatgcaaaacagtgagaatttgaagtatgatgatgttgtcaaagaagttggggaggaattgtatgaacgtgtaaggaatgcagagttatgtggtgttcccgcatggaggatggttcttgatccagggatcgggttttcaaagaaaaccgaagataatttggagatattgatgggactaaagaggtttagaagtgagattggacaaaagagcttaggggtgtctcgtgcacctttgttaatcggaccttcaagaaaaaggtttttgggtgagatttgtggtcggccttctgctgttgagagagatccagggactgtagctgctgttaccagtgcgattttgggtggtgccaatatcgttagggttcataacattggacataatgtggatgctgttaagctttgtgattcaatgttggatcgagctggtagatcttagtagcttaaagttatttgacatcttgttttttattcaattttctggacattactttttgtatttctgtctggcaaatgattttacagggatatcctagaggctaaattgtatattacattcttgattttctcacaaagttttactttttcagtttttcttgttcagttttgtttttgaaaatttgctagatcagattaaatcgttgctttatgatcttcgtgtcttttttgggacgctgcgagtttcatttagtggtattgtaaactccttttaaagtttgtacacacttggtatttatgcttctagttttcgtctatgtctgtcctatattgtgtatagtatgtttaaagttcataaaaagaggataaacgaatagcacgtaaatcttataattcgattccacagagtgcaagttatgctttcctaaatgaaagcaagagtctagacagtttggtcaacgttaacatgagactattgtagaatacaacaccacaagaaaccaaaactgtcttgtgcatttctctcttttccaggtttaaaactcaataccaagacttgatatcaactattattctgcttcaatgattcagcttcagctatagccataggatcatctttggtgtcaggttcatccttctcaccatctcgttcctctgccagtttttttctgtgcaattcctctgcaacttgctttgatgcttggtcttctttttgaaggcgttgcatctcttcttcttgttgctgtcgttcaaaccatgtatccgcatcggcccaaactgtaatttagagccaaaacaacatgattagaatgcagattccagaagccaaattcaaactgtaagaaaaaaaatgtagtgatgatttaactcaaaacaactcacacagctccaaaatggatgggggttcaaattagaggatagcctaatagatagaagcttctaattatggggaaaaaaaaacgatcatggcaatgtattacacaatctaaagcaaccactatgaataataaagataaatctttccgagagtccacatgccttgatatttcataagataggctttttcagaaatcagaatcttcttggcaaatataacactaaaatcagtctaatgcatgttattaagtaaatctttatttgtccatagtataatgccatagattatcgtcaattatccacatcgtcattaaatatcaagtaaccacattatgaattgacttgtttaatcaaagttcatcttacttcttcatagatgaaaaagagctatgcaaatacagaacacatcagttgttctaacatataattactgctagctgtaggaagaaaccaagatgaaacttatctaggattcaaattcatggggatctgttatttagatgaatttagagaaataagaaaataagttgagtgaagtcagtttcgaggttccaaatttgtctttattagttgagctaacaatattaacctctgaagcatgtatagtgtacttgtatgaaccacttaaggatataaatgaagccaaatcagtttcattcgctaattgagcaaatgcacgttctgtttgttagtattgaaatttctttacaggatttctttcaactaaaagcatcacatgtccgactctcggtttgatatttgtgacaagtctaattggttcatcagatgtacttatggctaaatttcgaaacttgaaacaataagagcttagcactcttaaccataaagggttgctgttaatcttacttgttcttgaaagcaattccacagtgaagcatttgttaattgggaagttttgcaatgtttctaaagcttcgatgatattttatattttcaaacatttttcatcacatgtgttaacaaaagccataagaataccggggtaatcaactaaagtctatttaaagttgcatgctcgagagcaagaaaaaaccatcaaatttacataaactgaagggcaaatgtacg

SEQ ID NO: 31 - DNA, 5446 bp, Conyza canadensis
ttttcttcttctagttggagctcttttacgtggtgtcttcttttccgagtcatcttcttccccatcagatggattttctgcagccttttttctacgaactgctgtaatagcaaatgtactacgcaatctaacatctgggaattgtacaaagtttattgaagatagtgaaggcagattcgactgccacctacacatataagaaaccattaacttctatacttaactcacaatataaaacatcatttgcatgttcgtcaaaaacaataccatttcttatctttgctcaatggatatgaaatctgccaatacaagattattcatttgacctcaatctagcatctgatgtctattcaaggtatcgtttataatgaagattattgtacttcaaaatactttcttgtgtagtggtatcaaattttgagttaaagttactccatttgacctcaaaagccaaataatgagcaacaaacaacacaatttcacctcccttataccctgcgactatattttatcttcaaatcaatccatttccatgtttatcctctccatacagctacataataagttttagggaaaaaacaaaactaaactaaactaagtttacagaattcccatttcttatcctaaaatttacaagcaattcaacatatattctttatttacaactataaatagcatatgggtatgttggtatatagatatagatatagatatacatatgtagatgtgtattatatgtgtgtaaatggggagaaaaaaggaaacatatattacctgtgtaaagaacaaaattgagtaaaagaaggcaaagaagccatttctaaccactaaaatcttgggctttcaagaatcccacttcaaagctaccatttcacataccaaacccccctttctatgggtttaaagatgagggctttacatttatctcttttacaatccaaattttccaaaaccgaaataatgtacgacattcggtgcacaaggtacaaacagaaaaaactacttttgtattttagatctcaagaaaggagtggcctttttggtaataacatactttattcaattgccacttaccttcaagaaattaacaaaattacccacaattaaaaaagggtttcccagtttcactcacacttattcaagattgatgcaaaataaacccataaataagaaggttaaaatagtgttaaagccttaaaggtgcttgctttttgtgttttttgtgatgcctataaaaagattcttattgttttcaaatttttttatgtcaagttttcgtttttaacagtttatatagttatataatttgcagaaagccataaagctctgaacttgaatgttttatagcagctctttgcagtaatatcttgtagaggtaaacctgttaacccatatttattaatttgtacataaaattgttgaattgaatagagctaccactgggacttgtgtagtcaaactgtagagcatccctgtaatgctggaagctttatgacctaaaaggatttgtagacttgttatctgtggattggtataaagattttcaagttgttattttgaaactttgaatagtatattatgctcattactgaatgatacaatagggagtcgcgaatgttagaattgataagaattaggatgaagtttgtgtttgaacgttccttctgtttataatcttcatgtcacttccatagtaccataaccctagggcaacacgagcctttagattatgaatgggcacacccaattgtttagaccttgtataaatgaagtgaatatcattgttataagcattctggtggtggttttgcagatttacctttctctatctaatttgaaggtttgttgattaaggtgatacaaatgagtgttttcaagcagctaatagcttcaagattagtacgcactcatgtctgtggatcttatagaggtacacttcctcgaacattatatagcgatgttattgttaagttttgaatctgtagcaacattatatataattttttttgcattgttttcctgcagcatccttttcactctttcattcgtcctcggatacttcaatacaagtgcattctccagaacaagaagtagtaattgctttaggtagcaatgtgggtaatagacttaataactttaatgaagccttatcccagatgaagaaatcggggatagaaataacaaggcatgcttgtctatacgaaactgaaccagcttatgtgacggaccagcctctttttctcaactctgccgttagagccactacaaagcttggccctcatgagctactgtccattctcaagaaaattgaaaaggaaatgggtcgaaccaaagggcttaggtacggcccacgacccattgacctagatatattgttctatggtaaatgcaaagttaactctgatattctaactgttcctcatgaaagaatctttgagaggccattcgttatggctccattagttgacttgttaggatcagatgtagacaatgatacggttctatgctggcattctttttcaaaaaatgggctttttggatcttgggaaacattaggtggtgaatcttccataggaaaagatggtttaagaagggttttacctgtcaatgatcgtttatgggattggtcaaagaaaacttccgtcatgggtattttgaatataactcctgatagttttagtgatggagggaagtttgattccatggggtccgctttatctcgtgttcagaccatgatatctgaaggggttgacataattgatctaggagctcaatccacacgcccaatggcgaccaagatctcagtcgaagaggaactagataggctaatacccgttcttgaaaagattcttgaattacctgaaattgaaggaaagttgttgtctgtggacacattttactcggaagttgcttcagaggcaatcaagaaaggggctcatatggtcaatgatgtatcgggtggaatgttagattctgatatgcttcatgttgtggctgatctaaatgttccatatatcactatgcacatgagaggggacccatccacaatgcaaaacagtgagaatttgaagtatgatgatgttgtcaaagaagttggggaggaattgtatgaacgtgtaaggaatgcagagttatgtggtgttcccgcatggaggatggttcttgatccagggatcgggttttcaaagaaaaccgaagataatttggagatattgatgggactaaagaggtttagaagtgagattggacaaaagagcttaggggtgtctcgtgcacctttgttaatcggaccttcaagaaaaaggtttttgggtgagatttgtggtcggccttctgctgttgagagagatccagggactgtagctgctgttaccagtgcgattttgggtggtgccaatatcgttagggttcataacattggacataatgtggatgctgttaagctttgtgattcaatgttggatcgagctggtagatcttagtagcttaaagttatttgacatcttgttttttattcaattttctggacattactttttgtatttctgtctggcaaatgattttacagggatatcctagaggctaaattgtatattacattcttgattttctcacaaagttttactttttcagtttttcttgttcagttttgtttttgaaaatttgctagatcagattaaatcgttgctttatgatcttcgtgtcttttttgggacgctgcgagtttcatttagtggtattgtaaactccttttaaagtttgtacacacttggtatttatgcttctagttttcgtctatgtctgtcctatattgtgtatagtatgtttaaagttcataaaaagaggataaacgaatagcacgtaaatcttataattcgattccacagagtgcaagttatgctttcctaaatgaaagcaagagtctagacagtttggtcaacgttaacatgagactattgtagaatacaacaccacaagaaaccaaaactgtcttgtgcatttctctcttttccaggtttaaaactcaataccaagacttgatatcaactattattctgcttcaatgattcagcttcagctatagccataggatcatctttggtgtcaggttcatccttctcaccatctcgttcctctgccagtttttttctgtgcaattcctctgcaacttgctttgatgcttggtcttctttttgaaggcgttgcatctcttcttcttgttgctgtcgttcaaaccatgtatccgcatcggcccaaactgtaatttagagccaaaacaacatgattagaatgcagattccagaagccaaattcaaactgtaagaaaaaaaatgtagtgatgatttaactcaaaacaactcacacagctccaaaatggatgggggttcaaattagaggatagcctaatagatagaagcttctaattatggggaaaaaaaaacgatcatggcaatgtattacacaatctaaagcaaccactatgaataataaagataaatctttccgagagtccacatgccttgatatttcataagataggctttttcagaaatcagaatcttcttggcaaatataacactaaaatcagtctaatgcatgttattaagtaaatctttatttgtccatagtataatgccatagattatcgtcaattatccacatcgtcattaaatatcaagtaaccacattatgaattgacttgtttaatcaaagttcatcttacttcttcatagatgaaaaagagctatgcaaatacagaacacatcagttgttctaacatataattactgctagctgtaggaagaaaccaagatgaaacttatctaggattcaaattcatggggatctgttatttagatgaatttagagaaataagaaaataagttgagtgaagtcagtttcgaggttccaaatttgtctttattagttgagctaacaatattaacctctgaagcatgtatagtgtacttgtatgaaccacttaaggatataaatgaagccaaatcagtttcattcgctaattgagcaaatgcacgttctgtttgttagtattgaaatttctttacaggatttctttcaactaaaagcatcacatgtccgactctcggtttgatatttgtgacaagtctaattggttcatcagatgtacttatggctaaatttcgaaacttgaaacaataagagcttagcactcttaaccataaagggttgctgttaatcttacttgttcttgaaagcaattccacagtgaagcatttgttaattgggaagttttgcaatgtttctaaagcttcgatgatattttatattttcaaacatttttcatcacatgtgttaacaaaagccataagaataccggggtaatcaactaaagtctatttaaagttgcatgctcgagatcaagaaaaaaccatcaaatttacataaactgaagggcaaatgtacg

SEQ ID NO: 32 - DNA, 1401 bp, Conyza canadensis
tcgtcctcggatacttcaatacaagtgcattctccagaacaagaagtagtaattgctttaggtagcaatgtgggtaatagacttaataactttaatgaagccttatcccagatgaagaaatcggggatagaaataacaaggcatgcttgtctatacgaaactgaaccagcttatgtgacggaccagcctctttttctcaactctgccgttagagccactacaaagcttggccctcatgagctactgtccattctcaagaaaattgaaaaggaaatgggtcgaaccaaagggcttaggtacggcccacgacccattgacctagatatattgttctatggtaaatgcaaagttaactctgatattctaactgttcctcatgaaagaatctttgagaggccattcgttatggctccattagttgacttgttaggatcagatgtagacaatgatacggttctatgctggcattctttttcaaaaaatgggctttttggatcttgggaaacattaggtggtgaatcttccataggaaaagatggtttaagaagggttttacctgtcaatgatcgtttatgggattggtcaaagaaaacttccgtcatgggtattttgaatataactcctgatagttttagtgatggagggaagtttgattccatggggtccgctttatctcgtgttcagaccatgatatctgaaggggttgacataattgatctaggagctcaatccacacgcccaatggcgaccaagatctcagtcgaagaggaactagataggctaatacccgttcttgaaaagattcttgaattacctgaaattgaaggaaagttgttgtctgtggacacattttactcggaagttgcttcagaggcaatcaagaaaggggctcatatggtcaatgatgtatcgggtggaatgttagattctgatatgcttcatgttgtggctgatctaaatgttccatatatcactatgcacatgagaggggacccatccacaatgcaaaacagtgagaatttgaagtatgatgatgttgtcaaagaagttggggaggaattgtatgaacgtgtaaggaatgcagagttatgtggtgttcccgcatggaggatggttcttgatccagggatcgggttttcaaagaaaaccgaagataatttggagatattgatgggactaaagaggtttagaagtgagattggacaaaagagcttaggggtgtctcgtgcacctttgttaatcggaccttcaagaaaaaggtttttgggtgagatttgtggtcggccttctgctgttgagagagatccagggactgtagctgctgttaccagtgcgattttgggtggtgccaatatcgttagggttcataacattggacataatgtggatgctgttaagctt

SEQ ID NO: 33 - DNA, 1262 bp, Conyza canadensis
gaaataacaaggcatgcttgtctatacgaaactgaaccagcttatgtgacggaccagcctctttttctcaactctgccgttagagccactacaaagcttggccctcatgagctactgtccattctcaagaaaattgaaaaggaaatgggtcgaaccaaagggcttaggtacggcccacgacccattgacctagatatattgttctatggtaaatgcaaagttaactctgatattctaactgttcctcatgaaagaatctttgagaggccattcgttatggctccattagttgacttgttaggatcagatgtagacaatgatacggttctatgctggcattctttttcaaaaaatgggctttttggatcttgggaaacattaggtggtgaatcttccataggaaaagatggtttaagaagggttttacctgtcaatgatcgtttatgggattggtcaaagaaaacttccgtcatgggtattttgaatataactcctgatagttttagtgatggagggaagtttgattccatggggtccgctttatctcgtgttcagaccatgatatctgaaggggttgacataattgatctaggagctcaatccacacgcccaatggcgaccaagatctcagtcgaagaggaactagataggctaatacccgttcttgaaaagattcttgaattacctgaaattgaaggaaagttgttgtctgtggacacattttactcggaagttgcttcagaggcaatcaagaaaggggctcatatggtcaatgatgtatcgggtggaatgttagattctgatatgcttcatgttgtggctgatctaaatgttccatatatcactatgcacatgagaggggacccatccacaatgcaaaacagtgagaatttgaagtatgatgatgttgtcaaagaagttggggaggaattgtatgaacgtgtaaggaatgcagagttatgtggtgttcccgcatggaggatggttcttgatccagggatcgggttttcaaagaaaaccgaagataatttggagatattgatgggactaaagaggtttagaagtgagattggacaaaagagcttaggggtgtctcgtgcacctttgttaatcggaccttcaagaaaaaggtttttgggtgagatttgtggtcggccttctgctgttgagagagatccagggactgtagctgctgttaccagtgcgattttgggtggtgccaatatcgttagggttcataacattggacataatgtggatgc

SEQ ID NO: 34 - DNA, 4643 bp, Euphorbia heterophylla
tttgtatattaaaacaataaaaatgatccagattaataaatagaaaaagtattaataacccgaaaatatcaaatatcaaaatataatgaaacatgaacacgaaatcacaataaagaagttggtatcacaaacacaaagacacccttaaagctcctatgtttgtggagagttgtaagtcgactaatttcagattgcaaaagttaaaaatggccaacttttttagctatgaggttagattagatacgagaatgccttggtgaaaagctggtcactgtttcttcattcgcgtggcaattaacccggcataatcgatacccaattcacggacaaggtcaaaggtatcagctttactcaacccttcgctttgatactaaattgctgattcttattctgattatgctttatttcgttacttcattttattgtctatgcacataagctgttcgacaaaaagcctgaatgaaactgtgagtgctggtacgcggttacaatactcctgattatgtccaatgattatttgggtactttaataccctaatttgtgaagttcactagcgaaagggtagtttcttccccggttatttatcattttatgaatccccaagtctttgaagtacagctgattaaaatgcaagtactcataagttgtcattgtatgtttgaagtccttacatttttggtattttacagaatttgtactgaagaagcataaattcatacctagttaggcttccttgttcctaatatgcttctcctcaagcggctggtgtccactaaacaagggttcaataatgccatcaaccgttttggaggtaatatagttatgcctatgatgctttaagtcgcttttcatcgatttgttacattaccaggctaagattctgcaattgaagttagaaatcgacgagtcttgtcttgaaaagtatggattttgttttgtcaccctggggccggttcaagattgctttcccaggttgctgtcttggcatcacgtcctaatccaaaaccctaaggtgatgggttaatgggtcccttcgcagttatatatcattcacttaactcattaacttccatgtggataatcaactccacacttgccccaacactgtataaacgtagattttgacctttggaaatcattagctggacaaatcctaactaattagtatcaagagccaaaatcttttgaccaagaccaaaatatgttgtttcattgttgcagcattgtcatcatcctttctccataccgaaccaaatagttcggtggaagttcattctcaagagcaagaagtagtaattgcattaggaagcaatgtgggagatagagttcataatttcaaccaagcattgcaattaatgaaaagttccggcattaacataactagacatggttgtttgtaccagacagcacctgcttatgtcactgaccaacctcactttctcaactcagcagttcgagctttcacgaaactcggaccccacgagttattaggagttttaaagcagatcgaaaaggacttgggccgtaccaaagggattaggtacggaccaaggccaatcgatttggatatacttttctatggaaagttccggattaattccgatacacttattgttcctcatgagagaatatgggagagaccctttgtgatggccccattggtggatttactcggctcggaaatcgagaatgacacggtcgccagctggcattccttgtccggcggtctttttgaatcatgggaaaagttgggcggcgaaagcctgatcgggaaggacggaatgaaaagagttaccccgatcggaaaccatttatgggattggtcggaaaagacttctgtaatgggaattatcaatttaacccccgatagtttcagcgatgggggcaagttgacaactattgattctatagtttctaaggttcgctcgatgatttccgaaggggcggatattgtcgattttggtgctcaatcgacacgccctatggctaaaaggatatccccgcaagaggaaatggataggctaatccctgtattggaagcggttgtgaaaatacccgagatgaccgaaaagctcatatcggtcgacacattttactcggaagtcgccttagaagcggtccaaagaggagcaaatctcgtaaatgacgtatctggggggcaattagatccaaaaatgacggaaaatgtcgctaatctcgaggtaccatatatcttaatgcacatgaggggggaccccaccacaatgcagaacactcagaatctaaaatacgataatgtttgtaaagaggttgcctttgagttgttttcgagggttaaagaagccgagatgtccggaattccagcctggcgaatgtttattgaccctggaattgggttttcgaagaacacgaatcagaatctggaaatcctgatgggaattccaaggatccgggccgagattgggaggaaaagcgtgggtatttctcgtgggccgattcttattgggccttcgagaaagaggtttttgggcgaaatttgcgaacgccctgaagcaactgaaagagacccggcaactgttgcttctgttactactgggattctgggaggggcaaatattgttagagttcataatgttagggataatgtggatgctgtgaagctatgtgactcgatgatgaaactaaagaggtctacttaaaaattatcgggtatagttcgatttctttctttccgaatcttttgtttgactttcagattgtctttgattggcaaaacagtttcggctgcggttactgtttgctgttgcagtttaccgttgttgtttctaattgtgattttaagttattgttgttagttgatttgtacttagtgcttggtgaaaatataattcacggttgcggttggaggtataaattactaaaatagacatattttaaataaattagaagttatattgaagggtatttaggataaataaaagtccaaaccacaaaaaaaaaaaaccgtaaaactattgatttggtgaggtttggaaaaggagataaatcccttatgaaaagcaactccatatatgttttgaaacttaaccaaacacttgtatacctccttttccttttcaaacctcaaaagacctttttatagtaagctcttttgaagtatatacgctattgtttccgaatcattttggacttttcgtcgaaatcaacaatactgttagctagtcatatttcaagatatttgggatgtaaagatcaaggtagttgttgttgtattctgttctcatcagcaaaatttgttataactccaccaagatttaattttttccaatctgaaaatgtcttatgttatagtaagaagtgagatgagtttatgtctttgtgaaaagattttgcccaattaatgcaaaaattttgtgacaagacaagaggggtgaaagagacaagcaacaagagaaagcagtgaaaggggcaaatgttaggaaactgttagctagaaaagggtagagggtggcctactttaccaatttgagcattaaaaaagcacatggctatgtattttagaccaaagtccctattgttggcagatgaactaagcaaccatatggttcattaactaactaactagttattgcaaatttgactgttccaattcatcaaaggactctccccatagaccattcaacacttgggttctagccctccctctcaccctcagacaacccttgtgaacctcattttcatcattggtccccttttttgttaggactacacttgtctctataaaatctcttctagccatgtgttatgtgtagactttttcggtttgaggattgggtcgggttcgggctgggtctcgtcgggctttttactgcaaacatcaaactagggttctaaacccatacttcagtactctggttcggattgaacttgcgagatcattctaaattcaatttacggctcatagattatatttgagagttttgagttttgggtggattagattgaaaacgactattttttgttgtataaatatatctaaaaagttatataattacattctttattggaattcatcaatatatataagaatttgactttgttgtagccattttgaaagttcgataagctactcccttcgtctagtttcaatagttcatttaaaagtttcattaattctgttttcaattgtcaatttaaattttcataatatttttcaccatttttttttccaatttgcccttgtatttattgagaaagagattatgttatgatgaaagtataagttgctaatcaagaagaaagagattttaatgtaattatttaaataataaattcctaccattttaattgtgtgacaattggttataatgattccgttgcatcgacaactaaaactagatagaaggatcaaaatataaaattttaacataattaatacatataatatataaatgtataatagatacaattattattcctcgaattaaaattggctaattatataataatataatccaacctataaaaacttttgagcttttaagcttgagatgagctttgtaaaaatgattgtacaagcccgaccatgaaagagagccaagtagggtcaaatgcataccccggcttgaatataaattcaatatatgttctttcaaatcttgcc

SEQ ID NO: 35 - DNA, 4556 bp, Euphorbia heterophylla
aaatataaatataaatataaaaataaaaataaattataataatataaaatataaaaattaatttaaaccatgttttaaaaatagtgttatgaaaaactccgcttgggcgccgtctagatagtttacttaggctgatttttaaaacaaaaccccccgcttaacccgcctaaatcggtcaattgtttatatccttatgactacttaaaatcgcagcggtgttatggtgaattatcatgttttaatgagcttatgaaatgcaataataaaaaaataaccattaataatttgatatacatttatcgggaaaaatgaaataataaaaaataaaaaaatatcaaaaaaatctataaaatatttgtacctttttcaattagtacccattttgtatattaaaacaataaaaatgatccagattaataaatagaaaaagtattaataacccgaaaatatcaaatatcaaaatataatgaaacatgaacacgaaaacacaataaagaagttggtatcacaaacacaaagacacccttaaagctcctatgtttgcggagagttgtaagtcgactaatttcagattgaaaaagttaaaaatggccaacttttttagctatgaggttagattagatacgagaatgcctttgtgaaaagctggtcactatttcttcattcgcgtggcaattaacccggcataatcgatagccaattcacggacaaggtcaaaggtatcagctttactcaacccttcgctttgatactaaattgctgattcttattctgattatgctttatttcgttacttcattttattgtctatgcacataagctgttcgacaaaaagcctgaatgaaactgtgagtgctggtacgcggttacaatactcctgattatgtccaatgattatttgggtactttaataccctaatttgtgaagttcactagcgaaagggtagtttcttccccggttatttatcattttatgaatccccaagtctttgaagtacagctgattaaaatgcaagtactcataagttgtcattgtatgtttgaaatccttacgttgttggtattttacagaatttgtactgaagaagcataaattcatacctagttaggcttccttgttcctaatatgcttctcctcaagcggctggtgtccactaaacaagggttcaataatgccatcaaccgttttggaggtaatatagttatgcctatgatgctttaagtcgcttttcatcgatttgttacattaccaggctaagattctgcaattgaagttagaaatcgacgagtcttatcttgaaaagtatggattttgttttgtcaccctggggccagttcaagattgctttcccaggttgctgtctcggcaccacatcctaatccaaaaccctaaggtgatgggttaatgggtcccttcgcagttatatttcattcacttaactcattagcttccatgtggataatcaactccacacttgccccaaaactgtataaacgtagattttgacctttggaaatcattagctggacaaatcctaactaattagtatcaagagccaaaatcttttgaccaagaccaaatatgttgtttcattgttgcagcattgtcatcatcctttctccataccgaaccaaatagttcggtggaagttcattctcaagagcaagaagtagtaattgcattaggaagcaatgtgggagatagagttcataatttcaaccaagcattgcgattaatgaaaagttccggcattaacgtaactagacatggttgtttgtaccagacagcacctgcttatgtcactgaccaacctcactttctcaactcagcagttcgagctttcacgaaactcggaccccacgagttattaggagttttaaagcagatcgaaaaggacttgggccgtaccaaagggattaggtacggaccaaggccaatcgatttggatatacttttctatggaaagttccggattaattccgatacacttattgttcctcatgagagaatatgggagagaccctttgtgatggccccattggtggatttactcggctcggaaatcgagaatgacacggtcgccagctggcattccttgtccggcggtctttttgaatcatgggaaaagttgggcggcgaaagcctgatcgggaaggacggaatgaaaagagttaccccgatcggaaaccatttatgggattggtcggaaaagacttctgtaatgggaattatcaatttaacccccgatagtttcagcgatgggggcaagttgacaactattgattctatagtttctaaggttcgctcgatgatttccgaaggggcggatattgtcgattttggtgctcaatcgacacgccctatggctaaaaggatatccccgcaagaggaaatggataggctaatccctgtattggaagcggttgtgaaaatacctgagatgaccggaaagctcatatcggtcgacacattttactcggaagtcgccttagaagcggtccaaagaggagcaaatctcgtaaatgacgtatctggggggcaattagatccaaaaatgacggaaaatgtcgctaatctcgaggtaccatatatcttaatgcacatgaggggggaccccaccacaatgcagaacactcagaatctaaaatacgataatgtttgtaaagaggttgcctttgagttgttttcgagggttaaagaagccgagatgtccggaattccagcctggcgaatgattattgaccctggaattgggttttcgaagaacacgaatcagaatctggaaatcctgatgggaattccaaggatccgggccgagattgggagggaaagcgtgggtatttctcgtgggccgattcttattgggccttcgagaaagaggtttttgggcgaaatttgcgaacgccctaaagcaactgaaagagacccggcaactgttgcttctgttactactgggattctgggaggggcaaatattgttagagttcataatgttagggataatgtggatgctgtgaagctatgcgactcgatgatgaaactaaagaggtctacttaaaaagtatcgggtatagttcgatttctttctttccgaatcttttgtttgactttcagattgtctttgattggcaaaacagtttcggctgcggttactgttgttgtttgttgttgcagtttagtgttgttgtttctaattgtgattttaagttattgttgttagctgatttgtacttattgtttggtgaaaatataattcacggttgcggttggaggtataaattactaaaatagacatattttaaataaattataagttatattgaagggtatttaggataaataaaaatccaaaccacaaaaaaaaacccgtaaaactattgatttggtgaggtttggaaaaggagataaatcccttataaaaaaaacaacttcatatatgttttgaaacttaaccaaacacttgtatacctccttttccttttcaaacctcaaaagacctttttatagtaagctcttttgaagtctatacgctattgtttccgaatcattttggacttttcatcgaaatcaacaatactgctagctagtcatatttcaagatatttgggatgtaaagatcaaggtagttgttgttgtattctgttctcatcagcaaaatttgttataactccaccaagatttaattttttccaatctgaaaatgtcttatgttatagtaagaagtgagatgagtttatgtctttgtgaaaagattttgcccaattaatgcaaaaattttgtgacaagacaagaggggtgaaagagacaagcaacaagagaaagcagtgaaaggggcaaatgttaggaaactgttagctagaaaagggtagagggtggcctactttaccaatttgagcattaaaaaagcacatggctatgtattttagaccaaagtccctattgttggcagatgaactaagcaaccatatggttcattaactaactaactagttattgcaaatttgactgttccaattcatcaaaggactctccccatagaccattcaacacttgggttctagccctccctctcaccctcagacaacccttgtgaacctcattttcatcattggtccccttttttgttaggactacacttgtctctataaaatctcttctagccatgtgttatgtgtagactttttcggtttgaggattgggtcgggttcgggctgggtctcgtcggactttttactgcaaacatcaaactagggttctaaacccatacttcagtactctggttcggattgaacttgcgagatcattctaaattcaatttacggctcatagattatatttgagagttttgagttttgggtggattagattgaaaacgactattttttgttgtataaatatatctaaaaagttatataattacattctttattggaattcatcaatatatataagaatttgactttgttgtagccattttgaaagttcgataagctactcccttcgtctagtttcaatagttcatttaaaagtttcattaattctgttttcaattgtc

SEQ ID NO: 36 - DNA, 1425 bp, Euphorbia heterophylla
gcagcattgtcatcatcctttctccataccgaaccaaatagttcggtggaagttcattctcaagagcaagaagtagtaattgcattaggaagcaatgtgggagatagagttcataatttcaaccaagcattgcgattaatgaaaagttccggcattaacgtaactagacatggttgtttgtaccagacagcacctgcttatgtcactgaccaacctcactttctcaactcagcagttcgagctttcacgaaactcggaccccacgagttattaggagttttaaagcagatcgaaaaggacttgggccgtaccaaagggattaggtacggaccaaggccaatcgatttggatatacttttctatggaaagttccggattaattccgatacacttattgttcctcatgagagaatatgggagagaccctttgtgatggccccattggtggatttactcggctcggaaatcgagaatgacacggtcgccagctggcattccttgtccggcggtctttttgaatcatgggaaaagttgggcggcgaaagcctgatcgggaaggacggaatgaaaagagttaccccgatcggaaaccatttatgggattggtcggaaaagacttctgtaatgggaattatcaatttaacccccgatagtttcagcgatgggggcaagttgacaactattgattctatagtttctaaggttcgctcgatgatttccgaaggggcggatattgtcgattttggtgctcaatcgacacgccctatggctaaaaggatatccccgcaagaggaaatggataggctaatccctgtattggaagcggttgtgaaaatacctgagatgaccggaaagctcatatcggtcgacacattttactcggaagtcgccttagaagcggtccaaagaggagcaaatctcgtaaatgacgtatctggggggcaattagatccaaaaatgacggaaaatgtcgctaatctcgaggtaccatatatcttaatgcacatgaggggggaccccaccacaatgcagaacactcagaatctaaaatacgataatgtttgtaaagaggttgcctttgagttgttttcgagggttaaagaagccgagatgtccggaattccagcctggcgaatgattattgaccctggaattgggttttcgaagaacacgaatcagaatctggaaatcctgatgggaattccaaggatccgggccgagattgggagggaaagcgtgggtatttctcgtgggccgattcttattgggccttcgagaaagaggtttttgggcgaaatttgcgaacgccctaaagcaactgaaagagacccggcaactgttgcttctgttactactgggattctgggaggggcaaatattgttagagttcataatgttagggataatgtggatgctgtgaagcta

SEQ ID NO: 37 - DNA, 1425 bp, Euphorbia heterophylla
gcagcattgtcatcatcctttctccataccgaaccaaatagttcggtggaagttcattctcaagagcaagaagtagtaattgcattaggaagcaatgtgggagatagagttcataatttcaaccaagcattgcaattaatgaaaagttccggcattaacataactagacatggttgtttgtaccagacagcacctgcttatgtcactgaccaacctcactttctcaactcagcagttcgagctttcacgaaactcggaccccacgagttattaggagttttaaagcagatcgaaaaggacttgggccgtaccaaagggattaggtacggaccaaggccaatcgatttggatatacttttctatggaaagttccggattaattccgatacacttattgttcctcatgagagaatatgggagagaccctttgtgatggccccattggtggatttactcggctcggaaatcgagaatgacacggtcgccagctggcattccttgtccggcggtctttttgaatcatgggaaaagttgggcggcgaaagcctgatcgggaaggacggaatgaaaagagttaccccgatcggaaaccatttatgggattggtcggaaaagacttctgtaatgggaattatcaatttaacccccgatagtttcagcgatgggggcaagttgacaactattgattctatagtttctaaggttcgctcgatgatttccgaaggggcggatattgtcgattttggtgctcaatcgacacgccctatggctaaaaggatatccccgcaagaggaaatggataggctaatccctgtattggaagcggttgtgaaaatacccgagatgaccgaaaagctcatatcggtcgacacattttactcggaagtcgccttagaagcggtccaaagaggagcaaatctcgtaaatgacgtatctggggggcaattagatccaaaaatgacggaaaatgtcgctaatctcgaggtaccatatatcttaatgcacatgaggggggaccccaccacaatgcagaacactcagaatctaaaatacgataatgtttgtaaagaggttgcctttgagttgttttcgagggttaaagaagccgagatgtccggaattccagcctggcgaatgtttattgaccctggaattgggttttcgaagaacacgaatcagaatctggaaatcctgatgggaattccaaggatccgggccgagattgggaggaaaagcgtgggtatttctcgtgggccgattcttattgggccttcgagaaagaggtttttgggcgaaatttgcgaacgccctgaagcaactgaaagagacccggcaactgttgcttctgttactactgggattctgggaggggcaaatattgttagagttcataatgttagggataatgtggatgctgtgaagcta

SEQ ID NO: 38 - DNA, 681 bp, Euphorbia heterophylla
ttaaagcagatcgaaaaggacttgggccgtaccaaagggattaggtacggaccaaggccaatcgatttggatatactttttctatggaaagttccggattaattccgatacacttattgttcctcatgagagaatatgggagagaccctttgtgatggccccattggtggatttactcggctcggaaatcgagaatgacacggtcgccagctggcattccttgtccggcggtctttttgaatcatgggaaaagttgggcggcgaaagcctgatcgggaaggacggaatgaaaagagttaccccgatcggaaaccatttatgggattggtcggaaaagacttctgtaatgggaattatcaatttaacccccgatagtttcagcgatgggggcaagttgacaactattgattctatagtttctaaggttcgctcgatgatttccgaaggggcggatattgtcgattttggtgctcaatcgacacgccctatggctaaaaggatatccccgcaagaggaaatggataggctaatccctgtattggaagcggttgtgaaaatacccgagatgaccgaaaagctcatatcggtcgacacattttactcggaagtcgccttagaagcggtccaaagaggagcgaatctcgtaaatgacgtatctggggggcaattagatccaaaaat

SEQ ID NO: 39 - DNA, 444 bp, Euphorbia heterophylla
tctggggagtgatggttggaatagatgcttattcctggcagagatcgaaatgctccattttagtttcgggttttgttaaaatggggatttgttaaattgtttcaatggataattcactgttgtattagacttgatattgttagcagacactgatttttatgtattcgtgaactgaaaaggttgattttcttccatgttttgtctgatttatgatggaacttcatttcttagtggttaatgttggtatttccttctgttgtttgatttgtatctgtaattggtaccatgttatatgcaatcttttaaaaaatgttttaaaaagcgctatacgtggcagctgagactgttgagacttgaaaacgcacgggcatgtgcggggtttaggcggatttgtattttttattttttaaaatattttttaatatataaaaatataaatataaa

SEQ ID NO: 40 - DNA, 379 bp, Euphorbia heterophylla
gttatgatgaaagtataagttgctaatcaagaagaaagagattttaatgtaattatttaaataataaattcctaccattttaattgtgtgacaattggttataatgattccgttgcatcgacaactaaaactagatagaaggatcaaaatataaaattttaacataattaatacatataatatataaatgtataatagatacaattattattcctcgaattaaaattggctaattatataataatataatccaacctataaaaacttttgagcttttaagcttgagatgagctttgtaaaaatgattgtacaagcccgaccatgaaagagagccaagtagggtcaaatgcataccctggcttgaatataaattcaatat

SEQ ID NO: 41 - DNA, 4031 bp, Digitaria sanguinalis
aaatcgtaaaatttataactaacatgttccttcatgaatccaaaagccaagtaaacaggtccatgagtgcctttgtgagattgcttcgaatagaatatacctacggctcttaatttcagattctcttaggtagtgtttaggtagcgtttggaagtagatatttaggcttggaattaggaattggcattgagatctacgaatacctgccgtttggatgtctaagaatttggaattggaattgcagcccaataccaaccggcattccttggcctctcatctgcctgccctcagtatagatagagcttatctccttcatcctcgcggagtggtggcaagcggcggcgagatccggaccacatggggtgtgggagcgcggtggcagagtggtggcaagcgatggcgacggcaccgtgccgccgtgccacattggcacggcgacggacgagtttggatggtgtcagtacagaccactgcccaattctatccttttgtgcatccaaacataaaatctgaattaggatgctcttttgattctacaaggcaatgtaatgaccatccaaacaacagattctgaattggagtcaattcaatgttacgattgtattggcatcggacctaattcaatgtcatgccctctagtatctacattcaaacggagcggaaaaatctacctccgtcccaagattccggcccaagatcccgactcttaatacttttttcatctttcgctgaaatttcagcccaagatcccggcccgcctttttgcctccacgctgggcctggtgggacgcggccccttcacgagagtcgagaggggaaagcggaaagggaaacaaaaaaactcaacgaatcgccgccgccgccttgccctgcagcgaacaattaagggccccgtacctctctctgcccgttcttccgctgcactccgcatctgagcggacgggtggggacaccgccgacggcgccggggtggcgcctccgtctgtgctccaaccaaggcagtgggtgctggttgctgccaatcgaccggccgggcatgctctgaccatcgagaaaatcttcgggtaacacaacgttgttcatttcgtttgtatcgtggcatagttgatttccctaacttttaggatgcgtttcgattcattgtatctgtataccaaaacagagttcgataacttagattgaacggtaggcttcagcatctaaggcagcaggtgcaggtctattctatcactctgaaaattagttttaactgtctgatgataggaggattagctaatgttgttatatgaggaggtttcttgctgactagagagctatttgaccacctgatagattgcctcgttagcaacttagcattatgacgacgatggcagcgagccatttcatgtagaaaacgaacatgagctgagaagcttaatcgtataagcttcaattggcttcctggctggggaggtttgctagagaacttctgtattttgttcattcatttaatctgatgtatgttggcagctctcctgtcaggtacagaaatttagacggccctgaaaccaagcagggcaacctcaccaataggggggcagtcgcaccacaaaggttgcttgcctccagtggggcatctcctctccttacattgagaggtttatttgacgtctaagatatctgaactcctgcctccagttagagaaaaggcccttgttcatacctcaaatttgtctttgctcttctattgaccaggtgtgatggagtgatgcttgcgtgaaatatcctacaaggctacaatttatttgaagcatttgctttcttaattatcatatgttcaacgatgctcctccatgctaaggagtcccttaagaagatgtatcctgttgctatgaactactttggagggctcctcccatcgcattcgttttcaggtactgttcaaatcttttcttttcaaatcccctatcatgcgatcaagagcagctctaagcttctaactcacccctttgcatttctaaacagtgtctgatcttgttcatccgctgaggttcccaaacagagctcctagacatgctattccattcagggctcgttcgtttacgcgatgttcgcttgagggatgttcaactgaccaagagattgtgattgctatgggaagcaacgtgggtgatggagtcagtacatttgacagggcattacaaatgatgaaaagctcaggcgtgaacatcactaggcatgcctgtctatatgagactgcccctgcttatgtgaccgatcagccacggttcctgaactctgccattcgaggtacgactaggctgggacctcacgagttgcttaagaaactaaaggaaattgagaaggatataggacgcactggtggaataaggtatggcccaagaccaatcgatctagatatacttctgtacggcaattcccagattgatactgaggctctaattgtgccacatgaacgcatccaggagaggccatttgttctagcacctcttgttgaccttctaggtgcatctggcaatgatagtgtcgaaacaagctggcactctctttcaaagtgcagtggtggtttctttgaattatggaataaacttgggggtgaatctataattggaacagaaagtattaaaagggtattacctgttgggaatcatttgtttgattggtgtgagagaaccctcgtcatgggggtccttaatctaacaccagacagctttagtgatggaggtaagtttcaacaagtggaagctgctatttctcaggctaaattattaatttcagaaggtgcagatatcatagatattggtgctcaatctaccaggccttttgcaaagagattatctccaaacgaagaaattgaaagattggttcctgttctggatgtgattacgaaaattcctgaaatggagggaaagttgctctcagtggatacattctatgcagaagttgcatgtgaagctgtgaaaagaggagttcacatgatcaatgatgtatctggtggacagcttgaccccaaaattcttaaagttgctgctgaactcaaagttccgtatgttgcaatgcacatgaggggagatccatcaacaatgcaaagtgaacaaaatttacagtatgacgatgtctgcaaggaagttgcttcagagctatatgcacaggtgagagaagcagagttatctgggattccattgtggaggatagttctagatccaggcattgggttctccaagaaatccaaacataaccttgaagtaattatgggattggaatccattaggagggagatgagtaaaatgagtattggtgcttcacatgtgccaatattactgggaccctctaggaaaagatttttgggtgaaatatgcaatcgtgaaaatccagttgagagagatgttgctactgttgcagctgtgacggcgggtattttgaatggtgctaacatagtaagggtacataatgcaggatacagttcagatgctgcaaagttttgtgatgcattgaataagagaagaagaatggaagactgaaccagctgatcaaaaagataccgagctctgattttattcgagaaaatggtgatgcaggatagttactctgctgctcaatgggattctcatattacatcatttgtggagtattgtttttgtaataaataaaccagggatgacgtttttcctgtgtcatctcctatctagtttctagatacttcaatgaagcatggttcaactcagatcacttggaaagagtagtcaaggaataaggttacagaatcagttagtaattacagattcttacctcttgtggtcctattttagatgattttccagaacattttctttccaatttaataatatcagaagggcctaatttgtgtgtatcttttctagtccagacatatttgataagttggggttttacctgcttctccctttacctttgtattttgcactttgtttgagctgtgcatcattgtattagaggaagtgtattttgtacgttggtatacccaacgaggaaacctcatgttttgcaagtgc

SEQ ID NO: 42 - DNA, 1428 bp, Digitaria sanguinalis
gctcgttcgtttacgcgatgttcgcttgagggatgttcaactgaccaagagattgtgattgctatgggaagcaacgtgggtgatggagtcagtacatttgacagggcattacaaatgatgaaaagctcaggcgtgaacatcactaggcatgcctgtctatatgagactgcccctgcttatgtgaccgatcagccacggttcctgaactctgccattcgaggtacgactaggctgggacctcacgagttgcttaagaaactaaaggaaattgagaaggatataggacgcactggtggaataaggtatggcccaagaccaatcgatctagatatacttctgtacggcaattcccagattgatactgaggctctaattgtgccacatgaacgcatccaggagaggccatttgttctagcacctcttgttgaccttctaggtgcatctggcaatgatagtgtcgaaacaagctggcactctctttcaaagtgcagtggtggtttctttgaattatggaataaacttgggggtgaatctataattggaacagaaagtattaaaagggtattacctgttgggaatcatttgtttgattggtgtgagagaaccctcgtcatgggggtccttaatctaacaccagacagctttagtgatggaggtaagtttcaacaagtggaagctgctatttctcaggctaaattattaatttcagaaggtgcagatatcatagatattggtgctcaatctaccaggccttttgcaaagagattatctccaaacgaagaaattgaaagattggttcctgttctggatgtgattacgaaaattcctgaaatggagggaaagttgctctcagtggatacattctatgcagaagttgcatgtgaagctgtgaaaagaggagttcacatgatcaatgatgtatctggtggacagcttgaccccaaaattcttaaagttgctgctgaactcaaagttccgtatgttgcaatgcacatgaggggagatccatcaacaatgcaaagtgaacaaaatttacagtatgacgatgtctgcaaggaagttgcttcagagctatatgcacaggtgagagaagcagagttatctgggattccattgtggaggatagttctagatccaggcattgggttctccaagaaatccaaacataaccttgaagtaattatgggattggaatccattaggagggagatgagtaaaatgagtattggtgcttcacatgtgccaatattactgggaccctctaggaaaagatttttgggtgaaatatgcaatcgtgaaaatccagttgagagagatgttgctactgttgcagctgtgacggcgggtattttgaatggtgctaacatagtaagggtacataatgcaggatacagttcagatgctgcaaagttttgtgatgcattgaat

SEQ ID NO: 43 - DNA, 4007 bp, Kochia scoparia
gaatgtgcttaggcggctgcaatcagccagtttgatttggaagggaatttccaagtatgatagaggtaaagcacagttgtttcattttttgactatatatttaatttgggaatctgtactgtttcattttgtaactttattttgatcctgagttattgaatctgtcatgtctcattttgagacggtattttgaatctgagtctggatatgaaattatgaataggccataggtatttataaatatagggtttaagttgatgatctggatgctatcagagctctattatgcaggattacaaatcataatttcattgtgaaatttacttctttatgtgtattcacttgaagattcataattttgatgtattcataaatttgatgtgccctaaacagaaagagtcagcatgtgctgaaagtataagggggaaaaccaagatgtataaatcttttccttgatgagttgcttatatgattgatgtcatgattctaaaatgcctttacctaattcaaatggattttaaagattacgtatgtacttttgcagcttcaagtcttgcttttttgcactcatcaccagaaactactattgaggtttgttctaaagagcatgaagttgtaattgctctagggagcaatgtaggagacagattagataactttaaccaagctctgcagctaatgaagaaattaggagtaaatatcacaaggcatggctgcttatacgagacagatcctgcctatgtgactgatcaaccaaagtttcttaactctgcagtgaggggcttcacaaaacttgggcctctagaactattagggatgctgaagaaaattgagaaggatatgggtcgaactaatggaataagatatggccctaggccaattgacttagacattcttttttatgggaagtttagggtaagctctgagaaacttacagttccgcatgaaaggatatgggaaagaccctttgtgatggcaccactaattgatataattggatcggatgtagaaaatgacactgttgctgcatggcattcattatcaaaattttctggtggactatttgaagcgtgggataaacttggtggagattcactcattgggaaggacggaatgagtagggttttgccagttgggaaccacttgtgggattggtcgtgcaaaacctctgtaatgggagtcttgaatttgactccggacagctttagtgatggaggaaagtttctacctgtagaaaatgcagtttctcaggttcgtcaaatgatctcagaaggtgctgacataattgatattggagcgcaatcaacaaggcccatggcaactaggatttctgctgaggaagagctggaaagactagtccctgttttagaaggtgtcaaggatgttatcgaggaagaaggaagaatgttatcagtggatacattttactcaaaagttgcttctgaagctgtcaataagggagcacatatggtgaatgatgtttcgagtggaaagctagatcctgagatgttcaatgttgttgcagggcttaaagtgccttatatagcaatgcacatgcgaggtgatccttcttcaatgcaaaatgctgataatttgacatacaatgatgtttgtaaggaggtggctttagagttgagctctaggattacagatgcagaattatcaggtattcctgcttggaggatagttattgatcctggcattggattttccaagaatacaaagcaaaatttggaaattctcacagggttgaaaagaattcggcaagaaatagcaaaaaagagtttggctgtggctcatggtcccttactgattggaccttcaagaaagaggtttttgggtgagatctgcaatcgtcctgtagcatctgatagagatccggcaactgttgcttctatcaccgctggagttttgggtggtgcaaacattgtaagagtacataatgttcgggatagccttgatggtgtcaagctatgtgatgcaatgcttgaatatcagtataactgaagattcttagattcttagattcttagattctaagagttgattttatagattattgtctttgttgtgtgccttcggcagatatgaataatatgattgtaattttgttgcttatgattttcttcctacctttcttttatcatagaatttagattaaaaaatgtacttgaatttcataaatggtgaaataaagtttgtcggtgcttcatgcaatacgacttttctagtttgcattgctctatcataggggctgtaagcagattagcccagtcaattcacattagatttagacggccggtgcaaaaacagcataatgttgctaacgctgtttatacatttaaactatatgatctctaagagcaaaatacaaaataatacaatgtgtttcatgaagatgtccaatcatttctccctcaaaaaaagatcgtgatgaacaatcaggtactttgattttgttgtagaacctcagcttcagccctagccatgggatcatctttttctccttctcgctcctcagccaatttctttctgtgcatctcctctgcagcttctattgaagctttgtgctcagcctggagacgctgcatttcctcttccatctgctgtctttcgaaccatgtatcagcatccgcccaaactgcagaaataatttaaacaagttaatcatacggagtgtgcagcatggcaaattggcaataagatggtgcaagtaacaagctcaaatgatccaccatagccaaattcctaaccattggactgtgtcatatactcatatcagctagtagaaacattctcaccataaccctaactattcgaggataagtgataccattcaacaatagtaaagatcaaatcaggtattactagacctatcaaagtaatacggagtagttgttattgttacagagttaggaagaatcctattttcccatttctcctatcaatcctaaacttttgtgactgaactatcgacatatagtgtgaagaacatgaatggcagaagtcacaactctgattcctgagtcctgactataaactattggatgactgtgatacatcattagtcctttaagcattgagttactctattctacatagttaaattatatagattccaagatccgaatccatgaattatcaattaccataaattgatcttcttcctttgagttttgtcaacagttcctactccgaaatatgtcttaaatctagaagcaatagacacttatcaatgatacagcaatagacgaattcatctacccaaggcagcatcaacaacagctgccgtttgcgataatcgaaactatcatagtgtccttttcactattagtgcagatgttatctagggtctaaacctctatgtttctacattcctggaaaatcttattctgtactgcataatcctctcacaaaaaattcgccaatgaatgacaaattagctatccagaattgcagattgattgaatcaaattgatcatggcagcagcacaattgcagcagttatcaattgaagcacaatattacctctcaatcataacaatcaaagcacagaaattccaatcaataactccacaatatagtatcaatctctaactcagaaactaaacagaagcaaaaacccattcaatcaaagcaacagtaaaattagggtttgaattagaaagaaagagagggaagagaaacatactgggttgcgcagcccatccctgttcaccacccttaatcttctcgggaagagcgtaattgacagtgagaacacgaccgtaaagctcagcaccatccatgttatccatggcagaagaagcgtcgtctctctccaagaaagtgacgaaaccgaaagaacgatgcttgttggtggccaaatcaagaggggttttgacgtctttgatttctccaaaagggatgaaggcagcatgaaggattgattcgttgacttcttctgctaatcctcctacgtataatgtgttcttctgcattccttgttgattcgtcgccatttttcg

SEQ ID NO: 44 - DNA, 3505 bp, Kochia scoparia
taattcaaatggattttaaagattacgtatgtacttttgcagcttcaagtcttgcttttttgcactcatcaccagaaactactattgaggtttgttctaaagagcatgaagttgtaattgctctagggagcaatgtaggagacagattagataactttaaccaagctctgcagctaatgaagaaattaggagtaaatatcacaaggcatggctgcttatacgagacagatcctgcctatgtgactgatcaaccaaagtttcttaactctgcagtgaggggcttcacaaaacttgggcctctagaactattagggatgctgaagaaaattgagaaggatatgggtcgaactaatggaataagatatggccctaggccaattgacttagacattcttttttatgggaagtttagggtaagctctgagaaacttacagttccgcatgaaaggatatgggaaagaccctttgtgatggcaccactaattgatataattggatcggatgtagaaaatgacactgttgctgcatggcattcattatcaaaattttctggtggactatttgaagcgtgggataaacttggtggagattcactcattgggaaggacggaatgagtagggttttgccagttgggaaccacttgtgggattggtcgtgcaaaacctctgtaatgggagtcttgaatttgactccggacagctttagtgatggaggaaagtttctacctgtagaaaatgcagtttctcaggttcgtcaaatgatctcagaaggtgctgacataattgatattggagcgcaatcaacaaggcccatggcaactaggatttctgctgaggaagagctggaaagactagtccctgttttagaaggtgtcaaggatgttatcgaggaagaaggaagaatgttatcagtggatacattttactcaaaagttgcttctgaagctgtcaataagggagcacatatggtgaatgatgtttcgagtggaaagctagatcctgagatgttcaatgttgttgcagggcttaaagtgccttatatagcaatgcacatgcgaggtgatccttcttcaatgcaaaatgctgataatttgacatacaatgatgtttgtaaggaggtggctttagagttgagctctaggattacagatgcagaattatcaggtattcctgcttggaggatagttattgatcctggcattggattttccaagaatacaaagcaaaatttggaaattctcacagggttgaaaagaattcggcaagaaatagcaaaaaagagtttggctgtggctcatggtcccttactgattggaccttcaagaaagaggtttttgggtgagatctgcaatcgtcctgtagcatctgatagagatccggcaactgttgcttctatcaccgctggagttttgggtggtgcaaacattgtaagagtacataatgttcgggatagccttgatggtgtcaagctatgtgatgcaatgcttgaatatcagtataactgaagattcttagattcttagattcttagattctaagagttgattttatagattattgtctttgttgtgtgccttcggcagatatgaataatatgattgtaattttgttgcttatgattttcttcctacctttcttttatcatagaatttagattaaaaaatgtacttgaatttcataaatggtgaaataaagtttgtcggtgcttcatgcaatacgacttttctagtttgcattgctctatcataggggctgtaagcagattagcccagtcaattcacattagatttagacggccggtgcaaaaacagcataatgttgctaacgctgtttatacatttaaactatatgatctctaagagcaaaatacaaaataatacaatgtgtttcatgaagatgtccaatcatttctccctcaaaaaaagatcgtgatgaacaatcaggtactttgattttgttgtagaacctcagcttcagccctagccatgggatcatctttttctccttctcgctcctcagccaatttctttctgtgcatctcctctgcagcttctattgaagctttgtgctcagcctggagacgctgcatttcctcttccatctgctgtctttcgaaccatgtatcagcatccgcccaaactgcagaaataatttaaacaagttaatcatacggagtgtgcagcatggcaaattggcaataagatggtgcaagtaacaagctcaaatgatccaccatagccaaattcctaaccattggactgtgtcatatactcatatcagctagtagaaacattctcaccataaccctaactattcgaggataagtgataccattcaacaatagtaaagatcaaatcaggtattactagacctatcaaagtaatacggagtagttgttattgttacagagttaggaagaatcctattttcccatttctcctatcaatcctaaacttttgtgactgaactatcgacatatagtgtgaagaacatgaatggcagaagtcacaactctgattcctgagtcctgactataaactattggatgactgtgatacatcattagtcctttaagcattgagttactctattctacatagttaaattatatagattccaagatccgaatccatgaattatcaattaccataaattgatcttcttcctttgagttttgtcaacagttcctactccgaaatatgtcttaaatctagaagcaatagacacttatcaatgatacagcaatagacgaattcatctacccaaggcagcatcaacaacagctgccgtttgcgataatcgaaactatcatagtgtccttttcactattagtgcagatgttatctagggtctaaacctctatgtttctacattcctggaaaatcttattctgtactgcataatcctctcacaaaaaattcgccaatgaatgacaaattagctatccagaattgcagattgattgaatcaaattgatcatggcagcagcacaattgcagcagttatcaattgaagcacaatattacctctcaatcataacaatcaaagcacagaaattccaatcaataactccacaatatagtatcaatctctaactcagaaactaaacagaagcaaaaacccattcaatcaaagcaacagtaaaattagggtttgaattagaaagaaagagagggaagagaaacatactgggttgcgcagcccatccctgttcaccacccttaatcttctcgggaagagcgtaattgacagtgagaacacgaccgtaaagctcagcaccatccatgttatccatggcagaagaagcgtcgtctctctccaagaaagtgacgaaaccgaaagaacgatgcttgttggtggccaaatcaagaggggttttgacgtctttgatttctccaaaagggatgaaggcagcatgaaggattgattcgttgacttcttctgctaatcctcctacgtataatgtgttcttctgcattccttgttgattcgtcgccatttttcg

SEQ ID NO: 45 - DNA, 1446 bp, Kochia scoparia
gcagcttcaagtcttgcttttttgcactcatcaccagaaactactattgaggtttgttctaaagagcatgaagttgtaattgctctagggagcaatgtaggagacagattagataactttaaccaagctctgcagctaatgaagaaattaggagtaaatatcacaaggcatggctgcttatacgagacagatcctgcctatgtgactgatcaaccaaagtttcttaactctgcagtgaggggcttcacaaaacttgggcctctagaactattagggatgctgaagaaaattgagaaggatatgggtcgaactaatggaataagatatggccctaggccaattgacttagacattcttttttatgggaagtttagggtaagctctgagaaacttacagttccgcatgaaaggatatgggaaagaccctttgtgatggcaccactaattgatataattggatcggatgtagaaaatgacactgttgctgcatggcattcattatcaaaattttctggtggactatttgaagcgtgggataaacttggtggagattcactcattgggaaggacggaatgagtagggttttgccagttgggaaccacttgtgggattggtcgtgcaaaacctctgtaatgggagtcttgaatttgactccggacagctttagtgatggaggaaagtttctacctgtagaaaatgcagtttctcaggttcgtcaaatgatctcagaaggtgctgacataattgatattggagcgcaatcaacaaggcccatggcaactaggatttctgctgaggaagagctggaaagactagtccctgttttagaaggtgtcaaggatgttatcgaggaagaaggaagaatgttatcagtggatacattttactcaaaagttgcttctgaagctgtcaataagggagcacatatggtgaatgatgtttcgagtggaaagctagatcctgagatgttcaatgttgttgcagggcttaaagtgccttatatagcaatgcacatgcgaggtgatccttcttcaatgcaaaatgctgataatttgacatacaatgatgtttgtaaggaggtggctttagagttgagctctaggattacagatgcagaattatcaggtattcctgcttggaggatagttattgatcctggcattggattttccaagaatacaaagcaaaatttggaaattctcacagggttgaaaagaattcggcaagaaatagcaaaaaagagtttggctgtggctcatggtcccttactgattggaccttcaagaaagaggtttttgggtgagatctgcaatcgtcctgtagcatctgatagagatccggcaactgttgcttctatcaccgctggagttttgggtggtgcaaacattgtaagagtacataatgttcgggatagccttgatggtgtcaagctatgtgatgcaatg

SEQ ID NO: 46 - DNA, 1446 bp, Kochia scoparia; identical to SEQ ID NO: 45

SEQ ID NO: 47 - DNA, 3920 bp, Lolium multiflorum
acgaaagctttgaactctcaagtggtgcaacatagatagattatatatatttcgaatataattcgaatgaacataaaaattacatataaaaactttaaaacaaacttaaactacttcttcttcctcgatggccccgcctcatcgtcggggcggcgacgcttccggctcgtcacctcctcgtcggaggaagttgggtcctcctcctcgtcagtgtcctcagcctcttcgtcgtcctcctcctcttcctcggcctcttcgtcggcctgctccttggcctcttcgtcctcctcctcgtcgtcatcccatgcgtcctcctcctcgtcgtcatcccattcgtccttgctggccggcgggggggaatcgtcgctgctggacgatgcagctttatcccaccaatggcgccatccaggcggcttcccctcgctgtcggtgtccgatggccaagagagatcgctcatggttgacggaggatggtgaggagagaacagatgaatggcgtcggccgtcgtaaaccgtatatgtaggtctctcgacgaagagagcggcggttgctcttccgcggagttcgtgctccattacagcggttctcgcatcgaggccacttcggccgttcccgacgagtcgtttgggctctccagaccactttgcgacggttcaatgcgtcgagggcactccgacgattgcccttcccggtgactgcgccgtcgctatgcacgcggtgtgtgcgcaaccatgggctcgcggctgggaaaatgggcctccccaggccacaaaatacatccatccggcgataactaacgccggatttcggcctgggagccccaacggctgggatgctcttagcccgtggaacacattcccgaatcggtaccgacggtctcatctctctcgccctgatctatctctcttccgcgatctggaagggccgacggcgtggcggcgcaaccacgggcgctgatgggcggcggcgcgcagtctgttaagcaggacgcctgcttctcgctccgtcctccctctccaaccgagcggcacgaggtagacccggagaccccaactccgtcctagcccagtggcgcccgtgaggcgtcatctccggcgccctctccatctccgtctgcttcgcacttgtatgcacatactcttcctccttaccccagtttcttggtcttggcttgtttgattacgatttctagggttcttggcatgctcggtagttgcagatttgtcttctgttgggtgtttgtttccatggccttggctgcatccatcgtttcgtattatgtagttgtttgtttccatgccattgcttgtatccatcaattggaaagggaagaaagggctacagggatattaggaattagatcagtccttcctagctgattttctctgacaggttgttagtctcgcgcatgtggtggacttcttgtgcttttggctagctccaagtctgatgtgtagctgctttctgagattttccacataaacgttgctatggctagtaagttgtggctaactaaatccttgggcacaagctagtaatctgaatggcatatccgcccccatgcctttaattgttgcctaacgatgggctaggttctcttatttattcatttttgttaaaagaagaacttgggcatgctaattcagcaaaatagaaaaaatatgtgttgtttctgtatatagaggttcttggttttcccttgccgattctgaccgatttgcgggttttttttttttttgtctttcctcatcgtcaggcaagaagagcaagatgtgacagaggatttaagcagcagcagaactagacctggctcctatgctctaagatgcttcgtgccaaggacacattcaggaagatgtactcagctgccaagggctactactgcggagggctagcgtctcctaatgccttctcaggtagcttgcccaaagttccccaataataactagggaacatctcttttccacctctttcacattccaaccaaagacagtcctaactcgtttcccctctgaacataccagcattggatcttcggcatcctctcagctcctcaagaaaaccaccaggccacgctaacctgttcagggtccgttcatacacgcaatgcgccctcaccaacgattccgccgaccaggaggtcgtgattgccctgggcagcaacgtgggagacagggtcagcatgttcgacagggcgctgcggctgatgaggagctcgggcatcaaggtcaccaggcacgcctgcctgtacgagaccgccccagcctacgtgaccgaccagccgcggttcctcaactctgccgttaggggcacgacgaagctggggcctcacgagttgctcaggaagctcaaggagatcgagaaggatataggacgtaccgccggggtaaggtacggcccgaggccgatcgatctggacattcttctgtatggggactcccggatcaagaccgagtctctgattgtgccgcatgaacgcatccatgagagaccgttcgtcttggcgcctcttgttgacctcctgggttcgtcggctgaggacggtatggagaaaagctggcactctctctcgaagtgcagtggcgggttctttgatttgtggaacaagctcggcggtgaatctgttgttggaacagaaggcattaaaagggtcatgtctgttggaaatacgctgttggactggcgtcagaggacccttgttatgggggtgcttaacctcacgccagacagcttcagcgacggaggtaagtttcaagaagtggaagctgccatttcccaggccaggttgttgatatcagaaggtgctgacataattgatattggtgcccagtccaccaggccctttgcaaggaggctatccgcagaagaagagcttgagaggctggtccctgttctggatgctatcatgaaactcccagagatggaagggaagttgctttcggtagacacgttctacgcgcaagtcgctgctgaagctgtaaaaagaggagccaccatgatcaatgatgtatctggtgggcagcttgacccaagtattcttcaagttgttgctgaactgggagttccgtatgttaccatgcacatgagaggcgatccatcaactatgcagaatgaacagaatctacagtatgatgatgtctgcaaagaagttgcttctgagttatacgcccggttgagagcagcagagctttctggaattcctttgtggaggattattcttgaccctggcattgggttttctaagaaatccacacagaatattgaagtaatcgcgggtttggaatccattagagaagagatgggtaaaatgagcctaggtgcttcacatgtgccaatattacttggaccctcgaggaaaagtttcctaggtgaaatatgtagtcgcgccgatccagttcagagagatgctgctactgcttctgccgttacaattgcgatcttgaatggcgctaatatagtaagggtccataatgttagatacaatgtggatgctgcaaaggtctctgatgcattactcaagtacagaagaaaataatagaaagtatacagcccggatagaagccataccagttgccagttttgtgcaagggaatgctgatgtggagctcaaaccatatggggtgtatcatcacttaataagatcaaagcatgcgtgaataactattacagtgaacaagagatatacagttttttcgtgcgcctcctccttcgtagaatcttcaaagaagcaaaggtaacacagacctaggcctctggggttggtatatgatgtaaccagttttgtgagggtttaataattatctaatagtgagacctaagagtaagacctttataatatactttgcttggaatttcctacttgagagcatcatttggggctacagagttgattttcgtctcacgcagttttctgagggtt

SEQ ID NO: 48 - DNA, 1467 bp, Lolium multiflorum
agctcctcaagaaaaccaccaggccacgctaacctgttcagggtccgttcatacacgcaatgcgccctcaccaacgattccgccgaccaggaggtcgtgattgccctgggcagcaacgtgggagacagggtcagcatgttcgacagggcgctgcggctgatgaggagctcgggcatcaaggtcaccaggcacgcctgcctgtacgagaccgccccagcctacgtgaccgaccagccgcggttcctcaactctgccgttaggggcacgacgaagctggggcctcacgagttgctcaggaagctcaaggagatcgagaaggatataggacgtaccgccggggtaaggtacggcccgaggccgatcgatctggacattcttctgtatggggactcccggatcaagaccgagtctctgattgtgccgcatgaacgcatccatgagagaccgttcgtcttggcgcctcttgttgacctcctgggttcgtcggctgaggacggtatggagaaaagctggcactctctctcgaagtgcagtggcgggttctttgatttgtggaacaagctcggcggtgaatctgttgttggaacagaaggcattaaaagggtcatgtctgttggaaatacgctgttggactggcgtcagaggacccttgttatgggggtgcttaacctcacgccagacagcttcagcgacggaggtaagtttcaagaagtggaagctgccatttcccaggccaggttgttgatatcagaaggtgctgacataattgatattggtgcccagtccaccaggccctttgcaaggaggctatccgcagaagaagagcttgagaggctggtccctgttctggatgctatcatgaaactcccagagatggaagggaagttgctttcggtagacacgttctacgcgcaagtcgctgctgaagctgtaaaaagaggagccaccatgatcaatgatgtatctggtgggcagcttgacccaagtattcttcaagttgttgctgaactgggagttccgtatgttaccatgcacatgagaggcgatccatcaactatgcagaatgaacagaatctacagtatgatgatgtctgcaaagaagttgcttctgagttatacgcccggttgagagcagcagagctttctggaattcctttgtggaggattattcttgaccctggcattgggttttctaagaaatccacacagaatattgaagtaatcgcgggtttggaatccattagagaagagatgggtaaaatgagcctaggtgcttcacatgtgccaatattacttggaccctcgaggaaaagtttcctaggtgaaatatgtagtcgcgccgatccagttcagagagatgctgctactgcttctgccgttacaattgcgatcttgaatggcgctaatatagtaagggtccataatgttagatacaatgtggatgctgcaaaggtctctgatgcatta

SEQ ID NO: 49 - DNA, 834 bp, Lolium multiflorum
agggtcatgtctgttggaaatacgctgttggattggcgggagaggaccctggttatgggggtgcttaacctcacgccagacagctttagcgatggaggtaagtttcaagaggtggaagctgccatttctcaggccaggctgttaatttcagaaggtgcaggcataattgatattggcgctcagtccaccaggccctttgcaaggtggctatctgcagaagaagagcttgagaggctggtccctgttcttaatgctatcatgaaactccccgagatggaagggaagttgctttcagtagatacgttctacgcgcaagtcgcggctgaagccgtgaaaagaggggccaccatgatcaatgatgtatctggtgggcagcttgacccagatattcttcaagttgttgctgaattgggagttccgtatgttgccatgcacatgagaggggatccatcaactatgcagaatgagcagaatttacagtatgatgatgtctgcaaagaagttgcttctgagttatactctcggttgagagcagcagagttgtctggaattcctttgtggaggattattcttgaccctggcattgggttttccaagaaatccacacagaatattgaagtaatcgcgggcttggaatccattagagaagagatgggtaaaatgagtctaggtgcttcacatgtgccaatattacttggaccctccaggaaacgtttcttagggcagatatgcaatcgcgctgatccagttgagagagatgcagctactgctgcttctgttacagttgggatattgaatggtgctaatattgtaagggtccataatgttagatac

SEQ ID NO: 50 - DNA, 834 bp, Lolium multiflorum; identical to SEQ ID NO: 49

SEQ ID NO: 51 - DNA, 212 bp, Lolium multiflorum
aggatatagggcgcaccgccgggataaggtacggtccgaggccgatcgatctggacattcttctgtatggggattcccggatcaagaccgagtctctgactgtgccgcatgaacgcatccatgagagaccgttcgtcttggcgcctcttgttgacctcctgggttcatcgggtgaggatggtatggagagaagatggcactctctctcgaag

SEQ ID NO: 52 - DNA, 210 bp, Lolium multiflorum
gatatagggcgcaccgccgggataaggtacggtccgaggccgatcgatctggacattcttctgtatggggattcccggatcaagaccgagtctctgactgtgccgcatgaacgcatccatgagagaccgttcgtcttggcgcctcttgttgacctcctgggttcatcgggtgaggatggtatggagagaagatggcactctctctcgaag

SEQ ID NO: 53 - DNA, 201 bp, Lolium multiflorum
cgctaacctgttcagggtccgttcgtacatgcaatgcgcggtcaccaacgattccgccgaccaagagatcgtgattgccctgggcagcaacgtgggagacagggtcagcacgttcgacagggcgctgcggctgatgaggagctcgggcatcaggatcaccaggcacgcctgcctgtacgagaccgcccctgcttacgtgac

SEQ ID NO: 54 - DNA, 150 bp, Lolium multiflorum
gattccgccgaccaagagatcgtgattgccctgggcagcaacgtgggagacagggtcagcacgttcgacagggcgctgcggctgatgaggagctcgggcatcaggatcaccaggcacgcctgcctgtacgagaccgcccctgcttacgtg

SEQ ID NO: 55 - DNA, 200 bp, Amaranthus hybridus
agatttgtgagcgccctgtagcagctgatagggatcctgctaccgttgcttcaataactgctggagttttaggtggtgcaaacattgtaagagtacataatgtgagggataaccttgatgctgtcaagttatgtgatgccatactcggaaaaactgattaactgcttatttgtaccaccttgtgaatgacgtctagtgga

SEQ ID NO: 56 - DNA, 200 bp, Amaranthus hybridus
tccactagacgtcattcacaaggtggtacaaataagcagttaatcagtttttccgagtatggcatcacataacttgacagcatcaaggttatccctcacattatgtactcttacaatgtttgcaccacctaaaactccagcagttattgaagcaacggtagcaggatccctatcagctgctacagggcgctcacaaatct

SEQ ID NO: 57 - DNA, 200 bp, Amaranthus hybridus
caccttgtgaatgacgtctagtggaaagttcgattgggatattgatgaagtccgttagtttgctcgaaagtagagctttagcccgtaaggttatagttcttacagatgtagaatcattggctatttgcctattcctgcttagaagatcatagcattgatccagactttggattccatgtagaattatgttgcgactcatg

SEQ ID NO: 58 - DNA, 200 bp, Amaranthus hybridus
catgagtcgcaacataattctacatggaatccaaagtctggatcaatgctatgatcttctaagcaggaataggcaaatagccaatgattctacatctgtaagaactataaccttacgggctaaagctctactttcgagcaaactaacggacttcatcaatatcccaatcgaactttccactagacgtcattcacaaggtg

SEQ ID NO: 59 - DNA, 200 bp, Amaranthus hybridus
atgtagaattatgttgcgactcatggtcgaattggccctttgaggaattgttgaagttccttgatctatggttcaatcggctcggttcgatgaaggttggactgtgtcgagacaagatgggtcaaagagaccaatggacaagaagtcgaccttgggaaagcttgatcaagttacttggacgagcagatagcagcaggact

SEQ ID NO: 60 - DNA, 200 bp, Amaranthus hybridus
agtcctgctgctatctgctcgtccaagtaacttgatcaagctttcccaaggtcgacttcttgtccattggtctctttgacccatcttgtctcgacacagtccaaccttcatcgaaccgagccgattgaaccatagatcaaggaacttcaacaattcctcaaagggccaattcgaccatgagtcgcaacataattctacat

SEQ ID NO: 61 - DNA, 200 bp, Amaranthus lividus
aagggaataaggtatggtcctaggccaattgacttggacatactattttatgggaaatttagggtgagctctgagagcctcactatcccccatgaaaggatatgggaaagaccatttgtgatggcaccattgattgattgtattgggtctgatgtagaaaatgacactatttgtacatggcattcattatcaaatttttt

SEQ ID NO: 62 - DNA, 200 bp, Amaranthus lividus
aaaaaatttgataatgaatgccatgtacaaatagtgtcattttctacatcagacccaatacaatcaatcaatggtgccatcacaaatggtctttcccatatcctttcatgggggatagtgaggctctcagagctcaccctaaatttcccataaaatagtatgtccaagtcaattggcctaggaccataccttattccctt

SEQ ID NO: 63 - DNA, 200 bp, Amaranthus lividus
catggcattcattatcaaattttttgggtggaatctttgaagcatggggtaaactcggtggaagttccctaatcgggaaggatggaatgaaaagggttttgcccgttggaaatcgcttatgggattggtctcataaaacctctgtaatgggagtcttgaacttgactcctgatagctttagtgatggcggaagttttcaa

SEQ ID NO: 64 - DNA, 200 bp, Amaranthus lividus
ttgaaaacttccgccatcactaaagctatcaggagtcaagttcaagactcccattacagaggttttatgagaccaatcccataagcgatttccaacgggcaaaacccttttcattccatccttcccgattagggaacttccaccgagtttaccccatgcttcaaagattccacccaaaaaatttgataatgaatgccatg

SEQ ID NO: 65 - DNA, 200 bp, Amaranthus palmeri
cggatctgttaaagggagccctctcatcctatgcttatcaatgactttttcatatgcctatcaaacagatcagattggatcgggttcgagttcaggtcatatataaacatgtcagcaaacccttgacccatacccaacccatttagttaattggcttgaaaatcacaaccttaattcgaccagcgacagatcaggttgac

SEQ ID NO: 66 - DNA, 200 bp, Amaranthus palmeri
gtcaacctgatctgtcgctggtcgaattaaggttgtgattttcaagccaattaactaaatgggttgggtatgggtcaagggtttgctgacatgtttatatatgacctgaactcgaacccgatccaatctgatctgtttgataggcatatgaaaaagtcattgataagcataggatgagagggctccctttaacagatccg

SEQ ID NO: 67 - DNA, 200 bp, Amaranthus palmeri
tcgaccagcgacagatcaggttgacctgatttcgacccacttaacccatttataatttttttttcgatttaagttttttaacattgattaaatctaattttttaggctttaattttaatttatgtttttttgtttaatttgtatttactatgaaaaataagaaactattatatgaactgagtttagcttacacttattga

SEQ ID NO: 68 - DNA, 200 bp, Amaranthus palmeri
tcaataagtgtaagctaaactcagttcatataatagtttcttatttttcatagtaaatacaaattaaacaaaaaaacataaattaaaattaaagcctaaaaaattagatttaatcaatgttaaaaaacttaaatcgaaaaaaaaattataaatgggttaagtgggtcgaaatcaggtcaacctgatctgtcgctggtcga

SEQ ID NO: 69 - DNA, 200 bp, Amaranthus palmeri
actgagtttagcttacacttattgatttaactcgaaattaacacatttaattaaatggttatatagggttttttagttttaaaatttcaacctgaacctaatctatttaataaaaagatcagatctgatcaacccatataattaattgggtcaaaatctcaactcaaacccatttatttcgaattaaattcagatcaaat

SEQ ID NO: 70 - DNA, 200 bp, Amaranthus palmeri
atttgatctgaatttaattcgaaataaatgggtttgagttgagattttgacccaattaattatatgggttgatcagatctgatctttttattaaatagattaggttcaggttgaaattttaaaactaaaaaaccctatataaccatttaattaaatgtgttaatttcgagttaaatcaataagtgtaagctaaactcagt

SEQ ID NO: 71 - DNA, 200 bp, Amaranthus palmeri
atttcgaattaaattcagatcaaattagcaggtcggatcaactagcgctaatttttcctctcaagttttttcatatgatgccccttgttttctttgaataaaattcaccaatttaaaatccctgcccttgatccgttggtattggtatttggtaaagcacatatggtaaattctactccattgttatctgtagttttggt

SEQ ID NO: 72 - DNA, 200 bp, Amaranthus palmeri
accaaaactacagataacaatggagtagaatttaccatatgtgctttaccaaataccaataccaacggatcaagggcagggattttaaattggtgaattttattcaaagaaaacaaggggcatcatatgaaaaaacttgagaggaaaaattagcgctagttgatccgacctgctaatttgatctgaatttaattcgaaat

SEQ ID NO: 73 - DNA, 200 bp, Amaranthus palmeri
ctccattgttatctgtagttttggttctaaaccccaaacaaatcctactcctagcaccactaatcctcattactttctgtttgctgtctttctctacattcctactaaattaagacctagatgcagtgttattctgttcgaggttgccgaaaacggtacaatagttgcctaatttgcaattcatgtttgttcttgcgttt

SEQ ID NO: 74 - DNA, 200 bp, Amaranthus palmeri
aaacgcaagaacaaacatgaattgcaaattaggcaactattgtaccgttttcggcaacctcgaacagaataacactgcatctaggtcttaatttagtaggaatgtagagaaagacagcaaacagaaagtaatgaggattagtggtgctaggagtaggatttgtttggggtttagaaccaaaactacagataacaatggag

SEQ ID NO: 75 - DNA, 200 bp, Amaranthus palmeri
gcaattcatgtttgttcttgcgttttctacattgtttactctattggaacaaaggttgaactttcagcctttcagggagtattttgatttttctgtcaaattaccttgccagatttatttggctattttgattgtgtgtatttctttgtcccttgtttccatggggctaaactccttttggttaatcttaaacatttctt

SEQ ID NO: 76 - DNA, 200 bp, Amaranthus palmeri
aagaaatgtttaagattaaccaaaaggagtttagccccatggaaacaagggacaaagaaatacacacaatcaaaatagccaaataaatctggcaaggtaatttgacagaaaaatcaaaatactccctgaaaggctgaaagttcaacctttgttccaatagagtaaacaatgtagaaaacgcaagaacaaacatgaattgc

SEQ ID NO: 77 - DNA, 200 bp, Amaranthus palmeri
ttttggttaatcttaaacatttcttctaattttgaattgcagtggtatgaagtgttgtttaatatacaatcaatttttctgaactgggttaatgaagagaagcattgtagacatgaggaatgtgttcaagcatctcaaaacaaccaaattaatcccaaatgggatcttcaactgtaatagaggtaaatcacatatattct

SEQ ID NO: 78 - DNA, 200 bp, Amaranthus palmeri
agaatatatgtgatttacctctattacagttgaagatcccatttgggattaatttggttgttttgagatgcttgaacacattcctcatgtctacaatgcttctcttcattaacccagttcagaaaaattgattgtatattaaacaacacttcataccactgcaattcaaaattagaagaaatgtttaagattaaccaaaa

SEQ ID NO: 79 - DNA, 200 bp, Amaranthus palmeri
aatagaggtaaatcacatatattctcatttaatggttattgttttattttaaggatgttattatgttgttatcaatgatagaaataagagaagcttgagcattcaaatttatctatcactcactggttgtggacatctaaaatatggaccaagagttttcttcattttttgtaccagaagctgcaaatgtatgattaaag

SEQ ID NO: 80 - DNA, 200 bp, Amaranthus palmeri
ctttaatcatacatttgcagcttctggtacaaaaaatgaagaaaactcttggtccatattttagatgtccacaaccagtgagtgatagataaatttgaatgctcaagcttctcttatttctatcattgataacaacataataacatccttaaaataaaacaataaccattaaatgagaatatatgtgatttacctctatt

SEQ ID NO: 81 - DNA, 200 bp, Amaranthus palmeri
agaagctgcaaatgtatgattaaagtttaggttggagttgcaataaaattttattaccccgacgctattaagtggctcacactgaggcggggtttgagggtcagatggtgcaaccttcagaagaggttctaagcattttcaacatgttcaaccgcatagggtcccgtaaaattggaggcttcaatattaaattatgtagt

SEQ ID NO: 82 - DNA, 200 bp, Amaranthus palmeri
actacataatttaatattgaagcctccaattttacgggaccctatgcggttgaacatgttgaaaatgcttagaacctcttctgaaggttgcaccatctgaccctcaaaccccgcctcagtgtgagccacttaatagcgtcggggtaataaaattttattgcaactccaacctaaactttaatcatacatttgcagcttct

SEQ ID NO: 83 - DNA, 200 bp, Amaranthus palmeri
aggcttcaatattaaattatgtagtatgtgttatgtgtttaaagatataaatatgttaatgaatacataatttttaaattgcacagggccttcaaagaaattttcgattttgctctgcccctggcaacctttggcaaatttggatagaattagagaagtttacttttatgtgtgtataatatgatgaatgaatgagtgaa

SEQ ID NO: 84 - DNA, 200 bp, Amaranthus palmeri
ttcactcattcattcatcatattatacacacataaaagtaaacttctctaattctatccaaatttgccaaaggttgccaggggcagagcaaaatcgaaaatttctttgaaggccctgtgcaatttaaaaattatgtattcattaacatatttatatctttaaacacataacacatactacataatttaatattgaagcct

SEQ ID NO: 85 - DNA, 200 bp, Amaranthus palmeri
ataatatgatgaatgaatgagtgaacaatgaagttcatttctgagtcaatgacctatatactatcgaggtttcgttaaaaaggatatataaataaaattagagacatcttttatccctaatgttgtctgttttcttttctgggcgagactgtttttggagaaaaacgttatgttgaatgcttgatttactcagaactgat

SEQ ID NO: 86 - DNA, 200 bp, Amaranthus palmeri
atcagttctgagtaaatcaagcattcaacataacgtttttctccaaaaacagtctcgcccagaaaagaaaacagacaacattagggataaaagatgtctctaattttatttatatatcctttttaacgaaacctcgatagtatataggtcattgactcagaaatgaacttcattgttcactcattcattcatcatattat

SEQ ID NO: 87 - DNA, 200 bp, Amaranthus palmeri
aatgcttgatttactcagaactgatgcaaacagatggataaaaatggtgttttcctattgtttatgtttcgataacaggtttcaaattccactcgtatgaagtttgatatactgctatgtgtttttgtagcttcgtgttttgctttccttcactcatcaccagtgaatgatgttgaagtttgttccaagaagcaagaagt

SEQ ID NO: 88 - DNA, 200 bp, Amaranthus palmeri
acttcttgcttcttggaacaaacttcaacatcattcactggtgatgagtgaaggaaagcaaaacacgaagctacaaaaacacatagcagtatatcaaacttcatacgagtggaatttgaaacctgttatcgaaacataaacaataggaaaacaccatttttatccatctgtttgcatcagttctgagtaaatcaagcatt

SEQ ID NO: 89 - DNA, 200 bp, Amaranthus palmeri
aagtttgttccaagaagcaagaagtcgtaattgcaatcgggagcaatgttggtgacagattagaaaatttcaaccaagctctgcaacaaatgaagaaattaggcatagacatcacaaggcatgggtgtttatatgagacggaacctgcatacgtgactgatcaaccgaagtttcttaactctgctttaagaggctttaca

SEQ ID NO: 90 - DNA, 200 bp, Amaranthus palmeri
tgtaaagcctcttaaagcagagttaagaaacttcggttgatcagtcacgtatgcaggttccgtctcatataaacacccatgccttgtgatgtctatgcctaatttcttcatttgttgcagagcttggttgaaattttctaatctgtcaccaacattgctcccgattgcaattacgacttcttgcttcttggaacaaactt

SEQ ID NO: 91 - DNA, 200 bp, Amaranthus palmeri
taactctgctttaagaggctttacaaaacttgggcctcatgaattattaggggtattaaagaaaattgagaaggatatgggtagaaccaagggaataaggtatggtcctaggccaattgacttggacatactattttatgggaagtttagggtgagctctgagagcctcactatcccccatgaaaggatatgggaaagac

SEQ ID NO: 92 - DNA, 200 bp, Amaranthus palmeri
gtctttcccatatcctttcatgggggatagtgaggctctcagagctcaccctaaacttcccataaaatagtatgtccaagtcaattggcctaggaccataccttattcccttggttctacccatatccttctcaattttctttaatacccctaataattcatgaggcccaagttttgtaaagcctcttaaagcagagtta

SEQ ID NO: 93 - DNA, 200 bp, Amaranthus palmeri
ccccatgaaaggatatgggaaagaccatttgtgatggcaccattgattgattgtattgggtctgatgtagaaaatgacactatttgtacgtggcattcattatcaaatttttcgggtggaatctttgaagcatggggtaaactcggtggaagttccctaatcgggaaggatggaatgaaaagggttttgcccgttggaaa

SEQ ID NO: 94 - DNA, 200 bp, Amaranthus palmeri
tttccaacgggcaaaacccttttcattccatccttcccgattagggaacttccaccgagtttaccccatgcttcaaagattccacccgaaaaatttgataatgaatgccacgtacaaatagtgtcattttctacatcagacccaatacaatcaatcaatggtgccatcacaaatggtctttcccatatcctttcatgggg

SEQ ID NO: 95 - DNA, 200 bp, Amaranthus palmeri
tgaaaagggttttgcccgttggaaatcgcttatgggattggtctcataaaacctctgttatgggagtcttgaacttgactcctgatagctttagtgatggcggaagttttcaatctgtcgatactgcggttgctaaggttcgtcagatgatctcagatggggcagatataattgacatcggggcacaatcaaccagaccc

SEQ ID NO: 96 - DNA, 200 bp, Amaranthus palmeri
gggtctggttgattgtgccccgatgtcaattatatctgccccatctgagatcatctgacgaaccttagcaaccgcagtatcgacagattgaaaacttccgccatcactaaagctatcaggagtcaagttcaagactcccataacagaggttttatgagaccaatcccataagcgatttccaacgggcaaaacccttttca

SEQ ID NO: 97 - DNA, 200 bp, Amaranthus palmeri
catcggggcacaatcaaccagacccatggcaactagaatttcggctgaagaagagctagaaagagtagtacctgtattggaagctgtcaaggatttgatcgaggaagaaggaagaatcttgtcagtggatacgttttattctaaagttgcttcggaggccgtcaagaagggggcacacattgtgaatgatgtctctagtg

SEQ ID NO: 98 - DNA, 200 bp, Amaranthus palmeri
cactagagacatcattcacaatgtgtgcccccttcttgacggcctccgaagcaactttagaataaaacgtatccactgacaagattcttccttcttcctcgatcaaatccttgacagcttccaatacaggtactactctttctagctcttcttcagccgaaattctagttgccatgggtctggttgattgtgccccgatg

SEQ ID NO: 99 - DNA, 200 bp, Amaranthus palmeri
cacattgtgaatgatgtctctagtgggaaactcgattccgagatgtttaatgttgttgcggaccttaaagttccttatatagcaatgcacatgcgaggagatccgacttcaatgcaaaactctgagaacttgacctacaatgatgtttgtaagcaagtggctttggagttgagttctagggtcatagatgcagaattatc

SEQ ID NO: 100 - DNA, 200 bp, Amaranthus palmeri
gataattctgcatctatgaccctagaactcaactccaaagccacttgcttacaaacatcattgtaggtcaagttctcagagttttgcattgaagtcggatctcctcgcatgtgcattgctatataaggaactttaaggtccgcaacaacattaaacatctcggaatcgagtttcccactagagacatcattcacaatgtg

SEQ ID NO: 101 - DNA, 200 bp, Amaranthus palmeri
ctagggtcatagatgcagaattatcgggaattcctgcttggaggatagttattgatcccggcatcggattttctaagaatacgaatcaaaatttggaaattcttagtggtttacaaaagatacgggaagagatagctaagaagagtttggcggtggctcattgccccttgctaattggaccttcaagaaagaggtttctg

SEQ ID NO: 102 - DNA, 200 bp, Amaranthus palmeri
cagaaacctctttcttgaaggtccaattagcaaggggcaatgagccaccgccaaactcttcttagctatctcttcccgtatcttttgtaaaccactaagaatttccaaattttgattcgtattcttagaaaatccgatgccgggatcaataactatcctccaagcaggaattcccgataattctgcatctatgaccctag

SEQ ID NO: 103 - DNA, 200 bp, Amaranthus palmeri
tggaccttcaagaaagaggtttctgggcgagatttgtgagcgccctgtagcagctgacagggatcctgctaccattgcttctataactgctggagttttaggtggtgcaaacattgtaagagtacataatgtgagggataaccttgatgctgtcaagttatgtgatgccatactcggaaaaactgattaactgcttgttt

SEQ ID NO: 104 - DNA, 200 bp, Amaranthus palmeri
aaacaagcagttaatcagtttttccgagtatggcatcacataacttgacagcatcaaggttatccctcacattatgtactcttacaatgtttgcaccacctaaaactccagcagttatagaagcaatggtagcaggatccctgtcagctgctacagggcgctcacaaatctcgcccagaaacctctttcttgaaggtcca

SEQ ID NO: 105 - DNA, 200 bp, Amaranthus palmeri
ggaaaaactgattaactgcttgtttgtaccaccttgtgaatgatgtctagtggaaggttcgattgggattatgatgaagtccgttagtttgctcgaaagaagagctttagcccgtaaggttatagttcttacagatgtagaatcattggctatttgcctatttctgcttagaagatcatagcattgatccagactttgga

SEQ ID NO: 106 - DNA, 200 bp, Amaranthus palmeri
tccaaagtctggatcaatgctatgatcttctaagcagaaataggcaaatagccaatgattctacatctgtaagaactataaccttacgggctaaagctcttctttcgagcaaactaacggacttcatcataatcccaatcgaaccttccactagacatcattcacaaggtggtacaaacaagcagttaatcagtttttcc

SEQ ID NO: 107 - DNA, 200 bp, Amaranthus palmeri
tcatagcattgatccagactttggattccatgtagaattatgttgcgactcatggtcgaattggccctttgaggaattgttgaagttccttgagtctatggttcaatcggctcggttcgatgaaggttggactgtgtcgagacaagatgggtcaaagagaccaatggacaagaagtcgaccttgggcaagcttgatcaag

SEQ ID NO: 108 - DNA, 200 bp, Amaranthus palmeri
cttgatcaagcttgcccaaggtcgacttcttgtccattggtctctttgacccatcttgtctcgacacagtccaaccttcatcgaaccgagccgattgaaccatagactcaaggaacttcaacaattcctcaaagggccaattcgaccatgagtcgcaacataattctacatggaatccaaagtctggatcaatgctatga

SEQ ID NO: 109 - DNA, 200 bp, Amaranthus palmeri
tcgaccttgggcaagcttgatcaagttacttggacaagcagataacagcaggctctggcgagcattagacatatcatttgttttttggagtcagatctgcaatcaatcagttgcaatggacaaaccatatacggttgttagttgttactgtcactgtttgagttgaattagatcatgcaaacactgtaggagtcttgatt

SEQ ID NO: 110 - DNA, 200 bp, Amaranthus palmeri
aatcaagactcctacagtgtttgcatgatctaattcaactcaaacagtgacagtaacaactaacaaccgtatatggtttgtccattgcaactgattgattgcagatctgactccaaaaaacaaatgatatgtctaatgctcgccagagcctgctgttatctgcttgtccaagtaacttgatcaagcttgcccaaggtcga

SEQ ID NO: 111 - DNA, 200 bp, Amaranthus palmeri
tgcaaacactgtaggagtcttgattatgtgaagctatgtgacactgtacttctcaaaatgaaatctttgatatgatgttagtttcaagagttgtaattcccccctcacatttctgttctatgttgtattgttgtgtgctttttggaattgtattcctgcaatagaattattcccgcaaaatggtattcctttatatcatc

SEQ ID NO: 112 - DNA, 200 bp, Amaranthus palmeri
gatgatataaaggaataccattttgcgggaataattctattgcaggaatacaattccaaaaagcacacaacaatacaacatagaacagaaatgtgaggggggaattacaactcttgaaactaacatcatatcaaagatttcattttgagaagtacagtgtcacatagcttcacataatcaagactcctacagtgtttgca

SEQ ID NO: 113 - DNA, 200 bp, Amaranthus palmeri
caaaatggtattcctttatatcatcaaaggagtttttgtacttctaatcattgatctttagatgcaaaactgaattaatacaagtattcttgatgaaggcttattgaagaactcactttttatttatgtctaaacttgttaaagatgttctgcttccgcccttgccacgggatgtcgggattgactaggggtggtaatcg

SEQ ID NO: 114 - DNA, 200 bp, Amaranthus palmeri
cgattaccacccctagtcaatcccgacatcccgtggcaagggcggaagcagaacatctttaacaagtttagacataaataaaaagtgagttcttcaataagccttcatcaagaatacttgtattaattcagttttgcatctaaagatcaatgattagaagtacaaaaactcctttgatgatataaaggaataccattttg

SEQ ID NO: 115 - DNA, 200 bp, Amaranthus palmeri
cgggattgactaggggtggtaatcgagggattcctatgcttgaattgactcgtttaagattttgcgttgattttaagtcaaacgttttgctaagcataaactattctagctcgacttgaataatgaagttttcgagtcaagtccgagctgttctataagtttacgtatcttgaagcttcttagttcttacttcttatcct

SEQ ID NO: 116 - DNA, 200 bp, Amaranthus palmeri
aggataagaagtaagaactaagaagcttcaagatacgtaaacttatagaacagctcggacttgactcgaaaacttcattattcaagtcgagctagaatagtttatgcttagcaaaacgtttgacttaaaatcaacgcaaaatcttaaacgagtcaattcaagcataggaatccctcgattaccacccctagtcaatcccg

SEQ ID NO: 117 - DNA, 200 bp, Amaranthus palmeri
cttcttagttcttacttcttatcctttattttttgacacattgaaccgcattgatgcttgcttgactcaactcgttaaagtcttgagttcatcacgaattcgagtcgagcaagtttaaacccaacattaactaagctctcgaggcttaatgaggcaggggtagctcaagtttgttattcaagcaacttaccatagtattc

SEQ ID NO: 118 - DNA, 200 bp, Amaranthus palmeri
gaatactatggtaagttgcttgaataacaaacttgagctacccctgcctcattaagcctcgagagcttagttaatgttgggtttaaacttgctcgactcgaattcgtgatgaactcaagactttaacgagttgagtcaagcaagcatcaatgcggttcaatgtgtcaaaaaataaaggataagaagtaagaactaagaag

SEQ ID NO: 119 - DNA, 200 bp, Amaranthus palmeri
attcaagcaacttaccatagtattcttttaaaaggcatatagtcagttagttatttgttaatttcagtagaacgaaagaagtacattattaaacttggccattgatgcttgagctagagagtttcggttgtgatcgaaacgaatcctgatttcgctcttgatctttgatgtgaagtgacaaggccttcataaaactcctt

SEQ ID NO: 120 - DNA, 200 bp, Amaranthus palmeri
aaggagttttatgaaggccttgtcacttcacatcaaagatcaagagcgaaatcaggattcgtttcgatcacaaccgaaactctctagctcaagcatcaatggccaagtttaataatgtacttctttcgttctactgaaattaacaaataactaactgactatatgccttttaaaagaatactatggtaagttgcttgaat

SEQ ID NO: 121 - DNA, 200 bp, Amaranthus palmeri
tgacaaggccttcataaaactcctttattcttcagcagtgcatacaaaaatcatgtgtgcccagctcctagctcacataccgcgtttttcccctaatctagtgtggttaaataaaaacagcaagatgtgttgatgtcatgtgcaatagtaattatcaggtgaagtaattcaatatgtagccagtcacctcatatcaaaaa

SEQ ID NO: 122 - DNA, 200 bp, Amaranthus palmeri
tttttgatatgaggtgactggctacatattgaattacttcacctgataattactattgcacatgacatcaacacatcttgctgtttttatttaaccacactagattaggggaaaaacgcggtatgtgagctaggagctgggcacacatgatttttgtatgcactgctgaagaataaaggagttttatgaaggccttgtca

SEQ ID NO: 123 - DNA, 200 bp, Amaranthus palmeri
gtagccagtcacctcatatcaaaaaaatgttgggaggaaaacgtattcgttcatcactaaacctgtttttatacatctaactgatcgatcagggtttcaaaatccaaattaatacaatggcatttgacgaagatatccagaacacatcccccttgtttctgctagataatcaactattctgattctgctgtaatacctca

SEQ ID NO: 124 - DNA, 200 bp, Amaranthus palmeri
tgaggtattacagcagaatcagaatagttgattatctagcagaaacaagggggatgtgttctggatatcttcgtcaaatgccattgtattaatttggattttgaaaccctgatcgatcagttagatgtataaaaacaggtttagtgatgaacgaatacgttttcctcccaacatttttttgatatgaggtgactggctac

SEQ ID NO: 125 - DNA, 200 bp, Amaranthus palmeri
attctgattctgctgtaatacctcagcttcggccctagccatgggatcgtcctctttttctccttcacgctcctcggccagtttctttctgtgcagttcttcagcagcttctattgaagctttgttctcggcctgaagacgctgcatttcctcttccatttgctgcctctcaaaccatgtatcagcatccgcccaaactg

SEQ ID NO: 126 - DNA, 200 bp, Amaranthus palmeri
cagtttgggcggatgctgatacatggtttgagaggcagcaaatggaagaggaaatgcagcgtcttcaggccgagaacaaagcttcaatagaagctgctgaagaactgcacagaaagaaactggccgaggagcgtgaaggagaaaaagaggacgatcccatggctagggccgaagctgaggtattacagcagaatcagaat

SEQ ID NO: 127 - DNA, 200 bp, Amaranthus palmeri
gaagctgtcaaggatttgatcgaggaagaaggaagaatcttgtcagtggatacgttttattctaaagttgcttcggaggccgtcaagaagggggcacacattgtgaatgatgtctctagtgggaaactcgattccgagatgtttaatgttgttgcggaccttaaagttccttatatagcaatgcacatgcgaggagatcc

SEQ ID NO: 128 - DNA, 200 bp, Amaranthus palmeri
ggatctcctcgcatgtgcattgctatataaggaactttaaggtccgcaacaacattaaacatctcggaatcgagtttcccactagagacatcattcacaatgtgtgcccccttcttgacggcctccgaagcaactttagaataaaacgtatccactgacaagattcttccttcttcctcgatcaaatccttgacagcttc

SEQ ID NO: 129 - DNA, 200 bp, Amaranthus palmeri
tagcaatgcacatgcgaggagatccgacttcaatgcaaaactctgagaacttgacctacaatgatgtttgtaagcaagtggctttggagttgagttctagggtcatagatgcagaattatcgggaattcctgcttggaggatagttattgatcccggcatcggattttctaagaatacgaatcaaaatttggaaattctt

SEQ ID NO: 130 - DNA, 200 bp, Amaranthus palmeri
aagaatttccaaattttgattcgtattcttagaaaatccgatgccgggatcaataactatcctccaagcaggaattcccgataattctgcatctatgaccctagaactcaactccaaagccacttgcttacaaacatcattgtaggtcaagttctcagagttttgcattgaagtcggatctcctcgcatgtgcattgcta

SEQ ID NO: 131 - DNA, 200 bp, Amaranthus palmeri
tacgaatcaaaatttggaaattcttagtggtttacaaaagatacgggaagagatagctaagaagagtttggcggtggctcattgccccttgctaattggaccttcaagaaagaggtttctgggcgagatttgtgagcgccctgtagcagctgacagggatcctgctaccattgcttctataactgctggagttttaggtg

SEQ ID NO: 132 - DNA, 200 bp, Amaranthus palmeri
cacctaaaactccagcagttatagaagcaatggtagcaggatccctgtcagctgctacagggcgctcacaaatctcgcccagaaacctctttcttgaaggtccaattagcaaggggcaatgagccaccgccaaactcttcttagctatctcttcccgtatcttttgtaaaccactaagaatttccaaattttgattcgta

SEQ ID NO: 133 - DNA, 200 bp, Amaranthus palmeri
tctataactgctggagttttaggtggtgcaaacattgtaagagtacataatgttagggataaccttgatgctgtcaagttatgtgatgccatactcggaaaaactgattaactgcttgtttgtaccaccttgtgaatgatgtctagtggaaggttcgattgggattatgatgaagtccgttagtttgctcgaaagtagag

SEQ ID NO: 134 - DNA, 200 bp, Amaranthus palmeri
ctctactttcgagcaaactaacggacttcatcataatcccaatcgaaccttccactagacatcattcacaaggtggtacaaacaagcagttaatcagtttttccgagtatggcatcacataacttgacagcatcaaggttatccctaacattatgtactcttacaatgtttgcaccacctaaaactccagcagttataga

SEQ ID NO: 135 - DNA, 200 bp, Amaranthus palmeri
tccgttagtttgctcgaaagtagagctttagcccgtaaggctatagttcttacagatgtagaatcattggctatttgcctatttctgcttagaagatcatagcattgatccagactttggattccatgtagaattatgttgcgactcatggtcgaattggccctttgaggaattgttgaagttccttgagtctatggttc

SEQ ID NO: 136 - DNA, 200 bp, Amaranthus palmeri
gaaccatagactcaaggaacttcaacaattcctcaaagggccaattcgaccatgagtcgcaacataattctacatggaatccaaagtctggatcaatgctatgatcttctaagcagaaataggcaaatagccaatgattctacatctgtaagaactatagccttacgggctaaagctctactttcgagcaaactaacgga

SEQ ID NO: 137 - DNA, 200 bp, Amaranthus palmeri
ttgaagttccttgagtctatggttcaatcggctcggttcgatgaaggttggactgtgtcgagacaagatgggtcaaagagaccaatggacaagaagtcgaccttgggcaagcttgatcaagttacttggacaagcagataacagcaggctctggcgagcattagacatatcatttgttttttggagtcagatctgcaatc

SEQ ID NO: 138 - DNA, 200 bp, Amaranthus palmeri
gattgcagatctgactccaaaaaacaaatgatatgtctaatgctcgccagagcctgctgttatctgcttgtccaagtaacttgatcaagcttgcccaaggtcgacttcttgtccattggtctctttgacccatcttgtctcgacacagtccaaccttcatcgaaccgagccgattgaaccatagactcaaggaacttcaa

SEQ ID NO: 139 - DNA, 200 bp, Amaranthus palmeri
gctttccttcactcatcaccagtgaatgatgttgaagtttgttccaagaagcaagaagtcgtaattgcaatcgggagcaatgttggtgacagattagaaaatttcaaccaagctctgcaacaaatgaagaaattaggcatagacatcacaaggcatgggtgtttatatgagacggaacctgcatacgtgactgatcaacc

SEQ ID NO: 140 - DNA, 200 bp, Amaranthus palmeri
ggttgatcagtcacgtatgcaggttccgtctcatataaacacccatgccttgtgatgtctatgcctaatttcttcatttgttgcagagcttggttgaaattttctaatctgtcaccaacattgctcccgattgcaattacgacttcttgcttcttggaacaaacttcaacatcattcactggtgatgagtgaaggaaagc

SEQ ID NO: 141 - DNA, 200 bp, Amaranthus palmeri
aacctgcatacgtgactgatcaaccgaagtttcttaactctgctttaagaggctttacaaaacttgggcctcatgaattattaggggtattaaagaaaattgagaaggatatgggtagaaccaagggaataaggtatggtcctaggccaattgacttggacatactattttatgggaagtttagggtgagctctgagagc

SEQ ID NO: 142 - DNA, 200 bp, Amaranthus palmeri
gctctcagagctcaccctaaacttcccataaaatagtatgtccaagtcaattggcctaggaccataccttattcccttggttctacccatatccttctcaattttctttaatacccctaataattcatgaggcccaagttttgtaaagcctcttaaagcagagttaagaaacttcggttgatcagtcacgtatgcaggtt

SEQ ID NO: 143 - DNA, 200 bp, Amaranthus palmeri
gaagtttagggtgagctctgagagcctcactatcccccatgaaaggatatgggaaagaccatttgtgatggcaccattgattgattgtattgggtctgatgtagaaaatgacactatttgtacgtggcattcattatcaaatttttcgggtggaatctttgaagcatggggtaaactcggtggaagttccctaatcggga

SEQ ID NO: 144 - DNA, 200 bp, Amaranthus palmeri
tcccgattagggaacttccaccgagtttaccccatgcttcaaagattccacccgaaaaatttgataatgaatgccacgtacaaatagtgtcattttctacatcagacccaatacaatcaatcaatggtgccatcacaaatggtctttcccatatcctttcatgggggatagtgaggctctcagagctcaccctaaacttc

SEQ ID NO: 145 - DNA, 200 bp, Amaranthus palmeri
ctcggtggaagttccctaatcgggaaggatggaatgaaaagggttttgcccgttggaaatcgcttatgggattggtctcataaaacctctgttatgggagtcttgaacttgactcctgatagctttagtgatggcggaagttttcaatctgtcgatactgcggttgctaaggttcgtcagatgatctcagatggggcaga

SEQ ID NO: 146 - DNA, 200 bp, Amaranthus palmeri
tctgccccatctgagatcatctgacgaaccttagcaaccgcagtatcgacagattgaaaacttccgccatcactaaagctatcaggagtcaagttcaagactcccataacagaggttttatgagaccaatcccataagcgatttccaacgggcaaaacccttttcattccatccttcccgattagggaacttccaccgag

SEQ ID NO: 147 - DNA, 200 bp, Amaranthus palmeri
gtcagatgatctcagatggggcagatataattgacatcggggcacaatcaaccagacccatggcaactagaatttcggctgaagaagagctagaaagagtagtacctgtattggaagctgtcaaggatttgatcgaggaagaaggaagaatcttgtcagtggatacgttttattctaaagttgcttcggaggccgtcaag

SEQ ID NO: 148 - DNA, 200 bp, Amaranthus palmeri
cttgacggcctccgaagcaactttagaataaaacgtatccactgacaagattcttccttcttcctcgatcaaatccttgacagcttccaatacaggtactactctttctagctcttcttcagccgaaattctagttgccatgggtctggttgattgtgccccgatgtcaattatatctgccccatctgagatcatctgac

SEQ ID NO: 149 - DNA, 200 bp, Amaranthus palmeri
taaagttgcttcggaggccgtcaagaagggggcacacattgtgaatgatgtctctagtgggaaactcgattccgagatgtttaatgttgttgcggaccttaaagttccttatatagcaatgcacatgcgaggagatccgacttcaatgcaaaactctgagaacttgacctacaatgatgtttgtaagcaagtggctttgg

SEQ ID NO: 150 - DNA, 200 bp, Amaranthus palmeri
ccaaagccacttgcttacaaacatcattgtaggtcaagttctcagagttttgcattgaagtcggatctcctcgcatgtgcattgctatataaggaactttaaggtccgcaacaacattaaacatctcggaatcgagtttcccactagagacatcattcacaatgtgtgcccccttcttgacggcctccgaagcaacttta

SEQ ID NO: 151 - DNA, 200 bp, Amaranthus palmeri
gatgtttgtaagcaagtggctttggagttgagttctagggtcatagatgcagaattatcgggaattcctgcttggaggatagttattgatcccggcatcggattttctaagaatacgaatcaaaatttggaaattcttagtggtttacaaaagatacgggaagagatagctaagaagagtttggcggtggctcattgccc

SEQ ID NO: 152 - DNA, 200 bp, Amaranthus palmeri
gggcaatgagccaccgccaaactcttcttagctatctcttcccgtatcttttgtaaaccactaagaatttccaaattttgattcgtattcttagaaaatccgatgccgggatcaataactatcctccaagcaggaattcccgataattctgcatctatgaccctagaactcaactccaaagccacttgcttacaaacatc

SEQ ID NO: 153 - DNA, 200 bp, Amaranthus palmeri
agagtttggcggtggctcattgccccttgctaattggaccttcaagaaagaggtttctgggcgagatttgtgagcgccctgtagcagctgacagggatcctgctaccattgcttctataactgctggagttttaggtggtgcaaacattgtaagagtacataatgtgagggataaccttgatgctgtcaagttatgtgat

SEQ ID NO: 154 - DNA, 200 bp, Amaranthus palmeri
atcacataacttgacagcatcaaggttatccctcacattatgtactcttacaatgtttgcaccacctaaaactccagcagttatagaagcaatggtagcaggatccctgtcagctgctacagggcgctcacaaatctcgcccagaaacctctttcttgaaggtccaattagcaaggggcaatgagccaccgccaaactct

SEQ ID NO: 155 - DNA, 200 bp, Amaranthus palmeri
gctttccttcactcttcaccagggaacgttgttgaagtttgttccaagaagcaagaagtcgtaattgcaatcgggagcaatgttggtgacagattagaaaatttcaaccaagctctgcaacaaatgaagaaattaggcatagacatcacaaggcatggttgtttatatgagacggaacctgcatacgtgactgatcaacc

SEQ ID NO: 156 - DNA, 200 bp, Amaranthus palmeri
ggttgatcagtcacgtatgcaggttccgtctcatataaacaaccatgccttgtgatgtctatgcctaatttcttcatttgttgcagagcttggttgaaattttctaatctgtcaccaacattgctcccgattgcaattacgacttcttgcttcttggaacaaacttcaacaacgttccctggtgaagagtgaaggaaagc

SEQ ID NO: 157 - DNA, 200 bp, Amaranthus palmeri
aacctgcatacgtgactgatcaaccgaagtttcttaactctgctttaagaggctttacaagacttggacctcatgaattattaggggtattgaagaaaattgagaaggatatgggtagaaccaagggaataaggtatggtcctaggccaattgacttggacatactattttatgggaagtttagggtgagctctgagagc

SEQ ID NO: 158 - DNA, 200 bp, Amaranthus palmeri
gctctcagagctcaccctaaacttcccataaaatagtatgtccaagtcaattggcctaggaccataccttattcccttggttctacccatatccttctcaattttcttcaatacccctaataattcatgaggtccaagtcttgtaaagcctcttaaagcagagttaagaaacttcggttgatcagtcacgtatgcaggtt

SEQ ID NO: 159 - DNA, 200 bp, Amaranthus palmeri
gaagtttagggtgagctctgagagcctcactatcccccatgaaaggatatgggaaagaccatttgtgatggcaccattgattgattgtattgggtctgatgtagaaaatgacactatttgtacgtggcattcattatcaaatttttcgggtggaatctttgaagcatggggtaaactcggtggaagctccctaatcggga

SEQ ID NO: 160 - DNA, 200 bp, Amaranthus palmeri
tcccgattagggagcttccaccgagtttaccccatgcttcaaagattccacccgaaaaatttgataatgaatgccacgtacaaatagtgtcattttctacatcagacccaatacaatcaatcaatggtgccatcacaaatggtctttcccatatcctttcatgggggatagtgaggctctcagagctcaccctaaacttc

SEQ ID NO: 161 - DNA, 200 bp, Amaranthus palmeri
ctcggtggaagctccctaatcgggaaggatggaatgaaaagggttttgcccgttggaaatctcttatgggattggtctcataaaacctctgttatgggagtcttgaacttgactcctgatagctttagtgatggcggaagttttcaatctgtcaatactgcggttgctaaggttcgtcagatgatctcagatggggcaga

SEQ ID NO: 162 - DNA, 200 bp, Amaranthus palmeri
tctgccccatctgagatcatctgacgaaccttagcaaccgcagtattgacagattgaaaacttccgccatcactaaagctatcaggagtcaagttcaagactcccataacagaggttttatgagaccaatcccataagagatttccaacgggcaaaacccttttcattccatccttcccgattagggagcttccaccgag

SEQ ID NO: 163 - DNA, 200 bp, Amaranthus palmeri
gtcagatgatctcagatggggcagatataattgacatcggggcacaatcaaccagacccatggcaactagaatttcggctgaagaagagctagcaagagtagtacctgtattagaagctgtcaaggatttgatcgaggaagaaggaagaatcttgtcagtggatacgttttattctaaagttgctttggaggccgtcaag

SEQ ID NO: 164 - DNA, 200 bp, Amaranthus palmeri
cttgacggcctccaaagcaactttagaataaaacgtatccactgacaagattcttccttcttcctcgatcaaatccttgacagcttctaatacaggtactactcttgctagctcttcttcagccgaaattctagttgccatgggtctggttgattgtgccccgatgtcaattatatctgccccatctgagatcatctgac

SEQ ID NO: 165 - DNA, 200 bp, Amaranthus palmeri
taaagttgctttggaggccgtcaagaagggggcacacattgtgaatgatgtctctagtggtaaactcgattccgagatgtttaatgttgttgcggaccttaaagttccttatatagcaatgcacatgcgaggagatccgacttcaatgcaaaactctgagaacttgacctacaatgatgtttgtaagcaagtggcttcgg

SEQ ID NO: 166 - DNA, 200 bp, Amaranthus palmeri
ccgaagccacttgcttacaaacatcattgtaggtcaagttctcagagttttgcattgaagtcggatctcctcgcatgtgcattgctatataaggaactttaaggtccgcaacaacattaaacatctcggaatcgagtttaccactagagacatcattcacaatgtgtgcccccttcttgacggcctccaaagcaacttta

SEQ ID NO: 167 - DNA, 200 bp, Amaranthus palmeri
gatgtttgtaagcaagtggcttcggagttgagttctagggtcatagatgcagaattatcgggaattcctgcttggaggatagttattgatcccggcatcggattttctaagaatacgaatcaaaatttggaaattcttagtggtttacaaaagatacgggaagagatagctaagaagagtttggcggtggctcattgccc

SEQ ID NO: 168 - DNA, 200 bp, Amaranthus palmeri
gggcaatgagccaccgccaaactcttcttagctatctcttcccgtatcttttgtaaaccactaagaatttccaaattttgattcgtattcttagaaaatccgatgccgggatcaataactatcctccaagcaggaattcccgataattctgcatctatgaccctagaactcaactccgaagccacttgcttacaaacatc

SEQ ID NO: 169 - DNA, 200 bp, Amaranthus palmeri
agagtttggcggtggctcattgccccttgctaattggaccttcaagaaagaggtttctgggcgagatttgtgagcgccctgtagcagctgacagggatcctgctaccattgcttctataactgctggagttttaggtggtgcaaacattgtaagagtacataatgttagggataaccttgatgctgtcaagttatgtgat

SEQ ID NO: 170 - DNA, 200 bp, Amaranthus palmeri
atcacataacttgacagcatcaaggttatccctaacattatgtactcttacaatgtttgcaccacctaaaactccagcagttatagaagcaatggtagcaggatccctgtcagctgctacagggcgctcacaaatctcgcccagaaacctctttcttgaaggtccaattagcaaggggcaatgagccaccgccaaactct

SEQ ID NO: 171 - DNA, 200 bp, Amaranthus palmeri
ggtgtttatttgggcatggccttatgtgtgatgttggtaaattttgtggcttcgtccactaatctgatgtatcggatctgttaaagggagccctctcatcctatgcttatcaatgactttttcatatgcctatcaaacagatcagattggatcgggttcgagttcaggtcatatataaacatgtcagcaaacccttgacc

SEQ ID NO: 172 - DNA, 200 bp, Amaranthus palmeri
ggtcaagggtttgctgacatgtttatatatgacctgaactcgaacccgatccaatctgatctgtttgataggcatatgaaaaagtcattgataagcataggatgagagggctccctttaacagatccgatacatcagattagtggacgaagccacaaaatttaccaacatcacacataaggccatgcccaaataaacacc

SEQ ID NO: 173 - DNA, 200 bp, Amaranthus palmeri
taaacatgtcagcaaacccttgacccatacccaacccatttagttaattggcttgaaaatcacaaccttaattcgaccagcgacagatcaggttgacctgatttcgacccacttaacccatttataatttttttttcgatttaagttttttaacattgattaaatctaattttttaggctttaattttaattttatgttt

SEQ ID NO: 174 - DNA, 200 bp, Amaranthus palmeri
aaacataaaattaaaattaaagcctaaaaaattagatttaatcaatgttaaaaaacttaaatcgaaaaaaaaattataaatgggttaagtgggtcgaaatcaggtcaacctgatctgtcgctggtcgaattaaggttgtgattttcaagccaattaactaaatgggttgggtatgggtcaagggtttgctgacatgttta

SEQ ID NO: 175 - DNA, 200 bp, Amaranthus palmeri
aggctttaattttaattttatgtttttttgtttaatttgtatttactatgaaaaataagaaactattatatgaactgagtttagcttacacttattaatttaactcgaaattaacacatttaattaaatggttatatagggttttttagttttaaaatttcaacctgaacctaatctatttaataaaaagatcagatctg

SEQ ID NO: 176 - DNA, 200 bp, Amaranthus palmeri
cagatctgatctttttattaaatagattaggttcaggttgaaattttaaaactaaaaaaccctatataaccatttaattaaatgtgttaatttcgagttaaattaataagtgtaagctaaactcagttcatataatagtttcttatttttcatagtaaatacaaattaaacaaaaaaacataaaattaaaattaaagcct

SEQ ID NO: 177 - DNA, 200 bp, Amaranthus palmeri
ctatttaataaaaagatcagatctgatcaacccatataattaattgggtcaaaatctcaactcaaacccatttatttcgaattaaattcagatcaaattagcaggtcggatcaactagcgctaatttttcctctcaagttttttcatatgatgccccttgttttctttgaataaaattcaccaatttaaaatccctgccc

SEQ ID NO: 178 - DNA, 200 bp, Amaranthus palmeri
gggcagggattttaaattggtgaattttattcaaagaaaacaaggggcatcatatgaaaaaacttgagaggaaaaattagcgctagttgatccgacctgctaatttgatctgaatttaattcgaaataaatgggtttgagttgagattttgacccaattaattatatgggttgatcagatctgatctttttattaaatag

SEQ ID NO: 179 - DNA, 200 bp, Amaranthus palmeri
attcaccaatttaaaatccctgcccttgatccgttggtattggtatttggtaaagcacatatggtaaattctactccattgttatctgtagttttggttctaaaccccaaacaaatcctactcctagcaccactaatcctcattactttctgtttgctgtctttctctacattcctactaaattaagacctagatgcagt

SEQ ID NO: 180 - DNA, 200 bp, Amaranthus palmeri
actgcatctaggtcttaatttagtaggaatgtagagaaagacagcaaacagaaagtaatgaggattagtggtgctaggagtaggatttgtttggggtttagaaccaaaactacagataacaatggagtagaatttaccatatgtgctttaccaaataccaataccaacggatcaagggcagggattttaaattggtgaat

SEQ ID NO: 181 - DNA, 200 bp, Amaranthus palmeri
tactaaattaagacctagatgcagtgttattctgttcgaggttgccgaaaacggtacaatagttgcctaatttgcaattcatgtttgttcttgcgttttctacattgtttactctattggaacaaaggttgaactttcagcctttcagggagtattttgatttttctgtcaaattaccttgccagatttatttggctatt

SEQ ID NO: 182 - DNA, 200 bp, Amaranthus palmeri
aatagccaaataaatctggcaaggtaatttgacagaaaaatcaaaatactccctgaaaggctgaaagttcaacctttgttccaatagagtaaacaatgtagaaaacgcaagaacaaacatgaattgcaaattaggcaactattgtaccgttttcggcaacctcgaacagaataacactgcatctaggtcttaatttagta

SEQ ID NO: 183 - DNA, 200 bp, Amaranthus palmeri
accttgccagatttatttggctattttgattgtgtgtatttctttgtcccttgtttccatggggctaaactccttttggttaatcttaaacatttcttctaattttgaattgcagtggtatgaagtgttgtttaatatacaatcaatttttctgaactgggttaatgaagagaagcattgtagacatgaggaatgtgttc

SEQ ID NO: 184 - DNA, 200 bp, Amaranthus palmeri
gaacacattcctcatgtctacaatgcttctcttcattaacccagttcagaaaaattgattgtatattaaacaacacttcataccactgcaattcaaaattagaagaaatgtttaagattaaccaaaaggagtttagccccatggaaacaagggacaaagaaatacacacaatcaaaatagccaaataaatctggcaaggt

SEQ ID NO: 185 - DNA, 200 bp, Amaranthus palmeri
cattgtagacatgaggaatgtgttcaagcatctcaaaacaaccaaattaatcccaaatgggatcttcaactgtaatagaggtaaatcacatatattctcatttaatggttattgttttattttaaggatgttattatgttgttatcaatgatagaaataagagaagcttgagcattcaaatgtatctatcactcactggt

SEQ ID NO: 186 - DNA, 200 bp, Amaranthus palmeri
accagtgagtgatagatacatttgaatgctcaagcttctcttatttctatcattgataacaacataataacatccttaaaataaaacaataaccattaaatgagaatatatgtgatttacctctattacagttgaagatcccatttgggattaatttggttgttttgagatgcttgaacacattcctcatgtctacaatg

SEQ ID NO: 187 - DNA, 200 bp, Amaranthus palmeri
tcaaatgtatctatcactcactggttgtggacatctaaaatatggaccaagagttttcttcatttcttgtaccagaatttgcaaatgtatgattaaagtttaggttggagttgcaataaaatttcattaccccgacgctattaagtggctcacactgaggcggggtttgagggtcggatggtgcaaccttcaggagaggt

SEQ ID NO: 188 - DNA, 200 bp, Amaranthus palmeri
acctctcctgaaggttgcaccatccgaccctcaaaccccgcctcagtgtgagccacttaatagcgtcggggtaatgaaattttattgcaactccaacctaaactttaatcatacatttgcaaattctggtacaagaaatgaagaaaactcttggtccatattttagatgtccacaaccagtgagtgatagatacatttga

SEQ ID NO: 189 - DNA, 200 bp, Amaranthus palmeri
ggatggtgcaaccttcaggagaggttctaagcattttcaacatgttcaaccgcagagggtcccgtaaaattggaggcttcaatattaaattatgtagtatgtgttaagtgtttaaagatataaaaatgttaatgaatacataatttttaaaattgcacagggctttcaaagaaatttttgattttgctccgcccatggca

SEQ ID NO: 190 - DNA, 200 bp, Amaranthus palmeri
tgccatgggcggagcaaaatcaaaaatttctttgaaagccctgtgcaattttaaaaattatgtattcattaacatttttatatctttaaacacttaacacatactacataatttaatattgaagcctccaattttacgggaccctctgcggttgaacatgttgaaaatgcttagaacctctcctgaaggttgcaccatcc

SEQ ID NO: 191 - DNA, 200 bp, Amaranthus palmeri
ttttgattttgctccgcccatggcaacctttggcaaatctggatagaattagagaaatttacttttatgtatgtataatctgatgaatgaatgaatgaacaatgaagttcatttctgagtcaatgacctatatactatcgaggtttcgttaaataggatatataaataaaattagagacatcttttatccctaatgttgt

SEQ ID NO: 192 - DNA, 200 bp, Amaranthus palmeri
acaacattagggataaaagatgtctctaattttatttatatatcctatttaacgaaacctcgatagtatataggtcattgactcagaaatgaacttcattgttcattcattcattcatcagattatacatacataaaagtaaatttctctaattctatccagatttgccaaaggttgccatgggcggagcaaaatcaaaa

SEQ ID NO: 193 - DNA, 200 bp, Amaranthus palmeri
agacatcttttatccctaatgttgtctgttttcttttctgggcgagactgtttttggagaaaaacgttatgttgaatgcttgatttactcagaactgatgcaaacagatggataaaaaatggtgttttcctagtgtttatgtttcataaaaggatttcaaattccaatcgtatgaagtttgaaatactgctttttgtttt

SEQ ID NO: 194 - DNA, 200 bp, Amaranthus palmeri
aaaacaaaaagcagtatttcaaacttcatacgattggaatttgaaatccttttatgaaacataaacactaggaaaacaccattttttatccatctgtttgcatcagttctgagtaaatcaagcattcaacataacgtttttctccaaaaacagtctcgcccagaaaagaaaacagacaacattagggataaaagatgtct

SEQ ID NO: 195 - DNA, 200 bp, Amaranthus palmeri
agtttgaaatactgctttttgtttttgtagcttcgtgttttgctttccttcactcttcaccagggaacgttgttgaagtttgttccaagaagcaagaagtcgtaattgcaatcgggagcaatgttggtgacagattagaaaatttcaaccaagctctgcaacaaatgaagaaattaggcatagacatcacaaggcatggt

SEQ ID NO: 196 - DNA, 200 bp, Amaranthus palmeri
accatgccttgtgatgtctatgcctaatttcttcatttgttgcagagcttggttgaaattttctaatctgtcaccaacattgctcccgattgcaattacgacttcttgcttcttggaacaaacttcaacaacgttccctggtgaagagtgaaggaaagcaaaacacgaagctacaaaaacaaaaagcagtatttcaaact

SEQ ID NO: 197 - DNA, 200 bp, Amaranthus palmeri
aggcatagacatcacaaggcatggttgtttatatgagacggaacctgcatacgtgactgatcaaccgaagtttcttaactctgctttaagaggctttacaagacttggacctcatgaattattaggggtattgaagaaaattgagaaggatatgggtagaaccaagggaataaggtatggtcctaggccaattgacttgg

SEQ ID NO: 198 - DNA, 200 bp, Amaranthus palmeri
ccaagtcaattggcctaggaccataccttattcccttggttctacccatatccttctcaattttcttcaatacccctaataattcatgaggtccaagtcttgtaaagcctcttaaagcagagttaagaaacttcggttgatcagtcacgtatgcaggttccgtctcatataaacaaccatgccttgtgatgtctatgcct

SEQ ID NO: 199 - DNA, 200 bp, Amaranthus palmeri
tatggtcctaggccaattgacttggacatactattttatgggaagtttagggtgagctctgagagcctcactatcccccatgaaaggatatgggaaagaccatttgtgatggcaccattgattgattgtattgggtctgatgtagaaaatgacactatttgtacgtggcattcattatcaaatttttcgggtggaatctt

SEQ ID NO: 200 - DNA, 200 bp, Amaranthus palmeri
aagattccacccgaaaaatttgataatgaatgccacgtacaaatagtgtcattttctacatcagacccaatacaatcaatcaatggtgccatcacaaatggtctttcccatatcctttcatgggggatagtgaggctctcagagctcaccctaaacttcccataaaatagtatgtccaagtcaattggcctaggaccata

SEQ ID NO: 201 - DNA, 200 bp, Amaranthus palmeri
tatcaaatttttcgggtggaatctttgaagcatggggtaaactcggtggaagctccctaatcgggaaggatggaatgaaaagggttttgcccgttggaaatctcttatgggattggtctcataaaacctctgttatgggagtcttgaacttgactcctgatagctttagtgatggcggaagttttcaatctgtcaatact

SEQ ID NO: 202 - DNA, 200 bp, Amaranthus palmeri
agtattgacagattgaaaacttccgccatcactaaagctatcaggagtcaagttcaagactcccataacagaggttttatgagaccaatcccataagagatttccaacgggcaaaacccttttcattccatccttcccgattagggagcttccaccgagtttaccccatgcttcaaagattccacccgaaaaatttgata

SEQ ID NO: 203 - DNA, 200 bp, Amaranthus palmeri
cggaagttttcaatctgtcaatactgcggttgctaaggttcgtcagatgatctcagatggggcagatataattgacatcggggcacaatcaaccagacccatggcaactagaatttcggctgaagaagagctagcaagagtagtacctgtattagaagctgtcaaggatttgatcgaggaagaaggaagaatcttgtcag

SEQ ID NO: 204 - DNA, 200 bp, Amaranthus palmeri
ctgacaagattcttccttcttcctcgatcaaatccttgacagcttctaatacaggtactactcttgctagctcttcttcagccgaaattctagttgccatgggtctggttgattgtgccccgatgtcaattatatctgccccatctgagatcatctgacgaaccttagcaaccgcagtattgacagattgaaaacttccg

SEQ ID NO: 205 - DNA, 200 bp, Amaranthus palmeri
gaggaagaaggaagaatcttgtcagtggatacgttttattctaaagttgctttggaggccgtcaagaagggggcacacattgtgaatgatgtctctagtggtaaactcgattccgagatgtttaatgttgttgcggaccttaaagttccttatatagcaatgcacatgcgaggagatccgacttcaatgcaaaactctga

SEQ ID NO: 206 - DNA, 200 bp, Amaranthus palmeri
tcagagttttgcattgaagtcggatctcctcgcatgtgcattgctatataaggaactttaaggtccgcaacaacattaaacatctcggaatcgagtttaccactagagacatcattcacaatgtgtgcccccttcttgacggcctccaaagcaactttagaataaaacgtatccactgacaagattcttccttcttcctc

SEQ ID NO: 207 - DNA, 200 bp, Amaranthus palmeri
atccgacttcaatgcaaaactctgagaacttgacctacaatgatgtttgtaagcaagtggcttcggagttgagttctagggtcatagatgcagaattatcgggaattcctgcttggaggatagttattgatcccggcatcggattttctaagaatacgaatcaaaatttggaaattcttagtggtttacaaaagatacgg

SEQ ID NO: 208 - DNA, 200 bp, Amaranthus palmeri
ccgtatcttttgtaaaccactaagaatttccaaattttgattcgtattcttagaaaatccgatgccgggatcaataactatcctccaagcaggaattcccgataattctgcatctatgaccctagaactcaactccgaagccacttgcttacaaacatcattgtaggtcaagttctcagagttttgcattgaagtcggat

SEQ ID NO: 209 - DNA, 200 bp, Amaranthus palmeri
tcttagtggtttacaaaagatacgggaagagatagctaagaagagtttggcggtggctcattgccccttgctaattggaccttcaagaaagaggtttctgggcgagatttgtgagcgccctgtagcagctgacagggatcctgctaccattgcttctataactgctggagttttaggtggtgcaaacattgtaagagtac

SEQ ID NO: 210 - DNA, 200 bp, Amaranthus palmeri
gtactcttacaatgtttgcaccacctaaaactccagcagttatagaagcaatggtagcaggatccctgtcagctgctacagggcgctcacaaatctcgcccagaaacctctttcttgaaggtccaattagcaaggggcaatgagccaccgccaaactcttcttagctatctcttcccgtatcttttgtaaaccactaaga

SEQ ID NO: 211 - DNA, 200 bp, Amaranthus palmeri
ggtggtgcaaacattgtaagagtacataatgttagggataaccttgatgctgtcaagttatgtgatgccatactcggaaaaactgattaactgcttgtttgtaccaccttgtgaatgatgtctagtggaaggttcgattgggattatgatgaagtccgttagtttgctcgaaagaagagctttagcccgtaaggttatag

SEQ ID NO: 212 - DNA, 200 bp, Amaranthus palmeri
ctataaccttacgggctaaagctcttctttcgagcaaactaacggacttcatcataatcccaatcgaaccttccactagacatcattcacaaggtggtacaaacaagcagttaatcagtttttccgagtatggcatcacataacttgacagcatcaaggttatccctaacattatgtactcttacaatgtttgcaccacc

SEQ ID NO: 213 - DNA, 200 bp, Amaranthus palmeri
agagctttagcccgtaaggttatagttcttacagatgtagaatcattggctatttgcctatttctgcttagaagatcatagcattgatccagactttggattccatgtagaattatgttgcgactcatggtcgaattggccctttgaggaattgttgaagttccttgagtctatggttcaatcggctcggttcgatgaag

SEQ ID NO: 214 - DNA, 200 bp, Amaranthus palmeri
cttcatcgaaccgagccgattgaaccatagactcaaggaacttcaacaattcctcaaagggccaattcgaccatgagtcgcaacataattctacatggaatccaaagtctggatcaatgctatgatcttctaagcagaaataggcaaatagccaatgattctacatctgtaagaactataaccttacgggctaaagctct

SEQ ID NO: 215 - DNA, 200 bp, Amaranthus palmeri
gttcaatcggctcggttcgatgaaggttggactgtgtcgagacaagatgggtcaaagagaccaatggacaagaagtcgaccttgggcaagcttgatcaagttacttggacgagcagataacagcaggctctggcgagcattagacatacatatcatttgttttttgaagtcagatctgcaatcaatcagttgcaatggac

SEQ ID NO: 216 - DNA, 200 bp, Amaranthus palmeri
gtccattgcaactgattgattgcagatctgacttcaaaaaacaaatgatatgtatgtctaatgctcgccagagcctgctgttatctgctcgtccaagtaacttgatcaagcttgcccaaggtcgacttcttgtccattggtctctttgacccatcttgtctcgacacagtccaaccttcatcgaaccgagccgattgaac

SEQ ID NO: 217 - DNA, 200 bp, Amaranthus palmeri
ctgcaatcaatcagttgcaatggacagaccatatacggttgttagttgttcctgtcactctttgagttgaattagatcatgcaaacactgtaagagtcttgattttgtgaagctatgtgatactgtacttctcaaaatgaaatctttgatatgatgttagcttcaagagttgtttttccccccctcacattctgttctat

SEQ ID NO: 218 - DNA, 200 bp, Amaranthus palmeri
atagaacagaatgtgagggggggaaaaacaactcttgaagctaacatcatatcaaagatttcattttgagaagtacagtatcacatagcttcacaaaatcaagactcttacagtgtttgcatgatctaattcaactcaaagagtgacaggaacaactaacaaccgtatatggtctgtccattgcaactgattgattgcag

SEQ ID NO: 219 - DNA, 200 bp, Amaranthus palmeri
ttccccccctcacattctgttctatgttgtattgttgtgtgctttttggaattgtattcctgtaattagaattattcctgcaaaatggtattcctttatatcatcaaaggagtttttgtacttctaatcattgatctttagatgcaaaactgaattaatacaagtgttcttgatgaaggcttattgaagaactcactttt

SEQ ID NO: 220 - DNA, 200 bp, Amaranthus palmeri
aaaagtgagttcttcaataagccttcatcaagaacacttgtattaattcagttttgcatctaaagatcaatgattagaagtacaaaaactcctttgatgatataaaggaataccattttgcaggaataattctaattacaggaatacaattccaaaaagcacacaacaatacaacatagaacagaatgtgagggggggaa

SEQ ID NO: 221 - DNA, 200 bp, Amaranthus palmeri
aaggcttattgaagaactcactttttatttatgtctaaacttgttaaagatgttctgcttccgcccttgccacgggatgtcgggattgactaagggtggtaatcgagggattcctatgcttgaattgactcgtttaagattttgcgttgattttaagtcaaatgttttgctaagcataaactattctagctcgacttgaa

SEQ ID NO: 222 - DNA, 200 bp, Amaranthus palmeri
ttcaagtcgagctagaatagtttatgcttagcaaaacatttgacttaaaatcaacgcaaaatcttaaacgagtcaattcaagcataggaatccctcgattaccacccttagtcaatcccgacatcccgtggcaagggcggaagcagaacatctttaacaagtttagacataaataaaaagtgagttcttcaataagcctt

SEQ ID NO: 223 - DNA, 200 bp, Amaranthus palmeri
ataaactattctagctcgacttgaataatgaagttttcgagtcaagtccgagctgttctgtaagtttatgtatcttgaagcttcttagttcttacttcttatccttatttgtttgacacattgaaccgcgttgatgcttgcttgactcaacccgttaaagtcttgaattcatcacgaattcgagtcgagcaagtttaaac

SEQ ID NO: 224 - DNA, 200 bp, Amaranthus palmeri
gtttaaacttgctcgactcgaattcgtgatgaattcaagactttaacgggttgagtcaagcaagcatcaacgcggttcaatgtgtcaaacaaataaggataagaagtaagaactaagaagcttcaagatacataaacttacagaacagctcggacttgactcgaaaacttcattattcaagtcgagctagaatagtttat

SEQ ID NO: 225 - DNA, 200 bp, Amaranthus palmeri
gaattcgagtcgagcaagtttaaacccaacattaactaagctctcgaggcttaatgaggcaggggtagctcaagtttgttattcaagcaacttaccatagtattcttttaaaaggcatatagtcagttagttatttgttaatttcagtagaacgaaagaagtacattattaaacttggccattgatgcttgagctagaga

SEQ ID NO: 226 - DNA, 200 bp, Amaranthus palmeri
tctctagctcaagcatcaatggccaagtttaataatgtacttctttcgttctactgaaattaacaaataactaactgactatatgccttttaaaagaatactatggtaagttgcttgaataacaaacttgagctacccctgcctcattaagcctcgagagcttagttaatgttgggtttaaacttgctcgactcgaattc

SEQ ID NO: 227 - DNA, 200 bp, Amaranthus palmeri
tggccattgatgcttgagctagagagcttcggttgtgatcgaaacgaatcctgatttcgctcttgatctttgatgtgaagtgacaaggccttcataaaactcctttattcttcagcagtgcatacaaaaatcatgtgtgcccagctcctagctcacataccgcgtttttcccctaatctagtgtggtcaaataaaaacag

SEQ ID NO: 228 - DNA, 200 bp, Amaranthus palmeri
ctgtttttatttgaccacactagattaggggaaaaacgcggtatgtgagctaggagctgggcacacatgatttttgtatgcactgctgaagaataaaggagttttatgaaggccttgtcacttcacatcaaagatcaagagcgaaatcaggattcgtttcgatcacaaccgaagctctctagctcaagcatcaatggcca

SEQ ID NO: 229 - DNA, 200 bp, Amaranthus palmeri
atctagtgtggtcaaataaaaacagcaagatgtgttgatgtcatgtgcaatagtaattatcaggtgaagtaatgcaatatgtagccagtcacctcatatcaaaaaaatgttgggaggaaaacgtattcgttcatcactaaacctgtgtttatacatctaactgatcgatcagggtttcaaaatccaaattaatacaatgg

SEQ ID NO: 230 - DNA, 200 bp, Amaranthus palmeri
ccattgtattaatttggattttgaaaccctgatcgatcagttagatgtataaacacaggtttagtgatgaacgaatacgttttcctcccaacatttttttgatatgaggtgactggctacatattgcattacttcacctgataattactattgcacatgacatcaacacatcttgctgtttttatttgaccacactagat

SEQ ID NO: 231 - DNA, 200 bp, Amaranthus palmeri
ttcaaaatccaaattaatacaatggcatttgacgaagatatccagaacacatcccccttgtttctgctagataatcaactattctgattctgctgtaatacctcagcttcggccctagccatgggatcgtcctctttttctccttcacgctcctcggccagtttctttctgtgcagttcttcagcagcttctattgaagc

SEQ ID NO: 232 - DNA, 200 bp, Amaranthus palmeri
gcttcaatagaagctgctgaagaactgcacagaaagaaactggccgaggagcgtgaaggagaaaaagaggacgatcccatggctagggccgaagctgaggtattacagcagaatcagaatagttgattatctagcagaaacaagggggatgtgttctggatatcttcgtcaaatgccattgtattaatttggattttgaa

SEQ ID NO: 233 - DNA, 200 bp, Amaranthus rudis
aatgggagtcttgaacttgactcctgatagctttagtgatggcggaaggtttcaatctgtcgatactgcggttgctaaggttcgtcagatgatctctgacggggcggacataattgacattggggcgcagtcaacaagacccatggcaactaggatttcggctgaagaagagctagaaagagtcgtacctgtattagaag

SEQ ID NO: 234 - DNA, 200 bp, Amaranthus rudis
cttctaatacaggtacgactctttctagctcttcttcagccgaaatcctagttgccatgggtcttgttgactgcgccccaatgtcaattatgtccgccccgtcagagatcatctgacgaaccttagcaaccgcagtatcgacagattgaaaccttccgccatcactaaagctatcaggagtcaagttcaagactcccatt

SEQ ID NO: 235 - DNA, 200 bp, Amaranthus rudis
gaaagagtcgtacctgtattagaagctgtcaaggatttgatcgaggaagaaggaagaatcttgtcagtggatacgttttattctaaagttgcttcggaggccgtcaagaagggggcacacattgtgaatgatgtctctagtgggaaactcgatcccgagatgtttaatgttgttgcggaccttaaagttccttatatagc

SEQ ID NO: 236 - DNA, 200 bp, Amaranthus rudis
gctatataaggaactttaaggtccgcaacaacattaaacatctcgggatcgagtttcccactagagacatcattcacaatgtgtgcccccttcttgacggcctccgaagcaactttagaataaaacgtatccactgacaagattcttccttcttcctcgatcaaatccttgacagcttctaatacaggtacgactctttc

SEQ ID NO: 237 - DNA, 200 bp, Amaranthus rudis
cggaccttaaagttccttatatagcaatgcacatgcgaggagatccgacttcaatgcaaaattctgagaacttgacgtacaatgatgtgtgtaagcaagtggcttcggagttgagctctagggtcgtagatgcagaattatcgggaattcctgcttggaggatggttattgatcccggcattggattttctaagaatacg

SEQ ID NO: 238 - DNA, 200 bp, Amaranthus rudis
cgtattcttagaaaatccaatgccgggatcaataaccatcctccaagcaggaattcccgataattctgcatctacgaccctagagctcaactccgaagccacttgcttacacacatcattgtacgtcaagttctcagaattttgcattgaagtcggatctcctcgcatgtgcattgctatataaggaactttaaggtccg

SEQ ID NO: 239 - DNA, 200 bp, Amaranthus rudis
cggcattggattttctaagaatacgaagcaaaatttggaaattcttagtggtttacaaaagatacggcaagagatagctaagaagagtttggcggtggctcattgccccttgctaattggaccttcaagaaagaggtttctgggcgagatttgcaatcgccctgtagcagctgatagggatcctgctaccgttgcttcta

SEQ ID NO: 240 - DNA, 200 bp, Amaranthus rudis
tagaagcaacggtagcaggatccctatcagctgctacagggcgattgcaaatctcgcccagaaacctctttcttgaaggtccaattagcaaggggcaatgagccaccgccaaactcttcttagctatctcttgccgtatcttttgtaaaccactaagaatttccaaattttgcttcgtattcttagaaaatccaatgccg

SEQ ID NO: 241 - DNA, 200 bp, Amaranthus rudis
aggatttcggctgaagaagagctagaaagagttgtacctgtattggaagctgttaaggatgtgatcgaggaagaaggaagaatcttatcggtggatacgttttattctaaagttgcttcggaggccgtcaagaagggggcacacattgtgaatgatgtttctagtgggaaactcgatcccgagatgtttaatgttgttgc

SEQ ID NO: 242 - DNA, 200 bp, Amaranthus rudis
gcaacaacattaaacatctcgggatcgagtttcccactagaaacatcattcacaatgtgtgcccccttcttgacggcctccgaagcaactttagaataaaacgtatccaccgataagattcttccttcttcctcgatcacatccttaacagcttccaatacaggtacaactctttctagctcttcttcagccgaaatcct

SEQ ID NO: 243 - DNA, 200 bp, Amaranthus rudis
atcccgagatgtttaatgttgttgcggaccttaaagttccttatatagcaatgcacatgcgaggagatccgacttcaatgcaaaattctgagaacttgacgtacaatgatgtgtgtaagcaagtggcttcggagttgagctctagggtcgtagatgcagaattatcgggaattcctgcttggaggatggttattgatccc

SEQ ID NO: 244 - DNA, 200 bp, Amaranthus rudis
gggatcaataaccatcctccaagcaggaattcccgataattctgcatctacgaccctagagctcaactccgaagccacttgcttacacacatcattgtacgtcaagttctcagaattttgcattgaagtcggatctcctcgcatgtgcattgctatataaggaactttaaggtccgcaacaacattaaacatctcgggat

SEQ ID NO: 245 - DNA, 200 bp, Amaranthus rudis
tgcttggaggatggttattgatcccggcattggattttctaagaatacgaagcaaaatttggaaattcttagtggtttacaaaagatacggcaagagatagctaagaagagtttggcggtggctcattgccccttgctaattggaccttcaagaaagaaggtttctgggcgagatttgcaatcgccctgtagcagctgat

SEQ ID NO: 246 - DNA, 200 bp, Amaranthus rudis
atcagctgctacagggcgattgcaaatctcgcccagaaaccttctttcttgaaggtccaattagcaaggggcaatgagccaccgccaaactcttcttagctatctcttgccgtatcttttgtaaaccactaagaatttccaaattttgcttcgtattcttagaaaatccaatgccgggatcaataaccatcctccaagca

SEQ ID NO: 247 - DNA, 200 bp, Amaranthus rudis
attagaggcatcttttatccctaatgctgtccgttttcttttctgggtgagactattttggaagaaaaaaatttatgttcaatgtttaatttactcagaattgatgcaaacagatggataaaaatggtgttttcttatggtttatgtttcaataaaaggatttcaaattctaatcatatgaagtttgaaatactatgtgt

SEQ ID NO: 248 - DNA, 200 bp, Amaranthus rudis
acacatagtatttcaaacttcatatgattagaatttgaaatccttttattgaaacataaaccataagaaaacaccatttttatccatctgtttgcatcaattctgagtaaattaaacattgaacataaatttttttcttccaaaatagtctcacccagaaaagaaaacggacagcattagggataaaagatgcctctaat

SEQ ID NO: 249 - DNA, 200 bp, Amaranthus rudis
atatgaagtttgaaatactatgtgtttttgtagcttcttgtattgctttccttcactcatcaccagtgaacgatgttgaagtttgttccaagaagcaagaagtcgtaattgcaatcgggagcaatgttggtgacagattagaaaatttcaacgaagctctgcaacaaatgaagaaattaggcatagacatcacaaggcat

SEQ ID NO: 250 - DNA, 200 bp, Amaranthus rudis
atgccttgtgatgtctatgcctaatttcttcatttgttgcagagcttcgttgaaattttctaatctgtcaccaacattgctcccgattgcaattacgacttcttgcttcttggaacaaacttcaacatcgttcactggtgatgagtgaaggaaagcaatacaagaagctacaaaaacacatagtatttcaaacttcatat

SEQ ID NO: 251 - DNA, 200 bp, Amaranthus rudis
attaggcatagacatcacaaggcatggttgtttatacgagacggaacctgcttacgtgactgatcagccgaagtttcttaactctgctttaagaggctttacaagacttgggcctcatgaattattaggggtattgaagagaatcgagaaggatatgggcagaaccaagggaataaggtatggtcctaggccaattgact

SEQ ID NO: 252 - DNA, 200 bp, Amaranthus rudis
agtcaattggcctaggaccataccttattcccttggttctgcccatatccttctcgattctcttcaatacccctaataattcatgaggcccaagtcttgtaaagcctcttaaagcagagttaagaaacttcggctgatcagtcacgtaagcaggttccgtctcgtataaacaaccatgccttgtgatgtctatgcctaat

SEQ ID NO: 253 - DNA, 200 bp, Amaranthus rudis
cgctactgctgcagctgtcaccgctgcaattttgaatggtgctaacatagtaagggtacataatgtaggatatagttcagatgctgcaaagttttgtgatgcacctatactcattttacccatctcgctcctaatggaccccagccccttaataacgtcaatgttgtgcccacatttcttggagaacccaatgccaggat

SEQ ID NO: 254 - DNA, 200 bp, Amaranthus rudis
atcctggcattgggttctccaagaaatgtgggcacaacattgacgttattaaggggctggggtccattaggagcgagatgggtaaaatgagtataggtgcatcacaaaactttgcagcatctgaactatatcctacattatgtacccttactatgttagcaccattcaaaattgcagcggtgacagctgcagcagtagcg

SEQ ID NO: 255 - DNA, 200 bp, Amaranthus rudis
ctgagagcctcactattccccatgaaaggatatgggaaagaccatttgtgatggcaccattaattgattgtattgggtctgatgtagaaaatgacactatttgtacatggcattcgttatcaaatttttcgggtggaatatttgaagcatgggataaactcggaggaagttccttaatcgggaaggatggaatgaaaagg

SEQ ID NO: 256 - DNA, 200 bp, Amaranthus rudis
ccttttcattccatccttcccgattaaggaacttcctccgagtttatcccatgcttcaaatattccacccgaaaaatttgataacgaatgccatgtacaaatagtgtcattttctacatcagacccaatacaatcaattaatggtgccatcacaaatggtctttcccatatcctttcatggggaatagtgaggctctcag

SEQ ID NO: 257 - DNA, 200 bp, Amaranthus rudis
aatcgggaaggatggaatgaaaagggttttgcccgttgggaaccgtttatgggattggtctcataaaacctcagtaatgggagtcttgaacttgactcctgatagctttagtgatggcggaaggtttcaatctgtcgatactgcggttgctaaggttcgtcagatgatctctgacggggcggacataattgacattgggg

SEQ ID NO: 258 - DNA, 200 bp, Amaranthus rudis
ccccaatgtcaattatgtccgccccgtcagagatcatctgacgaaccttagcaaccgcagtatcgacagattgaaaccttccgccatcactaaagctatcaggagtcaagttcaagactcccattactgaggttttatgagaccaatcccataaacggttcccaacgggcaaaacccttttcattccatccttcccgatt

SEQ ID NO: 259 - DNA, 200 bp, Amaranthus rudis
gcgtaccttcactcatcaccagtggacgatgttgaactttgttctaagaagcaagaagtcataattgcaatcgggagcaatgttggtgacagattagaaaattttaaccaagctctgcaacaaatgaagaaattaggcatagacatcacaaggcatggttgtttatatgagacagaacccgcatacgtgactgatcaacc

SEQ ID NO: 260 - DNA, 200 bp, Amaranthus rudis
ggttgatcagtcacgtatgcgggttctgtctcatataaacaaccatgccttgtgatgtctatgcctaatttcttcatttgttgcagagcttggttaaaattttctaatctgtcaccaacattgctcccgattgcaattatgacttcttgcttcttagaacaaagttcaacatcgtccactggtgatgagtgaaggtacgc

SEQ ID NO: 261 - DNA, 200 bp, Amaranthus rudis
aacccgcatacgtgactgatcaaccgaagtttcttaactctgctttaagaggctttacaagacttgggcctcatgaattattaggggtattgaagaaaattgagaagaatatgggtagaaccaagggaataaggtatggtcctaggccaattgacttggacatactattttatgggaagtttagggtgagctctgagagc

SEQ ID NO: 262 - DNA, 200 bp, Amaranthus rudis
gctctcagagctcaccctaaacttcccataaaatagtatgtccaagtcaattggcctaggaccataccttattcccttggttctacccatattcttctcaattttcttcaatacccctaataattcatgaggcccaagtcttgtaaagcctcttaaagcagagttaagaaacttcggttgatcagtcacgtatgcgggtt

SEQ ID NO: 263 - DNA, 200 bp, Amaranthus rudis
gaagtttagggtgagctctgagagcctcactattccccatgaaaggatatgggaaagaccatttgtgatggcaccattaattgattgtattgggtctggtgtagaaaatgacactatttgtacatggcattcgttatcaaatttttcgggtggaatctttgaagcatgggataaactcggtggaagttccctaatcggga

SEQ ID NO: 264 - DNA, 200 bp, Amaranthus rudis
tcccgattagggaacttccaccgagtttatcccatgcttcaaagattccacccgaaaaatttgataacgaatgccatgtacaaatagtgtcattttctacaccagacccaatacaatcaattaatggtgccatcacaaatggtctttcccatatcctttcatggggaatagtgaggctctcagagctcaccctaaacttc

SEQ ID NO: 265 - DNA, 200 bp, Amaranthus rudis
ctcggtggaagttccctaatcgggaaggatggaatgaaaagggttttgcccgttgggaaccgcttatgggattggtctcataaaacctctgtaatgggagtcttgaacttgactcctgatagctttagtgatggaggaagttttcaatctgtcgatactgcggttgctaaggttcgtcagatgatatcagatggggccga

SEQ ID NO: 266 - DNA, 200 bp, Amaranthus rudis
tcggccccatctgatatcatctgacgaaccttagcaaccgcagtatcgacagattgaaaacttcctccatcactaaagctatcaggagtcaagttcaagactcccattacagaggttttatgagaccaatcccataagcggttcccaacgggcaaaacccttttcattccatccttcccgattagggaacttccaccgag

SEQ ID NO: 267 - DNA, 200 bp, Amaranthus rudis
gtcagatgatatcagatggggccgacataattgacattggggcacagtcaacaagacccatggcaactaggatttcggctgaagaagagctagaaagagttgtacctgtattggaagctgttaaggatgtgatcgaggaagaaggaagaatcttatcggtggatactttttattcaaaagttgcttctgaggccgtcaag

SEQ ID NO: 268 - DNA, 200 bp, Amaranthus rudis
cttgacggcctcagaagcaacttttgaataaaaagtatccaccgataagattcttccttcttcctcgatcacatccttaacagcttccaatacaggtacaactctttctagctcttcttcagccgaaatcctagttgccatgggtcttgttgactgtgccccaatgtcaattatgtcggccccatctgatatcatctgac

SEQ ID NO: 269 - DNA, 200 bp, Amaranthus rudis
aaaagttgcttctgaggccgtcaagaagggggcacacattgtgaatgatgtctctagtgggaaattcgatcctgagatgttcaatgttgttgcggaccttaaagttccttatatagcaatgcacatgcgaggagatccgacttcaatgcaaaattctgagaacttgacgtacagtgatgtttgtaagcaagtggcttcgg

SEQ ID NO: 270 - DNA, 200 bp, Amaranthus rudis
ccgaagccacttgcttacaaacatcactgtacgtcaagttctcagaattttgcattgaagtcggatctcctcgcatgtgcattgctatataaggaactttaaggtccgcaacaacattgaacatctcaggatcgaatttcccactagagacatcattcacaatgtgtgcccccttcttgacggcctcagaagcaactttt

SEQ ID NO: 271 - DNA, 200 bp, Amaranthus rudis
gatgtttgtaagcaagtggcttcggagttgagctctagggtcatagatgcagaattatcgggaattcctgcttggagaatagttattgatcccggcattggattttctaagaatacgaagcaaaatttggaaattcttactggtttacaaaagatacggctagagatagctaagaagagtttggcggtggctcattgccc

SEQ ID NO: 272 - DNA, 200 bp, Amaranthus rudis
gggcaatgagccaccgccaaactcttcttagctatctctagccgtatcttttgtaaaccagtaagaatttccaaattttgcttcgtattcttagaaaatccaatgccgggatcaataactattctccaagcaggaattcccgataattctgcatctatgaccctagagctcaactccgaagccacttgcttacaaacatc

SEQ ID NO: 273 - DNA, 200 bp, Amaranthus rudis
agagtttggcggtggctcattgccccttgctaattggaccttcacgaaagaggtttctgggtgagatttgtaatcgccctgtagcagctgatagggatcctgctaccgtcgcttctatcactgctggagttttaggtggtgcaaacattgtaagagtacataatgtgagggataaccttgatgcagtcaagttatgtgat

SEQ ID NO: 274 - DNA, 200 bp, Amaranthus rudis
atcacataacttgactgcatcaaggttatccctcacattatgtactcttacaatgtttgcaccacctaaaactccagcagtgatagaagcgacggtagcaggatccctatcagctgctacagggcgattacaaatctcacccagaaacctctttcgtgaaggtccaattagcaaggggcaatgagccaccgccaaactct

SEQ ID NO: 275 - DNA, 200 bp, Amaranthus rudis
gtaatgggagtcttgaacttgactcctgatagctttagtgatggcggaaggtttcaatctgtcgatactgcggttgctaaggttcgtcagatgatctctgacggggcggacataattgacattggggcgcagtcaacaagacccttggcaactaggatttcggctgaagaagagctagaaagagtcgtacctgtattaga

SEQ ID NO: 276 - DNA, 200 bp, Amaranthus rudis
tctaatacaggtacgactctttctagctcttcttcagccgaaatcctagttgccaagggtcttgttgactgcgccccaatgtcaattatgtccgccccgtcagagatcatctgacgaaccttagcaaccgcagtatcgacagattgaaaccttccgccatcactaaagctatcaggagtcaagttcaagactcccattac

SEQ ID NO: 277 - DNA, 200 bp, Amaranthus rudis
tagaaagagtcgtacctgtattagaagctgtcaaggatttgatcgaggaagaaggaagaatcttgtcagtggatacgttttattctaaagttgcttcggaggccgtcaagaagggggcacacattgtgaatgatgtttctagtgggaaactcgatcccgagatgtttaatgttgttgcggaccttaaagttccttatata

SEQ ID NO: 278 - DNA, 200 bp, Amaranthus rudis
tatataaggaactttaaggtccgcaacaacattaaacatctcgggatcgagtttcccactagaaacatcattcacaatgtgtgcccccttcttgacggcctccgaagcaactttagaataaaacgtatccactgacaagattcttccttcttcctcgatcaaatccttgacagcttctaatacaggtacgactctttcta

SEQ ID NO: 279 - DNA, 200 bp, Amaranthus rudis
tgcggaccttaaagttccttatatagcaatgcacatgcgaggagatccgacttcaatgcaaaattctgagaacttgacgtacagtgatgtttgtaagcaagtggcttcggagttgagctctagggtcatagatgcagaattatcgggaattcctgcttggagaatagttattgatcccggcattggattttctaagaata

SEQ ID NO: 280 - DNA, 200 bp, Amaranthus rudis
tattcttagaaaatccaatgccgggatcaataactattctccaagcaggaattcccgataattctgcatctatgaccctagagctcaactccgaagccacttgcttacaaacatcactgtacgtcaagttctcagaattttgcattgaagtcggatctcctcgcatgtgcattgctatataaggaactttaaggtccgca

SEQ ID NO: 281 - DNA, 200 bp, Amaranthus rudis
cccggcattggattttctaagaatacgaagcaaaatttggaaattcttactggtttacaaaagatacggctagagatagctaagaagagtttggcggtggctcattgccccttgctaattggaccttcacgaaagaggtttctgggtgagatttgtaatcgccctgtagcagctgatagggatcctgctaccgtcgcttc

SEQ ID NO: 282 - DNA, 200 bp, Amaranthus rudis
gaagcgacggtagcaggatccctatcagctgctacagggcgattacaaatctcacccagaaacctctttcgtgaaggtccaattagcaaggggcaatgagccaccgccaaactcttcttagctatctctagccgtatcttttgtaaaccagtaagaatttccaaattttgcttcgtattcttagaaaatccaatgccggg

SEQ ID NO: 283 - DNA, 200 bp, Amaranthus rudis
tgatttactcagaattgatgcaaacaaatggataaaaatggtgttttcctatggtttatgtttcgataaaaggatttcaaatcccaatcgtatgaagtttgaaatacaactatgtgtttttgtagcttcttgtattgctttccttcactcatcaccagtgaacgatgttgaagtttgttccaagaagcaagaagtcgtaa

SEQ ID NO: 284 - DNA, 200 bp, Amaranthus rudis
ttacgacttcttgcttcttggaacaaacttcaacatcgttcactggtgatgagtgaaggaaagcaatacaagaagctacaaaaacacatagttgtatttcaaacttcatacgattgggatttgaaatccttttatcgaaacataaaccataggaaaacaccatttttatccatttgtttgcatcaattctgagtaaatca

SEQ ID NO: 285 - DNA, 200 bp, Amaranthus rudis
tgttccaagaagcaagaagtcgtaattgcaatcgggagcaatgttggtgacagattagaaaatttcaacgaagctctgcaacaaatgaagaaattaggcatagacatcacaaggcatggttgtttatacgagacggaacctgcttacgtgactgatcagccgaagtttcttaactctgctttaagaggctttacaagact

SEQ ID NO: 286 - DNA, 200 bp, Amaranthus rudis
agtcttgtaaagcctcttaaagcagagttaagaaacttcggctgatcagtcacgtaagcaggttccgtctcgtataaacaaccatgccttgtgatgtctatgcctaatttcttcatttgttgcagagcttcgttgaaattttctaatctgtcaccaacattgctcccgattgcaattacgacttcttgcttcttggaaca

SEQ ID NO: 287 - DNA, 200 bp, Amaranthus rudis
ctgctttaagaggctttacaagacttgggcctcatgaattattaggggtattgaagagaatcgagaaggatatgggcagaaccaagggaataaggtatggtcctaggccaattgacttggacatactattttatgggaagtttagggtgagctctgagagcctcactattccccatgaaaggatatgggaaagaccattt

SEQ ID NO: 288 - DNA, 200 bp, Amaranthus rudis
aaatggtctttcccatatcctttcatggggaatagtgaggctctcagagctcaccctaaacttcccataaaatagtatgtccaagtcaattggcctaggaccataccttattcccttggttctgcccatatccttctcgattctcttcaatacccctaataattcatgaggcccaagtcttgtaaagcctcttaaagcag

SEQ ID NO: 289 - DNA, 200 bp, Amaranthus rudis
tggcaactaggatttcggctgaagaagagctagaaagagtcgtacctgtattagaagctgtcaaggatttgatcgaggaagaaggaagaatcttgtcagtggatacgttttattctaaagttgcttcggaggccgtcaagaagggggcacacattgtgaatgatgtttctagtgggaaactcgatcccgagatgtttaat

SEQ ID NO: 290 - DNA, 200 bp, Amaranthus rudis
attaaacatctcgggatcgagtttcccactagaaacatcattcacaatgtgtgcccccttcttgacggcctccgaagcaactttagaataaaacgtatccactgacaagattcttccttcttcctcgatcaaatccttgacagcttctaatacaggtacgactctttctagctcttcttcagccgaaatcctagttgcca

SEQ ID NO: 291 - DNA, 200 bp, Amaranthus rudis
gaaactcgatcccgagatgtttaatgttgttgcggaccttaaagttccttatatagcaatgcacatgcgaggagatccgacttcaatgcaaaattctgagaacttgacgtacaatgatgtgtgtaagcaagtggcttcggagttgagctctagggtcgtagatgcagaattatcgggaattcctgcttggaggatggtta

SEQ ID NO: 292 - DNA, 200 bp, Amaranthus rudis
taaccatcctccaagcaggaattcccgataattctgcatctacgaccctagagctcaactccgaagccacttgcttacacacatcattgtacgtcaagttctcagaattttgcattgaagtcggatctcctcgcatgtgcattgctatataaggaactttaaggtccgcaacaacattaaacatctcgggatcgagtttc

SEQ ID NO: 293 - DNA, 200 bp, Amaranthus rudis
ggaattcctgcttggaggatggttattgatcccggcattggattttctaagaatacgaagcaaaatttggaaattcttagtggtttacaaaagatacggcaagagatagctaagaagagtttggcggtggctcattgccccttgctaattggaccttcaagaaagaggtttctgggcgagatttgcaatcgccctgtagc

SEQ ID NO: 294 - DNA, 200 bp, Amaranthus rudis
gctacagggcgattgcaaatctcgcccagaaacctctttcttgaaggtccaattagcaaggggcaatgagccaccgccaaactcttcttagctatctcttgccgtatcttttgtaaaccactaagaatttccaaattttgcttcgtattcttagaaaatccaatgccgggatcaataaccatcctccaagcaggaattcc

SEQ ID NO: 295 - DNA, 200 bp, Amaranthus rudis
gcgagatttgcaatcgccctgtagcagctgatagggatcctgctaccgttgcttctatcactgctggagttttaggtggtgcaaacattgtaagagtacataatgtgagggataaccttgatgcagtcaagttatgtgatgccatactcagaaaaacggattagccgatttttttggattccagagccgatgttccagat

SEQ ID NO: 296 - DNA, 200 bp, Amaranthus rudis
atctggaacatcggctctggaatccaaaaaaatcggctaatccgtttttctgagtatggcatcacataacttgactgcatcaaggttatccctcacattatgtactcttacaatgtttgcaccacctaaaactccagcagtgatagaagcaacggtagcaggatccctatcagctgctacagggcgattgcaaatctcgc

SEQ ID NO: 297 - DNA, 200 bp, Amaranthus rudis
tgtttcgataaaaggatttcaaatcccaatcgtatgaagtttgaaatacaactatgtgtttttgcagctttgttttttgcgtaccttcactcatcaccagtggacgatgttgaactttgttctaagaagcaagaagtcataattgcaatcgggagcaatgttggtgacagattagaaaattttaaccaagctctgcaaca

SEQ ID NO: 298 - DNA, 200 bp, Amaranthus rudis
tgttgcagagcttggttaaaattttctaatctgtcaccaacattgctcccgattgcaattatgacttcttgcttcttagaacaaagttcaacatcgtccactggtgatgagtgaaggtacgcaaaaaacaaagctgcaaaaacacatagttgtatttcaaacttcatacgattgggatttgaaatccttttatcgaaaca

SEQ ID NO: 299 - DNA, 200 bp, Amaranthus rudis
aaaattttaaccaagctctgcaacaaatgaagaaattaggcatagacatcacaaggcatggttgtttatatgagacagaacccgcatacgtgactgatcaaccgaagtttcttaactctgctttaagaggctttacaagacttgggcctcatgaattattaggggtattgaagaaaattgagaagaatatgggtagaacc

SEQ ID NO: 300 - DNA, 200 bp, Amaranthus rudis
ggttctacccatattcttctcaattttcttcaatacccctaataattcatgaggcccaagtcttgtaaagcctcttaaagcagagttaagaaacttcggttgatcagtcacgtatgcgggttctgtctcatataaacaaccatgccttgtgatgtctatgcctaatttcttcatttgttgcagagcttggttaaaatttt

SEQ ID NO: 301 - DNA, 200 bp, Amaranthus rudis
aattgagaagaatatgggtagaaccaagggaataaggtatggtcctaggccaattgacttggacatactattttatgggaagtttagggtgagctctgagagcctcactattccccatgaaaggatatgggaaagaccatttgtgatggcaccattaattgattgtattgggtctggtgtagaaaatgacactatttgta

SEQ ID NO: 302 - DNA, 200 bp, Amaranthus rudis
tacaaatagtgtcattttctacaccagacccaatacaatcaattaatggtgccatcacaaatggtctttcccatatcctttcatggggaatagtgaggctctcagagctcaccctaaacttcccataaaatagtatgtccaagtcaattggcctaggaccataccttattcccttggttctacccatattcttctcaatt

SEQ ID NO: 303 - DNA, 200 bp, Amaranthus rudis
ggtgtagaaaatgacactatttgtacatggcattcgttatcaaatttttcgggtggaatctttgaagcatgggataaactcggtggaagttccctaatcgggaaggatggaatgaaaagggttttgcccgttgggaaccgcttatgggattggtctcataaaacctctgtaatgggagtcttgaacttgactcctgatag

SEQ ID NO: 304 - DNA, 200 bp, Amaranthus rudis
ctatcaggagtcaagttcaagactcccattacagaggttttatgagaccaatcccataagcggttcccaacgggcaaaacccttttcattccatccttcccgattagggaacttccaccgagtttatcccatgcttcaaagattccacccgaaaaatttgataacgaatgccatgtacaaatagtgtcattttctacacc

SEQ ID NO: 305 - DNA, 200 bp, Amaranthus rudis
gagtcttgaacttgactcctgatagctttagtgatggaggaagttttcaatctgtcgatactgcggttgctaaggttcgtcagatgatatcagatggggccgacataattgacattggggcacagtcaacaagacccatggcaactaggatttcggctgaagaagagctagaaagagttgtacctgtattggaagctgtt

SEQ ID NO: 306 - DNA, 200 bp, Amaranthus rudis
aacagcttccaatacaggtacaactctttctagctcttcttcagccgaaatcctagttgccatgggtcttgttgactgtgccccaatgtcaattatgtcggccccatctgatatcatctgacgaaccttagcaaccgcagtatcgacagattgaaaacttcctccatcactaaagctatcaggagtcaagttcaagactc

SEQ ID NO: 307 - DNA, 200 bp, Amaranthus viridis
gcagctgatagggatcctgctaccgttgcttctataactgctggagttttaggtggtgcaaacattgtaagagtacataatgtgagggataaccttgatgctgtcaagttatgtgatgccatactcggaaaaactgattaactgcttatttgtaccaccttgtgaatgacgtctagtggaaagttcgattgggatatgat

SEQ ID NO: 308 - DNA, 200 bp, Amaranthus viridis
atcatatcccaatcgaactttccactagacgtcattcacaaggtggtacaaataagcagttaatcagtttttccgagtatggcatcacataacttgacagcatcaaggttatccctcacattatgtactcttacaatgtttgcaccacctaaaactccagcagttatagaagcaacggtagcaggatccctatcagctgc

SEQ ID NO: 309 - DNA, 200 bp, Amaranthus viridis
gtggaaagttcgattgggatatgatgaagtccgttagtttgctcgaaagtagagctttagcccgtaaggttatagttcttacagatgtagaatcattggctatttgcctatttttgcttagaagatcatagcattgatccagactttggattccatctagaattatgttgcgactcatggtcgaattggccctttgagga

SEQ ID NO: 310 - DNA, 200 bp, Amaranthus viridis
tcctcaaagggccaattcgaccatgagtcgcaacataattctagatggaatccaaagtctggatcaatgctatgatcttctaagcaaaaataggcaaatagccaatgattctacatctgtaagaactataaccttacgggctaaagctctactttcgagcaaactaacggacttcatcatatcccaatcgaactttccac

SEQ ID NO: 311 - DNA, 200 bp, Amaranthus viridis
catggtcgaattggccctttgaggaattgttgaagttccttgatctatggttcaatcggcttggttcgatgaaggttggactgtgtcgagacaagatgggtcaaagagaccaatggacaagaagtcgaccttgggaaagcttgatcaagttacttggacgagcagatagcagcaggactctggcgagcattagacataca

SEQ ID NO: 312 - DNA, 200 bp, Amaranthus viridis
tgtatgtctaatgctcgccagagtcctgctgctatctgctcgtccaagtaacttgatcaagctttcccaaggtcgacttcttgtccattggtctctttgacccatcttgtctcgacacagtccaaccttcatcgaaccaagccgattgaaccatagatcaaggaacttcaacaattcctcaaagggccaattcgaccatg

SEQ ID NO: 313 - DNA, 200 bp, Amaranthus viridis
tctgtataacctaaagattgaaacttttgaagatatttttgaagaagtactctttggctttttgatccagaaaatcatattggtatgaagtgttgtttaatatacaatcaatttttctgaactgggttattaaagagaagcattgtagacatgaggaatgtgttcaagcatctcaaaacaaccaaattaatcccaaatgg

SEQ ID NO: 314 - DNA, 200 bp, Amaranthus viridis
ccatttgggattaatttggttgttttgagatgcttgaacacattcctcatgtctacaatgcttctctttaataacccagttcagaaaaattgattgtatattaaacaacacttcataccaatatgattttctggatcaaaaagccaaagagtacttcttcaaaaatatcttcaaaagtttcaatctttaggttatacaga

SEQ ID NO: 315 - DNA, 200 bp, Amaranthus viridis
aaacaaccaaattaatcccaaatgggatcttcaactgtaatagagcttcgtgttttgctttccttcactcatcaccggtgaacgatgttgaagtttgttccaagaagcaagaagtcgtaattgcaatcgggagcaatgttggtgacagattagaaaatttcaaccaagctctgcaacaaatgaagaaattaggcatagac

SEQ ID NO: 316 - DNA, 200 bp, Amaranthus viridis
gtctatgcctaatttcttcatttgttgcagagcttggttgaaattttctaatctgtcaccaacattgctcccgattgcaattacgacttcttgcttcttggaacaaacttcaacatcgttcaccggtgatgagtgaaggaaagcaaaacacgaagctctattacagttgaagatcccatttgggattaatttggttgttt

SEQ ID NO: 317 - DNA, 200 bp, Amaranthus viridis
acaaatgaagaaattaggcatagacatcacaaggcatgggtgtttatatgagacggaacctgcatacgtgactgatcaaccgaagtttcttaactctgctttaagaggctttacaaaacttgggcctcatgaattattaggggttttaaagaaaattgagaaggatatgggtagaaccaagggaataaggtatggtccta

SEQ ID NO: 318 - DNA, 200 bp, Amaranthus viridis
taggaccataccttattcccttggttctacccatatccttctcaattttctttaaaacccctaataattcatgaggcccaagttttgtaaagcctcttaaagcagagttaagaaacttcggttgatcagtcacgtatgcaggttccgtctcatataaacacccatgccttgtgatgtctatgcctaatttcttcatttgt

SEQ ID NO: 319 - DNA, 200 bp, Amaranthus viridis
accaagggaataaggtatggtcctaggccaattgacttggacatactattttatgggaaatttagggtgagctctgagagcctcactatcccccatgaaaggatatgggaaagaccatttgtgatggcaccattgattgattgtattgggtctgatgtagaaaatgacactatttgtacatggcattcattatcaaattt

SEQ ID NO: 320 - DNA, 200 bp, Amaranthus viridis
aaatttgataatgaatgccatgtacaaatagtgtcattttctacatcagacccaatacaatcaatcaatggtgccatcacaaatggtctttcccatatcctttcatgggggatagtgaggctctcagagctcaccctaaatttcccataaaatagtatgtccaagtcaattggcctaggaccataccttattcccttggt

SEQ ID NO: 321 - DNA, 200 bp, Amaranthus viridis
gtacatggcattcattatcaaattttttgggtggaatctttgaagcatggggtaaactcggtggaagttccctaatcgggaaggatggaatgaaaagggttttgcccgttggaaatcgcttatgggattggtctcataaaacctctgtaatgggagtcttgaacttgactcctgatagctttagtgatggcggaagtttt

SEQ ID NO: 322 - DNA, 200 bp, Amaranthus viridis
aaaacttccgccatcactaaagctatcaggagtcaagttcaagactcccattacagaggttttatgagaccaatcccataagcgatttccaacgggcaaaacccttttcattccatccttcccgattagggaacttccaccgagtttaccccatgcttcaaagattccacccaaaaaatttgataatgaatgccatgtac

SEQ ID NO: 323 - DNA, 200 bp, Amaranthus viridis
tagctttagtgatggcggaagttttcaatctgtcgatactgcggttgctaaggttcgtcagatgatctcagatggggcagatataattgacatcggggcacaatcaaccagacccatggcaactaggatttcggctgaagaagagctagaaagagtagtacctgtattggaagctgtcaaggatttgatcgaggaagaag

SEQ ID NO: 324 - DNA, 200 bp, Amaranthus viridis
cttcttcctcgatcaaatccttgacagcttccaatacaggtactactctttctagctcttcttcagccgaaatcctagttgccatgggtctggttgattgtgccccgatgtcaattatatctgccccatctgagatcatctgacgaaccttagcaaccgcagtatcgacagattgaaaacttccgccatcactaaagcta

SEQ ID NO: 325 - DNA, 200 bp, Amaranthus viridis
gtcaaggatttgatcgaggaagaaggaagaatcttgtcagtggatacgttttattctaaagttgcttcggaggccgtcaagaagggggcacacattgtgaatgatgtctctagtgggaaactcgattccgagatgtttaatgttgttgcggaccttaaagttccttatatagcaatgcacatgcgaggagatccgacttc

SEQ ID NO: 326 - DNA, 200 bp, Amaranthus viridis
gaagtcggatctcctcgcatgtgcattgctatataaggaactttaaggtccgcaacaacattaaacatctcggaatcgagtttcccactagagacatcattcacaatgtgtgcccccttcttgacggcctccgaagcaactttagaataaaacgtatccactgacaagattcttccttcttcctcgatcaaatccttgac

SEQ ID NO: 327 - DNA, 200 bp, Amaranthus viridis
tgcacatgcgaggagatccgacttcaatgcaaaattctgagaacttgacgtacaatgatgtttgtaagcaagtggctacggagttgagctctagggtcatagatgcagaattatcgggaattcctgcttggaggatagttattgatcccggcattggattttctaagaatacgaatcaaaatttggaaattcttcgtggt

SEQ ID NO: 328 - DNA, 200 bp, Amaranthus viridis
accacgaagaatttccaaattttgattcgtattcttagaaaatccaatgccgggatcaataactatcctccaagcaggaattcccgataattctgcatctatgaccctagagctcaactccgtagccacttgcttacaaacatcattgtacgtcaagttctcagaattttgcattgaagtcggatctcctcgcatgtgca

SEQ ID NO: 329 - DNA, 200 bp, Ambrosia trifida
aatgaagccttaagtcaaatgaaaaaatcaggcatagaaataacaagacatgcatgtttatacgaaaccgagccagcttacgtgactgatcaacctcttttcctcaattctgccatcagaggcgttacaaagctgggcccacatgagctactatcggccctcaagaaaattgaaaaagaaatgggccgaaccaaaggtat

SEQ ID NO: 330 - DNA, 200 bp, Ambrosia trifida
atacctttggttcggcccatttctttttcaattttcttgagggccgatagtagctcatgtgggcccagctttgtaacgcctctgatggcagaattgaggaaaagaggttgatcagtcacgtaagctggctcggtttcgtataaacatgcatgtcttgttatttctatgcctgattttttcatttgacttaaggcttcatt

SEQ ID NO: 331 - DNA, 200 bp, Ambrosia trifida
aagaaatgggccgaaccaaaggtattagatacggcccgcgacccattgacttagatatactgttttatggtaaacacagagttaactcggaaattctcactgttccacatgaaagaatcttcgagaggccatttgttatggctccgttagttgacttattggggtcggaaattgacaatgatacggttctatgctggcat

SEQ ID NO: 332 - DNA, 200 bp, Ambrosia trifida
atgccagcatagaaccgtatcattgtcaatttccgaccccaataagtcaactaacggagccataacaaatggcctctcgaagattctttcatgtggaacagtgagaatttccgagttaactctgtgtttaccataaaacagtatatctaagtcaatgggtcgcgggccgtatctaatacctttggttcggcccatttctt

SEQ ID NO: 333 - DNA, 200 bp, Ambrosia trifida
caatgatacggttctatgctggcattctttctcaaaaagaggactttttgaatcttgggaaaaattaggtggcgaatctttgataggcaaagatgggttaagaagagttttaccagtcaataatcggttatgggattggtcaaagaaaacctctgtcatgggaattttgaacttgactcccgatagttttagcgacggag

SEQ ID NO: 334 - DNA, 200 bp, Ambrosia trifida
ctccgtcgctaaaactatcgggagtcaagttcaaaattcccatgacagaggttttctttgaccaatcccataaccgattattgactggtaaaactcttcttaacccatctttgcctatcaaagattcgccacctaatttttcccaagattcaaaaagtcctctttttgagaaagaatgccagcatagaaccgtatcattg

SEQ ID NO: 335 - DNA, 200 bp, Ambrosia trifida
actcccgatagttttagcgacggaggaaagtttgacactgtggggtccgctatatctcgtgttagggccatgatatctgaaggggcagacataattgatctgggagctcaatcgacacgtccaatggcaaccaagatttcagtccaagaagaactagataggttaatccctgttctcgaaaagattcttgaattacccga

SEQ ID NO: 336 - DNA, 200 bp, Ambrosia trifida
tcgggtaattcaagaatcttttcgagaacagggattaacctatctagttcttcttggactgaaatcttggttgccattggacgtgtcgattgagctcccagatcaattatgtctgccccttcagatatcatggccctaacacgagatatagcggaccccacagtgtcaaactttcctccgtcgctaaaactatcgggagt

SEQ ID NO: 337 - DNA, 200 bp, Ambrosia trifida
tcgaaaagattcttgaattacccgaaattgaaggaaaactgctctctatcgacacgttttactcagaagttgctttagaagcgattaagaaaggggctcatataatcaatgatgtatcgggtggaaatctagattctgatatgtttcgggtagttgctaatcttgatgttccatatattgctatgcacatgagaggggac

SEQ ID NO: 338 - DNA, 200 bp, Ambrosia trifida
gtcccctctcatgtgcatagcaatatatggaacatcaagattagcaactacccgaaacatatcagaatctagatttccacccgatacatcattgattatatgagcccctttcttaatcgcttctaaagcaacttctgagtaaaacgtgtcgatagagagcagttttccttcaatttcgggtaattcaagaatcttttcga

SEQ ID NO: 339 - DNA, 200 bp, Ambrosia trifida
tattgctatgcacatgagaggggacccatccacaatgcaaaacagtgagaatttgaagtatgatgatgtttgtaaagaagttggtgatgagttgtatgagcgtgtaagagctgcagagttatgcggtgtgcctgcatggaggatcattcttgacccagggatcgggttttcaaagaaaaccgaagataatttggatattt

SEQ ID NO: 340 - DNA, 200 bp, Ambrosia trifida
aaatatccaaattatcttcggttttctttgaaaacccgatccctgggtcaagaatgatcctccatgcaggcacaccgcataactctgcagctcttacacgctcatacaactcatcaccaacttctttacaaacatcatcatacttcaaattctcactgttttgcattgtggatgggtcccctctcatgtgcatagcaata

SEQ ID NO: 341 - DNA, 200 bp, Ambrosia trifida
aaaaccgaagataatttggatattttgatgggattaaagagaattaggagtgagattgcacggaagagcttaggggtgtcacgtgcacctttgttaatcggtccttcaagaaagagatttttgggggagatttgtggtcgggcttctgctgtcgagagagatccagcgactgttgctgctgttacatgtgcggttttggg

SEQ ID NO: 342 - DNA, 200 bp, Ambrosia trifida
cccaaaaccgcacatgtaacagcagcaacagtcgctggatctctctcgacagcagaagcccgaccacaaatctcccccaaaaatctctttcttgaaggaccgattaacaaaggtgcacgtgacacccctaagctcttccgtgcaatctcactcctaattctctttaatcccatcaaaatatccaaattatcttcggtttt

SEQ ID NO: 343 - DNA, 200 bp, Ambrosia trifida
acctcatcacctccacaaatcaccaccacccatctccaccctccaccaccacccacttcattctccccacccaccgtatctcatcaccatcatacctggtggattgcagtttgactctgtggggtccgctatatctcgtgttaggaccatgatatctgaaggggcggacataattgatctgggagctcaatcgacacgtc

SEQ ID NO: 344 - DNA, 200 bp, Ambrosia trifida
gacgtgtcgattgagctcccagatcaattatgtccgccccttcagatatcatggtcctaacacgagatatagcggaccccacagagtcaaactgcaatccaccaggtatgatggtgatgagatacggtgggtggggagaatgaagtgggtggtggtggagggtggagatgggtggtggtgatttgtggaggtgatgaggt

SEQ ID NO: 345 - DNA, 200 bp, Ambrosia trifida
gacccatccacaatgcaaaacagtgagaatttgaagtataatgatgtttgtaaagaagttggtgatgagttgtatgagcgtgtaagagctgcagagttatgcggtgtgcctgcatggaggatcattcttgacccagggatcgggttttcaaagaaaaccgaagataatttggatattttgatgggattaaagagaattag

SEQ ID NO: 346 - DNA, 200 bp, Ambrosia trifida
ctaattctctttaatcccatcaaaatatccaaattatcttcggttttctttgaaaacccgatccctgggtcaagaatgatcctccatgcaggcacaccgcataactctgcagctcttacacgctcatacaactcatcaccaacttctttacaaacatcattatacttcaaattctcactgttttgcattgtggatgggtc

SEQ ID NO: 347 - DNA, 200 bp, Ambrosia trifida; identical to SEQ ID NO: 345

SEQ ID NO: 348 - DNA, 200 bp, Ambrosia trifida; identical to SEQ ID NO: 346

SEQ ID NO: 349 - DNA, 200 bp, Ambrosia trifida
ttttgatgggattaaagagaattaggagtgagattgcccggaagagcttaggggtgtcacgtgcacctttgttaatcggtccttcaagaaagagatttttgggagagatttgtggtcgggcttctgctggtgagagagatccaacaactgttgctgctgttacatgtgcggttttgggtggtgctaatgttgttcgcgtt

SEQ ID NO: 350 - DNA, 200 bp, Ambrosia trifida
aacgcgaacaacattagcaccacccaaaaccgcacatgtaacagcagcaacagttgttggatctctctcaccagcagaagcccgaccacaaatctctcccaaaaatctctttcttgaaggaccgattaacaaaggtgcacgtgacacccctaagctcttccgggcaatctcactcctaattctctttaatcccatcaaaa

SEQ ID NO: 351 - DNA, 200 bp, Ambrosia trifida
gatttgtggtcgggcttctgctggtgagagagatccaacaactgttgctgctgttacatgtgcggttttgggtggtgctaatgttgttcgcgttcataatgttggagatgatgctgatgctgtaaagctttgtgattcaatgttgaaccgggttggaagaacgtaacagctttttgaaatttgattgtgggttttggtcc

SEQ ID NO: 352 - DNA, 200 bp, Ambrosia trifida
ggaccaaaacccacaatcaaatttcaaaaagctgttacgttcttccaacccggttcaacattgaatcacaaagctttacagcatcagcatcatctccaacattatgaacgcgaacaacattagcaccacccaaaaccgcacatgtaacagcagcaacagttgttggatctctctcaccagcagaagcccgaccacaaatc

SEQ ID NO: 353 - DNA, 200 bp, Ambrosia trifida
gccttaagtcaaatgaagaaatcaggcatagagataacaagacatgcatgtttatacaaaaccgagccagcttatgtaactgatcaacctctttttcttaattctgccatcagaggcgttacaaagctgggcccacatgaactactatcggccctcaagaaaatcgaaaaagaattgggccgaaccaaaggtattagata

SEQ ID NO: 354 - DNA, 200 bp, Ambrosia trifida
tatctaatacctttggttcggcccaattctttttcgattttcttgagggccgatagtagttcatgtgggcccagctttgtaacgcctctgatggcagaattaagaaaaagaggttgatcagttacataagctggctcggttttgtataaacatgcatgtcttgttatctctatgcctgatttcttcatttgacttaaggc

SEQ ID NO: 355 - DNA, 200 bp, Ambrosia trifida
tgggccgaaccaaaggtattagatacggcccgcgacccattgacttagatatactgttttatggtaaacacagaattaactcggaaattctcactgttccacatgaaagaatcttcgagaggccatttgttatggctccgttagttgacttattggggtcggatattgacaatgatacggttctatgctggcattctttc

SEQ ID NO: 356 - DNA, 200 bp, Ambrosia trifida
gaaagaatgccagcatagaaccgtatcattgtcaatatccgaccccaataagtcaactaacggagccataacaaatggcctctcgaagattctttcatgtggaacagtgagaatttccgagttaattctgtgtttaccataaaacagtatatctaagtcaatgggtcgcgggccgtatctaatacctttggttcggccca

SEQ ID NO: 357 - DNA, 101 bp, Ambrosia trifida; identical to SEQ ID NO: 29

SEQ ID NO: 358 - DNA, 101 bp, Ambrosia trifida
ggacgtgtcgattgagctcccagatcaattatgtccgccccttcagatatcatggtcctaacacgagatatagcggaccccacagagtcaaactgcaatcc

SEQ ID NO: 359 - DNA, 200 bp, Ambrosia trifida
tcgttctttcatttctcttcggatgcttcgatacaagtccattctcaagaacaagaagtagtaattgctttaggtagcaatgtgggtgataggcttaataactttaatgaagccttaagtcaaatgaaaaaatcaggcatagaaataacaagacatgcatgtttatacgaaaccgagccagcttacgtgactgatcaacc

SEQ ID NO: 360 - DNA, 200 bp, Ambrosia trifida
ggttgatcagtcacgtaagctggctcggtttcgtataaacatgcatgtcttgttatttctatgcctgattttttcatttgacttaaggcttcattaaagttattaagcctatcacccacattgctacctaaagcaattactacttcttgttcttgagaatggacttgtatcgaagcatccgaagagaaatgaaagaacga

SEQ ID NO: 361 - DNA, 200 bp, Ambrosia trifida
agccagcttacgtgactgatcaacctcttttcctcaattctgccatcagaggcgttacaaagctgggcccacatgagctactatcggccctcaagaaaattgaaaaagaaatgggccgaaccaaaggtattagatacggcccgcgacccattgacttagatatactgttttatggtaaacacagagttaactcggaaatt

SEQ ID NO: 362 - DNA, 200 bp, Ambrosia trifida
aatttccgagttaactctgtgtttaccataaaacagtatatctaagtcaatgggtcgcgggccgtatctaatacctttggttcggcccatttctttttcaattttcttgagggccgatagtagctcatgtgggcccagctttgtaacgcctctgatggcagaattgaggaaaagaggttgatcagtcacgtaagctggct

SEQ ID NO: 363 - DNA, 200 bp, Ambrosia trifida
taaacacagagttaactcggaaattctcactgttccacatgaaagaatcttcgagaggccatttgttatggctccgttagttgacttattggggtcggaaattgacaatgatacggttctatgctggcattctttctcaaaaagaggactttttgaatcttgggaaaaattaggtggcgaatctttgataggcaaagatg

SEQ ID NO: 364 - DNA, 200 bp, Ambrosia trifida
catctttgcctatcaaagattcgccacctaatttttcccaagattcaaaaagtcctctttttgagaaagaatgccagcatagaaccgtatcattgtcaatttccgaccccaataagtcaactaacggagccataacaaatggcctctcgaagattctttcatgtggaacagtgagaatttccgagttaactctgtgttta

SEQ ID NO: 365 - DNA, 200 bp, Ambrosia trifida
ggcgaatctttgataggcaaagatgggttaagaagagttttaccagtcaataatcggttatgggattggtcaaagaaaacctctgtcatgggaattttgaacttgactcccgatagttttagcgacggaggaaagtttgacactgtggggtccgctatatctcgtgttagggccatgatatctgaaggggcagacataat

SEQ ID NO: 366 - DNA, 200 bp, Ambrosia trifida
attatgtctgccccttcagatatcatggccctaacacgagatatagcggaccccacagtgtcaaactttcctccgtcgctaaaactatcgggagtcaagttcaaaattcccatgacagaggttttctttgaccaatcccataaccgattattgactggtaaaactcttcttaacccatctttgcctatcaaagattcgcc

SEQ ID NO: 367 - DNA, 200 bp, Ambrosia trifida
tgatatctgaaggggcagacataattgatctgggagctcaatcgacacgtccaatggcaaccaagatttcagtccaagaagaactagataggttaatccctgttctcgaaaagattcttgaattacccgaaattgaaggaaaactgctctctatcgacacgttttactcagaagttgctttagaagcgattaagaaaggg

SEQ ID NO: 368 - DNA, 200 bp, Ambrosia trifida
ccctttcttaatcgcttctaaagcaacttctgagtaaaacgtgtcgatagagagcagttttccttcaatttcgggtaattcaagaatcttttcgagaacagggattaacctatctagttcttcttggactgaaatcttggttgccattggacgtgtcgattgagctcccagatcaattatgtctgccccttcagatatca

SEQ ID NO: 369 - DNA, 200 bp, Ambrosia trifida
tgctttagaagcgattaagaaaggggctcatataatcaatgatgtatcgggtggaaatctagattctgatatgtttcgggtagttgctaatcttgatgttccatatattgctatgcacatgagaggggacccatccacaatgcaaaacagtgagaatttgaagtatgatgatgtttgtaaagaagttggtgatgagttgt

SEQ ID NO: 370 - DNA, 200 bp, Ambrosia trifida
acaactcatcaccaacttctttacaaacatcatcatacttcaaattctcactgttttgcattgtggatgggtcccctctcatgtgcatagcaatatatggaacatcaagattagcaactacccgaaacatatcagaatctagatttccacccgatacatcattgattatatgagcccctttcttaatcgcttctaaagca

SEQ ID NO: 371 - DNA, 200 bp, Ambrosia trifida
tgtaaagaagttggtgatgagttgtatgagcgtgtaagagctgcagagttatgcggtgtgcctgcatggaggatcattcttgacccagggatcgggttttcaaagaaaaccgaagataatttggatattttgatgggattaaagagaattaggagtgagattgcacggaagagcttaggggtgtcacgtgcacctttgtt

SEQ ID NO: 372 - DNA, 200 bp, Ambrosia trifida
aacaaaggtgcacgtgacacccctaagctcttccgtgcaatctcactcctaattctctttaatcccatcaaaatatccaaattatcttcggttttctttgaaaacccgatccctgggtcaagaatgatcctccatgcaggcacaccgcataactctgcagctcttacacgctcatacaactcatcaccaacttctttaca

SEQ ID NO: 373 - DNA, 200 bp, Ambrosia trifida
ttcgtgtatctcatgatctctgtataatgttaaacttattatagttcaatataagtcactgtgtgatctaatttcgtatacaattactttatttgtgttgattctgatcctaaatattgctaatagtttgtgcttaactatatatcccgttaaacatcacatttactttatccctagaagatttgggcaagtttttacaa

SEQ ID NO: 374 - DNA, 200 bp, Ambrosia trifida
ttgtaaaaacttgcccaaatcttctagggataaagtaaatgtgatgtttaacgggatatatagttaagcacaaactattagcaatatttaggatcagaatcaacacaaataaagtaattgtatacgaaattagatcacacagtgacttatattgaactataataagtttaacattatacagagatcatgagatacacgaa

SEQ ID NO: 375 - DNA, 200 bp, Ambrosia trifida
agaagatttgggcaagtttttacaaatttatggcgacgttattgtaattttttaaatcattttcgtgcagcatctttttcgttctttcatttctcttcggatgcttcgatacaagtccattctcaagaacaagaagtagtaattgctttaggtagcaatgtgggtgataggcttaataactttaatgaagccttaagtca

SEQ ID NO: 376 - DNA, 200 bp, Ambrosia trifida
tgacttaaggcttcattaaagttattaagcctatcacccacattgctacctaaagcaattactacttcttgttcttgagaatggacttgtatcgaagcatccgaagagaaatgaaagaacgaaaaagatgctgcacgaaaatgatttaaaaaattacaataacgtcgccataaatttgtaaaaacttgcccaaatcttct

SEQ ID NO: 377 - DNA, 200 bp, Conyza canadensis
gaaataacaaggcatgcttgtctatacgaaactgaaccagcttatgtgacggaccagcctctttttctcaactctgccgttagagccactacaaagcttggccctcatgagctactgtccattctcaagaaaattgaaaaggaaatgggtcgaaccaaagggcttaggtacggcccacgacccattgacctagatatatt

SEQ ID NO: 378 - DNA, 200 bp, Conyza canadensis
aatatatctaggtcaatgggtcgtgggccgtacctaagccctttggttcgacccatttccttttcaattttcttgagaatggacagtagctcatgagggccaagctttgtagtggctctaacggcagagttgagaaaaagaggctggtccgtcacataagctggttcagtttcgtatagacaagcatgccttgttatttc

SEQ ID NO: 379 - DNA, 200 bp, Conyza canadensis
cacgacccattgacctagatatattgttctatggtaaatgcaaagttaactctgatattctaactgttcctcatgaaagaatctttgagaggccattcgttatggctccattagttgacttgttaggatcagatgtagacaatgatacggttctatgctggcattctttttcaaaaaatgggctttttggatcttgggaa

SEQ ID NO: 380 - DNA, 200 bp, Conyza canadensis
ttcccaagatccaaaaagcccattttttgaaaaagaatgccagcatagaaccgtatcattgtctacatctgatcctaacaagtcaactaatggagccataacgaatggcctctcaaagattctttcatgaggaacagttagaatatcagagttaactttgcatttaccatagaacaatatatctaggtcaatgggtcgtg

SEQ ID NO: 381 - DNA, 200 bp, Conyza canadensis
aaatgggctttttggatcttgggaaacattaggtggtgaatcttccataggaaaagatggtttaagaagggttttacctgtcaatgatcgtttatgggattggtcaaagaaaacttccgtcatgggtattttgaatataactcctgatagttttagtgatggagggaagtttgattccatggggtccgctttatctcgtg

SEQ ID NO: 382 - DNA, 200 bp, Conyza canadensis
cacgagataaagcggaccccatggaatcaaacttccctccatcactaaaactatcaggagttatattcaaaatacccatgacggaagttttctttgaccaatcccataaacgatcattgacaggtaaaacccttcttaaaccatcttttcctatggaagattcaccacctaatgtttcccaagatccaaaaagcccattt

SEQ ID NO: 383 - DNA, 200 bp, Conyza canadensis
tccatggggtccgctttatctcgtgttcagaccatgatatctgaaggggttgacataattgatctaggagctcaatccacacgcccaatggcgaccaagatctcagtcgaagaggaactagataggctaatacccgttcttgaaaagattcttgaattacctgaaattgaaggaaagttgttgtctgtggacacatttta

SEQ ID NO: 384 - DNA, 200 bp, Conyza canadensis
taaaatgtgtccacagacaacaactttccttcaatttcaggtaattcaagaatcttttcaagaacgggtattagcctatctagttcctcttcgactgagatcttggtcgccattgggcgtgtggattgagctcctagatcaattatgtcaaccccttcagatatcatggtctgaacacgagataaagcggaccccatgga

SEQ ID NO: 385 - DNA, 200 bp, Conyza canadensis
agttgttgtctgtggacacattttactcggaagttgcttcagaggcaatcaagaaaggggctcatatggtcaatgatgtatcgggtggaatgttagattctgatatgcttcatgttgtggctgatctaaatgttccatatatcactatgcacatgagaggggacccatccacaatgcaaaacagtgagaatttgaagtat

SEQ ID NO: 386 - DNA, 200 bp, Conyza canadensis
atacttcaaattctcactgttttgcattgtggatgggtcccctctcatgtgcatagtgatatatggaacatttagatcagccacaacatgaagcatatcagaatctaacattccacccgatacatcattgaccatatgagcccctttcttgattgcctctgaagcaacttccgagtaaaatgtgtccacagacaacaact

SEQ ID NO: 387 - DNA, 200 bp, Conyza canadensis
gcaaaacagtgagaatttgaagtatgatgatgttgtcaaagaagttggggaggaattgtatgaacgtgtaaggaatgcagagttatgtggtgttcccgcatggaggatggttcttgatccagggatcgggttttcaaagaaaaccgaagataatttggagatattgatgggactaaagaggtttagaagtgagattggac

SEQ ID NO: 388 - DNA, 200 bp, Conyza canadensis
gtccaatctcacttctaaacctctttagtcccatcaatatctccaaattatcttcggttttctttgaaaacccgatccctggatcaagaaccatcctccatgcgggaacaccacataactctgcattccttacacgttcatacaattcctccccaacttctttgacaacatcatcatacttcaaattctcactgttttgc

SEQ ID NO: 389 - DNA, 200 bp, Conyza canadensis
aagaggtttagaagtgagattggacaaaagagcttaggggtgtctcgtgcacctttgttaatcggaccttcaagaaaaaggtttttgggtgagatttgtggtcggccttctgctgttgagagagatccagggactgtagctgctgttaccagtgcgattttgggtggtgccaatatcgttagggttcataacattggaca

SEQ ID NO: 390 - DNA, 200 bp, Conyza canadensis
tgtccaatgttatgaaccctaacgatattggcaccacccaaaatcgcactggtaacagcagctacagtccctggatctctctcaacagcagaaggccgaccacaaatctcacccaaaaacctttttcttgaaggtccgattaacaaaggtgcacgagacacccctaagctcttttgtccaatctcacttctaaacctctt

SEQ ID NO: 391 - DNA, 200 bp, Conyza canadensis
tcgtcctcggatacttcaatacaagtgcattctccagaacaagaagtagtaattgctttaggtagcaatgtgggtaatagacttaataactttaatgaagccttatcccagatgaagaaatcggggatagaaataacaaggcatgcttgtctatacgaaactgaaccagcttatgtgacggaccagcctctttttctcaa

SEQ ID NO: 392 - DNA, 200 bp, Conyza canadensis
ttgagaaaaagaggctggtccgtcacataagctggttcagtttcgtatagacaagcatgccttgttatttctatccccgatttcttcatctgggataaggcttcattaaagttattaagtctattacccacattgctacctaaagcaattactacttcttgttctggagaatgcacttgtattgaagtatccgaggacga

SEQ ID NO: 393 - DNA, 200 bp, Conyza canadensis
tgacggaccagcctctttttctcaactctgccgttagagccactacaaagcttggccctcatgagctactgtccattctcaagaaaattgaaaaggaaatgggtcgaaccaaagggcttaggtacggcccacgacccattgacctagatatattgttctatggtaaatgcaaagttaactctgatattctaactgttcct

SEQ ID NO: 394 - DNA, 200 bp, Conyza canadensis
aggaacagttagaatatcagagttaactttgcatttaccatagaacaatatatctaggtcaatgggtcgtgggccgtacctaagccctttggttcgacccatttccttttcaattttcttgagaatggacagtagctcatgagggccaagctttgtagtggctctaacggcagagttgagaaaaagaggctggtccgtca

SEQ ID NO: 395 - DNA, 200 bp, Conyza canadensis
taactctgatattctaactgttcctcatgaaagaatctttgagaggccattcgttatggctccattagttgacttgttaggatcagatgtagacaatgatacggttctatgctggcattctttttcaaaaaatgggctttttggatcttgggaaacattaggtggtgaatcttccataggaaaagatggtttaagaaggg

SEQ ID NO: 396 - DNA, 200 bp, Conyza canadensis
cccttcttaaaccatcttttcctatggaagattcaccacctaatgtttcccaagatccaaaaagcccattttttgaaaaagaatgccagcatagaaccgtatcattgtctacatctgatcctaacaagtcaactaatggagccataacgaatggcctctcaaagattctttcatgaggaacagttagaatatcagagtta

SEQ ID NO: 397 - DNA, 200 bp, Conyza canadensis
ataggaaaagatggtttaagaagggttttacctgtcaatgatcgtttatgggattggtcaaagaaaacttccgtcatgggtattttgaatataactcctgatagttttagtgatggagggaagtttgattccatggggtccgctttatctcgtgttcagaccatgatatctgaaggggttgacataattgatctaggagc

SEQ ID NO: 398 - DNA, 200 bp, Conyza canadensis
gctcctagatcaattatgtcaaccccttcagatatcatggtctgaacacgagataaagcggaccccatggaatcaaacttccctccatcactaaaactatcaggagttatattcaaaatacccatgacggaagttttctttgaccaatcccataaacgatcattgacaggtaaaacccttcttaaaccatcttttcctat

SEQ ID NO: 399 - DNA, 200 bp, Conyza canadensis
gggttgacataattgatctaggagctcaatccacacgcccaatggcgaccaagatctcagtcgaagaggaactagataggctaatacccgttcttgaaaagattcttgaattacctgaaattgaaggaaagttgttgtctgtggacacattttactcggaagttgcttcagaggcaatcaagaaaggggctcatatggtc

SEQ ID NO: 400 - DNA, 200 bp, Conyza canadensis
gaccatatgagcccctttcttgattgcctctgaagcaacttccgagtaaaatgtgtccacagacaacaactttccttcaatttcaggtaattcaagaatcttttcaagaacgggtattagcctatctagttcctcttcgactgagatcttggtcgccattgggcgtgtggattgagctcctagatcaattatgtcaaccc

SEQ ID NO: 401 - DNA, 200 bp, Conyza canadensis
aatcaagaaaggggctcatatggtcaatgatgtatcgggtggaatgttagattctgatatgcttcatgttgtggctgatctaaatgttccatatatcactatgcacatgagaggggacccatccacaatgcaaaacagtgagaatttgaagtatgatgatgttgtcaaagaagttggggaggaattgtatgaacgtgtaa

SEQ ID NO: 402 - DNA, 200 bp, Conyza canadensis
ttacacgttcatacaattcctccccaacttctttgacaacatcatcatacttcaaattctcactgttttgcattgtggatgggtcccctctcatgtgcatagtgatatatggaacatttagatcagccacaacatgaagcatatcagaatctaacattccacccgatacatcattgaccatatgagcccctttcttgatt

SEQ ID NO: 403 - DNA, 200 bp, Conyza canadensis
ggggaggaattgtatgaacgtgtaaggaatgcagagttatgtggtgttcccgcatggaggatggttcttgatccagggatcgggttttcaaagaaaaccgaagataatttggagatattgatgggactaaagaggtttagaagtgagattggacaaaagagcttaggggtgtctcgtgcacctttgttaatcggaccttc

SEQ ID NO: 404 - DNA, 200 bp, Conyza canadensis
gaaggtccgattaacaaaggtgcacgagacacccctaagctcttttgtccaatctcacttctaaacctctttagtcccatcaatatctccaaattatcttcggttttctttgaaaacccgatccctggatcaagaaccatcctccatgcgggaacaccacataactctgcattccttacacgttcatacaattcctcccc

SEQ ID NO: 405 - DNA, 200 bp, Conyza canadensis
tttcttcttctagttggagctcttttacgtggtgtcttcttttccgagtcatcttcttccccatcagatggattttctgcagccttttttctacgaactgctgtaatagcaaatgtactacgcaatctaacatctgggaattgtacaaagtttattgaagatagtgaaggcagattcgactgccacctacacatataaga

SEQ ID NO: 406 - DNA, 200 bp, Conyza canadensis
tcttatatgtgtaggtggcagtcgaatctgccttcactatcttcaataaactttgtacaattcccagatgttagattgcgtagtacatttgctattacagcagttcgtagaaaaaaggctgcagaaaatccatctgatggggaagaagatgactcggaaaagaagacaccacgtaaaagagctccaactagaagaagaaa

SEQ ID NO: 407 - DNA, 200 bp, Conyza canadensis
tcgactgccacctacacatataagaaaccattaacttctatacttaactcacaatataaaacatcatttgcatgttcgtcaaaaacaataccatttcttatctttgctcaatggatatgaaatctgccaatacaagattattcatttgacctcaatctagcatctgatgtctattcaaggtatcgtttataatgaagatt

SEQ ID NO: 408 - DNA, 200 bp, Conyza canadensis
aatcttcattataaacgataccttgaatagacatcagatgctagattgaggtcaaatgaataatcttgtattggcagatttcatatccattgagcaaagataagaaatggtattgtttttgacgaacatgcaaatgatgttttatattgtgagttaagtatagaagttaatggtttcttatatgtgtaggtggcagtcga

SEQ ID NO: 409 - DNA, 200 bp, Conyza canadensis
caaggtatcgtttataatgaagattattgtacttcaaaatactttcttgtgtagtggtatcaaattttgagttaaagttactccatttgacctcaaaagccaaataatgagcaacaaacaacacaatttcacctcccttataccctgcgactatattttatcttcaaatcaatccatttccatgtttatcctctccatac

SEQ ID NO: 410 - DNA, 200 bp, Conyza canadensis
gtatggagaggataaacatggaaatggattgatttgaagataaaatatagtcgcagggtataagggaggtgaaattgtgttgtttgttgctcattatttggcttttgaggtcaaatggagtaactttaactcaaaatttgataccactacacaagaaagtattttgaagtacaataatcttcattataaacgataccttg

SEQ ID NO: 411 - DNA, 200 bp, Conyza canadensis
atttccatgtttatcctctccatacagctacataataagttttagggaaaaaacaaaactaaactaaactaagtttacagaattcccatttcttatcctaaaatttacaagcaattcaacatatattctttatttacaactataaatagcatatgggtatgttggtatatagatatagatatagatatacatatgtagat

SEQ ID NO: 412 - DNA, 200 bp, Conyza canadensis
atctacatatgtatatctatatctatatctatataccaacatacccatatgctatttatagttgtaaataaagaatatatgttgaattgcttgtaaattttaggataagaaatgggaattctgtaaacttagtttagtttagttttgttttttccctaaaacttattatgtagctgtatggagaggataaacatggaaat

SEQ ID NO: 413 - DNA, 200 bp, Conyza canadensis
tagatatagatatacatatgtagatgtgtattatatgtgtgtaaatggggagaaaaaaggaaacatatattacctgtgtaaagaacaaaattgagtaaaagaaggcaaagaagccatttctaaccactaaaatcttgggctttcaagaatcccacttcaaagctaccatttcacataccaaacccccctttctatgggtt

SEQ ID NO: 414 - DNA, 200 bp, Conyza canadensis
aacccatagaaaggggggtttggtatgtgaaatggtagctttgaagtgggattcttgaaagcccaagattttagtggttagaaatggcttctttgccttcttttactcaattttgttctttacacaggtaatatatgtttccttttttctccccatttacacacatataatacacatctacatatgtatatctatatcta

SEQ ID NO: 415 - DNA, 200 bp, Conyza canadensis
taccaaacccccctttctatgggtttaaagatgagggctttacatttatctcttttacaatccaaattttccaaaaccgaaataatgtacgacattcggtgcacaaggtacaaacagaaaaaactacttttgtattttagatctcaagaaaggagtggcctttttggtaataacatactttattcaattgccacttacct

SEQ ID NO: 416 - DNA, 200 bp, Conyza canadensis
aggtaagtggcaattgaataaagtatgttattaccaaaaaggccactcctttcttgagatctaaaatacaaaagtagttttttctgtttgtaccttgtgcaccgaatgtcgtacattatttcggttttggaaaatttggattgtaaaagagataaatgtaaagccctcatctttaaacccatagaaaggggggtttggta

SEQ ID NO: 417 - DNA, 200 bp, Conyza canadensis
tactttattcaattgccacttaccttcaagaaattaacaaaattacccacaattaaaaaagggtttcccagtttcactcacacttattcaagattgatgcaaaataaacccataaataagaaggttaaaatagtgttaaagccttaaaggtgcttgctttttgtgttttttgtgatgcctataaaaagattcttattgtt

SEQ ID NO: 418 - DNA, 200 bp, Conyza canadensis
aacaataagaatctttttataggcatcacaaaaaacacaaaaagcaagcacctttaaggctttaacactattttaaccttcttatttatgggtttattttgcatcaatcttgaataagtgtgagtgaaactgggaaacccttttttaattgtgggtaattttgttaatttcttgaaggtaagtggcaattgaataaagta

SEQ ID NO: 419 - DNA, 200 bp, Conyza canadensis
tgcctataaaaagattcttattgttttcaaatttttttatgtcaagttttcgtttttaacagtttatatagttatataatttgcagaaagccataaagctctgaacttgaatgttttatagcagctctttgcagtaatatcttgtagaggtaaacctgttaacccatatttattaatttgtacataaaattgttgaattg

SEQ ID NO: 420 - DNA, 200 bp, Conyza canadensis
caattcaacaattttatgtacaaattaataaatatgggttaacaggtttacctctacaagatattactgcaaagagctgctataaaacattcaagttcagagctttatggctttctgcaaattatataactatataaactgttaaaaacgaaaacttgacataaaaaaatttgaaaacaataagaatctttttataggca

SEQ ID NO: 421 - DNA, 200 bp, Conyza canadensis
atttgtacataaaattgttgaattgaatagagctaccactgggacttgtgtagtcaaactgtagagcatccctgtaatgctggaagctttatgacctaaaaggatttgtagacttgttatctgtggattggtataaagattttcaagttgttattttgaaactttgaatagtatattatgctcattactgaatgatacaa

SEQ ID NO: 422 - DNA, 200 bp, Conyza canadensis
ttgtatcattcagtaatgagcataatatactattcaaagtttcaaaataacaacttgaaaatctttataccaatccacagataacaagtctacaaatccttttaggtcataaagcttccagcattacagggatgctctacagtttgactacacaagtcccagtggtagctctattcaattcaacaattttatgtacaaat

SEQ ID NO: 423 - DNA, 200 bp, Conyza canadensis
ttatgctcattactgaatgatacaatagggagtcgcgaatgttagaattgataagaattaggatgaagtttgtgtttgaacgttccttctgtttataatcttcatgtcacttccatagtaccataaccctagggcaacacgagcctttagattatgaatgggcacacccaattgtttagaccttgtataaatgaagtgaa

SEQ ID NO: 424 - DNA, 200 bp, Conyza canadensis
ttcacttcatttatacaaggtctaaacaattgggtgtgcccattcataatctaaaggctcgtgttgccctagggttatggtactatggaagtgacatgaagattataaacagaaggaacgttcaaacacaaacttcatcctaattcttatcaattctaacattcgcgactccctattgtatcattcagtaatgagcataa

SEQ ID NO: 425 - DNA, 200 bp, Conyza canadensis
ttagaccttgtataaatgaagtgaatatcattgttataagcattctggtggtggttttgcagatttacctttctctatctaatttgaaggtttgttgattaaggtgatacaaatgagtgttttcaagcagctaatagcttcaagattagtacgcactcatgtctgtggatcttatagaggtacacttcctcgaacattca

SEQ ID NO: 426 - DNA, 200 bp, Conyza canadensis
tgaatgttcgaggaagtgtacctctataagatccacagacatgagtgcgtactaatcttgaagctattagctgcttgaaaacactcatttgtatcaccttaatcaacaaaccttcaaattagatagagaaaggtaaatctgcaaaaccaccaccagaatgcttataacaatgatattcacttcatttatacaaggtctaa

SEQ ID NO: 427 - DNA, 200 bp, Conyza canadensis
agaggtacacttcctcgaacattcatatagcgatgttattgttaagttttgaatctgtagcaacattatatataattttttttgcattgttttcctgcagcatccttttcactctttcattcgtcctcggatacttcaatacaagtgcattctccagaacaagaagtagtaattgctttaggtagcaatgtgggtaatag

SEQ ID NO: 428 - DNA, 200 bp, Conyza canadensis
ctattacccacattgctacctaaagcaattactacttcttgttctggagaatgcacttgtattgaagtatccgaggacgaatgaaagagtgaaaaggatgctgcaggaaaacaatgcaaaaaaaattatatataatgttgctacagattcaaaacttaacaataacatcgctatatgaatgttcgaggaagtgtacctct

SEQ ID NO: 429 - DNA, 200 bp, Conyza canadensis
ctttaggtagcaatgtgggtaatagacttaataactttaatgaagccttatcccagatgaagaaatcggggatagaaataacaaggcatgcttgtctatacgaaactgaaccagcttatgtgacggaccagcctctttttctcaactctgccgttagagccactacaaagcttggccctcatgagctactgtccattctc

SEQ ID NO: 430 - DNA, 200 bp, Conyza canadensis
gagaatggacagtagctcatgagggccaagctttgtagtggctctaacggcagagttgagaaaaagaggctggtccgtcacataagctggttcagtttcgtatagacaagcatgccttgttatttctatccccgatttcttcatctgggataaggcttcattaaagttattaagtctattacccacattgctacctaaag

SEQ ID NO: 431 - DNA, 200 bp, Conyza canadensis
ccctcatgagctactgtccattctcaagaaaattgaaaaggaaatgggtcgaaccaaagggcttaggtacggcccacgacccattgacctagatatattgttctatggtaaatgcaaagttaactctgatattctaactgttcctcatgaaagaatctttgagaggccattcgttatggctccattagttgacttgttag

SEQ ID NO: 432 - DNA, 200 bp, Conyza canadensis
ctaacaagtcaactaatggagccataacgaatggcctctcaaagattctttcatgaggaacagttagaatatcagagttaactttgcatttaccatagaacaatatatctaggtcaatgggtcgtgggccgtacctaagccctttggttcgacccatttccttttcaattttcttgagaatggacagtagctcatgaggg

SEQ ID NO: 433 - DNA, 200 bp, Conyza canadensis
atggctccattagttgacttgttaggatcagatgtagacaatgatacggttctatgctggcattctttttcaaaaaatgggctttttggatcttgggaaacattaggtggtgaatcttccataggaaaagatggtttaagaagggttttacctgtcaatgatcgtttatgggattggtcaaagaaaacttccgtcatggg

SEQ ID NO: 434 - DNA, 200 bp, Conyza canadensis
cccatgacggaagttttctttgaccaatcccataaacgatcattgacaggtaaaacccttcttaaaccatcttttcctatggaagattcaccacctaatgtttcccaagatccaaaaagcccattttttgaaaaagaatgccagcatagaaccgtatcattgtctacatctgatcctaacaagtcaactaatggagccat

SEQ ID NO: 435 - DNA, 200 bp, Conyza canadensis
ggtcaaagaaaacttccgtcatgggtattttgaatataactcctgatagttttagtgatggagggaagtttgattccatggggtccgctttatctcgtgttcagaccatgatatctgaaggggttgacataattgatctaggagctcaatccacacgcccaatggcgaccaagatctcagtcgaagaggaactagatagg

SEQ ID NO: 436 - DNA, 200 bp, Conyza canadensis
cctatctagttcctcttcgactgagatcttggtcgccattgggcgtgtggattgagctcctagatcaattatgtcaaccccttcagatatcatggtctgaacacgagataaagcggaccccatggaatcaaacttccctccatcactaaaactatcaggagttatattcaaaatacccatgacggaagttttctttgacc

SEQ ID NO: 437 - DNA, 200 bp, Conyza canadensis
ctcagtcgaagaggaactagataggctaatacccgttcttgaaaagattcttgaattacctgaaattgaaggaaagttgttgtctgtggacacattttactcggaagttgcttcagaggcaatcaagaaaggggctcatatggtcaatgatgtatcgggtggaatgttagattctgatatgcttcatgttgtggctgatc

SEQ ID NO: 438 - DNA, 200 bp, Conyza canadensis
gatcagccacaacatgaagcatatcagaatctaacattccacccgatacatcattgaccatatgagcccctttcttgattgcctctgaagcaacttccgagtaaaatgtgtccacagacaacaactttccttcaatttcaggtaattcaagaatcttttcaagaacgggtattagcctatctagttcctcttcgactgag

SEQ ID NO: 439 - DNA, 200 bp, Conyza canadensis
gatatgcttcatgttgtggctgatctaaatgttccatatatcactatgcacatgagaggggacccatccacaatgcaaaacagtgagaatttgaagtatgatgatgttgtcaaagaagttggggaggaattgtatgaacgtgtaaggaatgcagagttatgtggtgttcccgcatggaggatggttcttgatccagggat

SEQ ID NO: 440 - DNA, 200 bp, Conyza canadensis
atccctggatcaagaaccatcctccatgcgggaacaccacataactctgcattccttacacgttcatacaattcctccccaacttctttgacaacatcatcatacttcaaattctcactgttttgcattgtggatgggtcccctctcatgtgcatagtgatatatggaacatttagatcagccacaacatgaagcatatc

SEQ ID NO: 441 - DNA, 200 bp, Conyza canadensis
ggaggatggttcttgatccagggatcgggttttcaaagaaaaccgaagataatttggagatattgatgggactaaagaggtttagaagtgagattggacaaaagagcttaggggtgtctcgtgcacctttgttaatcggaccttcaagaaaaaggtttttgggtgagatttgtggtcggccttctgctgttgagagagat

SEQ ID NO: 442 - DNA, 200 bp, Conyza canadensis
atctctctcaacagcagaaggccgaccacaaatctcacccaaaaacctttttcttgaaggtccgattaacaaaggtgcacgagacacccctaagctcttttgtccaatctcacttctaaacctctttagtcccatcaatatctccaaattatcttcggttttctttgaaaacccgatccctggatcaagaaccatcctcc

SEQ ID NO: 443 - DNA, 200 bp, Conyza canadensis
tcggccttctgctgttgagagagatccagggactgtagctgctgttaccagtgcgattttgggtggtgccaatatcgttagggttcataacattggacataatgtggatgctgttaagctttgtgattcaatgttggatcgagctggtagatcttagtagcttaaagttatttgacatcttgttttttattcaattttct

SEQ ID NO: 444 - DNA, 200 bp, Conyza canadensis
agaaaattgaataaaaaacaagatgtcaaataactttaagctactaagatctaccagctcgatccaacattgaatcacaaagcttaacagcatccacattatgtccaatgttatgaaccctaacgatattggcaccacccaaaatcgcactggtaacagcagctacagtccctggatctctctcaacagcagaaggccga

SEQ ID NO: 445 - DNA, 200 bp, Conyza canadensis
catcttgttttttattcaattttctggacattactttttgtatttctgtctggcaaatgattttacagggatatcctagaggctaaattgtatattacattcttgattttctcacaaagttttactttttcagtttttcttgttcagttttgtttttgaaaatttgctagatcagattaaatcgttgctttatgatcttc

SEQ ID NO: 446 - DNA, 200 bp, Conyza canadensis
gaagatcataaagcaacgatttaatctgatctagcaaattttcaaaaacaaaactgaacaagaaaaactgaaaaagtaaaactttgtgagaaaatcaagaatgtaatatacaatttagcctctaggatatccctgtaaaatcatttgccagacagaaatacaaaaagtaatgtccagaaaattgaataaaaaacaagatg

SEQ ID NO: 447 - DNA, 200 bp, Conyza canadensis
attaaatcgttgctttatgatcttcgtgtcttttttgggacgctgcgagtttcatttagtggtattgtaaactccttttaaagtttgtacacacttggtatttatgcttctagttttcgtctatgtctgtcctatattgtgtatagtatgtttaaagttcataaaaagaggataaacgaatagcacgtaaatcttataat

SEQ ID NO: 448 - DNA, 200 bp, Conyza canadensis
attataagatttacgtgctattcgtttatcctctttttatgaactttaaacatactatacacaatataggacagacatagacgaaaactagaagcataaataccaagtgtgtacaaactttaaaaggagtttacaataccactaaatgaaactcgcagcgtcccaaaaaagacacgaagatcataaagcaacgatttaat

SEQ ID NO: 449 - DNA, 200 bp, Conyza canadensis
acgaatagcacgtaaatcttataattcgattccacagagtgcaagttatgctttcctaaatgaaagcaagagtctagacagtttggtcaacgttaacatgagactattgtagaatacaacaccacaagaaaccaaaactgtcttgtgcatttctctcttttccaggtttaaaactcaataccaagacttgatatcaacta

SEQ ID NO: 450 - DNA, 200 bp, Conyza canadensis
tagttgatatcaagtcttggtattgagttttaaacctggaaaagagagaaatgcacaagacagttttggtttcttgtggtgttgtattctacaatagtctcatgttaacgttgaccaaactgtctagactcttgctttcatttaggaaagcataacttgcactctgtggaatcgaattataagatttacgtgctattcgt

SEQ ID NO: 451 - DNA, 200 bp, Conyza canadensis
caataccaagacttgatatcaactattattctgcttcaatgattcagcttcagctatagccataggatcatctttggtgtcaggttcatccttctcaccatctcgttcctctgccagtttttttctgtgcaattcctctgcaacttgctttgatgcttggtcttctttttgaaggcgttgcatctcttcttcttgttgct

SEQ ID NO: 452 - DNA, 200 bp, Conyza canadensis
agcaacaagaagaagagatgcaacgccttcaaaaagaagaccaagcatcaaagcaagttgcagaggaattgcacagaaaaaaactggcagaggaacgagatggtgagaaggatgaacctgacaccaaagatgatcctatggctatagctgaagctgaatcattgaagcagaataatagttgatatcaagtcttggtattg

SEQ ID NO: 453 - DNA, 200 bp, Conyza canadensis
cgttgcatctcttcttcttgttgctgtcgttcaaaccatgtatccgcatcggcccaaactgtaatttagagccaaaacaacatgattagaatgcagattccagaagccaaattcaaactgtaagaaaaaaaatgtagtgatgatttaactcaaaacaactcacacagctccaaaatggatgggggttcaaattagaggat

SEQ ID NO: 454 - DNA, 200 bp, Conyza canadensis
atcctctaatttgaacccccatccattttggagctgtgtgagttgttttgagttaaatcatcactacattttttttcttacagtttgaatttggcttctggaatctgcattctaatcatgttgttttggctctaaattacagtttgggccgatgcggatacatggtttgaacgacagcaacaagaagaagagatgcaacg

SEQ ID NO: 455 - DNA, 200 bp, Conyza canadensis
tggatgggggttcaaattagaggatagcctaatagatagaagcttctaattatggggaaaaaaaaacgatcatggcaatgtattacacaatctaaagcaaccactatgaataataaagataaatctttccgagagtccacatgccttgatatttcataagataggctttttcagaaatcagaatcttcttggcaaatata

SEQ ID NO: 456 - DNA, 200 bp, Conyza canadensis
tatatttgccaagaagattctgatttctgaaaaagcctatcttatgaaatatcaaggcatgtggactctcggaaagatttatctttattattcatagtggttgctttagattgtgtaatacattgccatgatcgtttttttttccccataattagaagcttctatctattaggctatcctctaatttgaacccccatcca

SEQ ID NO: 457 - DNA, 200 bp, Conyza canadensis
aatcagaatcttcttggcaaatataacactaaaatcagtctaatgcatgttattaagtaaatctttatttgtccatagtataatgccatagattatcgtcaattatccacatcgtcattaaatatcaagtaaccacattatgaattgacttgtttaatcaaagttcatcttacttcttcatagatgaaaaagagctatgc

SEQ ID NO: 458 - DNA, 200 bp, Conyza canadensis
gcatagctctttttcatctatgaagaagtaagatgaactttgattaaacaagtcaattcataatgtggttacttgatatttaatgacgatgtggataattgacgataatctatggcattatactatggacaaataaagatttacttaataacatgcattagactgattttagtgttatatttgccaagaagattctgatt

SEQ ID NO: 459 - DNA, 200 bp, Conyza canadensis
cttcatagatgaaaaagagctatgcaaatacagaacacatcagttgttctaacatataattactgctagctgtaggaagaaaccaagatgaaacttatctaggattcaaattcatggggatctgttatttagatgaatttagagaaataagaaaataagttgagtgaagtcagtttcgaggttccaaatttgtctttatt

SEQ ID NO: 460 - DNA, 200 bp, Conyza canadensis
aataaagacaaatttggaacctcgaaactgacttcactcaacttattttcttatttctctaaattcatctaaataacagatccccatgaatttgaatcctagataagtttcatcttggtttcttcctacagctagcagtaattatatgttagaacaactgatgtgttctgtatttgcatagctctttttcatctatgaag

SEQ ID NO: 461 - DNA, 200 bp, Conyza canadensis
tcgaggttccaaatttgtctttattagttgagctaacaatattaacctctgaagcatgtatagtgtacttgtatgaaccacttaaggatataaatgaagccaaatcagtttcattcgctaattgagcaaatgcacgttctgtttgttagtattgaaatttctttacaggatttctttcaactaaaagcatcacatgtccg

SEQ ID NO: 462 - DNA, 200 bp, Conyza canadensis
cggacatgtgatgcttttagttgaaagaaatcctgtaaagaaatttcaatactaacaaacagaacgtgcatttgctcaattagcgaatgaaactgatttggcttcatttatatccttaagtggttcatacaagtacactatacatgcttcagaggttaatattgttagctcaactaataaagacaaatttggaacctcga

SEQ ID NO: 463 - DNA, 200 bp, Conyza canadensis
ttcaactaaaagcatcacatgtccgactctcggtttgatatttgtgacaagtctaattggttcatcagatgtacttatggctaaatttcgaaacttgaaacaataagagcttagcactcttaaccataaagggttgctgttaatcttacttgttcttgaaagcaattccacagtgaagcatttgttaattgggaagtttt

SEQ ID NO: 464 - DNA, 200 bp, Conyza canadensis
aaaacttcccaattaacaaatgcttcactgtggaattgctttcaagaacaagtaagattaacagcaaccctttatggttaagagtgctaagctcttattgtttcaagtttcgaaatttagccataagtacatctgatgaaccaattagacttgtcacaaatatcaaaccgagagtcggacatgtgatgcttttagttgaa

SEQ ID NO: 465 - DNA, 200 bp, Conyza canadensis
ttttcttcttctagttggagctcttttacgtggtgtcttcttttccgagtcatcttcttccccatcagatggattttctgcagccttttttctacgaactgctgtaatagcaaatgtactacgcaatctaacatctgggaattgtacaaagtttattgaagatagtgaaggcagattcgactgccacctacacatataag

SEQ ID NO: 466 - DNA, 200 bp, Conyza canadensis
cttatatgtgtaggtggcagtcgaatctgccttcactatcttcaataaactttgtacaattcccagatgttagattgcgtagtacatttgctattacagcagttcgtagaaaaaaggctgcagaaaatccatctgatggggaagaagatgactcggaaaagaagacaccacgtaaaagagctccaactagaagaagaaaa

SEQ ID NO: 467 - DNA, 200 bp, Conyza canadensis
ttcgactgccacctacacatataagaaaccattaacttctatacttaactcacaatataaaacatcatttgcatgttcgtcaaaaacaataccatttcttatctttgctcaatggatatgaaatctgccaatacaagattattcatttgacctcaatctagcatctgatgtctattcaaggtatcgtttataatgaagat

SEQ ID NO: 468 - DNA, 200 bp, Conyza canadensis
atcttcattataaacgataccttgaatagacatcagatgctagattgaggtcaaatgaataatcttgtattggcagatttcatatccattgagcaaagataagaaatggtattgtttttgacgaacatgcaaatgatgttttatattgtgagttaagtatagaagttaatggtttcttatatgtgtaggtggcagtcgaa

SEQ ID NO: 469 - DNA, 200 bp, Conyza canadensis
tcaaggtatcgtttataatgaagattattgtacttcaaaatactttcttgtgtagtggtatcaaattttgagttaaagttactccatttgacctcaaaagccaaataatgagcaacaaacaacacaatttcacctcccttataccctgcgactatattttatcttcaaatcaatccatttccatgtttatcctctccata

SEQ ID NO: 470 - DNA, 200 bp, Conyza canadensis
tatggagaggataaacatggaaatggattgatttgaagataaaatatagtcgcagggtataagggaggtgaaattgtgttgtttgttgctcattatttggcttttgaggtcaaatggagtaactttaactcaaaatttgataccactacacaagaaagtattttgaagtacaataatcttcattataaacgataccttga

SEQ ID NO: 471 - DNA, 200 bp, Conyza canadensis
catttccatgtttatcctctccatacagctacataataagttttagggaaaaaacaaaactaaactaaactaagtttacagaattcccatttcttatcctaaaatttacaagcaattcaacatatattctttatttacaactataaatagcatatgggtatgttggtatatagatatagatatagatatacatatgtaga

SEQ ID NO: 472 - DNA, 200 bp, Conyza canadensis
tctacatatgtatatctatatctatatctatataccaacatacccatatgctatttatagttgtaaataaagaatatatgttgaattgcttgtaaattttaggataagaaatgggaattctgtaaacttagtttagtttagttttgttttttccctaaaacttattatgtagctgtatggagaggataaacatggaaatg

SEQ ID NO: 473 - DNA, 200 bp, Conyza canadensis
atagatatagatatacatatgtagatgtgtattatatgtgtgtaaatggggagaaaaaaggaaacatatattacctgtgtaaagaacaaaattgagtaaaagaaggcaaagaagccatttctaaccactaaaatcttgggctttcaagaatcccacttcaaagctaccatttcacataccaaacccccctttctatgggt

SEQ ID NO: 474 - DNA, 200 bp, Conyza canadensis
acccatagaaaggggggtttggtatgtgaaatggtagctttgaagtgggattcttgaaagcccaagattttagtggttagaaatggcttctttgccttcttttactcaattttgttctttacacaggtaatatatgtttccttttttctccccatttacacacatataatacacatctacatatgtatatctatatctat

SEQ ID NO: 475 - DNA, 200 bp, Conyza canadensis
ataccaaacccccctttctatgggtttaaagatgagggctttacatttatctcttttacaatccaaattttccaaaaccgaaataatgtacgacattcggtgcacaaggtacaaacagaaaaaactacttttgtattttagatctcaagaaaggagtggcctttttggtaataacatactttattcaattgccacttacc

SEQ ID NO: 476 - DNA, 200 bp, Conyza canadensis
ggtaagtggcaattgaataaagtatgttattaccaaaaaggccactcctttcttgagatctaaaatacaaaagtagttttttctgtttgtaccttgtgcaccgaatgtcgtacattatttcggttttggaaaatttggattgtaaaagagataaatgtaaagccctcatctttaaacccatagaaaggggggtttggtat

SEQ ID NO: 477 - DNA, 200 bp, Conyza canadensis
atactttattcaattgccacttaccttcaagaaattaacaaaattacccacaattaaaaaagggtttcccagtttcactcacacttattcaagattgatgcaaaataaacccataaataagaaggttaaaatagtgttaaagccttaaaggtgcttgctttttgtgttttttgtgatgcctataaaaagattcttattgt

SEQ ID NO: 478 - DNA, 200 bp, Conyza canadensis
acaataagaatctttttataggcatcacaaaaaacacaaaaagcaagcacctttaaggctttaacactattttaaccttcttatttatgggtttattttgcatcaatcttgaataagtgtgagtgaaactgggaaacccttttttaattgtgggtaattttgttaatttcttgaaggtaagtggcaattgaataaagtat

SEQ ID NO: 479 - DNA, 200 bp, Conyza canadensis
atgcctataaaaagattcttattgttttcaaatttttttatgtcaagttttcgtttttaacagtttatatagttatataatttgcagaaagccataaagctctgaacttgaatgttttatagcagctctttgcagtaatatcttgtagaggtaaacctgttaacccatatttattaatttgtacataaaattgttgaatt

SEQ ID NO: 480 - DNA, 200 bp, Conyza canadensis
aattcaacaattttatgtacaaattaataaatatgggttaacaggtttacctctacaagatattactgcaaagagctgctataaaacattcaagttcagagctttatggctttctgcaaattatataactatataaactgttaaaaacgaaaacttgacataaaaaaatttgaaaacaataagaatctttttataggcat

SEQ ID NO: 481 - DNA, 200 bp, Conyza canadensis
aatttgtacataaaattgttgaattgaatagagctaccactgggacttgtgtagtcaaactgtagagcatccctgtaatgctggaagctttatgacctaaaaggatttgtagacttgttatctgtggattggtataaagattttcaagttgttattttgaaactttgaatagtatattatgctcattactgaatgataca

SEQ ID NO: 482 - DNA, 200 bp, Conyza canadensis
tgtatcattcagtaatgagcataatatactattcaaagtttcaaaataacaacttgaaaatctttataccaatccacagataacaagtctacaaatccttttaggtcataaagcttccagcattacagggatgctctacagtttgactacacaagtcccagtggtagctctattcaattcaacaattttatgtacaaatt

SEQ ID NO: 483 - DNA, 200 bp, Conyza canadensis
attatgctcattactgaatgatacaatagggagtcgcgaatgttagaattgataagaattaggatgaagtttgtgtttgaacgttccttctgtttataatcttcatgtcacttccatagtaccataaccctagggcaacacgagcctttagattatgaatgggcacacccaattgtttagaccttgtataaatgaagtga

SEQ ID NO: 484 - DNA, 200 bp, Conyza canadensis
tcacttcatttatacaaggtctaaacaattgggtgtgcccattcataatctaaaggctcgtgttgccctagggttatggtactatggaagtgacatgaagattataaacagaaggaacgttcaaacacaaacttcatcctaattcttatcaattctaacattcgcgactccctattgtatcattcagtaatgagcataat

SEQ ID NO: 485 - DNA, 200 bp, Conyza canadensis
tttagaccttgtataaatgaagtgaatatcattgttataagcattctggtggtggttttgcagatttacctttctctatctaatttgaaggtttgttgattaaggtgatacaaatgagtgttttcaagcagctaatagcttcaagattagtacgcactcatgtctgtggatcttatagaggtacacttcctcgaacatta

SEQ ID NO: 486 - DNA, 200 bp, Conyza canadensis
taatgttcgaggaagtgtacctctataagatccacagacatgagtgcgtactaatcttgaagctattagctgcttgaaaacactcatttgtatcaccttaatcaacaaaccttcaaattagatagagaaaggtaaatctgcaaaaccaccaccagaatgcttataacaatgatattcacttcatttatacaaggtctaaa

SEQ ID NO: 487 - DNA, 200 bp, Conyza canadensis
tagaggtacacttcctcgaacattatatagcgatgttattgttaagttttgaatctgtagcaacattatatataattttttttgcattgttttcctgcagcatccttttcactctttcattcgtcctcggatacttcaatacaagtgcattctccagaacaagaagtagtaattgctttaggtagcaatgtgggtaatag

SEQ ID NO: 488 - DNA, 200 bp, Conyza canadensis
ctattacccacattgctacctaaagcaattactacttcttgttctggagaatgcacttgtattgaagtatccgaggacgaatgaaagagtgaaaaggatgctgcaggaaaacaatgcaaaaaaaattatatataatgttgctacagattcaaaacttaacaataacatcgctatataatgttcgaggaagtgtacctcta

SEQ ID NO: 489 - DNA, 200 bp, Conyza canadensis; identical to SEQ ID NO: 429

SEQ ID NO: 490 - DNA, 200 bp, Conyza canadensis; identical to SEQ ID NO: 430

SEQ ID NO: 491 - DNA, 200 bp, Conyza canadensis; identical to SEQ ID NO: 431

SEQ ID NO: 492 - DNA, 200 bp, Conyza canadensis; identical to SEQ ID NO: 432

SEQ ID NO: 493 - DNA, 200 bp, Conyza canadensis; identical to SEQ ID NO: 433

SEQ ID NO: 494 - DNA, 200 bp, Conyza canadensis; identical to SEQ ID NO: 434

SEQ ID NO: 495 - DNA, 200 bp, Conyza canadensis; identical to SEQ ID NO: 435

SEQ ID NO: 496 - DNA, 200 bp, Conyza canadensis; identical to SEQ ID NO: 436

SEQ ID NO: 497 - DNA, 200 bp, Conyza canadensis; identical to SEQ ID NO: 437

SEQ ID NO: 498 - DNA, 200 bp, Conyza canadensis; identical to SEQ ID NO: 438

SEQ ID NO: 499 - DNA, 200 bp, Conyza canadensis; identical to SEQ ID NO: 439

SEQ ID NO: 500 - DNA, 200 bp, Conyza canadensis; identical to SEQ ID NO: 440

SEQ ID NO: 501 - DNA, 200 bp, Conyza canadensis; identical to SEQ ID NO: 441

SEQ ID NO: 502 - DNA, 200 bp, Conyza canadensis; identical to SEQ ID NO: 442

SEQ ID NO: 503 - DNA, 200 bp, Conyza canadensis; identical to SEQ ID NO: 443

SEQ ID NO: 504 - DNA, 200 bp, Conyza canadensis; identical to SEQ ID NO: 444

SEQ ID NO: 505 - DNA, 200 bp, Conyza canadensis; identical to SEQ ID NO: 445

SEQ ID NO: 506 - DNA, 200 bp, Conyza canadensis; identical to SEQ ID NO: 446

SEQ ID NO: 507 - DNA, 200 bp, Conyza canadensis; identical to SEQ ID NO: 447

SEQ ID NO: 508 - DNA, 200 bp, Conyza canadensis; identical to SEQ ID NO: 448

SEQ ID NO: 509 - DNA, 200 bp, Conyza canadensis; identical to SEQ ID NO: 449

SEQ ID NO: 510 - DNA, 200 bp, Conyza canadensis; identical to SEQ ID NO: 450

SEQ ID NO: 511 - DNA, 200 bp, Conyza canadensis; identical to SEQ ID NO: 451

SEQ ID NO: 512 - DNA, 200 bp, Conyza canadensis; identical to SEQ ID NO: 452

SEQ ID NO: 513 - DNA, 200 bp, Conyza canadensis; identical to SEQ ID NO: 453

SEQ ID NO: 514 - DNA, 200 bp, Conyza canadensis; identical to SEQ ID NO: 454

SEQ ID NO: 515 - DNA, 200 bp, Conyza canadensis; identical to SEQ ID NO: 455

SEQ ID NO: 516 - DNA, 200 bp, Conyza canadensis; identical to SEQ ID NO: 456

SEQ ID NO: 517 - DNA, 200 bp, Conyza canadensis; identical to SEQ ID NO: 457

SEQ ID NO: 518 - DNA, 200 bp, Conyza canadensis; identical to SEQ ID NO: 458

SEQ ID NO: 519 - DNA, 200 bp, Conyza canadensis; identical to SEQ ID NO: 459

SEQ ID NO: 520 - DNA, 200 bp, Conyza canadensis; identical to SEQ ID NO: 460

SEQ ID NO: 521 - DNA, 200 bp, Conyza canadensis; identical to SEQ ID NO: 461

SEQ ID NO: 522 - DNA, 200 bp, Conyza canadensis; identical to SEQ ID NO: 462

SEQ ID NO: 523 - DNA, 200 bp, Conyza canadensis; identical to SEQ ID NO: 463

SEQ ID NO: 524 - DNA, 200 bp, Conyza canadensis; identical to SEQ ID NO: 464

SEQ ID NO: 525 - DNA, 200 bp, Digitaria sanguinalis
aaatcgtaaaatttataactaacatgttccttcatgaatccaaaagccaagtaaacaggtccatgagtgcctttgtgagattgcttcgaatagaatatacctacggctcttaatttcagattctcttaggtagtgtttaggtagcgtttggaagtagatatttaggcttggaattaggaattggcattgagatctacgaa

SEQ ID NO: 526 - DNA, 200 bp, Digitaria sanguinalis
ttcgtagatctcaatgccaattcctaattccaagcctaaatatctacttccaaacgctacctaaacactacctaagagaatctgaaattaagagccgtaggtatattctattcgaagcaatctcacaaaggcactcatggacctgtttacttggcttttggattcatgaaggaacatgttagttataaattttacgattt

SEQ ID NO: 527 - DNA, 200 bp, Digitaria sanguinalis
aggaattggcattgagatctacgaatacctgccgtttggatgtctaagaatttggaattggaattgcagcccaataccaaccggcattccttggcctctcatctgcctgccctcagtatagatagagcttatctccttcatcctcgcggagtggtggcaagcggcggcgagatccggaccacatggggtgtgggagcgcg

SEQ ID NO: 528 - DNA, 200 bp, Digitaria sanguinalis
cgcgctcccacaccccatgtggtccggatctcgccgccgcttgccaccactccgcgaggatgaaggagataagctctatctatactgagggcaggcagatgagaggccaaggaatgccggttggtattgggctgcaattccaattccaaattcttagacatccaaacggcaggtattcgtagatctcaatgccaattcct

SEQ ID NO: 529 - DNA, 200 bp, Digitaria sanguinalis
ggaccacatggggtgtgggagcgcggtggcagagtggtggcaagcgatggcgacggcaccgtgccgccgtgccacattggcacggcgacggacgagtttggatggtgtcagtacagaccactgcccaattctatccttttgtgcatccaaacataaaatctgaattaggatgctcttttgattctacaaggcaatgtaat

SEQ ID NO: 530 - DNA, 200 bp, Digitaria sanguinalis
attacattgccttgtagaatcaaaagagcatcctaattcagattttatgtttggatgcacaaaaggatagaattgggcagtggtctgtactgacaccatccaaactcgtccgtcgccgtgccaatgtggcacggcggcacggtgccgtcgccatcgcttgccaccactctgccaccgcgctcccacaccccatgtggtcc

SEQ ID NO: 531 - DNA, 200 bp, Digitaria sanguinalis
ttttgattctacaaggcaatgtaatgaccatccaaacaacagattctgaattggagtcaattcaatgttacgattgtattggcatcggacctaattcaatgtcatgccctctagtatctacattcaaacggagcggaaaaatctacctccgtcccaagattccggcccaagatcccgactcttaatacttttttcatctt

SEQ ID NO: 532 - DNA, 200 bp, Digitaria sanguinalis
aagatgaaaaaagtattaagagtcgggatcttgggccggaatcttgggacggaggtagatttttccgctccgtttgaatgtagatactagagggcatgacattgaattaggtccgatgccaatacaatcgtaacattgaattgactccaattcagaatctgttgtttggatggtcattacattgccttgtagaatcaaaa

SEQ ID NO: 533 - DNA, 200 bp, Digitaria sanguinalis
cgactcttaatacttttttcatctttcgctgaaatttcagcccaagatcccggcccgcctttttgcctccacgctgggcctggtgggacgcggccccttcacgagagtcgagaggggaaagcggaaagggaaacaaaaaaactcaacgaatcgccgccgccgccttgccctgcagcgaacaattaagggccccgtacctc

SEQ ID NO: 534 - DNA, 200 bp, Digitaria sanguinalis
gaggtacggggcccttaattgttcgctgcagggcaaggcggcggcggcgattcgttgagtttttttgtttccctttccgctttcccctctcgactctcgtgaaggggccgcgtcccaccaggcccagcgtggaggcaaaaaggcgggccgggatcttgggctgaaatttcagcgaaagatgaaaaaagtattaagagtcg

SEQ ID NO: 535 - DNA, 200 bp, Digitaria sanguinalis
cgaacaattaagggccccgtacctctctctgcccgttcttccgctgcactccgcatctgagcggacgggtggggacaccgccgacggcgccggggtggcgcctccgtctgtgctccaaccaaggcagtgggtgctggttgctgccaatcgaccggccgggcatgctctgaccatcgagaaaatcttcgggtaacacaacg

SEQ ID NO: 536 - DNA, 200 bp, Digitaria sanguinalis
cgttgtgttacccgaagattttctcgatggtcagagcatgcccggccggtcgattggcagcaaccagcacccactgccttggttggagcacagacggaggcgccaccccggcgccgtcggcggtgtccccacccgtccgctcagatgcggagtgcagcggaagaacgggcagagagaggtacggggcccttaattgttcg

SEQ ID NO: 537 - DNA, 200 bp, Digitaria sanguinalis
gagaaaatcttcgggtaacacaacgttgttcatttcgtttgtatcgtggcatagttgatttccctaacttttaggatgcgtttcgattcattgtatctgtataccaaaacagagttcgataacttagattgaacggtaggcttcagcatctaaggcagcaggtgcaggtctattctatcactctgaaaattagttttaac

SEQ ID NO: 538 - DNA, 200 bp, Digitaria sanguinalis
gttaaaactaattttcagagtgatagaatagacctgcacctgctgccttagatgctgaagcctaccgttcaatctaagttatcgaactctgttttggtatacagatacaatgaatcgaaacgcatcctaaaagttagggaaatcaactatgccacgatacaaacgaaatgaacaacgttgtgttacccgaagattttctc

SEQ ID NO: 539 - DNA, 200 bp, Digitaria sanguinalis
tatcactctgaaaattagttttaactgtctgatgataggaggattagctaatgttgttatatgaggaggtttcttgctgactagagagctatttgaccacctgatagattgcctcgttagcaacttagcattatgacgacgatggcagcgagccatttcatgtagaaaacgaacatgagctgagaagcttaatcgtataa

SEQ ID NO: 540 - DNA, 200 bp, Digitaria sanguinalis
ttatacgattaagcttctcagctcatgttcgttttctacatgaaatggctcgctgccatcgtcgtcataatgctaagttgctaacgaggcaatctatcaggtggtcaaatagctctctagtcagcaagaaacctcctcatataacaacattagctaatcctcctatcatcagacagttaaaactaattttcagagtgata

SEQ ID NO: 541 - DNA, 200 bp, Digitaria sanguinalis
tgagctgagaagcttaatcgtataagcttcaattggcttcctggctggggaggtttgctagagaacttctgtattttgttcattcatttaatctgatgtatgttggcagctctcctgtcaggtacagaaatttagacggccctgaaaccaagcagggcaacctcaccaataggggggcagtcgcaccacaaaggttgctt

SEQ ID NO: 542 - DNA, 200 bp, Digitaria sanguinalis
aagcaacctttgtggtgcgactgcccccctattggtgaggttgccctgcttggtttcagggccgtctaaatttctgtacctgacaggagagctgccaacatacatcagattaaatgaatgaacaaaatacagaagttctctagcaaacctccccagccaggaagccaattgaagcttatacgattaagcttctcagctca

SEQ ID NO: 543 - DNA, 200 bp, Digitaria sanguinalis
ggcagtcgcaccacaaaggttgcttgcctccagtggggcatctcctctccttacattgagaggtttatttgacgtctaagatatctgaactcctgcctccagttagagaaaaggcccttgttcatacctcaaatttgtctttgctcttctattgaccaggtgtgatggagtgatgcttgcgtgaaatatcctacaaggct

SEQ ID NO: 544 - DNA, 200 bp, Digitaria sanguinalis
agccttgtaggatatttcacgcaagcatcactccatcacacctggtcaatagaagagcaaagacaaatttgaggtatgaacaagggccttttctctaactggaggcaggagttcagatatcttagacgtcaaataaacctctcaatgtaaggagaggagatgccccactggaggcaagcaacctttgtggtgcgactgcc

SEQ ID NO: 545 - DNA, 200 bp, Digitaria sanguinalis
cttgcgtgaaatatcctacaaggctacaatttatttgaagcatttgctttcttaattatcatatgttcaacgatgctcctccatgctaaggagtcccttaagaagatgtatcctgttgctatgaactactttggagggctcctcccatcgcattcgttttcaggtactgttcaaatcttttcttttcaaatcccctatca

SEQ ID NO: 546 - DNA, 200 bp, Digitaria sanguinalis
tgataggggatttgaaaagaaaagatttgaacagtacctgaaaacgaatgcgatgggaggagccctccaaagtagttcatagcaacaggatacatcttcttaagggactccttagcatggaggagcatcgttgaacatatgataattaagaaagcaaatgcttcaaataaattgtagccttgtaggatatttcacgcaag

SEQ ID NO: 547 - DNA, 200 bp, Digitaria sanguinalis
tcttttcttttcaaatcccctatcatgcgatcaagagcagctctaagcttctaactcacccctttgcatttctaaacagtgtctgatcttgttcatccgctgaggttcccaaacagagctcctagacatgctattccattcagggctcgttcgtttacgcgatgttcgcttgagggatgttcaactgaccaagagattgt

SEQ ID NO: 548 - DNA, 200 bp, Digitaria sanguinalis
acaatctcttggtcagttgaacatccctcaagcgaacatcgcgtaaacgaacgagccctgaatggaatagcatgtctaggagctctgtttgggaacctcagcggatgaacaagatcagacactgtttagaaatgcaaaggggtgagttagaagcttagagctgctcttgatcgcatgataggggatttgaaaagaaaaga

SEQ ID NO: 549 - DNA, 200 bp, Digitaria sanguinalis
gatgttcaactgaccaagagattgtgattgctatgggaagcaacgtgggtgatggagtcagtacatttgacagggcattacaaatgatgaaaagctcaggcgtgaacatcactaggcatgcctgtctatatgagactgcccctgcttatgtgaccgatcagccacggttcctgaactctgccattcgaggtacgactagg

SEQ ID NO: 550 - DNA, 200 bp, Digitaria sanguinalis
cctagtcgtacctcgaatggcagagttcaggaaccgtggctgatcggtcacataagcaggggcagtctcatatagacaggcatgcctagtgatgttcacgcctgagcttttcatcatttgtaatgccctgtcaaatgtactgactccatcacccacgttgcttcccatagcaatcacaatctcttggtcagttgaacatc

SEQ ID NO: 551 - DNA, 200 bp, Digitaria sanguinalis
ctctgccattcgaggtacgactaggctgggacctcacgagttgcttaagaaactaaaggaaattgagaaggatataggacgcactggtggaataaggtatggcccaagaccaatcgatctagatatacttctgtacggcaattcccagattgatactgaggctctaattgtgccacatgaacgcatccaggagaggccat

SEQ ID NO: 552 - DNA, 200 bp, Digitaria sanguinalis
atggcctctcctggatgcgttcatgtggcacaattagagcctcagtatcaatctgggaattgccgtacagaagtatatctagatcgattggtcttgggccataccttattccaccagtgcgtcctatatccttctcaatttcctttagtttcttaagcaactcgtgaggtcccagcctagtcgtacctcgaatggcagag

SEQ ID NO: 553 - DNA, 200 bp, Digitaria sanguinalis
catgaacgcatccaggagaggccatttgttctagcacctcttgttgaccttctaggtgcatctggcaatgatagtgtcgaaacaagctggcactctctttcaaagtgcagtggtggtttctttgaattatggaataaacttgggggtgaatctataattggaacagaaagtattaaaagggtattacctgttgggaatca

SEQ ID NO: 554 - DNA, 200 bp, Digitaria sanguinalis
tgattcccaacaggtaatacccttttaatactttctgttccaattatagattcacccccaagtttattccataattcaaagaaaccaccactgcactttgaaagagagtgccagcttgtttcgacactatcattgccagatgcacctagaaggtcaacaagaggtgctagaacaaatggcctctcctggatgcgttcatg

SEQ ID NO: 555 - DNA, 200 bp, Digitaria sanguinalis
aaagggtattacctgttgggaatcatttgtttgattggtgtgagagaaccctcgtcatgggggtccttaatctaacaccagacagctttagtgatggaggtaagtttcaacaagtggaagctgctatttctcaggctaaattattaatttcagaaggtgcagatatcatagatattggtgctcaatctaccaggcctttt

SEQ ID NO: 556 - DNA, 200 bp, Digitaria sanguinalis
aaaaggcctggtagattgagcaccaatatctatgatatctgcaccttctgaaattaataatttagcctgagaaatagcagcttccacttgttgaaacttacctccatcactaaagctgtctggtgttagattaaggacccccatgacgagggttctctcacaccaatcaaacaaatgattcccaacaggtaatacccttt

SEQ ID NO: 557 - DNA, 200 bp, Digitaria sanguinalis
tggtgctcaatctaccaggccttttgcaaagagattatctccaaacgaagaaattgaaagattggttcctgttctggatgtgattacgaaaattcctgaaatggagggaaagttgctctcagtggatacattctatgcagaagttgcatgtgaagctgtgaaaagaggagttcacatgatcaatgatgtatctggtggac

SEQ ID NO: 558 - DNA, 200 bp, Digitaria sanguinalis
gtccaccagatacatcattgatcatgtgaactcctcttttcacagcttcacatgcaacttctgcatagaatgtatccactgagagcaactttccctccatttcaggaattttcgtaatcacatccagaacaggaaccaatctttcaatttcttcgtttggagataatctctttgcaaaaggcctggtagattgagcacca

SEQ ID NO: 559 - DNA, 200 bp, Digitaria sanguinalis
atgatcaatgatgtatctggtggacagcttgaccccaaaattcttaaagttgctgctgaactcaaagttccgtatgttgcaatgcacatgaggggagatccatcaacaatgcaaagtgaacaaaatttacagtatgacgatgtctgcaaggaagttgcttcagagctatatgcacaggtgagagaagcagagttatctgg

SEQ ID NO: 560 - DNA, 200 bp, Digitaria sanguinalis
ccagataactctgcttctctcacctgtgcatatagctctgaagcaacttccttgcagacatcgtcatactgtaaattttgttcactttgcattgttgatggatctcccctcatgtgcattgcaacatacggaactttgagttcagcagcaactttaagaattttggggtcaagctgtccaccagatacatcattgatcat

SEQ ID NO: 561 - DNA, 200 bp, Digitaria sanguinalis
aggtgagagaagcagagttatctgggattccattgtggaggatagttctagatccaggcattgggttctccaagaaatccaaacataaccttgaagtaattatgggattggaatccattaggagggagatgagtaaaatgagtattggtgcttcacatgtgccaatattactgggaccctctaggaaaagatttttgggt

SEQ ID NO: 562 - DNA, 200 bp, Digitaria sanguinalis
acccaaaaatcttttcctagagggtcccagtaatattggcacatgtgaagcaccaatactcattttactcatctccctcctaatggattccaatcccataattacttcaaggttatgtttggatttcttggagaacccaatgcctggatctagaactatcctccacaatggaatcccagataactctgcttctctcacct

SEQ ID NO: 563 - DNA, 200 bp, Digitaria sanguinalis
accctctaggaaaagatttttgggtgaaatatgcaatcgtgaaaatccagttgagagagatgttgctactgttgcagctgtgacggcgggtattttgaatggtgctaacatagtaagggtacataatgcaggatacagttcagatgctgcaaagttttgtgatgcattgaataagagaagaagaatggaagactgaacca

SEQ ID NO: 564 - DNA, 200 bp, Digitaria sanguinalis
tggttcagtcttccattcttcttctcttattcaatgcatcacaaaactttgcagcatctgaactgtatcctgcattatgtacccttactatgttagcaccattcaaaatacccgccgtcacagctgcaacagtagcaacatctctctcaactggattttcacgattgcatatttcacccaaaaatcttttcctagagggt

SEQ ID NO: 565 - DNA, 200 bp, Digitaria sanguinalis
agaagaagaatggaagactgaaccagctgatcaaaaagataccgagctctgattttattcgagaaaatggtgatgcaggatagttactctgctgctcaatgggattctcatattacatcatttgtggagtattgtttttgtaataaataaaccagggatgacgtttttcctgtgtcatctcctatctagtttctagatac

SEQ ID NO: 566 - DNA, 200 bp, Digitaria sanguinalis
gtatctagaaactagataggagatgacacaggaaaaacgtcatccctggtttatttattacaaaaacaatactccacaaatgatgtaatatgagaatcccattgagcagcagagtaactatcctgcatcaccattttctcgaataaaatcagagctcggtatctttttgatcagctggttcagtcttccattcttcttct

SEQ ID NO: 567 - DNA, 200 bp, Digitaria sanguinalis
catctcctatctagtttctagatacttcaatgaagcatggttcaactcagatcacttggaaagagtagtcaaggaataaggttacagaatcagttagtaattacagattcttacctcttgtggtcctattttagatgattttccagaacattttctttccaatttaataatatcagaagggcctaatttgtgtgtatctt

SEQ ID NO: 568 - DNA, 200 bp, Digitaria sanguinalis
aagatacacacaaattaggcccttctgatattattaaattggaaagaaaatgttctggaaaatcatctaaaataggaccacaagaggtaagaatctgtaattactaactgattctgtaaccttattccttgactactctttccaagtgatctgagttgaaccatgcttcattgaagtatctagaaactagataggagatg

SEQ ID NO: 569 - DNA, 200 bp, Digitaria sanguinalis
gctcgttcgtttacgcgatgttcgcttgagggatgttcaactgaccaagagattgtgattgctatgggaagcaacgtgggtgatggagtcagtacatttgacagggcattacaaatgatgaaaagctcaggcgtgaacatcactaggcatgcctgtctatatgagactgcccctgcttatgtgaccgatcagccacggtt

SEQ ID NO: 570 - DNA, 200 bp, Digitaria sanguinalis
aaccgtggctgatcggtcacataagcaggggcagtctcatatagacaggcatgcctagtgatgttcacgcctgagcttttcatcatttgtaatgccctgtcaaatgtactgactccatcacccacgttgcttcccatagcaatcacaatctcttggtcagttgaacatccctcaagcgaacatcgcgtaaacgaacgagc

SEQ ID NO: 571 - DNA, 200 bp, Digitaria sanguinalis
cttatgtgaccgatcagccacggttcctgaactctgccattcgaggtacgactaggctgggacctcacgagttgcttaagaaactaaaggaaattgagaaggatataggacgcactggtggaataaggtatggcccaagaccaatcgatctagatatacttctgtacggcaattcccagattgatactgaggctctaatt

SEQ ID NO: 572 - DNA, 200 bp, Digitaria sanguinalis
aattagagcctcagtatcaatctgggaattgccgtacagaagtatatctagatcgattggtcttgggccataccttattccaccagtgcgtcctatatccttctcaatttcctttagtttcttaagcaactcgtgaggtcccagcctagtcgtacctcgaatggcagagttcaggaaccgtggctgatcggtcacataag

SEQ ID NO: 573 - DNA, 200 bp, Digitaria sanguinalis
ccagattgatactgaggctctaattgtgccacatgaacgcatccaggagaggccatttgttctagcacctcttgttgaccttctaggtgcatctggcaatgatagtgtcgaaacaagctggcactctctttcaaagtgcagtggtggtttctttgaattatggaataaacttgggggtgaatctataattggaacagaaa

SEQ ID NO: 574 - DNA, 200 bp, Digitaria sanguinalis
tttctgttccaattatagattcacccccaagtttattccataattcaaagaaaccaccactgcactttgaaagagagtgccagcttgtttcgacactatcattgccagatgcacctagaaggtcaacaagaggtgctagaacaaatggcctctcctggatgcgttcatgtggcacaattagagcctcagtatcaatctgg

SEQ ID NO: 575 - DNA, 200 bp, Digitaria sanguinalis
ggtgaatctataattggaacagaaagtattaaaagggtattacctgttgggaatcatttgtttgattggtgtgagagaaccctcgtcatgggggtccttaatctaacaccagacagctttagtgatggaggtaagtttcaacaagtggaagctgctatttctcaggctaaattattaatttcagaaggtgcagatatcat

SEQ ID NO: 576 - DNA, 200 bp, Digitaria sanguinalis
atgatatctgcaccttctgaaattaataatttagcctgagaaatagcagcttccacttgttgaaacttacctccatcactaaagctgtctggtgttagattaaggacccccatgacgagggttctctcacaccaatcaaacaaatgattcccaacaggtaatacccttttaatactttctgttccaattatagattcacc

SEQ ID NO: 577 - DNA, 200 bp, Digitaria sanguinalis
taatttcagaaggtgcagatatcatagatattggtgctcaatctaccaggccttttgcaaagagattatctccaaacgaagaaattgaaagattggttcctgttctggatgtgattacgaaaattcctgaaatggagggaaagttgctctcagtggatacattctatgcagaagttgcatgtgaagctgtgaaaagagga

SEQ ID NO: 578 - DNA, 200 bp, Digitaria sanguinalis
tcctcttttcacagcttcacatgcaacttctgcatagaatgtatccactgagagcaactttccctccatttcaggaattttcgtaatcacatccagaacaggaaccaatctttcaatttcttcgtttggagataatctctttgcaaaaggcctggtagattgagcaccaatatctatgatatctgcaccttctgaaatta

SEQ ID NO: 579 - DNA, 200 bp, Digitaria sanguinalis
tgcatgtgaagctgtgaaaagaggagttcacatgatcaatgatgtatctggtggacagcttgaccccaaaattcttaaagttgctgctgaactcaaagttccgtatgttgcaatgcacatgaggggagatccatcaacaatgcaaagtgaacaaaatttacagtatgacgatgtctgcaaggaagttgcttcagagctat

SEQ ID NO: 580 - DNA, 200 bp, Digitaria sanguinalis
atagctctgaagcaacttccttgcagacatcgtcatactgtaaattttgttcactttgcattgttgatggatctcccctcatgtgcattgcaacatacggaactttgagttcagcagcaactttaagaattttggggtcaagctgtccaccagatacatcattgatcatgtgaactcctcttttcacagcttcacatgca

SEQ ID NO: 581 - DNA, 200 bp, Digitaria sanguinalis
tgcaaggaagttgcttcagagctatatgcacaggtgagagaagcagagttatctgggattccattgtggaggatagttctagatccaggcattgggttctccaagaaatccaaacataaccttgaagtaattatgggattggaatccattaggagggagatgagtaaaatgagtattggtgcttcacatgtgccaatatt

SEQ ID NO: 582 - DNA, 200 bp, Digitaria sanguinalis
aatattggcacatgtgaagcaccaatactcattttactcatctccctcctaatggattccaatcccataattacttcaaggttatgtttggatttcttggagaacccaatgcctggatctagaactatcctccacaatggaatcccagataactctgcttctctcacctgtgcatatagctctgaagcaacttccttgca

SEQ ID NO: 583 - DNA, 200 bp, Digitaria sanguinalis
ttggtgcttcacatgtgccaatattactgggaccctctaggaaaagatttttgggtgaaatatgcaatcgtgaaaatccagttgagagagatgttgctactgttgcagctgtgacggcgggtattttgaatggtgctaacatagtaagggtacataatgcaggatacagttcagatgctgcaaagttttgtgatgcattg

SEQ ID NO: 584 - DNA, 200 bp, Digitaria sanguinalis
caatgcatcacaaaactttgcagcatctgaactgtatcctgcattatgtacccttactatgttagcaccattcaaaatacccgccgtcacagctgcaacagtagcaacatctctctcaactggattttcacgattgcatatttcacccaaaaatcttttcctagagggtcccagtaatattggcacatgtgaagcaccaa

SEQ ID NO: 585 - DNA, 200 bp, Euphorbia heterophylla
aaatataaatataaatataaaaataaaaataaattataataatataaaatataaaaattaatttaaaccatgttttaaaaatagtgttatgaaaaactccgcttgggcgccgtctagatagtttacttaggctgatttttaaaacaaaaccccccgcttaacccgcctaaatcggtcaattgtttatatccttatgacta

SEQ ID NO: 586 - DNA, 200 bp, Euphorbia heterophylla
tagtcataaggatataaacaattgaccgatttaggcgggttaagcggggggttttgttttaaaaatcagcctaagtaaactatctagacggcgcccaagcggagtttttcataacactatttttaaaacatggtttaaattaatttttatattttatattattataatttatttttatttttatatttatatttatattt

SEQ ID NO: 587 - DNA, 200 bp, Euphorbia heterophylla
tcaattgtttatatccttatgactacttaaaatcgcagcggtgttatggtgaattatcatgttttaatgagcttatgaaatgcaataataaaaaaataaccattaataatttgatatacatttatcgggaaaaatgaaataataaaaaataaaaaaatatcaaaaaaatctataaaatatttgtacctttttcaattagt

SEQ ID NO: 588 - DNA, 200 bp, Euphorbia heterophylla
actaattgaaaaaggtacaaatattttatagatttttttgatatttttttattttttattatttcatttttcccgataaatgtatatcaaattattaatggttatttttttattattgcatttcataagctcattaaaacatgataattcaccataacaccgctgcgattttaagtagtcataaggatataaacaattga

SEQ ID NO: 589 - DNA, 200 bp, Euphorbia heterophylla
aatatttgtacctttttcaattagtacccattttgtatattaaaacaataaaaatgatccagattaataaatagaaaaagtattaataacccgaaaatatcaaatatcaaaatataatgaaacatgaacacgaaaacacaataaagaagttggtatcacaaacacaaagacacccttaaagctcctatgtttgcggagag

SEQ ID NO: 590 - DNA, 200 bp, Euphorbia heterophylla
ctctccgcaaacataggagctttaagggtgtctttgtgtttgtgataccaacttctttattgtgttttcgtgttcatgtttcattatattttgatatttgatattttcgggttattaatactttttctatttattaatctggatcatttttattgttttaatatacaaaatgggtactaattgaaaaaggtacaaatatt

SEQ ID NO: 591 - DNA, 200 bp, Euphorbia heterophylla
ttaaagctcctatgtttgcggagagttgtaagtcgactaatttcagattgaaaaagttaaaaatggccaacttttttagctatgaggttagattagatacgagaatgcctttgtgaaaagctggtcactatttcttcattcgcgtggcaattaacccggcataatcgatagccaattcacggacaaggtcaaaggtatca

SEQ ID NO: 592 - DNA, 200 bp, Euphorbia heterophylla
tgatacctttgaccttgtccgtgaattggctatcgattatgccgggttaattgccacgcgaatgaagaaatagtgaccagcttttcacaaaggcattctcgtatctaatctaacctcatagctaaaaaagttggccatttttaactttttcaatctgaaattagtcgacttacaactctccgcaaacataggagctttaa

SEQ ID NO: 593 - DNA, 200 bp, Euphorbia heterophylla
ttcacggacaaggtcaaaggtatcagctttactcaacccttcgctttgatactaaattgctgattcttattctgattatgctttatttcgttacttcattttattgtctatgcacataagctgttcgacaaaaagcctgaatgaaactgtgagtgctggtacgcggttacaatactcctgattatgtccaatgattattt

SEQ ID NO: 594 - DNA, 200 bp, Euphorbia heterophylla
aaataatcattggacataatcaggagtattgtaaccgcgtaccagcactcacagtttcattcaggctttttgtcgaacagcttatgtgcatagacaataaaatgaagtaacgaaataaagcataatcagaataagaatcagcaatttagtatcaaagcgaagggttgagtaaagctgatacctttgaccttgtccgtgaa

SEQ ID NO: 595 - DNA, 200 bp, Euphorbia heterophylla
tcctgattatgtccaatgattatttgggtactttaataccctaatttgtgaagttcactagcgaaagggtagtttcttccccggttatttatcattttatgaatccccaagtctttgaagtacagctgattaaaatgcaagtactcataagttgtcattgtatgtttgaaatccttacgttgttggtattttacagaatt

SEQ ID NO: 596 - DNA, 200 bp, Euphorbia heterophylla
aattctgtaaaataccaacaacgtaaggatttcaaacatacaatgacaacttatgagtacttgcattttaatcagctgtacttcaaagacttggggattcataaaatgataaataaccggggaagaaactaccctttcgctagtgaacttcacaaattagggtattaaagtacccaaataatcattggacataatcagga

SEQ ID NO: 597 - DNA, 200 bp, Euphorbia heterophylla
tacgttgttggtattttacagaatttgtactgaagaagcataaattcatacctagttaggcttccttgttcctaatatgcttctcctcaagcggctggtgtccactaaacaagggttcaataatgccatcaaccgttttggaggtaatatagttatgcctatgatgctttaagtcgcttttcatcgatttgttacattac

SEQ ID NO: 598 - DNA, 200 bp, Euphorbia heterophylla
gtaatgtaacaaatcgatgaaaagcgacttaaagcatcataggcataactatattacctccaaaacggttgatggcattattgaacccttgtttagtggacaccagccgcttgaggagaagcatattaggaacaaggaagcctaactaggtatgaatttatgcttcttcagtacaaattctgtaaaataccaacaacgta

SEQ ID NO: 599 - DNA, 200 bp, Euphorbia heterophylla
gcttttcatcgatttgttacattaccaggctaagattctgcaattgaagttagaaatcgacgagtcttatcttgaaaagtatggattttgttttgtcaccctggggccagttcaagattgctttcccaggttgctgtctcggcaccacatcctaatccaaaaccctaaggtgatgggttaatgggtcccttcgcagttat

SEQ ID NO: 600 - DNA, 200 bp, Euphorbia heterophylla
ataactgcgaagggacccattaacccatcaccttagggttttggattaggatgtggtgccgagacagcaacctgggaaagcaatcttgaactggccccagggtgacaaaacaaaatccatacttttcaagataagactcgtcgatttctaacttcaattgcagaatcttagcctggtaatgtaacaaatcgatgaaaagc

SEQ ID NO: 601 - DNA, 200 bp, Euphorbia heterophylla
ggttaatgggtcccttcgcagttatatttcattcacttaactcattagcttccatgtggataatcaactccacacttgccccaaaactgtataaacgtagattttgacctttggaaatcattagctggacaaatcctaactaattagtatcaagagccaaaatcttttgaccaagaccaaatatgttgtttcattgttgc

SEQ ID NO: 602 - DNA, 200 bp, Euphorbia heterophylla
gcaacaatgaaacaacatatttggtcttggtcaaaagattttggctcttgatactaattagttaggatttgtccagctaatgatttccaaaggtcaaaatctacgtttatacagttttggggcaagtgtggagttgattatccacatggaagctaatgagttaagtgaatgaaatataactgcgaagggacccattaacc

SEQ ID NO: 603 - DNA, 200 bp, Euphorbia heterophylla
accaaatatgttgtttcattgttgcagcattgtcatcatcctttctccataccgaaccaaatagttcggtggaagttcattctcaagagcaagaagtagtaattgcattaggaagcaatgtgggagatagagttcataatttcaaccaagcattgcgattaatgaaaagttccggcattaacgtaactagacatggttgt

SEQ ID NO: 604 - DNA, 200 bp, Euphorbia heterophylla
acaaccatgtctagttacgttaatgccggaacttttcattaatcgcaatgcttggttgaaattatgaactctatctcccacattgcttcctaatgcaattactacttcttgctcttgagaatgaacttccaccgaactatttggttcggtatggagaaaggatgatgacaatgctgcaacaatgaaacaacatatttggt

SEQ ID NO: 605 - DNA, 200 bp, Euphorbia heterophylla
cattaacgtaactagacatggttgtttgtaccagacagcacctgcttatgtcactgaccaacctcactttctcaactcagcagttcgagctttcacgaaactcggaccccacgagttattaggagttttaaagcagatcgaaaaggacttgggccgtaccaaagggattaggtacggaccaaggccaatcgatttggata

SEQ ID NO: 606 - DNA, 200 bp, Euphorbia heterophylla
tatccaaatcgattggccttggtccgtacctaatccctttggtacggcccaagtccttttcgatctgctttaaaactcctaataactcgtggggtccgagtttcgtgaaagctcgaactgctgagttgagaaagtgaggttggtcagtgacataagcaggtgctgtctggtacaaacaaccatgtctagttacgttaatg

SEQ ID NO: 607 - DNA, 200 bp, Euphorbia heterophylla
ggaccaaggccaatcgatttggatatacttttctatggaaagttccggattaattccgatacacttattgttcctcatgagagaatatgggagagaccctttgtgatggccccattggtggatttactcggctcggaaatcgagaatgacacggtcgccagctggcattccttgtccggcggtctttttgaatcatggga

SEQ ID NO: 608 - DNA, 200 bp, Euphorbia heterophylla
tcccatgattcaaaaagaccgccggacaaggaatgccagctggcgaccgtgtcattctcgatttccgagccgagtaaatccaccaatggggccatcacaaagggtctctcccatattctctcatgaggaacaataagtgtatcggaattaatccggaactttccatagaaaagtatatccaaatcgattggccttggtcc

SEQ ID NO: 609 - DNA, 200 bp, Euphorbia heterophylla
ccggcggtctttttgaatcatgggaaaagttgggcggcgaaagcctgatcgggaaggacggaatgaaaagagttaccccgatcggaaaccatttatgggattggtcggaaaagacttctgtaatgggaattatcaatttaacccccgatagtttcagcgatgggggcaagttgacaactattgattctatagtttctaag

SEQ ID NO: 610 - DNA, 200 bp, Euphorbia heterophylla
cttagaaactatagaatcaatagttgtcaacttgcccccatcgctgaaactatcgggggttaaattgataattcccattacagaagtcttttccgaccaatcccataaatggtttccgatcggggtaactcttttcattccgtccttcccgatcaggctttcgccgcccaacttttcccatgattcaaaaagaccgccgg

SEQ ID NO: 611 - DNA, 200 bp, Euphorbia heterophylla
aactattgattctatagtttctaaggttcgctcgatgatttccgaaggggcggatattgtcgattttggtgctcaatcgacacgccctatggctaaaaggatatccccgcaagaggaaatggataggctaatccctgtattggaagcggttgtgaaaatacctgagatgaccggaaagctcatatcggtcgacacatttt

SEQ ID NO: 612 - DNA, 200 bp, Euphorbia heterophylla
aaaatgtgtcgaccgatatgagctttccggtcatctcaggtattttcacaaccgcttccaatacagggattagcctatccatttcctcttgcggggatatccttttagccatagggcgtgtcgattgagcaccaaaatcgacaatatccgccccttcggaaatcatcgagcgaaccttagaaactatagaatcaatagtt

SEQ ID NO: 613 - DNA, 200 bp, Euphorbia heterophylla
aagctcatatcggtcgacacattttactcggaagtcgccttagaagcggtccaaagaggagcaaatctcgtaaatgacgtatctggggggcaattagatccaaaaatgacggaaaatgtcgctaatctcgaggtaccatatatcttaatgcacatgaggggggaccccaccacaatgcagaacactcagaatctaaaata

SEQ ID NO: 614 - DNA, 200 bp, Euphorbia heterophylla
tattttagattctgagtgttctgcattgtggtggggtcccccctcatgtgcattaagatatatggtacctcgagattagcgacattttccgtcatttttggatctaattgccccccagatacgtcatttacgagatttgctcctctttggaccgcttctaaggcgacttccgagtaaaatgtgtcgaccgatatgagctt

SEQ ID NO: 615 - DNA, 200 bp, Euphorbia heterophylla
tgcagaacactcagaatctaaaatacgataatgtttgtaaagaggttgcctttgagttgttttcgagggttaaagaagccgagatgtccggaattccagcctggcgaatgattattgaccctggaattgggttttcgaagaacacgaatcagaatctggaaatcctgatgggaattccaaggatccgggccgagattggg

SEQ ID NO: 616 - DNA, 200 bp, Euphorbia heterophylla
cccaatctcggcccggatccttggaattcccatcaggatttccagattctgattcgtgttcttcgaaaacccaattccagggtcaataatcattcgccaggctggaattccggacatctcggcttctttaaccctcgaaaacaactcaaaggcaacctctttacaaacattatcgtattttagattctgagtgttctgca

SEQ ID NO: 617 - DNA, 200 bp, Euphorbia heterophylla
tccaaggatccgggccgagattgggagggaaagcgtgggtatttctcgtgggccgattcttattgggccttcgagaaagaggtttttgggcgaaatttgcgaacgccctaaagcaactgaaagagacccggcaactgttgcttctgttactactgggattctgggaggggcaaatattgttagagttcataatgttaggg

SEQ ID NO: 618 - DNA, 200 bp, Euphorbia heterophylla
ccctaacattatgaactctaacaatatttgcccctcccagaatcccagtagtaacagaagcaacagttgccgggtctctttcagttgctttagggcgttcgcaaatttcgcccaaaaacctctttctcgaaggcccaataagaatcggcccacgagaaatacccacgctttccctcccaatctcggcccggatccttgga

SEQ ID NO: 619 - DNA, 200 bp, Euphorbia heterophylla
attgttagagttcataatgttagggataatgtggatgctgtgaagctatgcgactcgatgatgaaactaaagaggtctacttaaaaagtatcgggtatagttcgatttctttctttccgaatcttttgtttgactttcagattgtctttgattggcaaaacagtttcggctgcggttactgttgttgtttgttgttgcag

SEQ ID NO: 620 - DNA, 200 bp, Euphorbia heterophylla
ctgcaacaacaaacaacaacagtaaccgcagccgaaactgttttgccaatcaaagacaatctgaaagtcaaacaaaagattcggaaagaaagaaatcgaactatacccgatactttttaagtagacctctttagtttcatcatcgagtcgcatagcttcacagcatccacattatccctaacattatgaactctaacaat

SEQ ID NO: 621 - DNA, 200 bp, Euphorbia heterophylla
ttactgttgttgtttgttgttgcagtttagtgttgttgtttctaattgtgattttaagttattgttgttagctgatttgtacttattgtttggtgaaaatataattcacggttgcggttggaggtataaattactaaaatagacatattttaaataaattataagttatattgaagggtatttaggataaataaaaatcc

SEQ ID NO: 622 - DNA, 200 bp, Euphorbia heterophylla
ggatttttatttatcctaaatacccttcaatataacttataatttatttaaaatatgtctattttagtaatttatacctccaaccgcaaccgtgaattatattttcaccaaacaataagtacaaatcagctaacaacaataacttaaaatcacaattagaaacaacaacactaaactgcaacaacaaacaacaacagtaa

SEQ ID NO: 623 - DNA, 200 bp, Euphorbia heterophylla
gggtatttaggataaataaaaatccaaaccacaaaaaaaaacccgtaaaactattgatttggtgaggtttggaaaaggagataaatcccttataaaaaaaacaacttcatatatgttttgaaacttaaccaaacacttgtatacctccttttccttttcaaacctcaaaagacctttttatagtaagctcttttgaagtc

SEQ ID NO: 624 - DNA, 200 bp, Euphorbia heterophylla
gacttcaaaagagcttactataaaaaggtcttttgaggtttgaaaaggaaaaggaggtatacaagtgtttggttaagtttcaaaacatatatgaagttgttttttttataagggatttatctccttttccaaacctcaccaaatcaatagttttacgggtttttttttgtggtttggatttttatttatcctaaataccc

SEQ ID NO: 625 - DNA, 200 bp, Euphorbia heterophylla
ttttatagtaagctcttttgaagtctatacgctattgtttccgaatcattttggacttttcatcgaaatcaacaatactgctagctagtcatatttcaagatatttgggatgtaaagatcaaggtagttgttgttgtattctgttctcatcagcaaaatttgttataactccaccaagatttaattttttccaatctgaa

SEQ ID NO: 626 - DNA, 200 bp, Euphorbia heterophylla
ttcagattggaaaaaattaaatcttggtggagttataacaaattttgctgatgagaacagaatacaacaacaactaccttgatctttacatcccaaatatcttgaaatatgactagctagcagtattgttgatttcgatgaaaagtccaaaatgattcggaaacaatagcgtatagacttcaaaagagcttactataaaa

SEQ ID NO: 627 - DNA, 200 bp, Euphorbia heterophylla
aagatttaattttttccaatctgaaaatgtcttatgttatagtaagaagtgagatgagtttatgtctttgtgaaaagattttgcccaattaatgcaaaaattttgtgacaagacaagaggggtgaaagagacaagcaacaagagaaagcagtgaaaggggcaaatgttaggaaactgttagctagaaaagggtagagggt

SEQ ID NO: 628 - DNA, 200 bp, Euphorbia heterophylla
accctctacccttttctagctaacagtttcctaacatttgcccctttcactgctttctcttgttgcttgtctctttcacccctcttgtcttgtcacaaaatttttgcattaattgggcaaaatcttttcacaaagacataaactcatctcacttcttactataacataagacattttcagattggaaaaaattaaatctt

SEQ ID NO: 629 - DNA, 200 bp, Euphorbia heterophylla
tgttagctagaaaagggtagagggtggcctactttaccaatttgagcattaaaaaagcacatggctatgtattttagaccaaagtccctattgttggcagatgaactaagcaaccatatggttcattaactaactaactagttattgcaaatttgactgttccaattcatcaaaggactctccccatagaccattcaaca

SEQ ID NO: 630 - DNA, 200 bp, Euphorbia heterophylla
tgttgaatggtctatggggagagtcctttgatgaattggaacagtcaaatttgcaataactagttagttagttaatgaaccatatggttgcttagttcatctgccaacaatagggactttggtctaaaatacatagccatgtgcttttttaatgctcaaattggtaaagtaggccaccctctacccttttctagctaaca

SEQ ID NO: 631 - DNA, 200 bp, Euphorbia heterophylla
gactctccccatagaccattcaacacttgggttctagccctccctctcaccctcagacaacccttgtgaacctcattttcatcattggtccccttttttgttaggactacacttgtctctataaaatctcttctagccatgtgttatgtgtagactttttcggtttgaggattgggtcgggttcgggctgggtctcgtcg

SEQ ID NO: 632 - DNA, 200 bp, Euphorbia heterophylla
cgacgagacccagcccgaacccgacccaatcctcaaaccgaaaaagtctacacataacacatggctagaagagattttatagagacaagtgtagtcctaacaaaaaaggggaccaatgatgaaaatgaggttcacaagggttgtctgagggtgagagggagggctagaacccaagtgttgaatggtctatggggagagtc

SEQ ID NO: 633 - DNA, 200 bp, Euphorbia heterophylla
gtcgggttcgggctgggtctcgtcggactttttactgcaaacatcaaactagggttctaaacccatacttcagtactctggttcggattgaacttgcgagatcattctaaattcaatttacggctcatagattatatttgagagttttgagttttgggtggattagattgaaaacgactattttttgttgtataaatata

SEQ ID NO: 634 - DNA, 200 bp, Euphorbia heterophylla
tatatttatacaacaaaaaatagtcgttttcaatctaatccacccaaaactcaaaactctcaaatataatctatgagccgtaaattgaatttagaatgatctcgcaagttcaatccgaaccagagtactgaagtatgggtttagaaccctagtttgatgtttgcagtaaaaagtccgacgagacccagcccgaacccgac

SEQ ID NO: 635 - DNA, 200 bp, Euphorbia heterophylla
gcagcattgtcatcatcctttctccataccgaaccaaatagttcggtggaagttcattctcaagagcaagaagtagtaattgcattaggaagcaatgtgggagatagagttcataatttcaaccaagcattgcaattaatgaaaagttccggcattaacataactagacatggttgtttgtaccagacagcacctgctta

SEQ ID NO: 636 - DNA, 200 bp, Euphorbia heterophylla
taagcaggtgctgtctggtacaaacaaccatgtctagttatgttaatgccggaacttttcattaattgcaatgcttggttgaaattatgaactctatctcccacattgcttcctaatgcaattactacttcttgctcttgagaatgaacttccaccgaactatttggttcggtatggagaaaggatgatgacaatgctgc

SEQ ID NO: 637 - DNA, 200 bp, Euphorbia heterophylla
gtttgtaccagacagcacctgcttatgtcactgaccaacctcactttctcaactcagcagttcgagctttcacgaaactcggaccccacgagttattaggagttttaaagcagatcgaaaaggacttgggccgtaccaaagggattaggtacggaccaaggccaatcgatttggatatacttttctatggaaagttccgg

SEQ ID NO: 638 - DNA, 200 bp, Euphorbia heterophylla
ccggaactttccatagaaaagtatatccaaatcgattggccttggtccgtacctaatccctttggtacggcccaagtccttttcgatctgctttaaaactcctaataactcgtggggtccgagtttcgtgaaagctcgaactgctgagttgagaaagtgaggttggtcagtgacataagcaggtgctgtctggtacaaac

SEQ ID NO: 639 - DNA, 200 bp, Euphorbia heterophylla
tatacttttctatggaaagttccggattaattccgatacacttattgttcctcatgagagaatatgggagagaccctttgtgatggccccattggtggatttactcggctcggaaatcgagaatgacacggtcgccagctggcattccttgtccggcggtctttttgaatcatgggaaaagttgggcggcgaaagcctga

SEQ ID NO: 640 - DNA, 200 bp, Euphorbia heterophylla
tcaggctttcgccgcccaacttttcccatgattcaaaaagaccgccggacaaggaatgccagctggcgaccgtgtcattctcgatttccgagccgagtaaatccaccaatggggccatcacaaagggtctctcccatattctctcatgaggaacaataagtgtatcggaattaatccggaactttccatagaaaagtata

SEQ ID NO: 641 - DNA, 200 bp, Euphorbia heterophylla
gaaaagttgggcggcgaaagcctgatcgggaaggacggaatgaaaagagttaccccgatcggaaaccatttatgggattggtcggaaaagacttctgtaatgggaattatcaatttaacccccgatagtttcagcgatgggggcaagttgacaactattgattctatagtttctaaggttcgctcgatgatttccgaagg

SEQ ID NO: 642 - DNA, 200 bp, Euphorbia heterophylla
ccttcggaaatcatcgagcgaaccttagaaactatagaatcaatagttgtcaacttgcccccatcgctgaaactatcgggggttaaattgataattcccattacagaagtcttttccgaccaatcccataaatggtttccgatcggggtaactcttttcattccgtccttcccgatcaggctttcgccgcccaacttttc

SEQ ID NO: 643 - DNA, 200 bp, Euphorbia heterophylla
aggttcgctcgatgatttccgaaggggcggatattgtcgattttggtgctcaatcgacacgccctatggctaaaaggatatccccgcaagaggaaatggataggctaatccctgtattggaagcggttgtgaaaatacccgagatgaccgaaaagctcatatcggtcgacacattttactcggaagtcgccttagaagcg

SEQ ID NO: 644 - DNA, 200 bp, Euphorbia heterophylla
cgcttctaaggcgacttccgagtaaaatgtgtcgaccgatatgagcttttcggtcatctcgggtattttcacaaccgcttccaatacagggattagcctatccatttcctcttgcggggatatccttttagccatagggcgtgtcgattgagcaccaaaatcgacaatatccgccccttcggaaatcatcgagcgaacct

SEQ ID NO: 645 - DNA, 200 bp, Euphorbia heterophylla
ttactcggaagtcgccttagaagcggtccaaagaggagcaaatctcgtaaatgacgtatctggggggcaattagatccaaaaatgacggaaaatgtcgctaatctcgaggtaccatatatcttaatgcacatgaggggggaccccaccacaatgcagaacactcagaatctaaaatacgataatgtttgtaaagaggttg

SEQ ID NO: 646 - DNA, 200 bp, Euphorbia heterophylla
caacctctttacaaacattatcgtattttagattctgagtgttctgcattgtggtggggtcccccctcatgtgcattaagatatatggtacctcgagattagcgacattttccgtcatttttggatctaattgccccccagatacgtcatttacgagatttgctcctctttggaccgcttctaaggcgacttccgagtaa

SEQ ID NO: 647 - DNA, 200 bp, Euphorbia heterophylla
tacgataatgtttgtaaagaggttgcctttgagttgttttcgagggttaaagaagccgagatgtccggaattccagcctggcgaatgtttattgaccctggaattgggttttcgaagaacacgaatcagaatctggaaatcctgatgggaattccaaggatccgggccgagattgggaggaaaagcgtgggtatttctcg

SEQ ID NO: 648 - DNA, 200 bp, Euphorbia heterophylla
cgagaaatacccacgcttttcctcccaatctcggcccggatccttggaattcccatcaggatttccagattctgattcgtgttcttcgaaaacccaattccagggtcaataaacattcgccaggctggaattccggacatctcggcttctttaaccctcgaaaacaactcaaaggcaacctctttacaaacattatcgta

SEQ ID NO: 649 - DNA, 200 bp, Euphorbia heterophylla
ggaggaaaagcgtgggtatttctcgtgggccgattcttattgggccttcgagaaagaggtttttgggcgaaatttgcgaacgccctgaagcaactgaaagagacccggcaactgttgcttctgttactactgggattctgggaggggcaaatattgttagagttcataatgttagggataatgtggatgctgtgaagcta

SEQ ID NO: 650 - DNA, 200 bp, Euphorbia heterophylla
tagcttcacagcatccacattatccctaacattatgaactctaacaatatttgcccctcccagaatcccagtagtaacagaagcaacagttgccgggtctctttcagttgcttcagggcgttcgcaaatttcgcccaaaaacctctttctcgaaggcccaataagaatcggcccacgagaaatacccacgcttttcctcc

SEQ ID NO: 651 - DNA, 200 bp, Euphorbia heterophylla
gcagcattgtcatcatcctttctccataccgaaccaaatagttcggtggaagttcattctcaagagcaagaagtagtaattgcattaggaagcaatgtgggagatagagttcataatttcaaccaagcattgcgattaatgaaaagttccggcattaacgtaactagacatggttgtttgtaccagacagcacctgctta

SEQ ID NO: 652 - DNA, 200 bp, Euphorbia heterophylla
taagcaggtgctgtctggtacaaacaaccatgtctagttacgttaatgccggaacttttcattaatcgcaatgcttggttgaaattatgaactctatctcccacattgcttcctaatgcaattactacttcttgctcttgagaatgaacttccaccgaactatttggttcggtatggagaaaggatgatgacaatgctgc

SEQ ID NO: 653 - DNA, 200 bp, Euphorbia heterophylla; identical to SEQ ID NO: 637

SEQ ID NO: 654 - DNA, 200 bp, Euphorbia heterophylla; identical to SEQ ID NO: 638

SEQ ID NO: 655 - DNA, 200 bp, Euphorbia heterophylla; identical to SEQ ID NO: 639

SEQ ID NO: 656 - DNA, 200 bp, Euphorbia heterophylla; identical to SEQ ID NO: 640

SEQ ID NO: 657 - DNA, 200 bp, Euphorbia heterophylla; identical to SEQ ID NO: 641

SEQ ID NO: 658 - DNA, 200 bp, Euphorbia heterophylla; identical to SEQ ID NO: 642

SEQ ID NO: 659 - DNA, 200 bp, Euphorbia heterophylla
aggttcgctcgatgatttccgaaggggcggatattgtcgattttggtgctcaatcgacacgccctatggctaaaaggatatccccgcaagaggaaatggataggctaatccctgtattggaagcggttgtgaaaatacctgagatgaccggaaagctcatatcggtcgacacattttactcggaagtcgccttagaagcg

SEQ ID NO: 660 - DNA, 200 bp, Euphorbia heterophylla
cgcttctaaggcgacttccgagtaaaatgtgtcgaccgatatgagctttccggtcatctcaggtattttcacaaccgcttccaatacagggattagcctatccatttcctcttgcggggatatccttttagccatagggcgtgtcgattgagcaccaaaatcgacaatatccgccccttcggaaatcatcgagcgaacct

SEQ ID NO: 661 - DNA, 200 bp, Euphorbia heterophylla; identical to SEQ ID NO: 645

SEQ ID NO: 662 - DNA, 200 bp, Euphorbia heterophylla; identical to SEQ ID NO: 646

SEQ ID NO: 663 - DNA, 200 bp, Euphorbia heterophylla
tacgataatgtttgtaaagaggttgcctttgagttgttttcgagggttaaagaagccgagatgtccggaattccagcctggcgaatgattattgaccctggaattgggttttcgaagaacacgaatcagaatctggaaatcctgatgggaattccaaggatccgggccgagattgggagggaaagcgtgggtatttctcg

SEQ ID NO: 664 - DNA, 200 bp, Euphorbia heterophylla
cgagaaatacccacgctttccctcccaatctcggcccggatccttggaattcccatcaggatttccagattctgattcgtgttcttcgaaaacccaattccagggtcaataatcattcgccaggctggaattccggacatctcggcttctttaaccctcgaaaacaactcaaaggcaacctctttacaaacattatcgta

SEQ ID NO: 665 - DNA, 200 bp, Euphorbia heterophylla
ggagggaaagcgtgggtatttctcgtgggccgattcttattgggccttcgagaaagaggtttttgggcgaaatttgcgaacgccctaaagcaactgaaagagacccggcaactgttgcttctgttactactgggattctgggaggggcaaatattgttagagttcataatgttagggataatgtggatgctgtgaagcta

SEQ ID NO: 666 - DNA, 200 bp, Euphorbia heterophylla
tagcttcacagcatccacattatccctaacattatgaactctaacaatatttgcccctcccagaatcccagtagtaacagaagcaacagttgccgggtctctttcagttgctttagggcgttcgcaaatttcgcccaaaaacctctttctcgaaggcccaataagaatcggcccacgagaaatacccacgctttccctcc

SEQ ID NO: 667 - DNA, 200 bp, Euphorbia heterophylla
gttatgatgaaagtataagttgctaatcaagaagaaagagattttaatgtaattatttaaataataaattcctaccattttaattgtgtgacaattggttataatgattccgttgcatcgacaactaaaactagatagaaggatcaaaatataaaattttaacataattaatacatataatatataaatgtataatagat

SEQ ID NO: 668 - DNA, 200 bp, Euphorbia heterophylla
atctattatacatttatatattatatgtattaattatgttaaaattttatattttgatccttctatctagttttagttgtcgatgcaacggaatcattataaccaattgtcacacaattaaaatggtaggaatttattatttaaataattacattaaaatctctttcttcttgattagcaacttatactttcatcataac

SEQ ID NO: 669 - DNA, 200 bp, Euphorbia heterophylla
tataatatataaatgtataatagatacaattattattcctcgaattaaaattggctaattatataataatataatccaacctataaaaacttttgagcttttaagcttgagatgagctttgtaaaaatgattgtacaagcccgaccatgaaagagagccaagtagggtcaaatgcataccctggcttgaatataaattca

SEQ ID NO: 670 - DNA, 200 bp, Euphorbia heterophylla
tgaatttatattcaagccagggtatgcatttgaccctacttggctctctttcatggtcgggcttgtacaatcatttttacaaagctcatctcaagcttaaaagctcaaaagtttttataggttggattatattattatataattagccaattttaattcgaggaataataattgtatctattatacatttatatattata

SEQ ID NO: 671 - DNA, 200 bp, Euphorbia heterophylla
tctggggagtgatggttggaatagatgcttattcctggcagagatcgaaatgctccattttagtttcgggttttgttaaaatggggatttgttaaattgtttcaatggataattcactgttgtattagacttgatattgttagcagacactgatttttatgtattcgtgaactgaaaaggttgattttcttccatgtttt

SEQ ID NO: 672 - DNA, 200 bp, Euphorbia heterophylla
aaaacatggaagaaaatcaaccttttcagttcacgaatacataaaaatcagtgtctgctaacaatatcaagtctaatacaacagtgaattatccattgaaacaatttaacaaatccccattttaacaaaacccgaaactaaaatggagcatttcgatctctgccaggaataagcatctattccaaccatcactccccaga

SEQ ID NO: 673 - DNA, 200 bp, Euphorbia heterophylla
aaaggttgattttcttccatgttttgtctgatttatgatggaacttcatttcttagtggttaatgttggtatttccttctgttgtttgatttgtatctgtaattggtaccatgttatatgcaatcttttaaaaaatgttttaaaaagcgctatacgtggcagctgagactgttgagacttgaaaacgcacgggcatgtgc

SEQ ID NO: 674 - DNA, 200 bp, Euphorbia heterophylla
gcacatgcccgtgcgttttcaagtctcaacagtctcagctgccacgtatagcgctttttaaaacattttttaaaagattgcatataacatggtaccaattacagatacaaatcaaacaacagaaggaaataccaacattaaccactaagaaatgaagttccatcataaatcagacaaaacatggaagaaaatcaaccttt

SEQ ID NO: 675 - DNA, 200 bp, Euphorbia heterophylla
ttaaagcagatcgaaaaggacttgggccgtaccaaagggattaggtacggaccaaggccaatcgatttggatatactttttctatggaaagttccggattaattccgatacacttattgttcctcatgagagaatatgggagagaccctttgtgatggccccattggtggatttactcggctcggaaatcgagaatgaca

SEQ ID NO: 676 - DNA, 200 bp, Euphorbia heterophylla
tgtcattctcgatttccgagccgagtaaatccaccaatggggccatcacaaagggtctctcccatattctctcatgaggaacaataagtgtatcggaattaatccggaactttccatagaaaaagtatatccaaatcgattggccttggtccgtacctaatccctttggtacggcccaagtccttttcgatctgctttaa

SEQ ID NO: 677 - DNA, 200 bp, Euphorbia heterophylla
ctcggctcggaaatcgagaatgacacggtcgccagctggcattccttgtccggcggtctttttgaatcatgggaaaagttgggcggcgaaagcctgatcgggaaggacggaatgaaaagagttaccccgatcggaaaccatttatgggattggtcggaaaagacttctgtaatgggaattatcaatttaacccccgatag

SEQ ID NO: 678 - DNA, 200 bp, Euphorbia heterophylla
ctatcgggggttaaattgataattcccattacagaagtcttttccgaccaatcccataaatggtttccgatcggggtaactcttttcattccgtccttcccgatcaggctttcgccgcccaacttttcccatgattcaaaaagaccgccggacaaggaatgccagctggcgaccgtgtcattctcgatttccgagccgag

SEQ ID NO: 679 - DNA, 200 bp, Euphorbia heterophylla
gaattatcaatttaacccccgatagtttcagcgatgggggcaagttgacaactattgattctatagtttctaaggttcgctcgatgatttccgaaggggcggatattgtcgattttggtgctcaatcgacacgccctatggctaaaaggatatccccgcaagaggaaatggataggctaatccctgtattggaagcggtt

SEQ ID NO: 680 - DNA, 200 bp, Euphorbia heterophylla
aaccgcttccaatacagggattagcctatccatttcctcttgcggggatatccttttagccatagggcgtgtcgattgagcaccaaaatcgacaatatccgccccttcggaaatcatcgagcgaaccttagaaactatagaatcaatagttgtcaacttgcccccatcgctgaaactatcgggggttaaattgataattc

SEQ ID NO: 681 - DNA, 200 bp, Euphorbia heterophylla
tttgtatattaaaacaataaaaatgatccagattaataaatagaaaaagtattaataacccgaaaatatcaaatatcaaaatataatgaaacatgaacacgaaatcacaataaagaagttggtatcacaaacacaaagacacccttaaagctcctatgtttgtggagagttgtaagtcgactaatttcagattgcaaaag

SEQ ID NO: 682 - DNA, 200 bp, Euphorbia heterophylla
cttttgcaatctgaaattagtcgacttacaactctccacaaacataggagctttaagggtgtctttgtgtttgtgataccaacttctttattgtgatttcgtgttcatgtttcattatattttgatatttgatattttcgggttattaatactttttctatttattaatctggatcatttttattgttttaatatacaaa

SEQ ID NO: 683 - DNA, 200 bp, Euphorbia heterophylla
gtcgactaatttcagattgcaaaagttaaaaatggccaacttttttagctatgaggttagattagatacgagaatgccttggtgaaaagctggtcactgtttcttcattcgcgtggcaattaacccggcataatcgatacccaattcacggacaaggtcaaaggtatcagctttactcaacccttcgctttgatactaaa

SEQ ID NO: 684 - DNA, 200 bp, Euphorbia heterophylla
tttagtatcaaagcgaagggttgagtaaagctgatacctttgaccttgtccgtgaattgggtatcgattatgccgggttaattgccacgcgaatgaagaaacagtgaccagcttttcaccaaggcattctcgtatctaatctaacctcatagctaaaaaagttggccatttttaacttttgcaatctgaaattagtcgac

SEQ ID NO: 685 - DNA, 200 bp, Euphorbia heterophylla
ctcaacccttcgctttgatactaaattgctgattcttattctgattatgctttatttcgttacttcattttattgtctatgcacataagctgttcgacaaaaagcctgaatgaaactgtgagtgctggtacgcggttacaatactcctgattatgtccaatgattatttgggtactttaataccctaatttgtgaagttc

SEQ ID NO: 686 - DNA, 200 bp, Euphorbia heterophylla
gaacttcacaaattagggtattaaagtacccaaataatcattggacataatcaggagtattgtaaccgcgtaccagcactcacagtttcattcaggctttttgtcgaacagcttatgtgcatagacaataaaatgaagtaacgaaataaagcataatcagaataagaatcagcaatttagtatcaaagcgaagggttgag

SEQ ID NO: 687 - DNA, 200 bp, Euphorbia heterophylla
tttaataccctaatttgtgaagttcactagcgaaagggtagtttcttccccggttatttatcattttatgaatccccaagtctttgaagtacagctgattaaaatgcaagtactcataagttgtcattgtatgtttgaagtccttacatttttggtattttacagaatttgtactgaagaagcataaattcatacctagt

SEQ ID NO: 688 - DNA, 200 bp, Euphorbia heterophylla
actaggtatgaatttatgcttcttcagtacaaattctgtaaaataccaaaaatgtaaggacttcaaacatacaatgacaacttatgagtacttgcattttaatcagctgtacttcaaagacttggggattcataaaatgataaataaccggggaagaaactaccctttcgctagtgaacttcacaaattagggtattaaa

SEQ ID NO: 689 - DNA, 200 bp, Euphorbia heterophylla
gaagaagcataaattcatacctagttaggcttccttgttcctaatatgcttctcctcaagcggctggtgtccactaaacaagggttcaataatgccatcaaccgttttggaggtaatatagttatgcctatgatgctttaagtcgcttttcatcgatttgttacattaccaggctaagattctgcaattgaagttagaaa

SEQ ID NO: 690 - DNA, 200 bp, Euphorbia heterophylla
tttctaacttcaattgcagaatcttagcctggtaatgtaacaaatcgatgaaaagcgacttaaagcatcataggcataactatattacctccaaaacggttgatggcattattgaacccttgtttagtggacaccagccgcttgaggagaagcatattaggaacaaggaagcctaactaggtatgaatttatgcttcttc

SEQ ID NO: 691 - DNA, 200 bp, Euphorbia heterophylla
aagattctgcaattgaagttagaaatcgacgagtcttgtcttgaaaagtatggattttgttttgtcaccctggggccggttcaagattgctttcccaggttgctgtcttggcatcacgtcctaatccaaaaccctaaggtgatgggttaatgggtcccttcgcagttatatatcattcacttaactcattaacttccatg

SEQ ID NO: 692 - DNA, 200 bp, Euphorbia heterophylla
catggaagttaatgagttaagtgaatgatatataactgcgaagggacccattaacccatcaccttagggttttggattaggacgtgatgccaagacagcaacctgggaaagcaatcttgaaccggccccagggtgacaaaacaaaatccatacttttcaagacaagactcgtcgatttctaacttcaattgcagaatctt

SEQ ID NO: 693 - DNA, 200 bp, Euphorbia heterophylla
ttcacttaactcattaacttccatgtggataatcaactccacacttgccccaacactgtataaacgtagattttgacctttggaaatcattagctggacaaatcctaactaattagtatcaagagccaaaatcttttgaccaagaccaaaatatgttgtttcattgttgcagcattgtcatcatcctttctccataccga

SEQ ID NO: 694 - DNA, 200 bp, Euphorbia heterophylla
tcggtatggagaaaggatgatgacaatgctgcaacaatgaaacaacatattttggtcttggtcaaaagattttggctcttgatactaattagttaggatttgtccagctaatgatttccaaaggtcaaaatctacgtttatacagtgttggggcaagtgtggagttgattatccacatggaagttaatgagttaagtgaa

SEQ ID NO: 695 - DNA, 200 bp, Euphorbia heterophylla
tgtcatcatcctttctccataccgaaccaaatagttcggtggaagttcattctcaagagcaagaagtagtaattgcattaggaagcaatgtgggagatagagttcataatttcaaccaagcattgcaattaatgaaaagttccggcattaacataactagacatggttgtttgtaccagacagcacctgcttatgtcact

SEQ ID NO: 696 - DNA, 200 bp, Euphorbia heterophylla
agtgacataagcaggtgctgtctggtacaaacaaccatgtctagttatgttaatgccggaacttttcattaattgcaatgcttggttgaaattatgaactctatctcccacattgcttcctaatgcaattactacttcttgctcttgagaatgaacttccaccgaactatttggttcggtatggagaaaggatgatgaca

SEQ ID NO: 697 - DNA, 200 bp, Euphorbia heterophylla
ccagacagcacctgcttatgtcactgaccaacctcactttctcaactcagcagttcgagctttcacgaaactcggaccccacgagttattaggagttttaaagcagatcgaaaaggacttgggccgtaccaaagggattaggtacggaccaaggccaatcgatttggatatacttttctatggaaagttccggattaatt

SEQ ID NO: 698 - DNA, 200 bp, Euphorbia heterophylla
aattaatccggaactttccatagaaaagtatatccaaatcgattggccttggtccgtacctaatccctttggtacggcccaagtccttttcgatctgctttaaaactcctaataactcgtggggtccgagtttcgtgaaagctcgaactgctgagttgagaaagtgaggttggtcagtgacataagcaggtgctgtctgg

SEQ ID NO: 699 - DNA, 200 bp, Euphorbia heterophylla
ttctatggaaagttccggattaattccgatacacttattgttcctcatgagagaatatgggagagaccctttgtgatggccccattggtggatttactcggctcggaaatcgagaatgacacggtcgccagctggcattccttgtccggcggtctttttgaatcatgggaaaagttgggcggcgaaagcctgatcgggaa

SEQ ID NO: 700 - DNA, 200 bp, Euphorbia heterophylla
ttcccgatcaggctttcgccgcccaacttttcccatgattcaaaaagaccgccggacaaggaatgccagctggcgaccgtgtcattctcgatttccgagccgagtaaatccaccaatggggccatcacaaagggtctctcccatattctctcatgaggaacaataagtgtatcggaattaatccggaactttccatagaa

SEQ ID NO: 701 - DNA, 200 bp, Euphorbia heterophylla
tgggcggcgaaagcctgatcgggaaggacggaatgaaaagagttaccccgatcggaaaccatttatgggattggtcggaaaagacttctgtaatgggaattatcaatttaacccccgatagtttcagcgatgggggcaagttgacaactattgattctatagtttctaaggttcgctcgatgatttccgaaggggcggat

SEQ ID NO: 702 - DNA, 200 bp, Euphorbia heterophylla
atccgccccttcggaaatcatcgagcgaaccttagaaactatagaatcaatagttgtcaacttgcccccatcgctgaaactatcgggggttaaattgataattcccattacagaagtcttttccgaccaatcccataaatggtttccgatcggggtaactcttttcattccgtccttcccgatcaggctttcgccgccca

SEQ ID NO: 703 - DNA, 200 bp, Euphorbia heterophylla
ctcgatgatttccgaaggggcggatattgtcgattttggtgctcaatcgacacgccctatggctaaaaggatatccccgcaagaggaaatggataggctaatccctgtattggaagcggttgtgaaaatacccgagatgaccgaaaagctcatatcggtcgacacattttactcggaagtcgccttagaagcggtccaaa

SEQ ID NO: 704 - DNA, 200 bp, Euphorbia heterophylla
tttggaccgcttctaaggcgacttccgagtaaaatgtgtcgaccgatatgagcttttcggtcatctcgggtattttcacaaccgcttccaatacagggattagcctatccatttcctcttgcggggatatccttttagccatagggcgtgtcgattgagcaccaaaatcgacaatatccgccccttcggaaatcatcgag

SEQ ID NO: 705 - DNA, 200 bp, Euphorbia heterophylla
gaagtcgccttagaagcggtccaaagaggagcaaatctcgtaaatgacgtatctggggggcaattagatccaaaaatgacggaaaatgtcgctaatctcgaggtaccatatatcttaatgcacatgaggggggaccccaccacaatgcagaacactcagaatctaaaatacgataatgtttgtaaagaggttgcctttga

SEQ ID NO: 706 - DNA, 200 bp, Euphorbia heterophylla
tcaaaggcaacctctttacaaacattatcgtattttagattctgagtgttctgcattgtggtggggtcccccctcatgtgcattaagatatatggtacctcgagattagcgacattttccgtcatttttggatctaattgccccccagatacgtcatttacgagatttgctcctctttggaccgcttctaaggcgacttc

SEQ ID NO: 707 - DNA, 200 bp, Euphorbia heterophylla
atgtttgtaaagaggttgcctttgagttgttttcgagggttaaagaagccgagatgtccggaattccagcctggcgaatgtttattgaccctggaattgggttttcgaagaacacgaatcagaatctggaaatcctgatgggaattccaaggatccgggccgagattgggaggaaaagcgtgggtatttctcgtgggccg

SEQ ID NO: 708 - DNA, 200 bp, Euphorbia heterophylla
cggcccacgagaaatacccacgcttttcctcccaatctcggcccggatccttggaattcccatcaggatttccagattctgattcgtgttcttcgaaaacccaattccagggtcaataaacattcgccaggctggaattccggacatctcggcttctttaaccctcgaaaacaactcaaaggcaacctctttacaaacat

SEQ ID NO: 709 - DNA, 200 bp, Euphorbia heterophylla
aagcgtgggtatttctcgtgggccgattcttattgggccttcgagaaagaggtttttgggcgaaatttgcgaacgccctgaagcaactgaaagagacccggcaactgttgcttctgttactactgggattctgggaggggcaaatattgttagagttcataatgttagggataatgtggatgctgtgaagctatgtgact

SEQ ID NO: 710 - DNA, 200 bp, Euphorbia heterophylla
agtcacatagcttcacagcatccacattatccctaacattatgaactctaacaatatttgcccctcccagaatcccagtagtaacagaagcaacagttgccgggtctctttcagttgcttcagggcgttcgcaaatttcgcccaaaaacctctttctcgaaggcccaataagaatcggcccacgagaaatacccacgctt

SEQ ID NO: 711 - DNA, 200 bp, Euphorbia heterophylla
gtggatgctgtgaagctatgtgactcgatgatgaaactaaagaggtctacttaaaaattatcgggtatagttcgatttctttctttccgaatcttttgtttgactttcagattgtctttgattggcaaaacagtttcggctgcggttactgtttgctgttgcagtttaccgttgttgtttctaattgtgattttaagtta

SEQ ID NO: 712 - DNA, 200 bp, Euphorbia heterophylla
taacttaaaatcacaattagaaacaacaacggtaaactgcaacagcaaacagtaaccgcagccgaaactgttttgccaatcaaagacaatctgaaagtcaaacaaaagattcggaaagaaagaaatcgaactatacccgataatttttaagtagacctctttagtttcatcatcgagtcacatagcttcacagcatccac

SEQ ID NO: 713 - DNA, 200 bp, Euphorbia heterophylla
tgtttctaattgtgattttaagttattgttgttagttgatttgtacttagtgcttggtgaaaatataattcacggttgcggttggaggtataaattactaaaatagacatattttaaataaattagaagttatattgaagggtatttaggataaataaaagtccaaaccacaaaaaaaaaaaaccgtaaaactattgatt

SEQ ID NO: 714 - DNA, 200 bp, Euphorbia heterophylla
aatcaatagttttacggttttttttttttgtggtttggacttttatttatcctaaatacccttcaatataacttctaatttatttaaaatatgtctattttagtaatttatacctccaaccgcaaccgtgaattatattttcaccaagcactaagtacaaatcaactaacaacaataacttaaaatcacaattagaaaca

SEQ ID NO: 715 - DNA, 200 bp, Euphorbia heterophylla
aaaaaaaaccgtaaaactattgatttggtgaggtttggaaaaggagataaatcccttatgaaaagcaactccatatatgttttgaaacttaaccaaacacttgtatacctccttttccttttcaaacctcaaaagacctttttatagtaagctcttttgaagtatatacgctattgtttccgaatcattttggacttttc

SEQ ID NO: 716 - DNA, 200 bp, Euphorbia heterophylla
gaaaagtccaaaatgattcggaaacaatagcgtatatacttcaaaagagcttactataaaaaggtcttttgaggtttgaaaaggaaaaggaggtatacaagtgtttggttaagtttcaaaacatatatggagttgcttttcataagggatttatctccttttccaaacctcaccaaatcaatagttttacggtttttttt

SEQ ID NO: 717 - DNA, 200 bp, Euphorbia heterophylla
gtttccgaatcattttggacttttcgtcgaaatcaacaatactgttagctagtcatatttcaagatatttgggatgtaaagatcaaggtagttgttgttgtattctgttctcatcagcaaaatttgttataactccaccaagatttaattttttccaatctgaaaatgtcttatgttatagtaagaagtgagatgagttt

SEQ ID NO: 718 - DNA, 200 bp, Euphorbia heterophylla
aaactcatctcacttcttactataacataagacattttcagattggaaaaaattaaatcttggtggagttataacaaattttgctgatgagaacagaatacaacaacaactaccttgatctttacatcccaaatatcttgaaatatgactagctaacagtattgttgatttcgacgaaaagtccaaaatgattcggaaac

SEQ ID NO: 719 - DNA, 200 bp, Euphorbia heterophylla
ttatagtaagaagtgagatgagtttatgtctttgtgaaaagattttgcccaattaatgcaaaaattttgtgacaagacaagaggggtgaaagagacaagcaacaagagaaagcagtgaaaggggcaaatgttaggaaactgttagctagaaaagggtagagggtggcctactttaccaatttgagcattaaaaaagcaca

SEQ ID NO: 720 - DNA, 200 bp, Euphorbia heterophylla
tgtgcttttttaatgctcaaattggtaaagtaggccaccctctacccttttctagctaacagtttcctaacatttgcccctttcactgctttctcttgttgcttgtctctttcacccctcttgtcttgtcacaaaatttttgcattaattgggcaaaatcttttcacaaagacataaactcatctcacttcttactataa

SEQ ID NO: 721 - DNA, 200 bp, Euphorbia heterophylla
ccaatttgagcattaaaaaagcacatggctatgtattttagaccaaagtccctattgttggcagatgaactaagcaaccatatggttcattaactaactaactagttattgcaaatttgactgttccaattcatcaaaggactctccccatagaccattcaacacttgggttctagccctccctctcaccctcagacaac

SEQ ID NO: 722 - DNA, 200 bp, Euphorbia heterophylla
gttgtctgagggtgagagggagggctagaacccaagtgttgaatggtctatggggagagtcctttgatgaattggaacagtcaaatttgcaataactagttagttagttaatgaaccatatggttgcttagttcatctgccaacaatagggactttggtctaaaatacatagccatgtgcttttttaatgctcaaattgg

SEQ ID NO: 723 - DNA, 200 bp, Euphorbia heterophylla
gccctccctctcaccctcagacaacccttgtgaacctcattttcatcattggtccccttttttgttaggactacacttgtctctataaaatctcttctagccatgtgttatgtgtagactttttcggtttgaggattgggtcgggttcgggctgggtctcgtcgggctttttactgcaaacatcaaactagggttctaaa

SEQ ID NO: 724 - DNA, 200 bp, Euphorbia heterophylla
tttagaaccctagtttgatgtttgcagtaaaaagcccgacgagacccagcccgaacccgacccaatcctcaaaccgaaaaagtctacacataacacatggctagaagagattttatagagacaagtgtagtcctaacaaaaaaggggaccaatgatgaaaatgaggttcacaagggttgtctgagggtgagagggagggc

SEQ ID NO: 725 - DNA, 200 bp, Euphorbia heterophylla
gcaaacatcaaactagggttctaaacccatacttcagtactctggttcggattgaacttgcgagatcattctaaattcaatttacggctcatagattatatttgagagttttgagttttgggtggattagattgaaaacgactattttttgttgtataaatatatctaaaaagttatataattacattctttattggaat

SEQ ID NO: 726 - DNA, 200 bp, Euphorbia heterophylla
attccaataaagaatgtaattatataactttttagatatatttatacaacaaaaaatagtcgttttcaatctaatccacccaaaactcaaaactctcaaatataatctatgagccgtaaattgaatttagaatgatctcgcaagttcaatccgaaccagagtactgaagtatgggtttagaaccctagtttgatgtttgc

SEQ ID NO: 727 - DNA, 200 bp, Euphorbia heterophylla
atataattacattctttattggaattcatcaatatatataagaatttgactttgttgtagccattttgaaagttcgataagctactcccttcgtctagtttcaatagttcatttaaaagtttcattaattctgttttcaattgtcaatttaaattttcataatatttttcaccatttttttttccaatttgcccttgtat

SEQ ID NO: 728 - DNA, 200 bp, Euphorbia heterophylla
atacaagggcaaattggaaaaaaaaatggtgaaaaatattatgaaaatttaaattgacaattgaaaacagaattaatgaaacttttaaatgaactattgaaactagacgaagggagtagcttatcgaactttcaaaatggctacaacaaagtcaaattcttatatatattgatgaattccaataaagaatgtaattatat

SEQ ID NO: 729 - DNA, 200 bp, Euphorbia heterophylla
ttttttttccaatttgcccttgtatttattgagaaagagattatgttatgatgaaagtataagttgctaatcaagaagaaagagattttaatgtaattatttaaataataaattcctaccattttaattgtgtgacaattggttataatgattccgttgcatcgacaactaaaactagatagaaggatcaaaatataaaa

SEQ ID NO: 730 - DNA, 200 bp, Euphorbia heterophylla
ttttatattttgatccttctatctagttttagttgtcgatgcaacggaatcattataaccaattgtcacacaattaaaatggtaggaatttattatttaaataattacattaaaatctctttcttcttgattagcaacttatactttcatcataacataatctctttctcaataaatacaagggcaaattggaaaaaaaa

SEQ ID NO: 731 - DNA, 200 bp, Euphorbia heterophylla
tagatagaaggatcaaaatataaaattttaacataattaatacatataatatataaatgtataatagatacaattattattcctcgaattaaaattggctaattatataataatataatccaacctataaaaacttttgagcttttaagcttgagatgagctttgtaaaaatgattgtacaagcccgaccatgaaagaga

SEQ ID NO: 732 - DNA, 200 bp, Euphorbia heterophylla
tctctttcatggtcgggcttgtacaatcatttttacaaagctcatctcaagcttaaaagctcaaaagtttttataggttggattatattattatataattagccaattttaattcgaggaataataattgtatctattatacatttatatattatatgtattaattatgttaaaattttatattttgatccttctatcta

SEQ ID NO: 733 - DNA, 200 bp, Kochia scoparia
gaatgtgcttaggcggctgcaatcagccagtttgatttggaagggaatttccaagtatgatagaggtaaagcacagttgtttcattttttgactatatatttaatttgggaatctgtactgtttcattttgtaactttattttgatcctgagttattgaatctgtcatgtctcattttgagacggtattttgaatctgag

SEQ ID NO: 734 - DNA, 200 bp, Kochia scoparia
ctcagattcaaaataccgtctcaaaatgagacatgacagattcaataactcaggatcaaaataaagttacaaaatgaaacagtacagattcccaaattaaatatatagtcaaaaaatgaaacaactgtgctttacctctatcatacttggaaattcccttccaaatcaaactggctgattgcagccgcctaagcacattc

SEQ ID NO: 735 - DNA, 200 bp, Kochia scoparia
tttgagacggtattttgaatctgagtctggatatgaaattatgaataggccataggtatttataaatatagggtttaagttgatgatctggatgctatcagagctctattatgcaggattacaaatcataatttcattgtgaaatttacttctttatgtgtattcacttgaagattcataattttgatgtattcataaat

SEQ ID NO: 736 - DNA, 200 bp, Kochia scoparia
atttatgaatacatcaaaattatgaatcttcaagtgaatacacataaagaagtaaatttcacaatgaaattatgatttgtaatcctgcataatagagctctgatagcatccagatcatcaacttaaaccctatatttataaatacctatggcctattcataatttcatatccagactcagattcaaaataccgtctcaaa

SEQ ID NO: 737 - DNA, 200 bp, Kochia scoparia
tcataattttgatgtattcataaatttgatgtgccctaaacagaaagagtcagcatgtgctgaaagtataagggggaaaaccaagatgtataaatcttttccttgatgagttgcttatatgattgatgtcatgattctaaaatgcctttacctaattcaaatggattttaaagattacgtatgtacttttgcagcttcaa

SEQ ID NO: 738 - DNA, 200 bp, Kochia scoparia
ttgaagctgcaaaagtacatacgtaatctttaaaatccatttgaattaggtaaaggcattttagaatcatgacatcaatcatataagcaactcatcaaggaaaagatttatacatcttggttttcccccttatactttcagcacatgctgactctttctgtttagggcacatcaaatttatgaatacatcaaaattatga

SEQ ID NO: 739 - DNA, 200 bp, Kochia scoparia
tacgtatgtacttttgcagcttcaagtcttgcttttttgcactcatcaccagaaactactattgaggtttgttctaaagagcatgaagttgtaattgctctagggagcaatgtaggagacagattagataactttaaccaagctctgcagctaatgaagaaattaggagtaaatatcacaaggcatggctgcttatacga

SEQ ID NO: 740 - DNA, 200 bp, Kochia scoparia
tcgtataagcagccatgccttgtgatatttactcctaatttcttcattagctgcagagcttggttaaagttatctaatctgtctcctacattgctccctagagcaattacaacttcatgctctttagaacaaacctcaatagtagtttctggtgatgagtgcaaaaaagcaagacttgaagctgcaaaagtacatacgta

SEQ ID NO: 741 - DNA, 200 bp, Kochia scoparia
tcacaaggcatggctgcttatacgagacagatcctgcctatgtgactgatcaaccaaagtttcttaactctgcagtgaggggcttcacaaaacttgggcctctagaactattagggatgctgaagaaaattgagaaggatatgggtcgaactaatggaataagatatggccctaggccaattgacttagacattcttttt

SEQ ID NO: 742 - DNA, 200 bp, Kochia scoparia
aaaaagaatgtctaagtcaattggcctagggccatatcttattccattagttcgacccatatccttctcaattttcttcagcatccctaatagttctagaggcccaagttttgtgaagcccctcactgcagagttaagaaactttggttgatcagtcacataggcaggatctgtctcgtataagcagccatgccttgtga

SEQ ID NO: 743 - DNA, 200 bp, Kochia scoparia
gccaattgacttagacattcttttttatgggaagtttagggtaagctctgagaaacttacagttccgcatgaaaggatatgggaaagaccctttgtgatggcaccactaattgatataattggatcggatgtagaaaatgacactgttgctgcatggcattcattatcaaaattttctggtggactatttgaagcgtggg

SEQ ID NO: 744 - DNA, 200 bp, Kochia scoparia
cccacgcttcaaatagtccaccagaaaattttgataatgaatgccatgcagcaacagtgtcattttctacatccgatccaattatatcaattagtggtgccatcacaaagggtctttcccatatcctttcatgcggaactgtaagtttctcagagcttaccctaaacttcccataaaaaagaatgtctaagtcaattggc

SEQ ID NO: 745 - DNA, 200 bp, Kochia scoparia
tctggtggactatttgaagcgtgggataaacttggtggagattcactcattgggaaggacggaatgagtagggttttgccagttgggaaccacttgtgggattggtcgtgcaaaacctctgtaatgggagtcttgaatttgactccggacagctttagtgatggaggaaagtttctacctgtagaaaatgcagtttctca

SEQ ID NO: 746 - DNA, 200 bp, Kochia scoparia
tgagaaactgcattttctacaggtagaaactttcctccatcactaaagctgtccggagtcaaattcaagactcccattacagaggttttgcacgaccaatcccacaagtggttcccaactggcaaaaccctactcattccgtccttcccaatgagtgaatctccaccaagtttatcccacgcttcaaatagtccaccaga

SEQ ID NO: 747 - DNA, 200 bp, Kochia scoparia
tacctgtagaaaatgcagtttctcaggttcgtcaaatgatctcagaaggtgctgacataattgatattggagcgcaatcaacaaggcccatggcaactaggatttctgctgaggaagagctggaaagactagtccctgttttagaaggtgtcaaggatgttatcgaggaagaaggaagaatgttatcagtggatacattt

SEQ ID NO: 748 - DNA, 200 bp, Kochia scoparia
aaatgtatccactgataacattcttccttcttcctcgataacatccttgacaccttctaaaacagggactagtctttccagctcttcctcagcagaaatcctagttgccatgggccttgttgattgcgctccaatatcaattatgtcagcaccttctgagatcatttgacgaacctgagaaactgcattttctacaggta

SEQ ID NO: 749 - DNA, 200 bp, Kochia scoparia
aagaatgttatcagtggatacattttactcaaaagttgcttctgaagctgtcaataagggagcacatatggtgaatgatgtttcgagtggaaagctagatcctgagatgttcaatgttgttgcagggcttaaagtgccttatatagcaatgcacatgcgaggtgatccttcttcaatgcaaaatgctgataatttgacat

SEQ ID NO: 750 - DNA, 200 bp, Kochia scoparia
atgtcaaattatcagcattttgcattgaagaaggatcacctcgcatgtgcattgctatataaggcactttaagccctgcaacaacattgaacatctcaggatctagctttccactcgaaacatcattcaccatatgtgctcccttattgacagcttcagaagcaacttttgagtaaaatgtatccactgataacattctt

SEQ ID NO: 751 - DNA, 200 bp, Kochia scoparia
atgcaaaatgctgataatttgacatacaatgatgtttgtaaggaggtggctttagagttgagctctaggattacagatgcagaattatcaggtattcctgcttggaggatagttattgatcctggcattggattttccaagaatacaaagcaaaatttggaaattctcacagggttgaaaagaattcggcaagaaatagc

SEQ ID NO: 752 - DNA, 200 bp, Kochia scoparia
gctatttcttgccgaattcttttcaaccctgtgagaatttccaaattttgctttgtattcttggaaaatccaatgccaggatcaataactatcctccaagcaggaatacctgataattctgcatctgtaatcctagagctcaactctaaagccacctccttacaaacatcattgtatgtcaaattatcagcattttgcat

SEQ ID NO: 753 - DNA, 200 bp, Kochia scoparia
tgaaaagaattcggcaagaaatagcaaaaaagagtttggctgtggctcatggtcccttactgattggaccttcaagaaagaggtttttgggtgagatctgcaatcgtcctgtagcatctgatagagatccggcaactgttgcttctatcaccgctggagttttgggtggtgcaaacattgtaagagtacataatgttcgg

SEQ ID NO: 754 - DNA, 200 bp, Kochia scoparia
ccgaacattatgtactcttacaatgtttgcaccacccaaaactccagcggtgatagaagcaacagttgccggatctctatcagatgctacaggacgattgcagatctcacccaaaaacctctttcttgaaggtccaatcagtaagggaccatgagccacagccaaactcttttttgctatttcttgccgaattcttttca

SEQ ID NO: 755 - DNA, 200 bp, Kochia scoparia
cattgtaagagtacataatgttcgggatagccttgatggtgtcaagctatgtgatgcaatgcttgaatatcagtataactgaagattcttagattcttagattcttagattctaagagttgattttatagattattgtctttgttgtgtgccttcggcagatatgaataatatgattgtaattttgttgcttatgatttt

SEQ ID NO: 756 - DNA, 200 bp, Kochia scoparia
aaaatcataagcaacaaaattacaatcatattattcatatctgccgaaggcacacaacaaagacaataatctataaaatcaactcttagaatctaagaatctaagaatctaagaatcttcagttatactgatattcaagcattgcatcacatagcttgacaccatcaaggctatcccgaacattatgtactcttacaatg

SEQ ID NO: 757 - DNA, 200 bp, Kochia scoparia
ttgtaattttgttgcttatgattttcttcctacctttcttttatcatagaatttagattaaaaaatgtacttgaatttcataaatggtgaaataaagtttgtcggtgcttcatgcaatacgacttttctagtttgcattgctctatcataggggctgtaagcagattagcccagtcaattcacattagatttagacggcc

SEQ ID NO: 758 - DNA, 200 bp, Kochia scoparia
ggccgtctaaatctaatgtgaattgactgggctaatctgcttacagcccctatgatagagcaatgcaaactagaaaagtcgtattgcatgaagcaccgacaaactttatttcaccatttatgaaattcaagtacattttttaatctaaattctatgataaaagaaaggtaggaagaaaatcataagcaacaaaattacaa

SEQ ID NO: 759 - DNA, 200 bp, Kochia scoparia
caattcacattagatttagacggccggtgcaaaaacagcataatgttgctaacgctgtttatacatttaaactatatgatctctaagagcaaaatacaaaataatacaatgtgtttcatgaagatgtccaatcatttctccctcaaaaaaagatcgtgatgaacaatcaggtactttgattttgttgtagaacctcagct

SEQ ID NO: 760 - DNA, 200 bp, Kochia scoparia
agctgaggttctacaacaaaatcaaagtacctgattgttcatcacgatctttttttgagggagaaatgattggacatcttcatgaaacacattgtattattttgtattttgctcttagagatcatatagtttaaatgtataaacagcgttagcaacattatgctgtttttgcaccggccgtctaaatctaatgtgaattg

SEQ ID NO: 761 - DNA, 200 bp, Kochia scoparia
ttgattttgttgtagaacctcagcttcagccctagccatgggatcatctttttctccttctcgctcctcagccaatttctttctgtgcatctcctctgcagcttctattgaagctttgtgctcagcctggagacgctgcatttcctcttccatctgctgtctttcgaaccatgtatcagcatccgcccaaactgcagaaa

SEQ ID NO: 762 - DNA, 200 bp, Kochia scoparia
tttctgcagtttgggcggatgctgatacatggttcgaaagacagcagatggaagaggaaatgcagcgtctccaggctgagcacaaagcttcaatagaagctgcagaggagatgcacagaaagaaattggctgaggagcgagaaggagaaaaagatgatcccatggctagggctgaagctgaggttctacaacaaaatcaa

SEQ ID NO: 763 - DNA, 200 bp, Kochia scoparia
tcagcatccgcccaaactgcagaaataatttaaacaagttaatcatacggagtgtgcagcatggcaaattggcaataagatggtgcaagtaacaagctcaaatgatccaccatagccaaattcctaaccattggactgtgtcatatactcatatcagctagtagaaacattctcaccataaccctaactattcgaggata

SEQ ID NO: 764 - DNA, 200 bp, Kochia scoparia
tatcctcgaatagttagggttatggtgagaatgtttctactagctgatatgagtatatgacacagtccaatggttaggaatttggctatggtggatcatttgagcttgttacttgcaccatcttattgccaatttgccatgctgcacactccgtatgattaacttgtttaaattatttctgcagtttgggcggatgctga

SEQ ID NO: 765 - DNA, 200 bp, Kochia scoparia
ccataaccctaactattcgaggataagtgataccattcaacaatagtaaagatcaaatcaggtattactagacctatcaaagtaatacggagtagttgttattgttacagagttaggaagaatcctattttcccatttctcctatcaatcctaaacttttgtgactgaactatcgacatatagtgtgaagaacatgaatg

SEQ ID NO: 766 - DNA, 200 bp, Kochia scoparia
cattcatgttcttcacactatatgtcgatagttcagtcacaaaagtttaggattgataggagaaatgggaaaataggattcttcctaactctgtaacaataacaactactccgtattactttgataggtctagtaatacctgatttgatctttactattgttgaatggtatcacttatcctcgaatagttagggttatgg

SEQ ID NO: 767 - DNA, 200 bp, Kochia scoparia
acatatagtgtgaagaacatgaatggcagaagtcacaactctgattcctgagtcctgactataaactattggatgactgtgatacatcattagtcctttaagcattgagttactctattctacatagttaaattatatagattccaagatccgaatccatgaattatcaattaccataaattgatcttcttcctttgagt

SEQ ID NO: 768 - DNA, 200 bp, Kochia scoparia
actcaaaggaagaagatcaatttatggtaattgataattcatggattcggatcttggaatctatataatttaactatgtagaatagagtaactcaatgcttaaaggactaatgatgtatcacagtcatccaatagtttatagtcaggactcaggaatcagagttgtgacttctgccattcatgttcttcacactatatgt

SEQ ID NO: 769 - DNA, 200 bp, Kochia scoparia
ataaattgatcttcttcctttgagttttgtcaacagttcctactccgaaatatgtcttaaatctagaagcaatagacacttatcaatgatacagcaatagacgaattcatctacccaaggcagcatcaacaacagctgccgtttgcgataatcgaaactatcatagtgtccttttcactattagtgcagatgttatctag

SEQ ID NO: 770 - DNA, 200 bp, Kochia scoparia
ctagataacatctgcactaatagtgaaaaggacactatgatagtttcgattatcgcaaacggcagctgttgttgatgctgccttgggtagatgaattcgtctattgctgtatcattgataagtgtctattgcttctagatttaagacatatttcggagtaggaactgttgacaaaactcaaaggaagaagatcaatttat

SEQ ID NO: 771 - DNA, 200 bp, Kochia scoparia
cactattagtgcagatgttatctagggtctaaacctctatgtttctacattcctggaaaatcttattctgtactgcataatcctctcacaaaaaattcgccaatgaatgacaaattagctatccagaattgcagattgattgaatcaaattgatcatggcagcagcacaattgcagcagttatcaattgaagcacaatat

SEQ ID NO: 772 - DNA, 200 bp, Kochia scoparia
atattgtgcttcaattgataactgctgcaattgtgctgctgccatgatcaatttgattcaatcaatctgcaattctggatagctaatttgtcattcattggcgaattttttgtgagaggattatgcagtacagaataagattttccaggaatgtagaaacatagaggtttagaccctagataacatctgcactaatagtg

SEQ ID NO: 773 - DNA, 200 bp, Kochia scoparia
gcagttatcaattgaagcacaatattacctctcaatcataacaatcaaagcacagaaattccaatcaataactccacaatatagtatcaatctctaactcagaaactaaacagaagcaaaaacccattcaatcaaagcaacagtaaaattagggtttgaattagaaagaaagagagggaagagaaacatactgggttgcg

SEQ ID NO: 774 - DNA, 200 bp, Kochia scoparia
cgcaacccagtatgtttctcttccctctctttctttctaattcaaaccctaattttactgttgctttgattgaatgggtttttgcttctgtttagtttctgagttagagattgatactatattgtggagttattgattggaatttctgtgctttgattgttatgattgagaggtaatattgtgcttcaattgataactgc

SEQ ID NO: 775 - DNA, 200 bp, Kochia scoparia
gggaagagaaacatactgggttgcgcagcccatccctgttcaccacccttaatcttctcgggaagagcgtaattgacagtgagaacacgaccgtaaagctcagcaccatccatgttatccatggcagaagaagcgtcgtctctctccaagaaagtgacgaaaccgaaagaacgatgcttgttggtggccaaatcaagagg

SEQ ID NO: 776 - DNA, 200 bp, Kochia scoparia
cctcttgatttggccaccaacaagcatcgttctttcggtttcgtcactttcttggagagagacgacgcttcttctgccatggataacatggatggtgctgagctttacggtcgtgttctcactgtcaattacgctcttcccgagaagattaagggtggtgaacagggatgggctgcgcaacccagtatgtttctcttccc

SEQ ID NO: 777 - DNA, 200 bp, Kochia scoparia
gcagcttcaagtcttgcttttttgcactcatcaccagaaactactattgaggtttgttctaaagagcatgaagttgtaattgctctagggagcaatgtaggagacagattagataactttaaccaagctctgcagctaatgaagaaattaggagtaaatatcacaaggcatggctgcttatacgagacagatcctgccta

SEQ ID NO: 778 - DNA, 200 bp, Kochia scoparia
taggcaggatctgtctcgtataagcagccatgccttgtgatatttactcctaatttcttcattagctgcagagcttggttaaagttatctaatctgtctcctacattgctccctagagcaattacaacttcatgctctttagaacaaacctcaatagtagtttctggtgatgagtgcaaaaaagcaagacttgaagctgc

SEQ ID NO: 779 - DNA, 200 bp, Kochia scoparia
gcttatacgagacagatcctgcctatgtgactgatcaaccaaagtttcttaactctgcagtgaggggcttcacaaaacttgggcctctagaactattagggatgctgaagaaaattgagaaggatatgggtcgaactaatggaataagatatggccctaggccaattgacttagacattcttttttatgggaagtttagg

SEQ ID NO: 780 - DNA, 200 bp, Kochia scoparia
cctaaacttcccataaaaaagaatgtctaagtcaattggcctagggccatatcttattccattagttcgacccatatccttctcaattttcttcagcatccctaatagttctagaggcccaagttttgtgaagcccctcactgcagagttaagaaactttggttgatcagtcacataggcaggatctgtctcgtataagc

SEQ ID NO: 781 - DNA, 200 bp, Kochia scoparia
cattcttttttatgggaagtttagggtaagctctgagaaacttacagttccgcatgaaaggatatgggaaagaccctttgtgatggcaccactaattgatataattggatcggatgtagaaaatgacactgttgctgcatggcattcattatcaaaattttctggtggactatttgaagcgtgggataaacttggtggag

SEQ ID NO: 782 - DNA, 200 bp, Kochia scoparia
ctccaccaagtttatcccacgcttcaaatagtccaccagaaaattttgataatgaatgccatgcagcaacagtgtcattttctacatccgatccaattatatcaattagtggtgccatcacaaagggtctttcccatatcctttcatgcggaactgtaagtttctcagagcttaccctaaacttcccataaaaaagaatg

SEQ ID NO: 783 - DNA, 200 bp, Kochia scoparia
gaagcgtgggataaacttggtggagattcactcattgggaaggacggaatgagtagggttttgccagttgggaaccacttgtgggattggtcgtgcaaaacctctgtaatgggagtcttgaatttgactccggacagctttagtgatggaggaaagtttctacctgtagaaaatgcagtttctcaggttcgtcaaatgat

SEQ ID NO: 784 - DNA, 200 bp, Kochia scoparia
atcatttgacgaacctgagaaactgcattttctacaggtagaaactttcctccatcactaaagctgtccggagtcaaattcaagactcccattacagaggttttgcacgaccaatcccacaagtggttcccaactggcaaaaccctactcattccgtccttcccaatgagtgaatctccaccaagtttatcccacgcttc

SEQ ID NO: 785 - DNA, 200 bp, Kochia scoparia
cagtttctcaggttcgtcaaatgatctcagaaggtgctgacataattgatattggagcgcaatcaacaaggcccatggcaactaggatttctgctgaggaagagctggaaagactagtccctgttttagaaggtgtcaaggatgttatcgaggaagaaggaagaatgttatcagtggatacattttactcaaaagttgct

SEQ ID NO: 786 - DNA, 200 bp, Kochia scoparia
agcaacttttgagtaaaatgtatccactgataacattcttccttcttcctcgataacatccttgacaccttctaaaacagggactagtctttccagctcttcctcagcagaaatcctagttgccatgggccttgttgattgcgctccaatatcaattatgtcagcaccttctgagatcatttgacgaacctgagaaactg

SEQ ID NO: 787 - DNA, 200 bp, Kochia scoparia
ggatacattttactcaaaagttgcttctgaagctgtcaataagggagcacatatggtgaatgatgtttcgagtggaaagctagatcctgagatgttcaatgttgttgcagggcttaaagtgccttatatagcaatgcacatgcgaggtgatccttcttcaatgcaaaatgctgataatttgacatacaatgatgtttgta

SEQ ID NO: 788 - DNA, 200 bp, Kochia scoparia
tacaaacatcattgtatgtcaaattatcagcattttgcattgaagaaggatcacctcgcatgtgcattgctatataaggcactttaagccctgcaacaacattgaacatctcaggatctagctttccactcgaaacatcattcaccatatgtgctcccttattgacagcttcagaagcaacttttgagtaaaatgtatcc

SEQ ID NO: 789 - DNA, 200 bp, Kochia scoparia
aatttgacatacaatgatgtttgtaaggaggtggctttagagttgagctctaggattacagatgcagaattatcaggtattcctgcttggaggatagttattgatcctggcattggattttccaagaatacaaagcaaaatttggaaattctcacagggttgaaaagaattcggcaagaaatagcaaaaaagagtttggc

SEQ ID NO: 790 - DNA, 200 bp, Kochia scoparia
gccaaactcttttttgctatttcttgccgaattcttttcaaccctgtgagaatttccaaattttgctttgtattcttggaaaatccaatgccaggatcaataactatcctccaagcaggaatacctgataattctgcatctgtaatcctagagctcaactctaaagccacctccttacaaacatcattgtatgtcaaatt

SEQ ID NO: 791 - DNA, 200 bp, Kochia scoparia
aagaaatagcaaaaaagagtttggctgtggctcatggtcccttactgattggaccttcaagaaagaggtttttgggtgagatctgcaatcgtcctgtagcatctgatagagatccggcaactgttgcttctatcaccgctggagttttgggtggtgcaaacattgtaagagtacataatgttcgggatagccttgatggt

SEQ ID NO: 792 - DNA, 200 bp, Kochia scoparia
accatcaaggctatcccgaacattatgtactcttacaatgtttgcaccacccaaaactccagcggtgatagaagcaacagttgccggatctctatcagatgctacaggacgattgcagatctcacccaaaaacctctttcttgaaggtccaatcagtaagggaccatgagccacagccaaactcttttttgctatttctt

SEQ ID NO: 793 - DNA, 200 bp, Kochia scoparia
taattcaaatggattttaaagattacgtatgtacttttgcagcttcaagtcttgcttttttgcactcatcaccagaaactactattgaggtttgttctaaagagcatgaagttgtaattgctctagggagcaatgtaggagacagattagataactttaaccaagctctgcagctaatgaagaaattaggagtaaatatc

SEQ ID NO: 794 - DNA, 200 bp, Kochia scoparia
gatatttactcctaatttcttcattagctgcagagcttggttaaagttatctaatctgtctcctacattgctccctagagcaattacaacttcatgctctttagaacaaacctcaatagtagtttctggtgatgagtgcaaaaaagcaagacttgaagctgcaaaagtacatacgtaatctttaaaatccatttgaatta

SEQ ID NO: 795 - DNA, 200 bp, Kochia scoparia
aatgaagaaattaggagtaaatatcacaaggcatggctgcttatacgagacagatcctgcctatgtgactgatcaaccaaagtttcttaactctgcagtgaggggcttcacaaaacttgggcctctagaactattagggatgctgaagaaaattgagaaggatatgggtcgaactaatggaataagatatggccctaggc

SEQ ID NO: 796 - DNA, 200 bp, Kochia scoparia
gcctagggccatatcttattccattagttcgacccatatccttctcaattttcttcagcatccctaatagttctagaggcccaagttttgtgaagcccctcactgcagagttaagaaactttggttgatcagtcacataggcaggatctgtctcgtataagcagccatgccttgtgatatttactcctaatttcttcatt

SEQ ID NO: 797 - DNA, 200 bp, Kochia scoparia
aatggaataagatatggccctaggccaattgacttagacattcttttttatgggaagtttagggtaagctctgagaaacttacagttccgcatgaaaggatatgggaaagaccctttgtgatggcaccactaattgatataattggatcggatgtagaaaatgacactgttgctgcatggcattcattatcaaaattttc

SEQ ID NO: 798 - DNA, 200 bp, Kochia scoparia
gaaaattttgataatgaatgccatgcagcaacagtgtcattttctacatccgatccaattatatcaattagtggtgccatcacaaagggtctttcccatatcctttcatgcggaactgtaagtttctcagagcttaccctaaacttcccataaaaaagaatgtctaagtcaattggcctagggccatatcttattccatt

SEQ ID NO: 799 - DNA, 200 bp, Kochia scoparia
catggcattcattatcaaaattttctggtggactatttgaagcgtgggataaacttggtggagattcactcattgggaaggacggaatgagtagggttttgccagttgggaaccacttgtgggattggtcgtgcaaaacctctgtaatgggagtcttgaatttgactccggacagctttagtgatggaggaaagtttcta

SEQ ID NO: 800 - DNA, 200 bp, Kochia scoparia
tagaaactttcctccatcactaaagctgtccggagtcaaattcaagactcccattacagaggttttgcacgaccaatcccacaagtggttcccaactggcaaaaccctactcattccgtccttcccaatgagtgaatctccaccaagtttatcccacgcttcaaatagtccaccagaaaattttgataatgaatgccatg

SEQ ID NO: 801 - DNA, 200 bp, Kochia scoparia
ctttagtgatggaggaaagtttctacctgtagaaaatgcagtttctcaggttcgtcaaatgatctcagaaggtgctgacataattgatattggagcgcaatcaacaaggcccatggcaactaggatttctgctgaggaagagctggaaagactagtccctgttttagaaggtgtcaaggatgttatcgaggaagaaggaa

SEQ ID NO: 802 - DNA, 200 bp, Kochia scoparia
ttccttcttcctcgataacatccttgacaccttctaaaacagggactagtctttccagctcttcctcagcagaaatcctagttgccatgggccttgttgattgcgctccaatatcaattatgtcagcaccttctgagatcatttgacgaacctgagaaactgcattttctacaggtagaaactttcctccatcactaaag

SEQ ID NO: 803 - DNA, 200 bp, Kochia scoparia
aaggatgttatcgaggaagaaggaagaatgttatcagtggatacattttactcaaaagttgcttctgaagctgtcaataagggagcacatatggtgaatgatgtttcgagtggaaagctagatcctgagatgttcaatgttgttgcagggcttaaagtgccttatatagcaatgcacatgcgaggtgatccttcttcaat

SEQ ID NO: 804 - DNA, 200 bp, Kochia scoparia
attgaagaaggatcacctcgcatgtgcattgctatataaggcactttaagccctgcaacaacattgaacatctcaggatctagctttccactcgaaacatcattcaccatatgtgctcccttattgacagcttcagaagcaacttttgagtaaaatgtatccactgataacattcttccttcttcctcgataacatcctt

SEQ ID NO: 805 - DNA, 200 bp, Kochia scoparia
acatgcgaggtgatccttcttcaatgcaaaatgctgataatttgacatacaatgatgtttgtaaggaggtggctttagagttgagctctaggattacagatgcagaattatcaggtattcctgcttggaggatagttattgatcctggcattggattttccaagaatacaaagcaaaatttggaaattctcacagggttg

SEQ ID NO: 806 - DNA, 200 bp, Kochia scoparia
caaccctgtgagaatttccaaattttgctttgtattcttggaaaatccaatgccaggatcaataactatcctccaagcaggaatacctgataattctgcatctgtaatcctagagctcaactctaaagccacctccttacaaacatcattgtatgtcaaattatcagcattttgcattgaagaaggatcacctcgcatgt

SEQ ID NO: 807 - DNA, 200 bp, Kochia scoparia
aaatttggaaattctcacagggttgaaaagaattcggcaagaaatagcaaaaaagagtttggctgtggctcatggtcccttactgattggaccttcaagaaagaggtttttgggtgagatctgcaatcgtcctgtagcatctgatagagatccggcaactgttgcttctatcaccgctggagttttgggtggtgcaaaca

SEQ ID NO: 808 - DNA, 200 bp, Kochia scoparia
tgtttgcaccacccaaaactccagcggtgatagaagcaacagttgccggatctctatcagatgctacaggacgattgcagatctcacccaaaaacctctttcttgaaggtccaatcagtaagggaccatgagccacagccaaactcttttttgctatttcttgccgaattcttttcaaccctgtgagaatttccaaattt

SEQ ID NO: 809 - DNA, 200 bp, Kochia scoparia
gctggagttttgggtggtgcaaacattgtaagagtacataatgttcgggatagccttgatggtgtcaagctatgtgatgcaatgcttgaatatcagtataactgaagattcttagattcttagattcttagattctaagagttgattttatagattattgtctttgttgtgtgccttcggcagatatgaataatatgatt

SEQ ID NO: 810 - DNA, 200 bp, Kochia scoparia
aatcatattattcatatctgccgaaggcacacaacaaagacaataatctataaaatcaactcttagaatctaagaatctaagaatctaagaatcttcagttatactgatattcaagcattgcatcacatagcttgacaccatcaaggctatcccgaacattatgtactcttacaatgtttgcaccacccaaaactccagc

SEQ ID NO: 811 - DNA, 200 bp, Kochia scoparia
ttcggcagatatgaataatatgattgtaattttgttgcttatgattttcttcctacctttcttttatcatagaatttagattaaaaaatgtacttgaatttcataaatggtgaaataaagtttgtcggtgcttcatgcaatacgacttttctagtttgcattgctctatcataggggctgtaagcagattagcccagtca

SEQ ID NO: 812 - DNA, 200 bp, Kochia scoparia
tgactgggctaatctgcttacagcccctatgatagagcaatgcaaactagaaaagtcgtattgcatgaagcaccgacaaactttatttcaccatttatgaaattcaagtacattttttaatctaaattctatgataaaagaaaggtaggaagaaaatcataagcaacaaaattacaatcatattattcatatctgccgaa

SEQ ID NO: 813 - DNA, 200 bp, Kochia scoparia
ggctgtaagcagattagcccagtcaattcacattagatttagacggccggtgcaaaaacagcataatgttgctaacgctgtttatacatttaaactatatgatctctaagagcaaaatacaaaataatacaatgtgtttcatgaagatgtccaatcatttctccctcaaaaaaagatcgtgatgaacaatcaggtacttt

SEQ ID NO: 814 - DNA, 200 bp, Kochia scoparia
aaagtacctgattgttcatcacgatctttttttgagggagaaatgattggacatcttcatgaaacacattgtattattttgtattttgctcttagagatcatatagtttaaatgtataaacagcgttagcaacattatgctgtttttgcaccggccgtctaaatctaatgtgaattgactgggctaatctgcttacagcc

SEQ ID NO: 815 - DNA, 200 bp, Kochia scoparia
atcgtgatgaacaatcaggtactttgattttgttgtagaacctcagcttcagccctagccatgggatcatctttttctccttctcgctcctcagccaatttctttctgtgcatctcctctgcagcttctattgaagctttgtgctcagcctggagacgctgcatttcctcttccatctgctgtctttcgaaccatgtatc

SEQ ID NO: 816 - DNA, 200 bp, Kochia scoparia
gatacatggttcgaaagacagcagatggaagaggaaatgcagcgtctccaggctgagcacaaagcttcaatagaagctgcagaggagatgcacagaaagaaattggctgaggagcgagaaggagaaaaagatgatcccatggctagggctgaagctgaggttctacaacaaaatcaaagtacctgattgttcatcacgat

SEQ ID NO: 817 - DNA, 200 bp, Kochia scoparia
tctgctgtctttcgaaccatgtatcagcatccgcccaaactgcagaaataatttaaacaagttaatcatacggagtgtgcagcatggcaaattggcaataagatggtgcaagtaacaagctcaaatgatccaccatagccaaattcctaaccattggactgtgtcatatactcatatcagctagtagaaacattctcacc

SEQ ID NO: 818 - DNA, 200 bp, Kochia scoparia
ggtgagaatgtttctactagctgatatgagtatatgacacagtccaatggttaggaatttggctatggtggatcatttgagcttgttacttgcaccatcttattgccaatttgccatgctgcacactccgtatgattaacttgtttaaattatttctgcagtttgggcggatgctgatacatggttcgaaagacagcaga

SEQ ID NO: 819 - DNA, 200 bp, Kochia scoparia
atcagctagtagaaacattctcaccataaccctaactattcgaggataagtgataccattcaacaatagtaaagatcaaatcaggtattactagacctatcaaagtaatacggagtagttgttattgttacagagttaggaagaatcctattttcccatttctcctatcaatcctaaacttttgtgactgaactatcgac

SEQ ID NO: 820 - DNA, 200 bp, Kochia scoparia
gtcgatagttcagtcacaaaagtttaggattgataggagaaatgggaaaataggattcttcctaactctgtaacaataacaactactccgtattactttgataggtctagtaatacctgatttgatctttactattgttgaatggtatcacttatcctcgaatagttagggttatggtgagaatgtttctactagctgat

SEQ ID NO: 821 - DNA, 200 bp, Kochia scoparia
aaacttttgtgactgaactatcgacatatagtgtgaagaacatgaatggcagaagtcacaactctgattcctgagtcctgactataaactattggatgactgtgatacatcattagtcctttaagcattgagttactctattctacatagttaaattatatagattccaagatccgaatccatgaattatcaattaccat

SEQ ID NO: 822 - DNA, 200 bp, Kochia scoparia
atggtaattgataattcatggattcggatcttggaatctatataatttaactatgtagaatagagtaactcaatgcttaaaggactaatgatgtatcacagtcatccaatagtttatagtcaggactcaggaatcagagttgtgacttctgccattcatgttcttcacactatatgtcgatagttcagtcacaaaagttt

SEQ ID NO: 823 - DNA, 200 bp, Kochia scoparia
gaatccatgaattatcaattaccataaattgatcttcttcctttgagttttgtcaacagttcctactccgaaatatgtcttaaatctagaagcaatagacacttatcaatgatacagcaatagacgaattcatctacccaaggcagcatcaacaacagctgccgtttgcgataatcgaaactatcatagtgtccttttca

SEQ ID NO: 824 - DNA, 200 bp, Kochia scoparia
tgaaaaggacactatgatagtttcgattatcgcaaacggcagctgttgttgatgctgccttgggtagatgaattcgtctattgctgtatcattgataagtgtctattgcttctagatttaagacatatttcggagtaggaactgttgacaaaactcaaaggaagaagatcaatttatggtaattgataattcatggattc

SEQ ID NO: 825 - DNA, 200 bp, Kochia scoparia
cgaaactatcatagtgtccttttcactattagtgcagatgttatctagggtctaaacctctatgtttctacattcctggaaaatcttattctgtactgcataatcctctcacaaaaaattcgccaatgaatgacaaattagctatccagaattgcagattgattgaatcaaattgatcatggcagcagcacaattgcagc

SEQ ID NO: 826 - DNA, 200 bp, Kochia scoparia
gctgcaattgtgctgctgccatgatcaatttgattcaatcaatctgcaattctggatagctaatttgtcattcattggcgaattttttgtgagaggattatgcagtacagaataagattttccaggaatgtagaaacatagaggtttagaccctagataacatctgcactaatagtgaaaaggacactatgatagtttcg

SEQ ID NO: 827 - DNA, 200 bp, Kochia scoparia
atcatggcagcagcacaattgcagcagttatcaattgaagcacaatattacctctcaatcataacaatcaaagcacagaaattccaatcaataactccacaatatagtatcaatctctaactcagaaactaaacagaagcaaaaacccattcaatcaaagcaacagtaaaattagggtttgaattagaaagaaagagagg

SEQ ID NO: 828 - DNA, 200 bp, Kochia scoparia
cctctctttctttctaattcaaaccctaattttactgttgctttgattgaatgggtttttgcttctgtttagtttctgagttagagattgatactatattgtggagttattgattggaatttctgtgctttgattgttatgattgagaggtaatattgtgcttcaattgataactgctgcaattgtgctgctgccatgat

SEQ ID NO: 829 - DNA, 200 bp, Kochia scoparia
ggtttgaattagaaagaaagagagggaagagaaacatactgggttgcgcagcccatccctgttcaccacccttaatcttctcgggaagagcgtaattgacagtgagaacacgaccgtaaagctcagcaccatccatgttatccatggcagaagaagcgtcgtctctctccaagaaagtgacgaaaccgaaagaacgatgc

SEQ ID NO: 830 - DNA, 200 bp, Kochia scoparia
gcatcgttctttcggtttcgtcactttcttggagagagacgacgcttcttctgccatggataacatggatggtgctgagctttacggtcgtgttctcactgtcaattacgctcttcccgagaagattaagggtggtgaacagggatgggctgcgcaacccagtatgtttctcttccctctctttctttctaattcaaacc

SEQ ID NO: 831 - DNA, 200 bp, Lolium multiflorum
acgaaagctttgaactctcaagtggtgcaacatagatagattatatatatttcgaatataattcgaatgaacataaaaattacatataaaaactttaaaacaaacttaaactacttcttcttcctcgatggccccgcctcatcgtcggggcggcgacgcttccggctcgtcacctcctcgtcggaggaagttgggtcctc

SEQ ID NO: 832 - DNA, 200 bp, Lolium multiflorum
gaggacccaacttcctccgacgaggaggtgacgagccggaagcgtcgccgccccgacgatgaggcggggccatcgaggaagaagaagtagtttaagtttgttttaaagtttttatatgtaatttttatgttcattcgaattatattcgaaatatatataatctatctatgttgcaccacttgagagttcaaagctttcgt

SEQ ID NO: 833 - DNA, 200 bp, Lolium multiflorum
cctcgtcggaggaagttgggtcctcctcctcgtcagtgtcctcagcctcttcgtcgtcctcctcctcttcctcggcctcttcgtcggcctgctccttggcctcttcgtcctcctcctcgtcgtcatcccatgcgtcctcctcctcgtcgtcatcccattcgtccttgctggccggcgggggggaatcgtcgctgctggac

SEQ ID NO: 834 - DNA, 200 bp, Lolium multiflorum
gtccagcagcgacgattcccccccgccggccagcaaggacgaatgggatgacgacgaggaggaggacgcatgggatgacgacgaggaggaggacgaagaggccaaggagcaggccgacgaagaggccgaggaagaggaggaggacgacgaagaggctgaggacactgacgaggaggaggacccaacttcctccgacgagg

SEQ ID NO: 835 - DNA, 200 bp, Lolium multiflorum
cgggggggaatcgtcgctgctggacgatgcagctttatcccaccaatggcgccatccaggcggcttcccctcgctgtcggtgtccgatggccaagagagatcgctcatggttgacggaggatggtgaggagagaacagatgaatggcgtcggccgtcgtaaaccgtatatgtaggtctctcgacgaagagagcggcggtt

SEQ ID NO: 836 - DNA, 200 bp, Lolium multiflorum
aaccgccgctctcttcgtcgagagacctacatatacggtttacgacggccgacgccattcatctgttctctcctcaccatcctccgtcaaccatgagcgatctctcttggccatcggacaccgacagcgaggggaagccgcctggatggcgccattggtgggataaagctgcatcgtccagcagcgacgattcccccccg

SEQ ID NO: 837 - DNA, 200 bp, Lolium multiflorum
tctctcgacgaagagagcggcggttgctcttccgcggagttcgtgctccattacagcggttctcgcatcgaggccacttcggccgttcccgacgagtcgtttgggctctccagaccactttgcgacggttcaatgcgtcgagggcactccgacgattgcccttcccggtgactgcgccgtcgctatgcacgcggtgtgtg

SEQ ID NO: 838 - DNA, 200 bp, Lolium multiflorum
cacacaccgcgtgcatagcgacggcgcagtcaccgggaagggcaatcgtcggagtgccctcgacgcattgaaccgtcgcaaagtggtctggagagcccaaacgactcgtcgggaacggccgaagtggcctcgatgcgagaaccgctgtaatggagcacgaactccgcggaagagcaaccgccgctctcttcgtcgagaga

SEQ ID NO: 839 - DNA, 200 bp, Lolium multiflorum
gccgtcgctatgcacgcggtgtgtgcgcaaccatgggctcgcggctgggaaaatgggcctccccaggccacaaaatacatccatccggcgataactaacgccggatttcggcctgggagccccaacggctgggatgctcttagcccgtggaacacattcccgaatcggtaccgacggtctcatctctctcgccctgatct

SEQ ID NO: 840 - DNA, 200 bp, Lolium multiflorum
agatcagggcgagagagatgagaccgtcggtaccgattcgggaatgtgttccacgggctaagagcatcccagccgttggggctcccaggccgaaatccggcgttagttatcgccggatggatgtattttgtggcctggggaggcccattttcccagccgcgagcccatggttgcgcacacaccgcgtgcatagcgacggc

SEQ ID NO: 841 - DNA, 200 bp, Lolium multiflorum
ggtctcatctctctcgccctgatctatctctcttccgcgatctggaagggccgacggcgtggcggcgcaaccacgggcgctgatgggcggcggcgcgcagtctgttaagcaggacgcctgcttctcgctccgtcctccctctccaaccgagcggcacgaggtagacccggagaccccaactccgtcctagcccagtggcg

SEQ ID NO: 842 - DNA, 200 bp, Lolium multiflorum
cgccactgggctaggacggagttggggtctccgggtctacctcgtgccgctcggttggagagggaggacggagcgagaagcaggcgtcctgcttaacagactgcgcgccgccgcccatcagcgcccgtggttgcgccgccacgccgtcggcccttccagatcgcggaagagagatagatcagggcgagagagatgagacc

SEQ ID NO: 843 - DNA, 200 bp, Lolium multiflorum
ccaactccgtcctagcccagtggcgcccgtgaggcgtcatctccggcgccctctccatctccgtctgcttcgcacttgtatgcacatactcttcctccttaccccagtttcttggtcttggcttgtttgattacgatttctagggttcttggcatgctcggtagttgcagatttgtcttctgttgggtgtttgtttccat

SEQ ID NO: 844 - DNA, 200 bp, Lolium multiflorum
atggaaacaaacacccaacagaagacaaatctgcaactaccgagcatgccaagaaccctagaaatcgtaatcaaacaagccaagaccaagaaactggggtaaggaggaagagtatgtgcatacaagtgcgaagcagacggagatggagagggcgccggagatgacgcctcacgggcgccactgggctaggacggagttgg

SEQ ID NO: 845 - DNA, 200 bp, Lolium multiflorum
tcttctgttgggtgtttgtttccatggccttggctgcatccatcgtttcgtattatgtagttgtttgtttccatgccattgcttgtatccatcaattggaaagggaagaaagggctacagggatattaggaattagatcagtccttcctagctgattttctctgacaggttgttagtctcgcgcatgtggtggacttctt

SEQ ID NO: 846 - DNA, 200 bp, Lolium multiflorum
aagaagtccaccacatgcgcgagactaacaacctgtcagagaaaatcagctaggaaggactgatctaattcctaatatccctgtagccctttcttccctttccaattgatggatacaagcaatggcatggaaacaaacaactacataatacgaaacgatggatgcagccaaggccatggaaacaaacacccaacagaaga

SEQ ID NO: 847 - DNA, 200 bp, Lolium multiflorum
gtctcgcgcatgtggtggacttcttgtgcttttggctagctccaagtctgatgtgtagctgctttctgagattttccacataaacgttgctatggctagtaagttgtggctaactaaatccttgggcacaagctagtaatctgaatggcatatccgcccccatgcctttaattgttgcctaacgatgggctaggttctct

SEQ ID NO: 848 - DNA, 200 bp, Lolium multiflorum
agagaacctagcccatcgttaggcaacaattaaaggcatgggggcggatatgccattcagattactagcttgtgcccaaggatttagttagccacaacttactagccatagcaacgtttatgtggaaaatctcagaaagcagctacacatcagacttggagctagccaaaagcacaagaagtccaccacatgcgcgagac

SEQ ID NO: 849 - DNA, 200 bp, Lolium multiflorum
tgcctaacgatgggctaggttctcttatttattcatttttgttaaaagaagaacttgggcatgctaattcagcaaaatagaaaaaatatgtgttgtttctgtatatagaggttcttggttttcccttgccgattctgaccgatttgcgggttttttttttttttgtctttcctcatcgtcaggcaagaagagcaagatgt

SEQ ID NO: 850 - DNA, 200 bp, Lolium multiflorum
acatcttgctcttcttgcctgacgatgaggaaagacaaaaaaaaaaaaaacccgcaaatcggtcagaatcggcaagggaaaaccaagaacctctatatacagaaacaacacatattttttctattttgctgaattagcatgcccaagttcttcttttaacaaaaatgaataaataagagaacctagcccatcgttaggca

SEQ ID NO: 851 - DNA, 200 bp, Lolium multiflorum
tcgtcaggcaagaagagcaagatgtgacagaggatttaagcagcagcagaactagacctggctcctatgctctaagatgcttcgtgccaaggacacattcaggaagatgtactcagctgccaagggctactactgcggagggctagcgtctcctaatgccttctcaggtagcttgcccaaagttccccaataataactag

SEQ ID NO: 852 - DNA, 200 bp, Lolium multiflorum
ctagttattattggggaactttgggcaagctacctgagaaggcattaggagacgctagccctccgcagtagtagcccttggcagctgagtacatcttcctgaatgtgtccttggcacgaagcatcttagagcataggagccaggtctagttctgctgctgcttaaatcctctgtcacatcttgctcttcttgcctgacga

SEQ ID NO: 853 - DNA, 200 bp, Lolium multiflorum
cccaaagttccccaataataactagggaacatctcttttccacctctttcacattccaaccaaagacagtcctaactcgtttcccctctgaacataccagcattggatcttcggcatcctctcagctcctcaagaaaaccaccaggccacgctaacctgttcagggtccgttcatacacgcaatgcgccctcaccaacga

SEQ ID NO: 854 - DNA, 200 bp, Lolium multiflorum
tcgttggtgagggcgcattgcgtgtatgaacggaccctgaacaggttagcgtggcctggtggttttcttgaggagctgagaggatgccgaagatccaatgctggtatgttcagaggggaaacgagttaggactgtctttggttggaatgtgaaagaggtggaaaagagatgttccctagttattattggggaactttggg

SEQ ID NO: 855 - DNA, 200 bp, Lolium multiflorum
acacgcaatgcgccctcaccaacgattccgccgaccaggaggtcgtgattgccctgggcagcaacgtgggagacagggtcagcatgttcgacagggcgctgcggctgatgaggagctcgggcatcaaggtcaccaggcacgcctgcctgtacgagaccgccccagcctacgtgaccgaccagccgcggttcctcaactct

SEQ ID NO: 856 - DNA, 200 bp, Lolium multiflorum
agagttgaggaaccgcggctggtcggtcacgtaggctggggcggtctcgtacaggcaggcgtgcctggtgaccttgatgcccgagctcctcatcagccgcagcgccctgtcgaacatgctgaccctgtctcccacgttgctgcccagggcaatcacgacctcctggtcggcggaatcgttggtgagggcgcattgcgtgt

SEQ ID NO: 857 - DNA, 200 bp, Lolium multiflorum
cgaccagccgcggttcctcaactctgccgttaggggcacgacgaagctggggcctcacgagttgctcaggaagctcaaggagatcgagaaggatataggacgtaccgccggggtaaggtacggcccgaggccgatcgatctggacattcttctgtatggggactcccggatcaagaccgagtctctgattgtgccgcatg

SEQ ID NO: 858 - DNA, 200 bp, Lolium multiflorum
catgcggcacaatcagagactcggtcttgatccgggagtccccatacagaagaatgtccagatcgatcggcctcgggccgtaccttaccccggcggtacgtcctatatccttctcgatctccttgagcttcctgagcaactcgtgaggccccagcttcgtcgtgcccctaacggcagagttgaggaaccgcggctggtcg

SEQ ID NO: 859 - DNA, 200 bp, Lolium multiflorum
accgagtctctgattgtgccgcatgaacgcatccatgagagaccgttcgtcttggcgcctcttgttgacctcctgggttcgtcggctgaggacggtatggagaaaagctggcactctctctcgaagtgcagtggcgggttctttgatttgtggaacaagctcggcggtgaatctgttgttggaacagaaggcattaaaag

SEQ ID NO: 860 - DNA, 200 bp, Lolium multiflorum
cttttaatgccttctgttccaacaacagattcaccgccgagcttgttccacaaatcaaagaacccgccactgcacttcgagagagagtgccagcttttctccataccgtcctcagccgacgaacccaggaggtcaacaagaggcgccaagacgaacggtctctcatggatgcgttcatgcggcacaatcagagactcggt

SEQ ID NO: 861 - DNA, 200 bp, Lolium multiflorum
ttgttggaacagaaggcattaaaagggtcatgtctgttggaaatacgctgttggactggcgtcagaggacccttgttatgggggtgcttaacctcacgccagacagcttcagcgacggaggtaagtttcaagaagtggaagctgccatttcccaggccaggttgttgatatcagaaggtgctgacataattgatattggt

SEQ ID NO: 862 - DNA, 200 bp, Lolium multiflorum
accaatatcaattatgtcagcaccttctgatatcaacaacctggcctgggaaatggcagcttccacttcttgaaacttacctccgtcgctgaagctgtctggcgtgaggttaagcacccccataacaagggtcctctgacgccagtccaacagcgtatttccaacagacatgacccttttaatgccttctgttccaacaa

SEQ ID NO: 863 - DNA, 200 bp, Lolium multiflorum
aggtgctgacataattgatattggtgcccagtccaccaggccctttgcaaggaggctatccgcagaagaagagcttgagaggctggtccctgttctggatgctatcatgaaactcccagagatggaagggaagttgctttcggtagacacgttctacgcgcaagtcgctgctgaagctgtaaaaagaggagccaccatga

SEQ ID NO: 864 - DNA, 200 bp, Lolium multiflorum
tcatggtggctcctctttttacagcttcagcagcgacttgcgcgtagaacgtgtctaccgaaagcaacttcccttccatctctgggagtttcatgatagcatccagaacagggaccagcctctcaagctcttcttctgcggatagcctccttgcaaagggcctggtggactgggcaccaatatcaattatgtcagcacct

SEQ ID NO: 865 - DNA, 200 bp, Lolium multiflorum
gctgtaaaaagaggagccaccatgatcaatgatgtatctggtgggcagcttgacccaagtattcttcaagttgttgctgaactgggagttccgtatgttaccatgcacatgagaggcgatccatcaactatgcagaatgaacagaatctacagtatgatgatgtctgcaaagaagttgcttctgagttatacgcccggtt

SEQ ID NO: 866 - DNA, 200 bp, Lolium multiflorum
aaccgggcgtataactcagaagcaacttctttgcagacatcatcatactgtagattctgttcattctgcatagttgatggatcgcctctcatgtgcatggtaacatacggaactcccagttcagcaacaacttgaagaatacttgggtcaagctgcccaccagatacatcattgatcatggtggctcctctttttacagc

SEQ ID NO: 867 - DNA, 200 bp, Lolium multiflorum
ttgcttctgagttatacgcccggttgagagcagcagagctttctggaattcctttgtggaggattattcttgaccctggcattgggttttctaagaaatccacacagaatattgaagtaatcgcgggtttggaatccattagagaagagatgggtaaaatgagcctaggtgcttcacatgtgccaatattacttggaccc

SEQ ID NO: 868 - DNA, 200 bp, Lolium multiflorum
gggtccaagtaatattggcacatgtgaagcacctaggctcattttacccatctcttctctaatggattccaaacccgcgattacttcaatattctgtgtggatttcttagaaaacccaatgccagggtcaagaataatcctccacaaaggaattccagaaagctctgctgctctcaaccgggcgtataactcagaagcaa

SEQ ID NO: 869 - DNA, 200 bp, Lolium multiflorum
acatgtgccaatattacttggaccctcgaggaaaagtttcctaggtgaaatatgtagtcgcgccgatccagttcagagagatgctgctactgcttctgccgttacaattgcgatcttgaatggcgctaatatagtaagggtccataatgttagatacaatgtggatgctgcaaaggtctctgatgcattactcaagtaca

SEQ ID NO: 870 - DNA, 200 bp, Lolium multiflorum
tgtacttgagtaatgcatcagagacctttgcagcatccacattgtatctaacattatggacccttactatattagcgccattcaagatcgcaattgtaacggcagaagcagtagcagcatctctctgaactggatcggcgcgactacatatttcacctaggaaacttttcctcgagggtccaagtaatattggcacatgt

SEQ ID NO: 871 - DNA, 200 bp, Lolium multiflorum
gtctctgatgcattactcaagtacagaagaaaataatagaaagtatacagcccggatagaagccataccagttgccagttttgtgcaagggaatgctgatgtggagctcaaaccatatggggtgtatcatcacttaataagatcaaagcatgcgtgaataactattacagtgaacaagagatatacagttttttcgtgcg

SEQ ID NO: 872 - DNA, 200 bp, Lolium multiflorum
cgcacgaaaaaactgtatatctcttgttcactgtaatagttattcacgcatgctttgatcttattaagtgatgatacaccccatatggtttgagctccacatcagcattcccttgcacaaaactggcaactggtatggcttctatccgggctgtatactttctattattttcttctgtacttgagtaatgcatcagagac

SEQ ID NO: 873 - DNA, 200 bp, Lolium multiflorum
aagagatatacagttttttcgtgcgcctcctccttcgtagaatcttcaaagaagcaaaggtaacacagacctaggcctctggggttggtatatgatgtaaccagttttgtgagggtttaataattatctaatagtgagacctaagagtaagacctttataatatactttgcttggaatttcctacttgagagcatcattt

SEQ ID NO: 874 - DNA, 200 bp, Lolium multiflorum
aaatgatgctctcaagtaggaaattccaagcaaagtatattataaaggtcttactcttaggtctcactattagataattattaaaccctcacaaaactggttacatcatataccaaccccagaggcctaggtctgtgttacctttgcttctttgaagattctacgaaggaggaggcgcacgaaaaaactgtatatctctt

SEQ ID NO: 875 - DNA, 200 bp, Lolium multiflorum
agctcctcaagaaaaccaccaggccacgctaacctgttcagggtccgttcatacacgcaatgcgccctcaccaacgattccgccgaccaggaggtcgtgattgccctgggcagcaacgtgggagacagggtcagcatgttcgacagggcgctgcggctgatgaggagctcgggcatcaaggtcaccaggcacgcctgcct

SEQ ID NO: 876 - DNA, 200 bp, Lolium multiflorum
aggcaggcgtgcctggtgaccttgatgcccgagctcctcatcagccgcagcgccctgtcgaacatgctgaccctgtctcccacgttgctgcccagggcaatcacgacctcctggtcggcggaatcgttggtgagggcgcattgcgtgtatgaacggaccctgaacaggttagcgtggcctggtggttttcttgaggagct

SEQ ID NO: 877 - DNA, 200 bp, Lolium multiflorum
tcaaggtcaccaggcacgcctgcctgtacgagaccgccccagcctacgtgaccgaccagccgcggttcctcaactctgccgttaggggcacgacgaagctggggcctcacgagttgctcaggaagctcaaggagatcgagaaggatataggacgtaccgccggggtaaggtacggcccgaggccgatcgatctggacatt

SEQ ID NO: 878 - DNA, 200 bp, Lolium multiflorum
aatgtccagatcgatcggcctcgggccgtaccttaccccggcggtacgtcctatatccttctcgatctccttgagcttcctgagcaactcgtgaggccccagcttcgtcgtgcccctaacggcagagttgaggaaccgcggctggtcggtcacgtaggctggggcggtctcgtacaggcaggcgtgcctggtgaccttga

SEQ ID NO: 879 - DNA, 200 bp, Lolium multiflorum
cccgaggccgatcgatctggacattcttctgtatggggactcccggatcaagaccgagtctctgattgtgccgcatgaacgcatccatgagagaccgttcgtcttggcgcctcttgttgacctcctgggttcgtcggctgaggacggtatggagaaaagctggcactctctctcgaagtgcagtggcgggttctttgatt

SEQ ID NO: 880 - DNA, 200 bp, Lolium multiflorum
aatcaaagaacccgccactgcacttcgagagagagtgccagcttttctccataccgtcctcagccgacgaacccaggaggtcaacaagaggcgccaagacgaacggtctctcatggatgcgttcatgcggcacaatcagagactcggtcttgatccgggagtccccatacagaagaatgtccagatcgatcggcctcggg

SEQ ID NO: 881 - DNA, 200 bp, Lolium multiflorum
aagtgcagtggcgggttctttgatttgtggaacaagctcggcggtgaatctgttgttggaacagaaggcattaaaagggtcatgtctgttggaaatacgctgttggactggcgtcagaggacccttgttatgggggtgcttaacctcacgccagacagcttcagcgacggaggtaagtttcaagaagtggaagctgccat

SEQ ID NO: 882 - DNA, 200 bp, Lolium multiflorum
atggcagcttccacttcttgaaacttacctccgtcgctgaagctgtctggcgtgaggttaagcacccccataacaagggtcctctgacgccagtccaacagcgtatttccaacagacatgacccttttaatgccttctgttccaacaacagattcaccgccgagcttgttccacaaatcaaagaacccgccactgcactt

SEQ ID NO: 883 - DNA, 200 bp, Lolium multiflorum
agtttcaagaagtggaagctgccatttcccaggccaggttgttgatatcagaaggtgctgacataattgatattggtgcccagtccaccaggccctttgcaaggaggctatccgcagaagaagagcttgagaggctggtccctgttctggatgctatcatgaaactcccagagatggaagggaagttgctttcggtagac

SEQ ID NO: 884 - DNA, 200 bp, Lolium multiflorum
gtctaccgaaagcaacttcccttccatctctgggagtttcatgatagcatccagaacagggaccagcctctcaagctcttcttctgcggatagcctccttgcaaagggcctggtggactgggcaccaatatcaattatgtcagcaccttctgatatcaacaacctggcctgggaaatggcagcttccacttcttgaaact

SEQ ID NO: 885 - DNA, 200 bp, Lolium multiflorum
ggaagggaagttgctttcggtagacacgttctacgcgcaagtcgctgctgaagctgtaaaaagaggagccaccatgatcaatgatgtatctggtgggcagcttgacccaagtattcttcaagttgttgctgaactgggagttccgtatgttaccatgcacatgagaggcgatccatcaactatgcagaatgaacagaatc

SEQ ID NO: 886 - DNA, 200 bp, Lolium multiflorum
gattctgttcattctgcatagttgatggatcgcctctcatgtgcatggtaacatacggaactcccagttcagcaacaacttgaagaatacttgggtcaagctgcccaccagatacatcattgatcatggtggctcctctttttacagcttcagcagcgacttgcgcgtagaacgtgtctaccgaaagcaacttcccttcc

SEQ ID NO: 887 - DNA, 200 bp, Lolium multiflorum
tcaactatgcagaatgaacagaatctacagtatgatgatgtctgcaaagaagttgcttctgagttatacgcccggttgagagcagcagagctttctggaattcctttgtggaggattattcttgaccctggcattgggttttctaagaaatccacacagaatattgaagtaatcgcgggtttggaatccattagagaaga

SEQ ID NO: 888 - DNA, 200 bp, Lolium multiflorum
tcttctctaatggattccaaacccgcgattacttcaatattctgtgtggatttcttagaaaacccaatgccagggtcaagaataatcctccacaaaggaattccagaaagctctgctgctctcaaccgggcgtataactcagaagcaacttctttgcagacatcatcatactgtagattctgttcattctgcatagttga

SEQ ID NO: 889 - DNA, 200 bp, Lolium multiflorum
cgggtttggaatccattagagaagagatgggtaaaatgagcctaggtgcttcacatgtgccaatattacttggaccctcgaggaaaagtttcctaggtgaaatatgtagtcgcgccgatccagttcagagagatgctgctactgcttctgccgttacaattgcgatcttgaatggcgctaatatagtaagggtccataat

SEQ ID NO: 890 - DNA, 200 bp, Lolium multiflorum
attatggacccttactatattagcgccattcaagatcgcaattgtaacggcagaagcagtagcagcatctctctgaactggatcggcgcgactacatatttcacctaggaaacttttcctcgagggtccaagtaatattggcacatgtgaagcacctaggctcattttacccatctcttctctaatggattccaaacccg

SEQ ID NO: 891 - DNA, 200 bp, Lolium multiflorum
aggatatagggcgcaccgccgggataaggtacggtccgaggccgatcgatctggacattcttctgtatggggattcccggatcaagaccgagtctctgactgtgccgcatgaacgcatccatgagagaccgttcgtcttggcgcctcttgttgacctcctgggttcatcgggtgaggatggtatggagagaagatggcac

SEQ ID NO: 892 - DNA, 200 bp, Lolium multiflorum
gtgccatcttctctccataccatcctcacccgatgaacccaggaggtcaacaagaggcgccaagacgaacggtctctcatggatgcgttcatgcggcacagtcagagactcggtcttgatccgggaatccccatacagaagaatgtccagatcgatcggcctcggaccgtaccttatcccggcggtgcgccctatatcct

SEQ ID NO: 893 - DNA, 200 bp, Lolium multiflorum
agggtcatgtctgttggaaatacgctgttggattggcgggagaggaccctggttatgggggtgcttaacctcacgccagacagctttagcgatggaggtaagtttcaagaggtggaagctgccatttctcaggccaggctgttaatttcagaaggtgcaggcataattgatattggcgctcagtccaccaggccctttgc

SEQ ID NO: 894 - DNA, 200 bp, Lolium multiflorum
gcaaagggcctggtggactgagcgccaatatcaattatgcctgcaccttctgaaattaacagcctggcctgagaaatggcagcttccacctcttgaaacttacctccatcgctaaagctgtctggcgtgaggttaagcacccccataaccagggtcctctcccgccaatccaacagcgtatttccaacagacatgaccct

SEQ ID NO: 895 - DNA, 200 bp, Lolium multiflorum
gcgctcagtccaccaggccctttgcaaggtggctatctgcagaagaagagcttgagaggctggtccctgttcttaatgctatcatgaaactccccgagatggaagggaagttgctttcagtagatacgttctacgcgcaagtcgcggctgaagccgtgaaaagaggggccaccatgatcaatgatgtatctggtgggcag

SEQ ID NO: 896 - DNA, 200 bp, Lolium multiflorum
ctgcccaccagatacatcattgatcatggtggcccctcttttcacggcttcagccgcgacttgcgcgtagaacgtatctactgaaagcaacttcccttccatctcggggagtttcatgatagcattaagaacagggaccagcctctcaagctcttcttctgcagatagccaccttgcaaagggcctggtggactgagcgc

SEQ ID NO: 897 - DNA, 200 bp, Lolium multiflorum
gatcaatgatgtatctggtgggcagcttgacccagatattcttcaagttgttgctgaattgggagttccgtatgttgccatgcacatgagaggggatccatcaactatgcagaatgagcagaatttacagtatgatgatgtctgcaaagaagttgcttctgagttatactctcggttgagagcagcagagttgtctggaa

SEQ ID NO: 898 - DNA, 200 bp, Lolium multiflorum
ttccagacaactctgctgctctcaaccgagagtataactcagaagcaacttctttgcagacatcatcatactgtaaattctgctcattctgcatagttgatggatcccctctcatgtgcatggcaacatacggaactcccaattcagcaacaacttgaagaatatctgggtcaagctgcccaccagatacatcattgatc

SEQ ID NO: 899 - DNA, 200 bp, Lolium multiflorum
ttgagagcagcagagttgtctggaattcctttgtggaggattattcttgaccctggcattgggttttccaagaaatccacacagaatattgaagtaatcgcgggcttggaatccattagagaagagatgggtaaaatgagtctaggtgcttcacatgtgccaatattacttggaccctccaggaaacgtttcttagggca

SEQ ID NO: 900 - DNA, 200 bp, Lolium multiflorum
tgccctaagaaacgtttcctggagggtccaagtaatattggcacatgtgaagcacctagactcattttacccatctcttctctaatggattccaagcccgcgattacttcaatattctgtgtggatttcttggaaaacccaatgccagggtcaagaataatcctccacaaaggaattccagacaactctgctgctctcaa

SEQ ID NO: 901 - DNA, 200 bp, Lolium multiflorum
cgctaacctgttcagggtccgttcgtacatgcaatgcgcggtcaccaacgattccgccgaccaagagatcgtgattgccctgggcagcaacgtgggagacagggtcagcacgttcgacagggcgctgcggctgatgaggagctcgggcatcaggatcaccaggcacgcctgcctgtacgagaccgcccctgcttacgtga

SEQ ID NO: 902 - DNA, 200 bp, Lolium multiflorum
tcacgtaagcaggggcggtctcgtacaggcaggcgtgcctggtgatcctgatgcccgagctcctcatcagccgcagcgccctgtcgaacgtgctgaccctgtctcccacgttgctgcccagggcaatcacgatctcttggtcggcggaatcgttggtgaccgcgcattgcatgtacgaacggaccctgaacaggttagcg

SEQ ID NO: 903 - DNA, 200 bp, Lolium multiflorum
gatatagggcgcaccgccgggataaggtacggtccgaggccgatcgatctggacattcttctgtatggggattcccggatcaagaccgagtctctgactgtgccgcatgaacgcatccatgagagaccgttcgtcttggcgcctcttgttgacctcctgggttcatcgggtgaggatggtatggagagaagatggcactc

SEQ ID NO: 904 - DNA, 200 bp, Lolium multiflorum
gagtgccatcttctctccataccatcctcacccgatgaacccaggaggtcaacaagaggcgccaagacgaacggtctctcatggatgcgttcatgcggcacagtcagagactcggtcttgatccgggaatccccatacagaagaatgtccagatcgatcggcctcggaccgtaccttatcccggcggtgcgccctatatc

SEQ ID NO: 905 - DNA, 150 bp, Lolium multiflorum; identical to SEQ ID NO: 54

SEQ ID NO: 906 - DNA, 150 bp, Lolium multiflorum
cacgtaagcaggggcggtctcgtacaggcaggcgtgcctggtgatcctgatgcccgagctcctcatcagccgcagcgccctgtcgaacgtgctgaccctgtctcccacgttgctgcccagggcaatcacgatctcttggtcggcggaatc

SEQ ID NO: 907 - DNA, 21 bp, Unknown
  Found in multiple species
ttatgtactcttacaatgttt

SEQ ID NO: 908 - DNA, 21 bp, Unknown
  Found in multiple species
tgtactcttacaatgtttgca

SEQ ID NO: 909 - DNA, 21 bp, Unknown
  Found in multiple species
tggtgcaaacattgtaagagt

SEQ ID NO: 910 - DNA, 21 bp, Unknown
  Found in multiple species
tgaaaggatatgggaaagacc

SEQ ID NO: 911 - DNA, 21 bp, Unknown
  Found in multiple species
tctttcccatatcctttcatg

SEQ ID NO: 912 - DNA, 21 bp, Unknown
  Found in multiple species
tcttacaatgtttgcaccacc

SEQ ID NO: 913 - DNA, 21 bp, Unknown
  Found in multiple species
tatgtactcttacaatgtttg

SEQ ID NO: 914 - DNA, 21 bp, Unknown
  Found in multiple species
tactcttacaatgtttgcacc

SEQ ID NO: 915 - DNA, 21 bp, Unknown
  Found in multiple species
gtggtgcaaacattgtaagag

SEQ ID NO: 916 - DNA, 21 bp, Unknown
  Found in multiple species
gtgcaaacattgtaagagtac

SEQ ID NO: 917 - DNA, 21 bp, Unknown
  Found in multiple species
gtctttcccatatcctttcat

SEQ ID NO: 918 - DNA, 21 bp, Unknown
  Found in multiple species
gcaaacattgtaagagtacat

SEQ ID NO: 919 - DNA, 21 bp, Unknown
  Found in multiple species
cattgtaagagtacataatgt

SEQ ID NO: 920 - DNA, 21 bp, Unknown
  Found in multiple species
cattatgtactcttacaatgt

SEQ ID NO: 921 - DNA, 21 bp, Unknown
  Found in multiple species
attatgtactcttacaatgtt

SEQ ID NO: 922 - DNA, 21 bp, Unknown
  Found in multiple species
ttttctacatcagacccaata

SEQ ID NO: 923 - DNA, 21 bp, Unknown
  Found in multiple species
ttttcattccatccttcccga

SEQ ID NO: 924 - DNA, 21 bp, Unknown
  Found in multiple species
ttttcaatctgtcgatactgc

SEQ ID NO: 925 - DNA, 21 bp, Unknown
  Found in multiple species
ttttatgagaccaatcccata

SEQ ID NO: 926 - DNA, 21 bp, Unknown
  Found in multiple species
ttttaggtggtgcaaacattg

SEQ ID NO: 927 - DNA, 21 bp, Unknown
  Found in multiple species
tttgcaccacctaaaactcca

SEQ ID NO: 928 - DNA, 21 bp, Unknown
  Found in multiple species
tttctacatcagacccaatac

SEQ ID NO: 929 - DNA, 21 bp, Unknown
  Found in multiple species
tttcccatatcctttcatggg

SEQ ID NO: 930 - DNA, 21 bp, Unknown
  Found in multiple species
tttcattccatccttcccgat

SEQ ID NO: 931 - DNA, 21 bp, Unknown
  Found in multiple species
tttcaatctgtcgatactgcg

SEQ ID NO: 932 - DNA, 21 bp, Unknown
  Found in multiple species
tttatgagaccaatcccataa

SEQ ID NO: 933 - DNA, 21 bp, Unknown
  Found in multiple species
tttaggtggtgcaaacattgt

SEQ ID NO: 934 - DNA, 21 bp, Unknown
  Found in multiple species
tttagggtgagctctgagagc

SEQ ID NO: 935 - DNA, 21 bp, Unknown
  Found in multiple species
ttgtattgggtctgatgtaga

SEQ ID NO: 936 - DNA, 21 bp, Unknown
  Found in multiple species
ttgtaagagtacataatgtga

SEQ ID NO: 937 - DNA, 21 bp, Unknown
  Found in multiple species
ttggtctcataaaacctctgt

SEQ ID NO: 938 - DNA, 21 bp, Unknown
  Found in multiple species
ttgggtctgatgtagaaaatg

SEQ ID NO: 939 - DNA, 21 bp, Unknown
  Found in multiple species
ttggcctaggaccatacctta

SEQ ID NO: 940 - DNA, 21 bp, Unknown
  Found in multiple species
ttggacatactattttatggg

SEQ ID NO: 941 - DNA, 21 bp, Unknown
  Found in multiple species
ttgctaaggttcgtcagatga

SEQ ID NO: 942 - DNA, 21 bp, Unknown
  Found in multiple species
ttgcaccacctaaaactccag

SEQ ID NO: 943 - DNA, 21 bp, Unknown
  Found in multiple species
ttgattgtattgggtctgatg

SEQ ID NO: 944 - DNA, 21 bp, Unknown
  Found in multiple species
ttgacttggacatactatttt

SEQ ID NO: 945 - DNA, 21 bp, Unknown
  Found in multiple species
ttgactcctgatagctttagt

SEQ ID NO: 946 - DNA, 21 bp, Unknown
  Found in multiple species
ttgaacttgactcctgatagc

SEQ ID NO: 947 - DNA, 21 bp, Unknown
  Found in multiple species
ttctacatcagacccaataca

SEQ ID NO: 948 - DNA, 21 bp, Unknown
  Found in multiple species
ttccgccatcactaaagctat

SEQ ID NO: 949 - DNA, 21 bp, Unknown
  Found in multiple species
ttccctaatcgggaaggatgg

SEQ ID NO: 950 - DNA, 21 bp, Unknown
  Found in multiple species
ttcccgattagggaacttcca

SEQ ID NO: 951 - DNA, 21 bp, Unknown
  Found in multiple species
ttcccatatcctttcatgggg

SEQ ID NO: 952 - DNA, 21 bp, Unknown
  Found in multiple species
ttcccataaaatagtatgtcc

SEQ ID NO: 953 - DNA, 21 bp, Unknown
  Found in multiple species
ttccatccttcccgattaggg

SEQ ID NO: 954 - DNA, 21 bp, Unknown
  Found in multiple species
ttcattccatccttcccgatt

SEQ ID NO: 955 - DNA, 21 bp, Unknown
  Found in multiple species
ttcaatctgtcgatactgcgg

SEQ ID NO: 956 - DNA, 21 bp, Unknown
  Found in multiple species
ttcaagactcccattacagag

SEQ ID NO: 957 - DNA, 21 bp, Unknown
  Found in multiple species
ttatgagaccaatcccataag

SEQ ID NO: 958 - DNA, 21 bp, Unknown
  Found in multiple species
ttatccctcacattatgtact

SEQ ID NO: 959 - DNA, 21 bp, Unknown
  Found in multiple species
ttatatagcaatgcacatgcg

SEQ ID NO: 960 - DNA, 21 bp, Unknown
  Found in multiple species
ttaggtggtgcaaacattgta

SEQ ID NO: 961 - DNA, 21 bp, Unknown
  Found in multiple species
ttagggtgagctctgagagcc

SEQ ID NO: 962 - DNA, 21 bp, Unknown
  Found in multiple species
ttagggaacttccaccgagtt

SEQ ID NO: 963 - DNA, 21 bp, Unknown
  Found in multiple species
ttagcaaccgcagtatcgaca

SEQ ID NO: 964 - DNA, 21 bp, Unknown
  Found in multiple species
ttacaatgtttgcaccaccta

SEQ ID NO: 965 - DNA, 21 bp, Unknown
  Found in multiple species
tgtttgcaccacctaaaactc

SEQ ID NO: 966 - DNA, 21 bp, Unknown
  Found in multiple species
tgtgagggataaccttgatgc

SEQ ID NO: 967 - DNA, 21 bp, Unknown
  Found in multiple species
tgtccaagtcaattggcctag

SEQ ID NO: 968 - DNA, 21 bp, Unknown
  Found in multiple species
tgtcattttctacatcagacc

SEQ ID NO: 969 - DNA, 21 bp, Unknown
  Found in multiple species
tgtagaaaatgacactatttg

SEQ ID NO: 970 - DNA, 21 bp, Unknown
  Found in multiple species
tgtaagagtacataatgtgag

SEQ ID NO: 971 - DNA, 21 bp, Unknown
  Found in multiple species
tggtgccatcacaaatggtct

SEQ ID NO: 972 - DNA, 21 bp, Unknown
  Found in multiple species
tggtctttcccatatcctttc

SEQ ID NO: 973 - DNA, 21 bp, Unknown
  Found in multiple species
tggtcctaggccaattgactt

SEQ ID NO: 974 - DNA, 21 bp, Unknown
  Found in multiple species
tgggtctgatgtagaaaatga

SEQ ID NO: 975 - DNA, 21 bp, Unknown
  Found in multiple species
tgggattggtctcataaaacc

SEQ ID NO: 976 - DNA, 21 bp, Unknown
  Found in multiple species
tgggagtcttgaacttgactc

SEQ ID NO: 977 - DNA, 21 bp, Unknown
  Found in multiple species
tgggaaagaccatttgtgatg

SEQ ID NO: 978 - DNA, 21 bp, Unknown
  Found in multiple species
tggcctaggaccataccttat

SEQ ID NO: 979 - DNA, 21 bp, Unknown
  Found in multiple species
tggacatactattttatggga

SEQ ID NO: 980 - DNA, 21 bp, Unknown
  Found in multiple species
tggaatgaaaagggttttgcc

SEQ ID NO: 981 - DNA, 21 bp, Unknown
  Found in multiple species
tgctggagttttaggtggtgc

SEQ ID NO: 982 - DNA, 21 bp, Unknown
  Found in multiple species
tgctaaggttcgtcagatgat

SEQ ID NO: 983 - DNA, 21 bp, Unknown
  Found in multiple species
tgcggttgctaaggttcgtca

SEQ ID NO: 984 - DNA, 21 bp, Unknown
  Found in multiple species
tgccatcacaaatggtctttc

SEQ ID NO: 985 - DNA, 21 bp, Unknown
  Found in multiple species
tgcaccacctaaaactccagc

SEQ ID NO: 986 - DNA, 21 bp, Unknown
  Found in multiple species
tgattgtattgggtctgatgt

SEQ ID NO: 987 - DNA, 21 bp, Unknown
  Found in multiple species
tgatgtagaaaatgacactat

SEQ ID NO: 988 - DNA, 21 bp, Unknown
  Found in multiple species
tgatagctttagtgatggcgg

SEQ ID NO: 989 - DNA, 21 bp, Unknown
  Found in multiple species
tgaggctctcagagctcaccc

SEQ ID NO: 990 - DNA, 21 bp, Unknown
  Found in multiple species
tgagctctgagagcctcacta

SEQ ID NO: 991 - DNA, 21 bp, Unknown
  Found in multiple species
tgacttggacatactatttta

SEQ ID NO: 992 - DNA, 21 bp, Unknown
  Found in multiple species
tgactcctgatagctttagtg

SEQ ID NO: 993 - DNA, 21 bp, Unknown
  Found in multiple species
tgaacttgactcctgatagct

SEQ ID NO: 994 - DNA, 21 bp, Unknown
  Found in multiple species
tgaaaagggttttgcccgttg

SEQ ID NO: 995 - DNA, 21 bp, Unknown
  Found in multiple species
tcttgaacttgactcctgata

SEQ ID NO: 996 - DNA, 21 bp, Unknown
  Found in multiple species
tctgtcgatactgcggttgct

SEQ ID NO: 997 - DNA, 21 bp, Unknown
  Found in multiple species
tctgatgtagaaaatgacact

SEQ ID NO: 998 - DNA, 21 bp, Unknown
  Found in multiple species
tctgacgaaccttagcaaccg

SEQ ID NO: 999 - DNA, 21 bp, Unknown
  Found in multiple species
tcggtggaagttccctaatcg

SEQ ID NO: 1000 - DNA, 21 bp, Unknown
  Found in multiple species
tcgcatgtgcattgctatata

SEQ ID NO: 1001 - DNA, 21 bp, Unknown
  Found in multiple species
tcgatactgcggttgctaagg

SEQ ID NO: 1002 - DNA, 21 bp, Unknown
  Found in multiple species
tccttcccgattagggaactt

SEQ ID NO: 1003 - DNA, 21 bp, Unknown
  Found in multiple species
tcctgatagctttagtgatgg

SEQ ID NO: 1004 - DNA, 21 bp, Unknown
  Found in multiple species
tcctaggccaattgacttgga

SEQ ID NO: 1005 - DNA, 21 bp, Unknown
  Found in multiple species
tccgccatcactaaagctatc

SEQ ID NO: 1006 - DNA, 21 bp, Unknown
  Found in multiple species
tccctcacattatgtactctt

SEQ ID NO: 1007 - DNA, 21 bp, Unknown
  Found in multiple species
tccctaatcgggaaggatgga

SEQ ID NO: 1008 - DNA, 21 bp, Unknown
  Found in multiple species
tcccgattagggaacttccac

SEQ ID NO: 1009 - DNA, 21 bp, Unknown
  Found in multiple species
tcattccatccttcccgatta

SEQ ID NO: 1010 - DNA, 21 bp, Unknown
  Found in multiple species
tcaggagtcaagttcaagact

SEQ ID NO: 1011 - DNA, 21 bp, Unknown
  Found in multiple species
tcactaaagctatcaggagtc

SEQ ID NO: 1012 - DNA, 21 bp, Unknown
  Found in multiple species
tcacaaatggtctttcccata

SEQ ID NO: 1013 - DNA, 21 bp, Unknown
  Found in multiple species
tcaattggcctaggaccatac

SEQ ID NO: 1014 - DNA, 21 bp, Unknown
  Found in multiple species
tcaatctgtcgatactgcggt

SEQ ID NO: 1015 - DNA, 21 bp, Unknown
  Found in multiple species
tcaagttatgtgatgccatac

SEQ ID NO: 1016 - DNA, 21 bp, Unknown
  Found in multiple species
tcaaggttatccctcacatta

SEQ ID NO: 1017 - DNA, 21 bp, Unknown
  Found in multiple species
tcaagactcccattacagagg

SEQ ID NO: 1018 - DNA, 21 bp, Unknown
  Found in multiple species
tatgtccaagtcaattggcct

SEQ ID NO: 1019 - DNA, 21 bp, Unknown
  Found in multiple species
tatggtcctaggccaattgac

SEQ ID NO: 1020 - DNA, 21 bp, Unknown
  Found in multiple species
tatggcatcacataacttgac

SEQ ID NO: 1021 - DNA, 21 bp, Unknown
  Found in multiple species
tatgagaccaatcccataagc

SEQ ID NO: 1022 - DNA, 21 bp, Unknown
  Found in multiple species
tatcgacagattgaaaacttc

SEQ ID NO: 1023 - DNA, 21 bp, Unknown
  Found in multiple species
tatccctcacattatgtactc

SEQ ID NO: 1024 - DNA, 21 bp, Unknown
  Found in multiple species
tatagcaatgcacatgcgagg

SEQ ID NO: 1025 - DNA, 21 bp, Unknown
  Found in multiple species
tagtgtcattttctacatcag

SEQ ID NO: 1026 - DNA, 21 bp, Unknown
  Found in multiple species
tagtatgtccaagtcaattgg

SEQ ID NO: 1027 - DNA, 21 bp, Unknown
  Found in multiple species
tagggtgagctctgagagcct

SEQ ID NO: 1028 - DNA, 21 bp, Unknown
  Found in multiple species
tagggaacttccaccgagttt

SEQ ID NO: 1029 - DNA, 21 bp, Unknown
  Found in multiple species
taggccaattgacttggacat

SEQ ID NO: 1030 - DNA, 21 bp, Unknown
  Found in multiple species
taggaccataccttattccct

SEQ ID NO: 1031 - DNA, 21 bp, Unknown
  Found in multiple species
tagctttagtgatggcggaag

SEQ ID NO: 1032 - DNA, 21 bp, Unknown
  Found in multiple species
tagcaaccgcagtatcgacag

SEQ ID NO: 1033 - DNA, 21 bp, Unknown
  Found in multiple species
tagaaaatgacactatttgta

SEQ ID NO: 1034 - DNA, 21 bp, Unknown
  Found in multiple species
tactgcggttgctaaggttcg

SEQ ID NO: 1035 - DNA, 21 bp, Unknown
  Found in multiple species
tacatcagacccaatacaatc

SEQ ID NO: 1036 - DNA, 21 bp, Unknown
  Found in multiple species
tacataatgtgagggataacc

SEQ ID NO: 1037 - DNA, 21 bp, Unknown
  Found in multiple species
taaggttcgtcagatgatctc

SEQ ID NO: 1038 - DNA, 21 bp, Unknown
  Found in multiple species
taagagtacataatgtgaggg

SEQ ID NO: 1039 - DNA, 21 bp, Unknown
  Found in multiple species
taaagctatcaggagtcaagt

SEQ ID NO: 1040 - DNA, 21 bp, Unknown
  Found in multiple species
taaactcggtggaagttccct

SEQ ID NO: 1041 - DNA, 21 bp, Unknown
  Found in multiple species
gttttcaatctgtcgatactg

SEQ ID NO: 1042 - DNA, 21 bp, Unknown
  Found in multiple species
gttttatgagaccaatcccat

SEQ ID NO: 1043 - DNA, 21 bp, Unknown
  Found in multiple species
gttttaggtggtgcaaacatt

SEQ ID NO: 1044 - DNA, 21 bp, Unknown
  Found in multiple species
gtttgcaccacctaaaactcc

SEQ ID NO: 1045 - DNA, 21 bp, Unknown
  Found in multiple species
gttgctaaggttcgtcagatg

SEQ ID NO: 1046 - DNA, 21 bp, Unknown
  Found in multiple species
gttccctaatcgggaaggatg

SEQ ID NO: 1047 - DNA, 21 bp, Unknown
  Found in multiple species
gttatccctcacattatgtac

SEQ ID NO: 1048 - DNA, 21 bp, Unknown
  Found in multiple species
gtgtcattttctacatcagac

SEQ ID NO: 1049 - DNA, 21 bp, Unknown
  Found in multiple species
gtggaatctttgaagcatggg

SEQ ID NO: 1050 - DNA, 21 bp, Unknown
  Found in multiple species
gtgccatcacaaatggtcttt

SEQ ID NO: 1051 - DNA, 21 bp, Unknown
  Found in multiple species
gtgaggctctcagagctcacc

SEQ ID NO: 1052 - DNA, 21 bp, Unknown
  Found in multiple species
gtgagctctgagagcctcact

SEQ ID NO: 1053 - DNA, 21 bp, Unknown
  Found in multiple species
gtcttgaacttgactcctgat

SEQ ID NO: 1054 - DNA, 21 bp, Unknown
  Found in multiple species
gtcgatactgcggttgctaag

SEQ ID NO: 1055 - DNA, 21 bp, Unknown
  Found in multiple species
gtcctaggccaattgacttgg

SEQ ID NO: 1056 - DNA, 21 bp, Unknown
  Found in multiple species
gtccaagtcaattggcctagg

SEQ ID NO: 1057 - DNA, 21 bp, Unknown
  Found in multiple species
gtcattttctacatcagaccc

SEQ ID NO: 1058 - DNA, 21 bp, Unknown
  Found in multiple species
gtatgtccaagtcaattggcc

SEQ ID NO: 1059 - DNA, 21 bp, Unknown
  Found in multiple species
gtatcgacagattgaaaactt

SEQ ID NO: 1060 - DNA, 21 bp, Unknown
  Found in multiple species
gtagaaaatgacactatttgt

SEQ ID NO: 1061 - DNA, 21 bp, Unknown
  Found in multiple species
gtacaaatagtgtcattttct

SEQ ID NO: 1062 - DNA, 21 bp, Unknown
  Found in multiple species
gtaagagtacataatgtgagg

SEQ ID NO: 1063 - DNA, 21 bp, Unknown
  Found in multiple species
ggttgctaaggttcgtcagat

SEQ ID NO: 1064 - DNA, 21 bp, Unknown
  Found in multiple species
ggtggaatctttgaagcatgg

SEQ ID NO: 1065 - DNA, 21 bp, Unknown
  Found in multiple species
ggtggaagttccctaatcggg

SEQ ID NO: 1066 - DNA, 21 bp, Unknown
  Found in multiple species
ggtgccatcacaaatggtctt

SEQ ID NO: 1067 - DNA, 21 bp, Unknown
  Found in multiple species
ggtcctaggccaattgacttg

SEQ ID NO: 1068 - DNA, 21 bp, Unknown
  Found in multiple species
ggtatggtcctaggccaattg

SEQ ID NO: 1069 - DNA, 21 bp, Unknown
  Found in multiple species
gggtggaatctttgaagcatg

SEQ ID NO: 1070 - DNA, 21 bp, Unknown
  Found in multiple species
gggcaaaacccttttcattcc

SEQ ID NO: 1071 - DNA, 21 bp, Unknown
  Found in multiple species
gggattggtctcataaaacct

SEQ ID NO: 1072 - DNA, 21 bp, Unknown
  Found in multiple species
gggagtcttgaacttgactcc

SEQ ID NO: 1073 - DNA, 21 bp, Unknown
  Found in multiple species
gggaataaggtatggtcctag

SEQ ID NO: 1074 - DNA, 21 bp, Unknown
  Found in multiple species
gggaaggatggaatgaaaagg

SEQ ID NO: 1075 - DNA, 21 bp, Unknown
  Found in multiple species
gggaaagaccatttgtgatgg

SEQ ID NO: 1076 - DNA, 21 bp, Unknown
  Found in multiple species
ggcctaggaccataccttatt

SEQ ID NO: 1077 - DNA, 21 bp, Unknown
  Found in multiple species
ggattggtctcataaaacctc

SEQ ID NO: 1078 - DNA, 21 bp, Unknown
  Found in multiple species
ggatggaatgaaaagggtttt

SEQ ID NO: 1079 - DNA, 21 bp, Unknown
  Found in multiple species
ggatatgggaaagaccatttg

SEQ ID NO: 1080 - DNA, 21 bp, Unknown
  Found in multiple species
ggagtcttgaacttgactcct

SEQ ID NO: 1081 - DNA, 21 bp, Unknown
  Found in multiple species
ggaataaggtatggtcctagg

SEQ ID NO: 1082 - DNA, 21 bp, Unknown
  Found in multiple species
ggaagttttcaatctgtcgat

SEQ ID NO: 1083 - DNA, 21 bp, Unknown
  Found in multiple species
ggaagttccctaatcgggaag

SEQ ID NO: 1084 - DNA, 21 bp, Unknown
  Found in multiple species
ggaaggatggaatgaaaaggg

SEQ ID NO: 1085 - DNA, 21 bp, Unknown
  Found in multiple species
ggaaagaccatttgtgatggc

SEQ ID NO: 1086 - DNA, 21 bp, Unknown
  Found in multiple species
gctaaggttcgtcagatgatc

SEQ ID NO: 1087 - DNA, 21 bp, Unknown
  Found in multiple species
gcggttgctaaggttcgtcag

SEQ ID NO: 1088 - DNA, 21 bp, Unknown
  Found in multiple species
gcctaggaccataccttattc

SEQ ID NO: 1089 - DNA, 21 bp, Unknown
  Found in multiple species
gccatcactaaagctatcagg

SEQ ID NO: 1090 - DNA, 21 bp, Unknown
  Found in multiple species
gccaattgacttggacatact

SEQ ID NO: 1091 - DNA, 21 bp, Unknown
  Found in multiple species
gcatgtgcattgctatataag

SEQ ID NO: 1092 - DNA, 21 bp, Unknown
  Found in multiple species
gcaaccgcagtatcgacagat

SEQ ID NO: 1093 - DNA, 21 bp, Unknown
  Found in multiple species
gcaaaacccttttcattccat

SEQ ID NO: 1094 - DNA, 21 bp, Unknown
  Found in multiple species
gattggtctcataaaacctct

SEQ ID NO: 1095 - DNA, 21 bp, Unknown
  Found in multiple species
gattagggaacttccaccgag

SEQ ID NO: 1096 - DNA, 21 bp, Unknown
  Found in multiple species
gatgtagaaaatgacactatt

SEQ ID NO: 1097 - DNA, 21 bp, Unknown
  Found in multiple species
gatggaatgaaaagggttttg

SEQ ID NO: 1098 - DNA, 21 bp, Unknown
  Found in multiple species
gatatgggaaagaccatttgt

SEQ ID NO: 1099 - DNA, 21 bp, Unknown
  Found in multiple species
gatactgcggttgctaaggtt

SEQ ID NO: 1100 - DNA, 21 bp, Unknown
  Found in multiple species
gagtcttgaacttgactcctg

SEQ ID NO: 1101 - DNA, 21 bp, Unknown
  Found in multiple species
gagtatggcatcacataactt

SEQ ID NO: 1102 - DNA, 21 bp, Unknown
  Found in multiple species
gaggctctcagagctcaccct

SEQ ID NO: 1103 - DNA, 21 bp, Unknown
  Found in multiple species
gagctctgagagcctcactat

SEQ ID NO: 1104 - DNA, 21 bp, Unknown
  Found in multiple species
gacttggacatactattttat

SEQ ID NO: 1105 - DNA, 21 bp, Unknown
  Found in multiple species
gactcccattacagaggtttt

SEQ ID NO: 1106 - DNA, 21 bp, Unknown
  Found in multiple species
gacgaaccttagcaaccgcag

SEQ ID NO: 1107 - DNA, 21 bp, Unknown
  Found in multiple species
gaccatttgtgatggcaccat

SEQ ID NO: 1108 - DNA, 21 bp, Unknown
  Found in multiple species
gaatgaaaagggttttgcccg

SEQ ID NO: 1109 - DNA, 21 bp, Unknown
  Found in multiple species
gaagttccctaatcgggaagg

SEQ ID NO: 1110 - DNA, 21 bp, Unknown
  Found in multiple species
gaaggatggaatgaaaagggt

SEQ ID NO: 1111 - DNA, 21 bp, Unknown
  Found in multiple species
gaacttgactcctgatagctt

SEQ ID NO: 1112 - DNA, 21 bp, Unknown
  Found in multiple species
gaaccttagcaaccgcagtat

SEQ ID NO: 1113 - DNA, 21 bp, Unknown
  Found in multiple species
gaaaagggttttgcccgttgg

SEQ ID NO: 1114 - DNA, 21 bp, Unknown
  Found in multiple species
cttttcattccatccttcccg

SEQ ID NO: 1115 - DNA, 21 bp, Unknown
  Found in multiple species
ctttcccatatcctttcatgg

SEQ ID NO: 1116 - DNA, 21 bp, Unknown
  Found in multiple species
cttggacatactattttatgg

SEQ ID NO: 1117 - DNA, 21 bp, Unknown
  Found in multiple species
cttgactcctgatagctttag

SEQ ID NO: 1118 - DNA, 21 bp, Unknown
  Found in multiple species
cttgaacttgactcctgatag

SEQ ID NO: 1119 - DNA, 21 bp, Unknown
  Found in multiple species
cttacaatgtttgcaccacct

SEQ ID NO: 1120 - DNA, 21 bp, Unknown
  Found in multiple species
ctgctggagttttaggtggtg

SEQ ID NO: 1121 - DNA, 21 bp, Unknown
  Found in multiple species
ctgatagctttagtgatggcg

SEQ ID NO: 1122 - DNA, 21 bp, Unknown
  Found in multiple species
ctcgcatgtgcattgctatat

SEQ ID NO: 1123 - DNA, 21 bp, Unknown
  Found in multiple species
ctcctgatagctttagtgatg

SEQ ID NO: 1124 - DNA, 21 bp, Unknown
  Found in multiple species
ctacatcagacccaatacaat

SEQ ID NO: 1125 - DNA, 21 bp, Unknown
  Found in multiple species
ctaatcgggaaggatggaatg

SEQ ID NO: 1126 - DNA, 21 bp, Unknown
  Found in multiple species
ctaaggttcgtcagatgatct

SEQ ID NO: 1127 - DNA, 21 bp, Unknown
  Found in multiple species
cggtggaagttccctaatcgg

SEQ ID NO: 1128 - DNA, 21 bp, Unknown
  Found in multiple species
cgcttatgggattggtctcat

SEQ ID NO: 1129 - DNA, 21 bp, Unknown
  Found in multiple species
cgatactgcggttgctaaggt

SEQ ID NO: 1130 - DNA, 21 bp, Unknown
  Found in multiple species
ccttatatagcaatgcacatg

SEQ ID NO: 1131 - DNA, 21 bp, Unknown
  Found in multiple species
cctaatcgggaaggatggaat

SEQ ID NO: 1132 - DNA, 21 bp, Unknown
  Found in multiple species
catcaaggttatccctcacat

SEQ ID NO: 1133 - DNA, 21 bp, Unknown
  Found in multiple species
cataatgtgagggataacctt

SEQ ID NO: 1134 - DNA, 21 bp, Unknown
  Found in multiple species
cataaaatagtatgtccaagt

SEQ ID NO: 1135 - DNA, 21 bp, Unknown
  Found in multiple species
cagaggttttatgagaccaat

SEQ ID NO: 1136 - DNA, 21 bp, Unknown
  Found in multiple species
cacattatgtactcttacaat

SEQ ID NO: 1137 - DNA, 21 bp, Unknown
  Found in multiple species
cacaaatggtctttcccatat

SEQ ID NO: 1138 - DNA, 21 bp, Unknown
  Found in multiple species
caattgacttggacatactat

SEQ ID NO: 1139 - DNA, 21 bp, Unknown
  Found in multiple species
caatctgtcgatactgcggtt

SEQ ID NO: 1140 - DNA, 21 bp, Unknown
  Found in multiple species
caagttatgtgatgccatact

SEQ ID NO: 1141 - DNA, 21 bp, Unknown
  Found in multiple species
caaggttatccctcacattat

SEQ ID NO: 1142 - DNA, 21 bp, Unknown
  Found in multiple species
caagactcccattacagaggt

SEQ ID NO: 1143 - DNA, 21 bp, Unknown
  Found in multiple species
caaccgcagtatcgacagatt

SEQ ID NO: 1144 - DNA, 21 bp, Unknown
  Found in multiple species
attttctacatcagacccaat

SEQ ID NO: 1145 - DNA, 21 bp, Unknown
  Found in multiple species
attggcctaggaccatacctt

SEQ ID NO: 1146 - DNA, 21 bp, Unknown
  Found in multiple species
attgattgtattgggtctgat

SEQ ID NO: 1147 - DNA, 21 bp, Unknown
  Found in multiple species
attgacttggacatactattt

SEQ ID NO: 1148 - DNA, 21 bp, Unknown
  Found in multiple species
attagggaacttccaccgagt

SEQ ID NO: 1149 - DNA, 21 bp, Unknown
  Found in multiple species
atgtttgcaccacctaaaact

SEQ ID NO: 1150 - DNA, 21 bp, Unknown
  Found in multiple species
atgtagaaaatgacactattt

SEQ ID NO: 1151 - DNA, 21 bp, Unknown
  Found in multiple species
atggtctttcccatatccttt

SEQ ID NO: 1152 - DNA, 21 bp, Unknown
  Found in multiple species
atggtcctaggccaattgact

SEQ ID NO: 1153 - DNA, 21 bp, Unknown
  Found in multiple species
atgggagtcttgaacttgact

SEQ ID NO: 1154 - DNA, 21 bp, Unknown
  Found in multiple species
atgggaaagaccatttgtgat

SEQ ID NO: 1155 - DNA, 21 bp, Unknown
  Found in multiple species
atgaaaagggttttgcccgtt

SEQ ID NO: 1156 - DNA, 21 bp, Unknown
  Found in multiple species
atccttcccgattagggaact

SEQ ID NO: 1157 - DNA, 21 bp, Unknown
  Found in multiple species
atccctcacattatgtactct

SEQ ID NO: 1158 - DNA, 21 bp, Unknown
  Found in multiple species
atcactaaagctatcaggagt

SEQ ID NO: 1159 - DNA, 21 bp, Unknown
  Found in multiple species
atcaaggttatccctcacatt

SEQ ID NO: 1160 - DNA, 21 bp, Unknown
  Found in multiple species
agttttcaatctgtcgatact

SEQ ID NO: 1161 - DNA, 21 bp, Unknown
  Found in multiple species
aggttatccctcacattatgt

SEQ ID NO: 1162 - DNA, 21 bp, Unknown
  Found in multiple species
aggtatggtcctaggccaatt

SEQ ID NO: 1163 - DNA, 21 bp, Unknown
  Found in multiple species
aggatggaatgaaaagggttt

SEQ ID NO: 1164 - DNA, 21 bp, Unknown
  Found in multiple species
aggatatgggaaagaccattt

SEQ ID NO: 1165 - DNA, 21 bp, Unknown
  Found in multiple species
aggaccataccttattccctt

SEQ ID NO: 1166 - DNA, 21 bp, Unknown
  Found in multiple species
agactcccattacagaggttt

SEQ ID NO: 1167 - DNA, 21 bp, Unknown
  Found in multiple species
actgctggagttttaggtggt

SEQ ID NO: 1168 - DNA, 21 bp, Unknown
  Found in multiple species
actgcggttgctaaggttcgt

SEQ ID NO: 1169 - DNA, 21 bp, Unknown
  Found in multiple species
acgggcaaaacccttttcatt

SEQ ID NO: 1170 - DNA, 21 bp, Unknown
  Found in multiple species
accatttgtgatggcaccatt

SEQ ID NO: 1171 - DNA, 21 bp, Unknown
  Found in multiple species
aattgacttggacatactatt

SEQ ID NO: 1172 - DNA, 21 bp, Unknown
  Found in multiple species
aatggtctttcccatatcctt

SEQ ID NO: 1173 - DNA, 21 bp, Unknown
  Found in multiple species
aaggatggaatgaaaagggtt

SEQ ID NO: 1174 - DNA, 21 bp, Unknown
  Found in multiple species
aagactcccattacagaggtt

SEQ ID NO: 1175 - DNA, 21 bp, Unknown
  Found in multiple species
aacttgactcctgatagcttt

SEQ ID NO: 1176 - DNA, 25 bp, Amaranthus palmeri
ttttattctaaagttgcttcggagg

SEQ ID NO: 1177 - DNA, 25 bp, Amaranthus palmeri
ccgtcaagaagggggcacacattgt

SEQ ID NO: 1178 - DNA, 25 bp, Amaranthus palmeri
gaatgatgtctctagtgggaaactc

SEQ ID NO: 1179 - DNA, 25 bp, Amaranthus palmeri
gattccgagatgtttaatgttgttg

SEQ ID NO: 1180 - DNA, 25 bp, Amaranthus palmeri
cggaccttaaagttccttatatagc

SEQ ID NO: 1181 - DNA, 25 bp, Amaranthus palmeri
aatgcacatgcgaggagatccgact

SEQ ID NO: 1182 - DNA, 25 bp, Amaranthus palmeri
tcaatgcaaaactctgagaacttga

SEQ ID NO: 1183 - DNA, 25 bp, Amaranthus palmeri
cctacaatgatgtttgtaagcaagt

SEQ ID NO: 1184 - DNA, 25 bp, Amaranthus palmeri
ggcttcggagttgagttctagggtc

SEQ ID NO: 1185 - DNA, 25 bp, Amaranthus palmeri
atagatgcagaattatcgggaattc

SEQ ID NO: 1186 - DNA, 25 bp, Amaranthus palmeri
ctgcttggaggatagttattgatcc

SEQ ID NO: 1187 - DNA, 25 bp, Amaranthus palmeri
cggcatcggattttctaagaatacg

SEQ ID NO: 1188 - DNA, 25 bp, Amaranthus palmeri
aatcaaaatttggaaattcttagtg

SEQ ID NO: 1189 - DNA, 25 bp, Amaranthus palmeri
gtttacaaaagatacgggaagagat

SEQ ID NO: 1190 - DNA, 25 bp, Amaranthus palmeri
agctaagaagagtttggcggtggct

SEQ ID NO: 1191 - DNA, 25 bp, Amaranthus palmeri
cattgccccttgctaattggacctt

SEQ ID NO: 1192 - DNA, 25 bp, Amaranthus palmeri
caagaaagaggtttctgggcgagat

SEQ ID NO: 1193 - DNA, 25 bp, Amaranthus palmeri
ttgtgagcgccctgtagcagctgac

SEQ ID NO: 1194 - DNA, 25 bp, Amaranthus palmeri
agggatcctgctaccattgcttcta

SEQ ID NO: 1195 - DNA, 25 bp, Amaranthus palmeri
taactgctggagttttaggtggtgc

SEQ ID NO: 1196 - DNA, 25 bp, Amaranthus palmeri
aaacattgtaagagtacataatgtt

SEQ ID NO: 1197 - DNA, 25 bp, Amaranthus palmeri
agggataaccttgatgctgtcaagt

SEQ ID NO: 1198 - DNA, 25 bp, Amaranthus palmeri
tatgtgatgccatactcggaaaaac

SEQ ID NO: 1199 - DNA, 25 bp, Amaranthus palmeri
tgattaactgcttgtttgtaccacc

SEQ ID NO: 1200 - DNA, 25 bp, Amaranthus palmeri
ttgtgaatgatgtctagtggaaggt

SEQ ID NO: 1201 - DNA, 25 bp, Amaranthus palmeri
tcgattgggattatgatgaagtccg

SEQ ID NO: 1202 - DNA, 25 bp, Amaranthus palmeri
ttagtttgctcgaaagtagagcttt

SEQ ID NO: 1203 - DNA, 25 bp, Amaranthus palmeri
agcccgtaaggctatagttcttaca

SEQ ID NO: 1204 - DNA, 25 bp, Amaranthus palmeri
gatgtagaatcattggctatttgcc

SEQ ID NO: 1205 - DNA, 25 bp, Amaranthus palmeri
tatttctgcttagaagatcatagca

SEQ ID NO: 1206 - DNA, 25 bp, Amaranthus palmeri
ttgatccagactttggattccatgt

SEQ ID NO: 1207 - DNA, 25 bp, Amaranthus palmeri
agaattatgttgcgactcatggtcg

SEQ ID NO: 1208 - DNA, 25 bp, Amaranthus palmeri
aattggccctttgaggaattgttga

SEQ ID NO: 1209 - DNA, 25 bp, Amaranthus palmeri
agttccttgagtctatggttcaatc

SEQ ID NO: 1210 - DNA, 25 bp, Amaranthus palmeri
ggctcggttcgatgaaggttggact

SEQ ID NO: 1211 - DNA, 25 bp, Amaranthus palmeri
gtgtcgagacaagatgggtcaaaga

SEQ ID NO: 1212 - DNA, 25 bp, Amaranthus palmeri
gaccaatggacaagaagtcgacctt

SEQ ID NO: 1213 - DNA, 25 bp, Amaranthus palmeri
gggcaagcttgatcgagttacttgg

SEQ ID NO: 1214 - DNA, 25 bp, Amaranthus palmeri
acaagcagataacagcaggctctgg

SEQ ID NO: 1215 - DNA, 25 bp, Amaranthus palmeri
cgaccattagacatacatatcattt

SEQ ID NO: 1216 - DNA, 25 bp, Amaranthus palmeri
gttttttgaagtcagatctgcaatc

SEQ ID NO: 1217 - DNA, 25 bp, Amaranthus palmeri
aatcagttgcaatggacaaaccata

SEQ ID NO: 1218 - DNA, 25 bp, Amaranthus palmeri
tacggttattagttgttcctgtcac

SEQ ID NO: 1219 - DNA, 25 bp, Amaranthus palmeri
tgtttgagttgaattagatcatgca

SEQ ID NO: 1220 - DNA, 25 bp, Amaranthus palmeri
aacattgtaagaatcttgatcatgt

SEQ ID NO: 1221 - DNA, 25 bp, Amaranthus palmeri
gaagctatgtgatactgtacttctc

SEQ ID NO: 1222 - DNA, 25 bp, Amaranthus palmeri
aaaatgaaatctttgatatgatgtt